(19) United States
(12) Patent Application Publication
(10) Pub. No.: US 2021/0244712 A1
(43) Pub. Date: Aug. 12, 2021

(54) METHODS FOR TREATING PATIENTS WITH CANCER HAVING DEFECTS IN CYCLIN D REGULATION

(71) Applicant: NysnoBio Ireland DAC, Dundrum (IE)

(72) Inventor: Jennifer JOHNSTON, Mill Valley, CA (US)

(21) Appl. No.: 17/049,162

(22) PCT Filed: Apr. 22, 2019
(86) PCT No.: PCT/US2019/028554

§ 371 Date: Oct. 20, 2020

Related U.S. Application Data
(60) Provisional application No. 62/660,811, filed on Apr. 20, 2018.

(51) Int. Cl. A61K 31/4196; A61P 35/00

(52) U.S. Cl. CPC A61K 31/4196; A61P 35/00

(57) ABSTRACT

The present disclosure relates to methods of treating cancer comprising administering Parkin ligase activator or a pharmaceutically acceptable salt thereof to a subject who has a mutant form of a protein in the Rb checkpoint pathway. The Parkin ligase activator includes triazole compounds, such as compounds of formula (I), and pharmaceutically acceptable salts thereof as disclosed herein. R¹, R², R³, M¹, M², M³, L¹, L², and L³ are as defined herein.

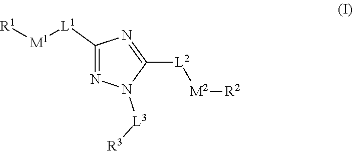

### CROSS-REFERENCE TO RELATED APPLICATION

This application claims the benefit of U.S. Provisional Application No. 62/660,811, filed on Apr. 20, 2018, the disclosures of which are hereby incorporated in their entireties for all purposes.

### FIELD OF THE INVENTION

The present invention relates to methods of treating cancer comprising administering Parkin ligase activator or a pharmaceutically acceptable salt thereof to a subject who has a defect in cyclin D regulation, such as a mutant form of a protein in the Rb checkpoint pathway.

The Parkin ligase activator includes triazole compounds and pharmaceutically acceptable salts thereof as disclosed herein.

### BACKGROUND OF THE INVENTION

The G1/S transition is a stage in the cell cycle between the G1 (GAP 1) phase, when the cell grows, and the S (Synthesis) phase, when DNA is replicated. The G1/S transition is a cell cycle check point where DNA integrity is assessed and the cell cycle can pause, for example, in response to improperly or partially replicated DNA. During this transition the cell can decide to become quiescent (enter G0), differentiate, make DNA repairs, or proliferate based on molecular signaling inputs and environmental inputs. The G1/S transition occurs late in G1 phase and the absence or improper application of this highly regulated check point can lead to cellular transformation and disease states such as cancer by deregulated state of proliferation.

The retinoblastoma tumor suppressor protein (Rb) governs this key cell-cycle checkpoint (at G1/S transition) that normally prevents G1 phase cells from entering S phase in the absence of appropriate mitogenic signals. Cancer cells frequently overcome or bypass Rb-dependent growth suppression via constitutive phosphorylation and inactivation of Rb function by cyclin-dependent kinase (CDK) 4 or CDK6 partnered with D-type cyclins (cyclin D).

During this G1/S transition, G1 cyclin D-CDK4/6 dimer complex phosphorylates Rb (phosphorylated Rb=pRb) releasing transcription factor E2F, which then drives the transition from G1 to S phase. Once the G1 cyclin D-CDK4/6 dimer complex causes pRb to release E2F by conformational change in E2F-bounded Rb upon phosphorylation, E2F drives the expression of other cyclins, such as cyclin E and cyclin A, which push the cell through the cell cycle by activating cyclin-dependent kinases, and a molecule called proliferating cell nuclear antigen, or PCNA, which speeds DNA replication and repair by helping to attach polymerase to DNA.

Thus, the Rb checkpoint pathway, including activity/expression level of cyclin D and control of the G1/S transition by Rb and/or pRb is critical for cell proliferation. Many cancers harbor mutations or other defects in the proteins present in or controlling this Rb checkpoint pathway that controls progression of the cell cycle. Thus, there is a need for treatment of cancer which is effective for treating subjects who have abnormally high levels in the Rb checkpoint pathway or harbor mutations in the proteins present in or controlling the Rb checkpoint pathway.

### SUMMARY OF THE INVENTION

Among other things, the compounds of the present disclosure can modulate or activate Parkin ligase and may be useful in treating various diseases and conditions as disclosed herein, including cancer. In one embodiment, the present disclosure provides a method of treating cancer in a subject in need thereof, comprising administering to the subject a Parkin ligase activator or a pharmaceutically acceptable salt thereof, wherein the subject has a mutant form of a protein in the Rb checkpoint pathway or p53.

In one embodiment, the present disclosure provides a method of inhibiting or reducing abnormal (e.g., overexpressed) wild-type or mutated cyclin D1 activity in a subject in need thereof, comprising administering a Parkin ligase activator or a pharmaceutically acceptable salt thereof.

In one embodiment, the present disclosure provides a method of inhibiting or reducing abnormal (e.g., overexpressed) wild-type or mutated cyclin D1 activity or expression in human cells, comprising contacting a Parkin ligase activator or a pharmaceutically acceptable salt thereof with the human cells.

In one embodiment, the present disclosure provides a method of inducing cell cycle arrest or senescence in a subject in need thereof, comprising administering a Parkin ligase activator or a pharmaceutically acceptable salt thereof.

In one embodiment of any of the methods disclosed herein, the subject has cancer. In one embodiment of any of the methods disclosed herein, the cancer is selected from any cancer disclosed herein. In one embodiment, the cancer is lymphoma, colon cancer, lung cancer, or ovarian cancer.

In one embodiment of any of the methods disclosed herein, the subject harbors mutated protein in the Rb checkpoint pathway or p53. In another embodiment of any of the methods disclosed herein, human cells harbor mutated protein in the Rb checkpoint pathway or p53. In one embodiment of any of the methods disclosed herein, the mutated protein is selected from one or more of the group consisting of CDK4, CDK6, E2F, Rb, pRb, cyclin D, cyclin D1, cyclin E, cyclin E1, Ki67, INK4, p53, p21, p27, p14, p15, and p16. In one embodiment, the mutated protein is selected from one or more of the group consisting of Rb, pRb, cyclin D1 and cyclin E. In some embodiments, the mutated protein is cyclin D1.

In one embodiment of any of the methods disclosed herein, the subject expresses inappropriate levels of a protein in the Rb checkpoint pathway or p53. In another embodiment of any of the methods disclosed herein, human cells express inappropriate levels of a protein in the Rb checkpoint pathway or p53. In one embodiment of any of the methods disclosed herein, the inappropriately expressed protein is selected from one or more of the group consisting of CDK4, CDK6, E2F, Rb, pRb, cyclin D, cyclin D1, cyclin E, cyclin E1, Ki67, INK4, p53, p21, p27, p14, p15, and p16. In one embodiment, the inappropriately expressed protein is selected from one or more of the group consisting of Rb, pRb, cyclin D1 and cyclin E. In some embodiments, the inappropriately expressed protein is cyclin D1.

In another embodiment, the subject has a loss of p16 or diminished activity relative to wildtype p16. In a specific embodiment, the loss or diminished activity is due to a mutation of p16. In another embodiment, the mutation is a point mutation. In another embodiment, the loss of activity is due to deletion. In another embodiment, the deletion may be either homozygous or heterozygous.

In one embodiment of any of the methods disclosed herein, the mutated protein is from a point mutation.

In another embodiment, mutated protein is cyclin D1. In another embodiment, cyclin D1 has at least one point mutation. In one embodiment, the mutated cyclin D1 comprises at least one point mutation on R260H, or T286I.

In another embodiment, the subject harbors a mutation in an upstream signaling pathway that leads to increased expression cyclin D. In some embodiments, the signaling pathways that increase expression by increasing transcription of cyclin D1 include MAP kinases, phosphoinositide 3-kinase/protein kinase B (PI3K/Akt) signaling, IKK/IκB/NF-κB pathway, Wnt/β-catenin signaling, STAT signaling, and nuclear hormone receptors. Consistent with these, the cyclin D1 promoter has consensus sequences for Ets, Fos/Jun, NF-κB, STAT, TCF, E2F, Sp1, EGR, and ERα.

In one embodiment, mutated protein is p21. In one embodiment, the mutated p21 comprises at least one point mutation on H83Y, D84Y, D84V, R19H, or R67L.

In one embodiment, mutated protein is Rb.

In one embodiment, mutated protein is p53. In one embodiment, the mutated p53 comprises at least one point mutation on R175H, R43H, M237I, R273H, C176W, R280K, L52R, L145R, R248W, and/or L130V. In one embodiment, the mutated p53 comprises mutations as described in Muller et al. Nature Cell Biology 2013 January; 15 (1):2-8, which is incorporated by reference herein in its entirety.

In one embodiment of any of the methods disclosed herein, the point mutation is on only one allele. In another embodiment, the point mutation is on two alleles.

In one embodiment of any of the methods disclosed herein, the mutation provides overexpression, amplification, transcriptional silencing or deletion of one or more protein coding genes of the Rb checkpoint pathway or p53. In one embodiment of any of the methods disclosed herein, the mutated protein promotes gene amplification and/or gene overexpression.

In one embodiment, the one or more protein coding genes is selected from a protein coding gene of CDK4, CDK6, E2F, Rb, pRb, cyclin D, cyclin D1, cyclin E, cyclin E1, Ki67, INK4, p53, p21, p27, p14, p15, and/or p16. In one embodiment, the mutation is on a protein coding gene selected from Rb, cyclin D1, p53, p16, p 15 and/or p21. In one embodiment, the gene is selected from CCND1, CDKN2A, CDKN2B, CDKN1A, RB, and/or TP53. In one embodiment, the mutated protein promotes gene amplification and/or overexpression of in cyclin D or cyclin E genes. In one embodiment, the mutation is on a protein, wherein the mutation provides overexpression of cyclin D1 gene.

In one embodiment of any of the methods disclosed herein, the mutant form of the protein is provided by chromosome translocation of one or more protein coding genes of the Rb checkpoint pathway or p53. In one embodiment, the one or more protein coding genes is selected from a protein coding gene of CDK4, CDK6, E2F, Rb, pRb, cyclin D, cyclin D1, cyclin E, cyclin E1, Ki67, INK4, p53, p21, p27, p14, p15, and/or p16. In one embodiment the chromosomal translocations results in amplification of said protein.

In one embodiment of any of the methods disclosed herein, the mutant form of the protein has a copy number variation (CNV) greater than 2. In one embodiment, the mutant form ofthe protein comprises CNV3, CNV4, CNV5, CNV6, CNV7, CNV8, CNV9, or CNV10.

In one embodiment of any of the methods disclosed herein, the subject is human.

In one embodiment of any of the methods disclosed herein, the Parkin ligase activator is a triazole compound. In one embodiment, the triazole compound is a compound of formula (I):

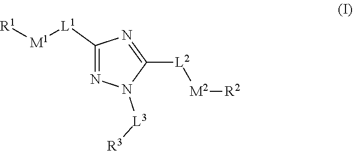

or a pharmaceutically acceptable salt or solvate thereof, wherein:

L¹, L² and L³ are each independently selected from a bond, alkylene, or alkenylene;

M¹ and M² are each independently selected from —NR⁴—, —NR⁴C(O)—, —N(C(O)R¹)—, —C(O)NR⁴—, —NR⁴C(O)NR⁴—, —C(O)—, —C(═NR⁴)—, —C(═NOR⁴)—, —OC(O)—, —C(O)O—, —OC(O)O—, —OC(O)NR⁴—, —NR4C(O)O—, —S(O)ₘ—, —S(O)ₘNR⁴—, or —NR⁴S(O)ₘ—, provided that M¹ and M² are not both —NR⁴—;

R¹ and R² are each independently selected from an alkyl, alkenyl, cycloalkyl, aryl, biphenyl, heterocyclyl heterocycloalkyl, heteroaryl, cycloalkylalkyl, arylalkyl, arylalkenyl, arylalkynyl, heterocyclylalkyl, heteroarylalkyl, heteroarylalkenyl, or heteroarylalkynyl, wherein each cycloalkyl, aryl, heteroaryl, and heterocyclyl portion is optionally substituted with one or more R⁵;

R³ is selected from an alkyl, alkenyl, cycloalkyl, aryl, biphenyl, heterocyclyl heterocycloalkyl, heteroaryl, cycloalkylalkyl, arylalkyl, arylalkenyl, arylalkynyl, heterocyclylalkyl, heteroarylalkyl, heteroarylalkenyl, or heteroarylalkynyl, wherein each cycloalkyl, aryl, heteroaryl, and heterocyclyl portion is optionally substituted with one or more R⁷;

R⁴ is each independently H, alkyl, wherein each alkyl is optionally substituted with one or more R⁵;

R⁵ is each independently I, Br, Cl, F, —CH₂F, —CHF₂, —CF₃, —OCF₃, —CN, -alkyl-CN, —CONH₂, —CONHR⁶, —CONR⁶R⁶, —COOH, —NIB, —NHR⁶, —NO₂, —NR⁶R⁶, —N₃, —OH, OR⁶, —COOR⁶, —OSO₃R⁶, oxo, R⁶, —SH, —SO₂R⁶, —SO₃H, —SO₃R⁶, or —SR⁶;

R⁶ is each independently alkyl, alkenyl, alkynyl, haloalkyl, haloalkenyl, haloalkynyl; or alternatively two R⁶ on the same N atom can together form a 3-6 membered N-heterocyclyl;

R⁷ is each independently I, Br, Cl, F, —CH₂F, —CHF₂, —CF₃, —OCF₃, —CN, -alkyl-CN, —CONH₂, —CONHR⁶, —CONR⁶R⁶, —COOH, —NH₂, —NHR⁶, —NO₂, —NR⁶R⁶, —N₃, —OH, OR⁶, —COOR⁶, —OSO₃R⁶, oxo, R⁶, —SH, —SO₂R⁶, —SO₃H, —SO₃R⁶, —SR⁶, cycloalkyl, heterocyclyl, aryl, or heteroaryl, wherein each cycloalkyl, heterocyclyl, aryl, and heteroaryl is optionally substituted with one or more R⁵; and

m is 0, 1, or 2.

In one embodiment of any of the methods disclosed herein, the Parkin ligase activator is a triazole compound of formula (I), (I′), (IA′), (IA), (IB), (IC), (ID), (IE), (IF), (IG), (II), (II′). (IIA), (IIB), (III), (IIIA), (IIIB), (IIIC), (IIID), and/or (IIIE) as disclosed herein.

In one embodiment of any of the methods disclosed herein, the Parkin ligase activator is selected from one or more from Table 1, Table IA, Table 2, Table 3, Table 3A, Table 3B, and/or Table 3C. In one embodiment of any of the methods disclosed herein, the Parkin ligase activator is

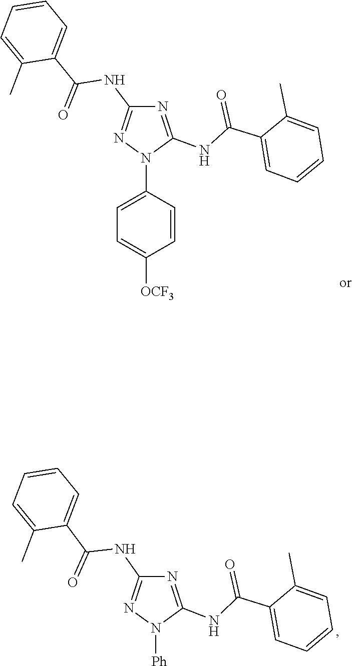 [SMILES: Cc1ccccc1C(=O)Nc1nc(NC(=O)c2ccccc2C)n(-c2ccc(OC(F)(F)F)cc2)n1 ; Cc1ccccc1C(=O)Nc1nc(NC(=O)c2ccccc2C)n(-c2ccccc2)n1]

or a pharmaceutically acceptable salt thereof.

### BRIEF DESCRIPTION OF THE FIGURES

FIG. 1A shows senescence quantification in Compound 42 and palbociclib treated cell lines A2780.

FIG. 1B shows senescence quantification in Compound 42 and palbociclib treated cell line OAW42.

FIG. 1C shows senescence quantification in Compound 42 and palbociclib treated cell line ES-2.

FIG. 2 shows the effect of Compound 42 and palbociclib on cellular biomarker pRb in cell lines A2780, SW626, OAW42, and ES-2.

FIG. 3 shows the effect of Compound 42 and palbociclib on cellular biomarker Rb in cell lines A2780, SW626, OAW42, and ES-2.

FIG. 4 shows the effect of Compound 42 and palbociclib on cellular biomarker cyclin D1 in cell lines A2780, SW626, OAW42, and ES-2.

FIG. 5 shows the effect of Compound 42 and palbociclib on cellular biomarker cyclin E1 in cell lines A2780, SW626, OAW42, and ES-2.

FIG. 6A shows the effect of Compound 42 and palbociclib on cellular biomarker pRb in cell line JEKO-1.

FIG. 6B shows the effect of Compound 42 and palbociclib on cellular biomarker cyclin E in cell line JEKO-1.

FIG. 6C shows the effect of Compound 42 and palbociclib on cellular biomarker cyclin D in cell line JEKO-1.

FIG. 7A shows senescence quantification in Compound 42 and palbociclib treated cell line OAW28.

FIG. 7B shows the effect of Compound 42 and palbociclib on cellular biomarker pRb in cell line OAW28.

FIG. 7C shows the effect of Compound 42 and palbociclib on cellular biomarker Rb in cell line OAW28.

FIG. 7D shows the effect of Compound 42 and palbociclib on cellular biomarker cyclin D in cell line OAW28.

FIG. 8A shows senescence quantification in Compound 42 and palbociclib treated cell line HTC116.

FIG. 8B shows the effect of Compound 42 and palbociclib on cellular biomarker pRb in cell line HTC116.

FIG. 8C shows the effect of Compound 42 and palbociclib on cellular biomarker Rb in cell line HTC116.

FIG. 8D shows the effect of Compound 42 and palbociclib on cellular biomarker cyclin D (i) and cyclin D1 (ii) in cell line HTC116.

FIG. 8E shows the effect of Compounds 5x, 6x, 7x, 10x, 11x, 13x, and 14x on cellular biomarker cyclin D1 in HTC-116 cell line.

FIG. 8F shows the effect of Compounds 5x, 6x, 7x, 10x, 11x, 13x, and 14x on cellular biomarker cyclin D1 in TOV-112D cell line.

FIG. 8G shows the effect of Compounds 5x, 6x, 7x, 10x, 11x, 13x, and 14x on cellular biomarker PRK8 in HTC-116 cell line.

FIG. 8H shows the effect of Compounds 5x, 6x, 7x, 10x, 11x, 13x, and 14x on cellular biomarker PRK8 in TOV-112D cell line.

FIG. 8I shows western blot analysis of Cyclin D1 on HCT-116 and TOV-112D cell lines following 24 hour treatment with Compounds 5x, 6x, 7x, 10x, 11x, 13x, and 14x.

FIG. 8J shows the effect of Compounds F, S3, 42, and 146 on cellular biomarker cyclin D1 in HTC-116 cell line.

FIG. 8K shows the effect of Compounds F, S3, 42, and 146 on cellular biomarker cyclin D1 in TOV-112 cell line.

FIG. 8L shows the effect of Compounds F, S3, 42, and 146 on cellular biomarker cyclin D1 in Mino cells.

FIG. 8M shows western blot analysis of Cyclin D1 on Mino cells following 24 hour treatment with F, S3, 42, and 146 on cellular biomarker cyclin D1 in HTC-116 cell line.

FIG. 9A shows change in average tumor volume in HCT116 mouse xenograft model after treatment with Compound 42, Compound F, Compound Q3, or palbociclib.

FIG. 9B shows harvested xenograft treated with Compound 42, Compound F, or palbociclib.

FIG. 9C shows change in average tumor volume in HCT116 mouse xenograft model after treatment with Compound 42, Compound F, Compound Q3, or control

FIG. 9D is a graph of animal body weight after treatment with 1 mg/kg of Compound 42, 5 mg/kg of Compound 42, or 25 mg/kg of Compound F in HCT116 mouse xenograft model.

FIG. 9E shows change in average tumor volume in HCT116 mouse xenograft model with treatment of 10 mg/kg or 25 mg/kg of Compound F.

FIG. 9F is a graph of animal body weight after treatment with 10 mg/kg or 25 mg/kg of Compound F in HCT116 mouse xenograft model.

FIG. 10A western blot shows that compound 42 alone decreases cyclin D levels via proteasomal degradation, and this proteasomal degradation is counterbalanced by adding epoxomicin, which inhibits the proteasome.

FIG. 10B western blot shows the effect of the epoxomicin inhibiting the proteasome, as evidenced by the overall increase of ubiquitinated proteins (using FK-2 antibody on the blot) in the lanes with epoxomicin.

FIG. 11A shows cellular viability of Compound 7x in HCT-116 cells.

FIG. 11B shows % cell senescence by Compound 7x treated in HCT-116 cells.

FIG. 12A shows cellular viability of Compound 10x in HCT-116 cells.

FIG. 12B shows % cell senescence by Compound 10x treated in HCT-116 cells.

FIG. 13A shows cellular viability of Compound S3 in HCT-116 cells.

FIG. 13B shows % cell senescence by Compound S3 treated in HCT-116 cells.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value or within standard deviation appropriate for the instrument or method used to obtain said numerical values and/or ranges.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “a kinase inhibitor” refers to one or more kinase inhibitors or at least one kinase inhibitor. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The term “treating” means one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “mutation” refers to a change in the gene of a protein that leads to substituted, inserted, or deleted amino acids in the amino acid sequence of a protein, one or more base pair insertions, deletion, or substitutions, gene amplification, gene promoter or enhancer modifications, changes to the 3′ or 5′ untranslated regions of the mRNA, chromosome translocations, or any other changes, including epigenetic changes, that result in altered expression of a protein, wherein altered expression includes increasing activity of the protein, reducing activity of a protein, altering the activity of the protein, or eliminating activity of the protein. For example, mutated or abnormal cyclin D1 activity can be associated to a mutation in the cyclin D1 protein, a mutation in the regulatory sequences controlling expression of Cyclin D1, a mutation in a protein that regulates cyclin D1 activity or a mutation in a gene or protein that alters the expression and/or activity of cyclin D1.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

As used herein, the “alignment” of two or more protein/amino acid sequences may be performed using the alignment program ClustalW2, available at www.ebi.ac.uk/Tools/msa/clustalw2/. The following default parameters may be used for Pairwise alignment: Protein Weight Matrix=Gonnet; Gap Open=10; Gap Extension=0.1.

As used herein, the term “Rb checkpoint pathway” refers to a cellular pathway in which Rb function is regulated primarily by its phosphorylation state at the G1/S transition. For example, Rb checkpoint pathway includes participation by CDK2, CDK4, CDK6, cyclin D, cyclin E, cyclin D-CDK 4/6 complex, p16, p14, p15, p21, and/or E2F transcription factors.

As used herein, the term “protein in the Rb checkpoint pathway” includes endogenous proteins that regulate Rb and/or pRb activity as a checkpoint in the G1/S transition or that directly bind to Rb and/or pRb. Non-limiting example of the protein in the Rb checkpoint pathway include proteins such as cyclin D1, CDK4, CDK6, pRb, Rb, p16, p14, p15, p21, E2F, E2F1, CDK2, and Cyclin E.

“Ubiquitin Proteasome Pathway System (UPS)” as used herein relates to the ubiquitin proteasome pathway, conserved from yeast to mammals, and is required for the targeted degradation of most short-lived proteins in the eukaryotic cell. Targets include cell cycle regulatory proteins, whose timely destruction is vital for controlled cell division, as well as proteins unable to fold properly within the endoplasmic reticulum. Ubiquitin modification is an ATP-dependent process carried out by three classes of enzymes. An “ubiquitin activating enzyme” (E1) forms athio-esterbond with ubiquitin, a highly conserved 76-amino acid protein. This reaction allows subsequent binding of ubiquitin to a “ubiquitin conjugating enzyme” (E2), followed by the formation of an isopeptide bond between the carboxy-terminus of ubiquitin and a lysine residue on the substrate protein. The latter reaction requires a “ubiquitin ligase” (E3). E3 ligases can be single- or multi-subunit enzymes. In some cases, the ubiquitin-binding and substrate binding domains reside on separate polypeptides brought together by adaptor proteins or culling. Numerous E3 ligases provide specificity in that each can modify only a subset of substrate proteins. Further specificity is achieved by post-translational modification of substrate proteins, including, but not limited to, phosphorylation. Effects of monoubiquitination include changes in subcellular localization. However, multiple ubiquitination cycles resulting in a polyubiquitin chain are required for targeting a protein to the proteasome for degradation. The multisubunit 26S proteasome recognizes, unfolds, and degrades polyubiquitinated substrates into small peptides. The reaction occurs within the cylindrical core of the proteasome complex, and peptide bond hydrolysis employs a core threonine residue as the catalytic nucleophile. It has been shown that an additional layer of complexity, in the form of multiubiquitin chain receptors, may lie between the polyubiquitination and degradation steps. These receptors react with a subset of polyubiquitinated substrates, aiding in their recognition by the 26S proteasome, and thereby promoting their degradation. This pathway is not only important in cellular homeostasis, but also in human disease. Because ubiquitin/proteasome-dependent degradation is often employed in control of the cell division cycle and cell growth, researchers have found that proteasome inhibitors hold some promise of being developed into potential cancer therapeutic agents.

Protein degradation through the ubiquitin-proteasome system is the major pathway of non-lysosomal proteolysis of intracellular proteins. It plays important roles in a variety of fundamental cellular processes such as regulation of cell cycle progression, division, development and differentiation, apoptosis, cell trafficking, and modulation of the immune and inflammatory responses. The central element of this system is the covalent linkage of ubiquitin to targeted proteins, which are then recognized by the 26S proteasome, an adenosine triphosphate-dependent, multi-catalytic protease. Damaged, oxidized, ormisfolded proteins as well as regulatory proteins that control many critical cellular functions are among the targets of this degradation process. Aberration of this system leads to the dysregulation of cellular homeostasis and the development of multiple diseases (Wang et al. Cell Mol Immunol. 2006 August; 3(4):255-61).

“Parkin ligase” or “Parkin” as used herein relates to a protein which in humans is encoded by the PARK2 gene. (Kitada T, Asakawa S, Hattori N, Matsumine H, Yamamura Y, Minoshima S, Yokochi M, Mizuno Y, Shimizu N (April 1998). “Mutations in the parkin gene cause autosomal recessive juvenile parkinsonism”. Nature 392 (6676): 605-608. doi: 10.1038/33416. PMID 9560156. Matsumine H, Yamamura Y, Hattori N, Kobayashi T, Kitada T, Yoritaka A, Mizuno Y (April 1998). “A microdeletion of D6S305 in a family of autosomal recessive juvenile parkinsonism (PARK2)”. Genomics 49 (1): 143-146. doi: 10.1006/geno. 1997.5196. PMID 9570960. The protein is a component of a multiprotein E3 ubiquitin ligase complex which in turn is part of the ubiquitin-proteasome system that mediates the targeting of proteins for degradation. Mutations in the PARK2 gene are known to cause a familial form of Parkinson's disease known as autosomal recessive juvenile Parkinson's disease (AR-JP).

“Ligase” as used herein, is an enzyme that can catalyze the joining of two or more compounds or biomolecules by bonding them together with a new chemical bond. The “ligation” of the two usually with accompanying hydrolysis of a small chemical group dependent to one of the larger compounds or biomolecules, or the enzyme catalyzing the linking together of two compounds, e.g., enzymes that catalyze joining of groups C—O, C—S, C—N, etc. Ubiquitin-protein (E3) ligases are a large family of highly diverse enzymes selecting proteins for ubiquitination.

“Ub Ligases” are involved in disease pathogenesis for oncology, inflammation & infectious disease. E3 ligase belonging to the RING-between-RING (RBR) family of E3 ligases containing both canonical RING domains and a catalytic cysteine residue usually restricted to HECT E3 ligases; termed ‘RING/HECT hybrid’ enzymes. Mutations in Parkin linked to Parkinson's disease, cancer and mycobacterial infection. Parkin is recognized as a neuroprotective protein with a role in mitochondrial integrity. Human genetic data implicate loss of Parkin activity as a mechanism for pathogenesis of Parkinson's disease (PD).

“Zinc Finger (ZnF) Domain” as used herein relates to a protein structure characterized by coordinating zinc ions to stabilize the functional activity. ZnF stabilize the binding of Ub, Deubiquitinating Enzymes (DUBs), and Ligases (E3) in the UPS.

“Ligands” as used herein bind to metal via one or more atoms in the ligand, and are often termed as chelating ligands. A ligand that binds through two sites is classified as bidentate, and three sites as tridentate. The “bite angle” refers to the angle between the two bonds of a bidentate chelate. Chelating ligands are commonly formed by linking donor groups via organic linkers. A classic bidentate ligand is ethylenediamine, which is derived by the linking of two ammonia groups with an ethylene (—CH2CH2-) linker. A classic example of a polydentate ligand is the hexadentate chelating agent EDTA, which is able to bond through six sites, completely surrounding some metals. The binding affinity of a chelating system depends on the chelating angle or bite angle. Many ligands are capable of binding metal ions through multiple sites, usually because the ligands have lone pairs on more than one atom. Some ligands can bond to a metal center through the same atom but with a different number of lone pairs. The bond order of the metal ligand bond can be in part distinguished through the metal ligand bond angle (M-X—R). This bond angle is often referred to as being linear or bent with further discussion concerning the degree to which the angle is bent. For example, an imido ligand in the ionic form has three lone pairs. One lone pair is used as a sigma X donor, the other two lone pairs are available as L type pi donors. If both lone pairs are used in pi bonds then the M-N—R geometry is linear. However, if one or both of these lone pairs are non-bonding then the M-N—R bond is bent and the extent of the bend speaks to how much pi bonding there may be. It was found that few heteroatoms, such as nitrogen, oxygen, and sulfur atoms, interacted with zinc, ideal distances between the zinc and these heteroatoms were identified. Whereas carboxylates bound to the zinc via both monodentate and bidentate interactions, the hydroxamates bound dominantly in a bidentate manner. These results aid in the design of new inhibitors with the potential to interact with zinc in the target protein. Virtually every molecule and every ion can serve as a ligand for (or “coordinate to”) metals. Monodentate ligands include virtually all anions and all simple Lewis bases. Thus, the halides and pseudohalides are important anionic ligands whereas ammonia, carbon monoxide, and water are particularly common charge-neutral ligands. Simple organic species are also very common, be they anionic (RO⁻ and RCO₂⁻) or neutral (R₂O, R₂S, RB—XNHX, and R₃P). Complexes of polydentate ligands are called chelate complexes. They tend to be more stable than complexes derived from monodentate ligands. This enhanced stability, the chelate effect, is usually attributed to effects of entropy, which favors the displacement of many ligands by one polydentate ligand. When the chelating ligand forms a large ring that at least partially surrounds the central atom and bonds to it, leaving the central atom at the center of a large ring. The more rigid and the higher its denticity, the more inert will be the macrocyclic complex.

“Chelator” as used herein relates to a binding agent that suppresses chemical activity by forming a chelate (a coordination compound in which a metal atom or ion is bound to a ligand at two or more points on the ligand, so as to form, for example, a heterocyclic ring containing a metal atom).

“Chelation” as used herein relates to a particular way that ions and molecules bind metal ions. According to the International Union of Pure and Applied Chemistry (IUPAC), chelation involves the formation or presence of two or more separate coordinate bonds between a polydentate (multiple bonded) ligand and a single central atom. Usually these ligands are organic compounds, and are called chelants, chelators, chelating agents, or sequestering agents.

“Electrophile” as used herein relates to species that is attracted to an electron rich center. In chemistry, an electrophile is a reagent attracted to electrons. It participates in a chemical reaction by accepting an electron pair in order to bond to a nucleophile. Because electrophiles accept electrons, they are Uewis acids. Most electrophiles are positively charged, have an atom that carries a partial positive charge, or have an atom that does not have an octet of electrons.

The terms below, as used herein, have the following meanings, unless indicated otherwise:

“Amino” refers to the —NH₂ radical.

“Cyano” refers to the —CN radical.

“Halo” or “halogen” refers to bromo, chloro, fluoro or iodo radical.

“Hydroxy” or “hydroxyl” refers to the —OH radical.

“Imino” refers to the ═NH substituent.

“Nitro” refers to the —NO₂ radical.

“Oxo” refers to the ═O substituent.

“Thioxo” refers to the ═S substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, i-butyl, n-pentyl, i-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes G, alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alknyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “Cw-Cz acyl” where w and z depicts the range of the number of carbon in Rₐ, as defined above. For example, “C1-C₁₀ acyl” refers to alkylcarbonyl group as defined above, where Rₐ is C₁-C₁₀ alkyl, C₁-C₁₀ alkenyl, or C₁-C₁₀ alkynyl radical as defined above. Unless stated otherwise specifically in the specification, an alkyl carbonyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” or “arylalkyl” refers to a radical of the formula —R_{b}—R_{c} where R_{b} is an alkylene group as defined above and R_{c} is one or more aryl radicals as defined above, for example, benzyl, diphenylmethyl and the like. Unless stated otherwise specifically in the specification, an aralkyl group can be optionally substituted.

“Aralkenyl” or “arylalkenyl” refers to a radical of the formula —R_{b}—R_{c} where R_{b} is an alkenylene o group as defined above and R_{c} is one or more aryl radicals as defined above. Unless stated otherwise specifically in the specification, an aralkenyl group can be optionally substituted.

“Aralkynyl” or “arylalkynyl” refers to a radical of the formula —R_{b}—R_{c} where R_{b} is an alkynylene group as defined above and R_{c} is one or more aryl radicals as defined above. Unless stated otherwise specifically in the specification, an aralkynyl group can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include aryls and cycloalkyl, cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbomyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkenyl group can be optionally substituted.

“Heterocyclyl,” “heterocyclic ring” or “heterocycle” refers to a stable 3- to 20-membered non-aromatic, partially aromatic, or aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heterocyclylalkyl” refers to a radical of the formula —R_{b}—Rₑ where R_{b} is an alkylene group as defined above and Rₑ is a heterocyclyl radical as defined above. Unless stated otherwise specifically in the specification, a heterocycloalkylalkyl group can be optionally substituted.

“Heterocyclylalkenyl” refers to a radical of the formula —R_{b}—Rₑ where R_{b} is an alkenylene group as defined above and Rₑ is a heterocyclyl radical as defined above. Unless stated otherwise specifically in the specification, a heterocycloalkylalkenyl group can be optionally substituted.

“Heterocyclylalkynyl” refers to a radical of the formula —R_{b}—Rₑ where R_{b} is an alkynylene group as defined above and Rₑ is a heterocyclyl radical as defined above. Unless stated otherwise specifically in the specification, a heterocycloalkylalkynyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Heteroarylalkenyl” refers to a radical of the formula —R_{b}—R_{f} where R_{b} is an alkenylene, chain as defined above and R_{f} is a heteroaryl radical as defined above. Unless stated otherwise specifically in the specification, a heteroarylalkenyl group can be optionally substituted.

“Heteroarylalkynyl” refers to a radical of the formula —R_{b}—R_{f} where R_{b} is an alkynylene chain as defined above and R_{f} is a heteroaryl radical as defined above. Unless stated otherwise specifically in the specification, a heteroarylalkynyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced

with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g} Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

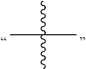

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

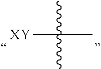

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

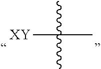

infers that when R³ is “XY”, the point of attachment bond is the same bond as the bond by which R³ is depicted as being bonded to CH₃.

The following description includes information that may be useful in understanding the present invention. It is not an admission that any of the information provided herein is prior art or relevant to the presently claimed inventions, or that any publication specifically or implicitly referenced is prior art.

### Parkin Ligase Activator Compounds of the Present Disclosure

A dysfunction of the Parkin ligase, such as PARK2 (an E3 ubiquitin ligase that targets proteins for destruction), has been associated with the advancement of Parkinsonism and human malignancies and its role in cancer progression (see Nat. Genet. 2010, 42, 77; Nat. Genet. 2000, 25, 302). In some cancer and in hereditary Parkinson's disease, PARK2 gene is altered by deletion, overexpression and/or mutation rendering the Parkin ligase inactive or dysfunctional (see PNAS 2010, 107, 15145). PARK2 has been demonstrated to target degradation of cyclin D and cyclin E, which are both key regulators for the G1/S transition of the cell cycle and Rb checkpoint pathway (see Gong, Y. et at. Nat Genet. 2014, 46(6), 588-594, which is hereby incorporated by reference, herein in its entirety). Degradation of cyclin D would result in deprivation of the G1 cyclin D-CDK4/6 dimer complex which could minimize phosphorylation of Rb and minimize release of E2F causing in cell cycle arrest and senescence. Dysregulation of Rb and cyclin D have been shown to play critical roles in different human cancers (see Sherr et al. Cancer Cell 2002, 2, 103). Thus, Parkin ligase activators can be useful in treating cancer in a subject who has a mutation in a protein in the Rb checkpoint pathway by regulating the G1/S transition of the cell cycle.

Parkin ligase modulators of the present disclosure and CDK 4/6 inhibitors can regulate cyclin D levels through distinct mechanism. While CDK 4/6 inhibitors prevent generation of G1 cyclin D-CDK4/6 complex, inducing senescence, Parkin ligase modulators degrade cyclin D, thereby inhibiting the formation of the G1 cyclin D-CDK4/6 complexes, which also leads to cell cycle arrest and senescence. In one embodiment, Parkin regulates cyclin D levels through proteasomal degradation (Example 7).

The compound of the present disclosure can be useful for modulating Parkin ligase. In a specific embodiment, the Parkin ligase is wild type, or still has its function resulting in the degradation of cyclin D. Further, the compound of the present disclosure can be useful for treating various diseases and conditions including, but not limited to, cancer, neurological disease, a disorder characterized by abnormal accumulation of α-synuclein, a disorder of an aging process, cardiovascular disease, bacterial infection, viral infection, mitochondrial related disease, mental retardation, deafness, blindness, diabetes, obesity, autoimmune disease, glaucoma, Leber's Hereditary Optic Neuropathy, and rheumatoid arthritis.

In one embodiment, the present disclosure provides a Parkin ligase activator is a triazole, pyradazinone, or pyrazolopyrimidine derivative, or a pharmaceutically acceptable salt thereof. In one embodiment, the Parkin ligase activator is selected from disclosures of International Patent Application Nos. WO 2016/090371, WO 2017/210694, WO 2017/210685, and/or WO 2017/210678, which are hereby incorporated by reference in their entireties.

In one embodiment, the present disclosure provides a Parkin ligase activator having the structure of formula (X):

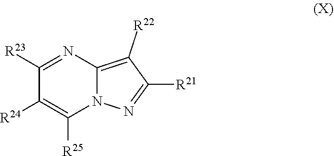

or a pharmaceutically acceptable salt or solvate thereof, wherein:

R²¹, R²², R²³, and R²⁴ are each independently selected from H, halogen, CN, C₁-C₆ alkyl, C₁-C₆ haloalkyl, C₃-C₈ cycloalkyl, C₃-C₈ cycloalkyl(C₁-C₃ alkyl), C₆-C₁₂ aryl, C₆-C₁₂ aryl(C₁-C₃ alkyl), 3-8 membered heterocyclyl, 3-8 membered heterocyclyl(C₁-C₃ alkyl), 5-6 membered heteroaryl, 5-6 membered heteroaryl(C₁-C₃ alkyl), —SH, —S—(C₁-C₆ alkyl), —OH, —O—(C₁-C₆ alkyl), —NH₂, —NHR⁴, —NR⁴R⁴, —NHC(O)R⁴, —NR⁴C(O)R⁴, —C(O)NHR⁴, —C(O)NR⁴R⁴, or —NO₂; wherein each alkyl, cycloalkyl, cycloalkylalkyl, aryl, arylalkyl, heterocyclyl, heterocyclylalkyl, heteroaryl, and heteroarylalkyl is optionally substituted with one or more R⁵;

R²⁵ is —OH, -alkyl-OH, —SH, -alkyl-SH, -alkyl-NH₂, or -alkyl-NHR⁶;

R⁴ is each independently H or alkyl;

R⁵ is each independently selected from I, Br, Cl, F, CN, NH₂, OH, OR⁶, R⁶, or SH; and

R⁶ is each independently alkyl.

In one embodiment, the Parkin ligase activator is

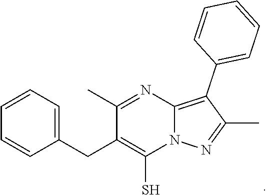 [SMILES: Cc1nc2c(-c3ccccc3)c(C)nn2c(S)c1Cc1ccccc1]

In one embodiment, the present disclosure provides a Parkin ligase activator comprising the structure of formula (I):

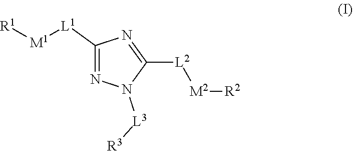

or a pharmaceutically acceptable salt or solvate thereof, wherein:

L¹, L² and L³ are each independently selected from a bond, alkylene, or alkenylene;

M¹ and M² are each independently selected from —NR⁴—, —NR═C(O)—, —N(C(O)R¹)—, —C(O)NR⁴—, —NR⁴C(O)NR⁴—, —C(O)—, —C(═NR⁴)—, —C(═NOR⁴)—, —OC(O)—, —C(O)O—, —OC(O)O—, —OC(O)NR⁴—, —NR⁴C(O)O—, —S(O)ₘ—, —S(O)ₘNR⁴—, or —NR⁴S(O)ₘ—, provided that M¹ and M² are not both —NR⁴—;

R¹ and R² are each independently selected from an alkyl, alkenyl, cycloalkyl, aryl, biphenyl, heterocyclyl heterocycloalkyl, heteroaryl, cycloalkylalkyl, arylalkyl, arylalkenyl, arylalkynyl, heterocyclylalkyl, heteroarylalkyl, heteroarylalkenyl, or heteroarylalkynyl, wherein each cycloalkyl, aryl, heteroaryl, and heterocyclyl portion is optionally substituted with one or more R⁵;

R³ is selected from an alkyl, alkenyl, cycloalkyl, aryl, biphenyl, heterocyclyl heterocycloalkyl, heteroaryl, cycloalkylalkyl, arylalkyl, arylalkenyl, arylalkynyl, heterocyclylalkyl, heteroarylalkyl, heteroarylalkenyl, or heteroarylalkynyl, wherein each cycloalkyl, aryl, heteroaryl, and heterocyclyl portion is optionally substituted with one or more R⁷;

R⁴ is each independently H, alkyl, wherein each alkyl is optionally substituted with one or more R⁵;

R⁵ is each independently I, Br, Cl, F, —CH₂F, —CHF₂, —CF₃, —OCF₃, —CN, -alkyl-CN, —CONH₂, —CONHR⁶, —CONR⁶R⁶, —COOH, —NH₂, —NHR⁶, —NO₂, —NR⁶R⁶, —N₃, —OH, OR⁶, —COOR⁶, —OSO₃R⁶, oxo, R⁶, —SH, —SO₂R⁶, —SO₃H, —SO₃R⁶, or —SR⁶;

R⁶ is each independently alkyl, alkenyl, alkynyl, haloalkyl, haloalkenyl, haloalkynyl; or alternatively two R⁶ on the same N atom can together form a 3-6 membered N-heterocyclyl;

R₇ is each independently I, Br, Cl, F, —CH₂F, —CHF₂, —CF₃, —OCF₃, —CN, -alkyl-CN, —CONH₂, —CONHR⁶, —CONR⁶R⁶, —COOH, —NH₂, —NHR⁶, —NO₂, —NR⁶R⁶, —N₃, —OH, OR⁶, —COOR⁶, —OSO₃R⁶, oxo, R⁶, —SH, —SO₂R⁶, —SO₃H, —SO₃R⁶, —SR⁶, cycloalkyl, heterocyclyl, aryl, or heteroaryl, wherein each cycloalkyl, heterocyclyl, aryl, and heteroaryl is optionally substituted with one or more R⁵; and

m is 0, 1, or 2.

As used herein, “the compound” refers to the Parkin ligase activators

In one embodiment of the compound of formula (I), L¹, L² and L³ are each independently a bond.

In one embodiment of the compound of formula (I), M¹ and M₂ are each independently selected from —NR⁴—, —NR⁴C(O)—, —C(O)NR⁴—, —N(C(O)R¹)—, or —NR⁴S(O)ₘ—. In one embodiment, M₁ and M² are each independently selected from —NR⁴—, —NR⁴C(O)— or —C(O)NR⁴—.

In one embodiment of the compound of formula (I), -M¹-R¹ is —NR⁴C(O)R¹. In one embodiment of the compound of formula (I), -M²-R² is —NR⁴C(O)R². In one embodiment of the compound of formula (I), -M₁-R¹ is —NR⁴C(O)R¹ and -M²-R² is —NR⁴C(O)R².

In one embodiment of the compound of formula (I), R⁴ at each occurrence is independently H or C₁-C₃ alkyl.

In one embodiment of the compound of formula (I), L³ is a bond and R³ is an aryl or a heteroaryl, optionally substituted with one or more R⁷.

In one embodiment of the compound of formula (I), R³ is a phenyl or phenyl fused bicycle, optionally substituted with one or more R⁷. In another embodiment, R³ is heteroaryl selected from imidazolyl or pyrazolyl, optionally substituted with one or more R⁷.

In one embodiment of the compound of formula (I), R⁷ is each independently I, Br, Cl, F, —CH₃, —CH₂F, —CHF₂, —CF₃, —OCF₃, —CN, —NH₂, —NMe₂, —NO₂, —N₃, —OH, OR⁶, R⁶, —SH, cycloalkyl, heterocyclyl, aryl, or heteroaryl, wherein each cycloalkyl, heterocyclyl, aryl, and heteroaryl is optionally substituted with one or more R⁵.

In one embodiment of the compound of formula (I), R³ is a phenyl substituted with a 4-6 membered heterocyclyl, which is optionally substituted with one or more R⁷.

In one embodiment of the compound of formula (I), R¹ and R² are each independently selected from phenyl, 6-10 membered aryl, 5-10 membered heteroaryl, 4-10 membered heterocyclyl, phenyl-(C₁-C₃ alkyl)-, phenyl-(C₂-C₃ alkenyl)-, 5-6 membered heteroaryl-(C₁-C₃ alkyl)-, or heteroaryl-(C₂-C₃ alkenyl)-, wherein each cycloalkyl, aryl, heteroaryl portion is optionally substituted with one or more R⁵. In some embodiments, the 6-10 membered aryl or 5-10 membered heteroaryl is a bicyclic ring.

In one embodiment of the compound of formula (I), R⁵ is selected from I, Br, Cl, F, C₁-C₆ alkyl, alkynyl, —CN, —(C₁-C₃ alkylene)-CN, —NH₂, —NO₂, —N₃, —OH, —OCF₃, —OMe, —NMe₂, or —NEt₂.

In one embodiment of the compound of formula (I), at least one of R¹, R², and R³ is phenyl and substituted with at least one of methyl, ethyl, —C≡CH, I, Br, Cl, F, —CF₃, —CN, —CH₂CN, —CH₂CH₂CN, —NH₂, —NO₂, —N₃, —OH, —OCF₃, —OMe or —NMe₂. In some embodiments, at least two of R¹, R², and R³ is phenyl and substituted with at least one of methyl, ethyl, —C≡CH, I, Br, Cl, F, —CF₃, —CN, —CH₂CN, —CH₂CH₂CN, —NH₂, —NO₂, —N₃, —OH, —OCF₃, —OMe or —NMe₂. In another embodiment, at least one of R¹, R², and R³ is pyridyl, optionally substituted with one or more of methyl, ethyl, —C≡CH, I, Br, Cl, F, —CF₃, —CN, —CH₂CN, —CH₂CH₂CN, —NH₂, —NO₂, —N₃, —OH, —OCF₃, —OMe or —NMe₂.

In one embodiment, the compound of formula (I) has the structure of formula (I′):

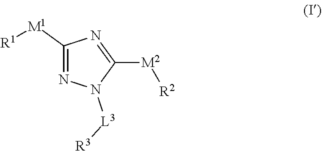

or a pharmaceutically acceptable salt or solvate thereof, wherein L³, M¹, M², R¹, R², and R³ are as defined for formula (I).

In one embodiment of the compound of formula (I′), M¹ and M² are each independently selected from —NR⁴—, —NR⁴C(O)—, —C(O)NR⁴—, —N(C(O)R¹)—, or —NR⁴S(O)ₘ—.

In one embodiment of the compound of formula (I′), -M¹-R¹ is —NR⁴C(O)R¹. In one embodiment of the compound of formula (I′), -M²-R² is —NR⁴C(O)R². In one embodiment of the compound of formula (I′), -M¹-R¹ is —NR⁴C(O)R¹ and -M²-R² is —NR⁴C(O)R².

In one embodiment of the compound of formula (I′), R¹ and R² are each independently selected from phenyl, 6-10 membered aryl, 5-10 membered heteroaryl, 4-10 membered heterocyclyl, phenyl-(C₁-C₃ alkyl)-, phenyl-(C₂-C₃ alkenyl)-, 5-6 membered heteroaryl-(C₁-C₃ alkyl)-, or heteroaryl-(C₂-C₃ alkenyl)-, wherein each cycloalkyl, aryl, heteroaryl portion is optionally substituted with one or more R⁵; and R³ is an aryl or a heteroaryl, optionally substituted with one or more R⁷.

In one embodiment, the compound of formula (I) has the structure of formula (IA′):

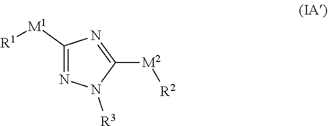

or a pharmaceutically acceptable salt or solvate thereof, wherein:

M¹ and M² are each independently selected from —NR⁴C(O)— or —C(O)NR⁴—;

R¹ and R² are each independently;

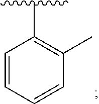 [SMILES: *c1ccccc1C]

R³ is selected from

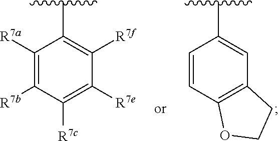

R⁴ is each independently H or C₁-C₃ alkyl; and

R^{7a}, R^{7b}, R^{7e}, and R^{7f} is each independently H, I, Br, Cl, F, —CH₂F, —CHF₂, —CF₃, —OCF₃, —N₃, —CN, —OH, methyl, ethyl, propyl, C₁-C₃ haloalkyl, C₁-C₃ alkoxy, or C₁-C₃ haloalkoxy;

R^{7c} is H, I, Br, Cl, F, —CH₂F, —CHF₂, —CF₃, —OCF₃, —N₃, —CN, —OH, C₁-C₃ alkyl, C₁-C₃ haloalkyl, C₁-C₃ alkoxy, C₁-C₃ haloalkoxy, 4-6 membered heterocyclyl, or 5-6 membered heteroaryl, wherein the heterocyclyl and heteroaryl is optionally substituted with one or more R⁵;

R⁵ is I, Br, Cl, F, —CH₂F, —CHF₂, —CF₃, —C₁-C₆ alkyl, alkynyl, —CN, —(C₁-C₃ alkylene)-CN, —NH₂, —NO₂, —N₃, —OH, —OCF₃, —OMe, —NMe₂,—NEt₂, or—C(O)O(C₁-C₆ alkyl);

wherein at least one of R^{7a}, R^{7b}, R^{7c}, R^{7e}, and R^{7f} is not H.

In one embodiment of the compound of formula (IA′), -M¹-R¹ is —NR⁴C(O)R¹. In one embodiment of the compound of formula (IA′), -M²-R² is —NR⁴C(O)R². In one embodiment of the compound of formula (IA′), -M¹-R¹ is —NR⁴C(O)R¹ and -M²-R² is —NR⁴C(O)R².

In one embodiment of the compound of formula (IA′), R³ is selected from R³ is

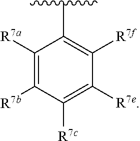

In one embodiment of the compound of formula (IA′), four of R^{7a}, R^{7b}, R^{7c}, R^{7e}, and R^{7f} is H. In another embodiment, three of R^{7a}, R^{7b}, R^{7c}, R^{7e}, and R^{7f} is H. In some embodiments, R^{7a}, R^{7b}, R^{7c}, R^{7e}, and R^{7f} is each independently H, I, Br, Cl, F, —CH₂F, —CHF₂, —CF₃, —OCF₃, —N₃, —CN, —OH, methyl, ethyl, propyl, —C≡CH; —CN, —NH₂, —NO₂, —N₃, —OH, —OCF₃, —OMe, —NMe₂, or —NEt₂.

In one embodiment of the compound of formula (IA′), R³ is

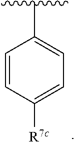

In some embodiments, R^{7c} is I, Br, —CH₂F, —CHF₂, —CF₃, methyl, ethyl, propyl, —C≡CH; —CN, —NH₂, —NO₂, —N₃, —OH, —OCF₃, —OMe, —NMe₂, or —NEt₂. In other embodiments, R^{7c} is I, Br, —CH₂F, —CHF₂, —CF₃, —OCF₃, or —OMe. In one embodiment, R^{7c} is azetidinyl, pyrrolidinyl, piperidinyl, morpholinyl, piperazinyl, or pyrazolyl, each optionally substituted with one or more R⁵.

In one embodiment, the compound of formula (I) has the structure of formula (IA):

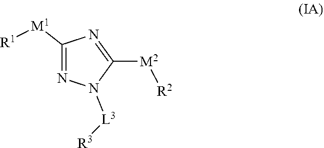

or a pharmaceutically acceptable salt or solvate thereof, wherein:

L³ is a bond;

M¹ and M² are each independently selected from —NR⁴C(O)— or —C(O)NR⁴—;

R¹ and R² are each phenyl, substituted with one or more R^{5a};

R³ is phenyl, optionally substituted with one or more R^{5b};

R⁴ is each independently H or C₁-C₃ alkyl;

R^{5a} is each independently I, Br, Cl, F, C₁-C₆ alkyl, C₁-C₃ haloalkyl, —(C₁-C₆)—O—(C₁-C₆), C₁-C₃ alkoxy, C₁-C₃ haloalkoxy, OH, or COOH;

R5b is each independently I, Br, Cl, F, CN, CONH₂, CONHR⁶, CONR⁶R⁶, COOH, NH₂, NHR⁶, NO₂, NR⁶R⁶, OH, OR⁶, —COOR⁶, OSO₃R⁶, oxo, R⁶, SH, SO₂R⁶, SO₃H, SO₃R⁶, or SR⁶; and

R⁶ is each independently alkyl or haloalkyl.

In one embodiment, the compound of formula (I) has the structure of formula (IB):

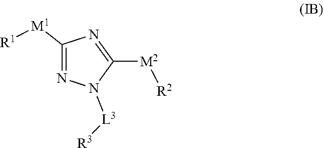

or a pharmaceutically acceptable salt or solvate thereof, wherein:

L3 is a bond;

M1 and M2 are each independently selected from —NR⁴C(O)— or —C(O)NR⁴—;

R1 and R2 are each phenyl, substituted with one or more R^{5a};

R3 is phenyl, optionally substituted with one or more R^{5b};

R4 is each independently H or C₁-C₃ alkyl;

R5a is each independently C₁-C₆ alkyl;

R5b is each independently I, Br, Cl, F, CN, CONH₂, CONHR⁶, CONR⁶R⁶, COOH, NH₂, NHR⁶, NO₂, NR⁶R⁶, OH, OR⁶, —COOR⁶, OSO₃R⁶, oxo, R₆, SH, SO₂R⁶, SO₃H, SO₃R⁶, or SR⁶; and

R⁶ is each independently alkyl or haloalkyl.

In one embodiment, the compound of formula (I) has the structure of formula (IC):

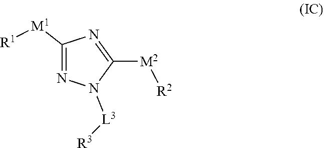

or a pharmaceutically acceptable salt or solvate thereof, wherein:

L³ is a bond;

M¹ and M² are each independently selected from —NR⁴C(O)— or —C(O)NR⁴—;

R¹ and R² are each phenyl, substituted with one or more R^{5a}, wherein at least one of R₁ and R₂ is

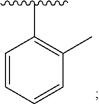 [SMILES: *c1ccccc1C]

R³ is phenyl, optionally substituted with one or more R^{5b};

R⁴ is each independently H or C₁-C₃ alkyl;

R5a is each independently I, Br, Cl, F, C1-C6 alkyl, C₁-C₃ haloalkyl, C₁-C₃ alkoxy, C₁-C₃ haloalkoxy, OH, or COOH;

R^{5b} is each independently I, Br, Cl, F, CN, COME, CONHR⁶, CONR⁶R⁶, COOH, NH2, NHR⁶, NO₂, NR⁶R⁶, OH, OR⁶, —COOR⁶, OSO₃R⁶, oxo, R⁶, SH, SO₂R⁶, SO₃H, SO₃R⁶, or SR₆; and

R⁶ is each independently alkyl or haloalkyl.

In one embodiment, the compound of formula (I) has the structure of formula (ID):

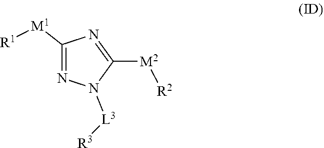

or a pharmaceutically acceptable salt or solvate thereof, wherein:

L³ is a bond;

M¹ and M₂ are each independently selected from —NR⁴C(O)— or —C(O)NR₄—;

R¹ and R² are each

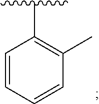 [SMILES: *c1ccccc1C]

R³ is phenyl, optionally substituted with one or more R^{5b};

R⁴ is each independently H or C₁-C₃ alkyl;

R^{5b} is each independently I, Br, Cl, F, CN, CONH2, CONHR⁶, CONR⁶R⁶, COOH, NH₂, NHR⁶, NO₂, NR⁶R⁶, OH, OR⁶, —COOR⁶, OSO₃R⁶, oxo, R⁶, SH, SO₂R⁶, SO₃H, SO₃R⁶, or SR⁶; and

R⁶ is each independently alkyl or haloalkyl.

In one embodiment, the compound of formula (I) has the structure of formula (IE):

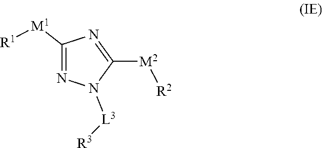

or a pharmaceutically acceptable salt or solvate thereof, wherein:

L³ is a bond;

M¹ and M² are each —NHC(O)—;

R¹ and R² are each

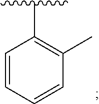 [SMILES: *c1ccccc1C]

R₃ is phenyl, optionally substituted with one or more R^{5b}; and

R^{5b} is each independently I, Br, Cl, F, C₁-C₃ alkyl, C₁-C₃ haloalkyl, C₁-C₃ alkoxy, C₁-C₃ haloalkoxy, OH, or COOH.

In one embodiment, the compound of formula (I) has the structure of formula (IF):

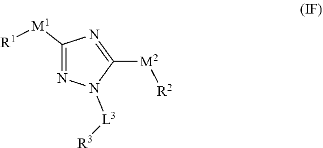

or a pharmaceutically acceptable salt or solvate thereof, wherein:

L³ is a bond;

M¹ and M² are each —NHC(O)—:

R¹ and R² are each

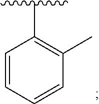 [SMILES: *c1ccccc1C]

R³ is phenyl, optionally substituted with one or more R^{5b}; and

R^{5b} is each independently C₁-C₃ alkyl, C₁-C₃ haloalkyl, C₁-C₃ alkoxy, C₁-C₃ haloalkoxy, OH, or COOH.

In one embodiment, the compound of formula (I) has the structure of formula (IG):

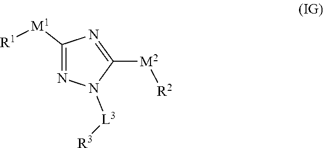

or a pharmaceutically acceptable salt or solvate thereof, wherein:

L³ is a bond;

M¹ and M² are each —NHC(O)—;

R¹ and R² are each

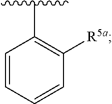

R³ is phenyl; and

R^{5a} is each independently C₁-C₆ alkyl, C₁-C₃ haloalkyl, C₁-C₃ alkoxy, C₁-C₃ haloalkoxy, OH, or COOH.

Various embodiments as described above for formula (I) also applies to formula (I′), (IA′), (IA), (IB), (IC), (ID), (IE), (IF), and (IG).

In one embodiment, the compound of formula (I), (I′), (IA′), (IA), (IB), (IC), (ID), (IE), (IF), or (IG) is not N,N′-(1-phenyl-1H-1,2,4-triazole-3,5-diyl)dibenzamide, N-(3-benzamido-1-phenyl-1H-1,2,4-triazol-5-yl)furan-2-carboxamide, N-(5-cinnamamido-1-phenyl-1H-1,2,4-triazol-3-yl)benzamide, N-(1-phenyl-5-(phenylamino)-1H-1,2,4-triazol-3-yl)benzamide, 4-fluoro-N-(5-(4-methoxybenzamido)-1-phenyl-1H-1,2,4-triazol-3-yl)benzamide, N,N′-(1-phenyl-1H-1,2,4-triazole-3,5-diyl)bis(4-methylbenzamide), N-(5-((2-chlorobenzyl)amino)-1-phenyl-1H-1,2,4-triazol-3-yl)-2-fluorobenzamide, N-(3-benzamido-1-phenyl-1H-1,2,4-triazol-5-yl)-4-fluorobenzamide, N-(3-benzamido-1-phenyl-1H-1,2,4-triazol-5-yl)-4-nitrobenzamide, A-(3-benzamido-1-phenyl-1H-1,2,4-triazol-5-yl)-3-nitrobenzamide, and 4-((3-benzamido-1-phenyl-1H-1,2,4-triazol-5-yl)carbamoyl)benzoic acid.

In one embodiment of the compound of formula (I), (I′), or (IA′), the compound is selected from Table 1 below, or a pharmaceutically acceptable salt or solvate thereof.

TABLE 1

| Compd ID | Structure |
|---|---|
| 1 | 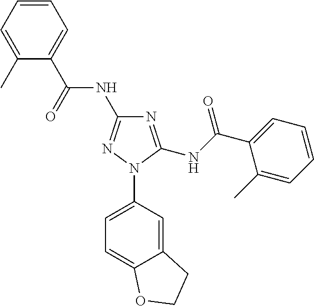 [SMILES: Cc1ccccc1C(=O)Nc1nc(NC(=O)c2ccccc2C)n(-c2ccc3c(c2)CCO3)n1] |
| 2 | 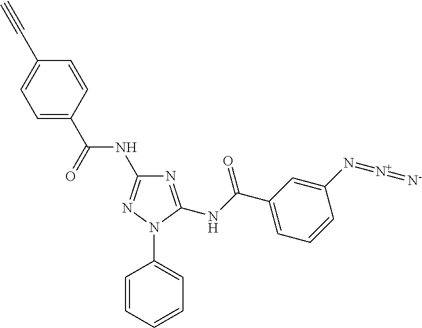 [SMILES: C#Cc1ccc(C(=O)Nc2nc(NC(=O)c3cccc(N=[N+]=[N-])c3)n(-c3ccccc3)n2)cc1] |
| 3 | 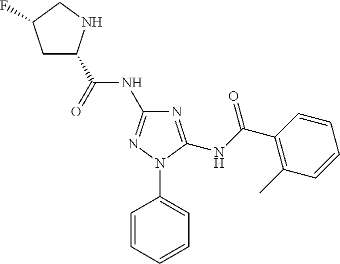 [SMILES: Cc1ccccc1C(=O)Nc1nc(NC(=O)[C@@H]2C[C@H](F)CN2)nn1-c1ccccc1] |
| 4 | 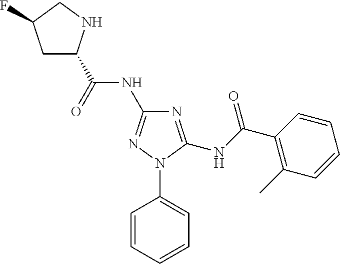 [SMILES: Cc1ccccc1C(=O)Nc1nc(NC(=O)[C@@H]2C[C@@H](F)CN2)nn1-c1ccccc1] |
| 5 | 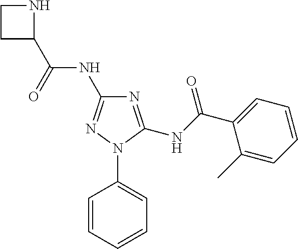 [SMILES: Cc1ccccc1C(=O)Nc1nc(NC(=O)C2CCN2)nn1-c1ccccc1] |
| 6 | 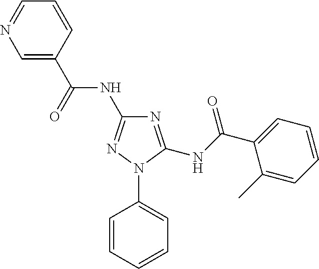 [SMILES: Cc1ccccc1C(=O)Nc1nc(NC(=O)c2cccnc2)nn1-c1ccccc1] |
| 7 | 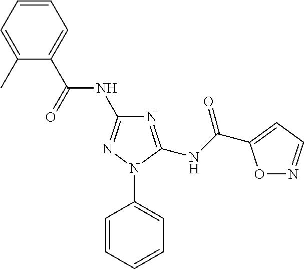 [SMILES: Cc1ccccc1C(=O)Nc1nc(NC(=O)c2ccno2)n(-c2ccccc2)n1] |
| 8 | 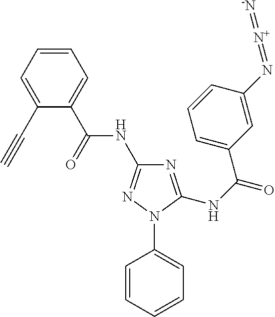 |
| 9 | 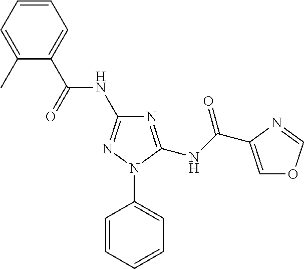 [SMILES: Cc1ccccc1C(=O)Nc1nc(NC(=O)c2cocn2)n(-c2ccccc2)n1] |
| 10 | 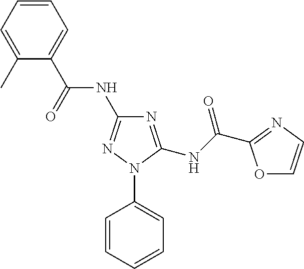 [SMILES: Cc1ccccc1C(=O)Nc1nc(NC(=O)c2ncco2)n(-c2ccccc2)n1] |
| 11 | 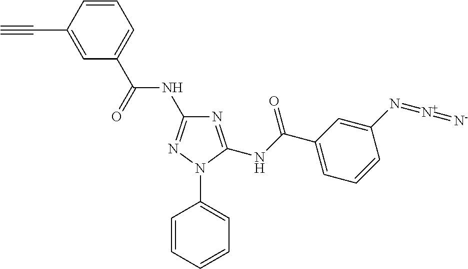 [SMILES: C#Cc1cccc(C(=O)Nc2nc(NC(=O)c3cccc(N=[N+]=[N-])c3)n(-c3ccccc3)n2)c1] |
| 12 | 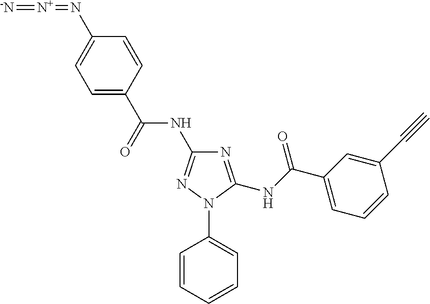 |
| 13 | 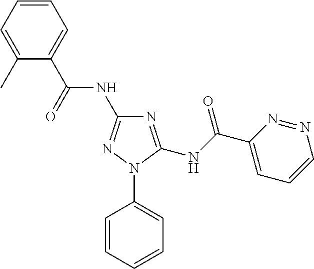 [SMILES: Cc1ccccc1C(=O)Nc1nc(NC(=O)c2cccnn2)n(-c2ccccc2)n1] |
| 14 | 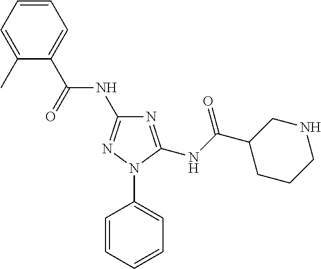 [SMILES: Cc1ccccc1C(=O)Nc1nc(NC(=O)C2CCCNC2)n(-c2ccccc2)n1] |
| 15 | 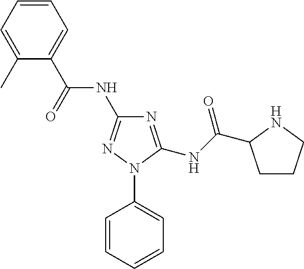 [SMILES: Cc1ccccc1C(=O)Nc1nc(NC(=O)C2CCCN2)n(-c2ccccc2)n1] |
| 16 | 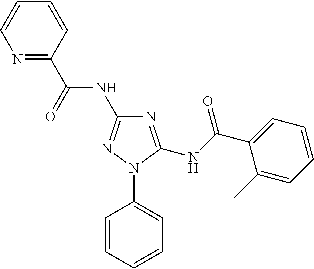 [SMILES: Cc1ccccc1C(=O)Nc1nc(NC(=O)c2ccccn2)nn1-c1ccccc1] |
| 17 | 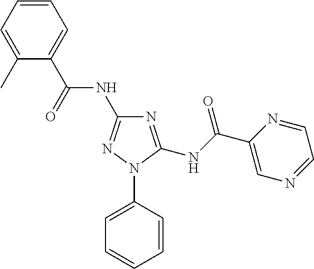 [SMILES: Cc1ccccc1C(=O)Nc1nc(NC(=O)c2cnccn2)n(-c2ccccc2)n1] |
| 18 | 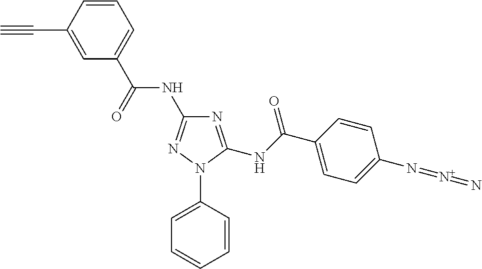 [SMILES: C#Cc1cccc(C(=O)Nc2nc(NC(=O)c3ccc(N=[N+]=N)cc3)n(-c3ccccc3)n2)c1] |
| 19 | 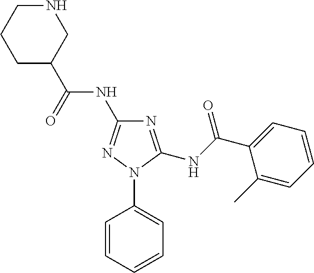 [SMILES: Cc1ccccc1C(=O)Nc1nc(NC(=O)C2CCCNC2)nn1-c1ccccc1] |
| 20 | 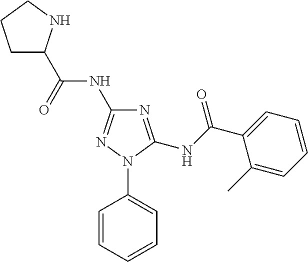 [SMILES: Cc1ccccc1C(=O)Nc1nc(NC(=O)C2CCCN2)nn1-c1ccccc1] |
| 21 | 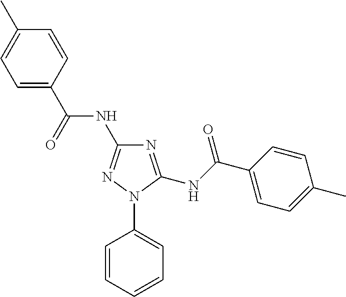 [SMILES: Cc1ccc(C(=O)Nc2nc(NC(=O)c3ccc(C)cc3)n(-c3ccccc3)n2)cc1] |
| 22 | 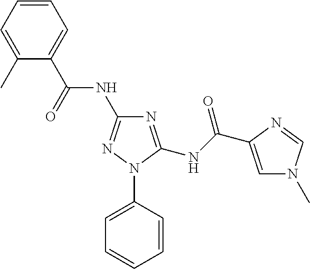 [SMILES: Cc1ccccc1C(=O)Nc1nc(NC(=O)c2cn(C)cn2)n(-c2ccccc2)n1] |
| 23 | 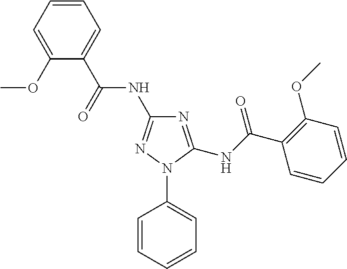 [SMILES: COc1ccccc1C(=O)Nc1nc(NC(=O)c2ccccc2OC)n(-c2ccccc2)n1] |
| 24 | 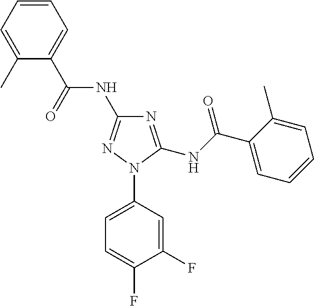 [SMILES: Cc1ccccc1C(=O)Nc1nc(NC(=O)c2ccccc2C)n(-c2ccc(F)c(F)c2)n1] |
| 25 | 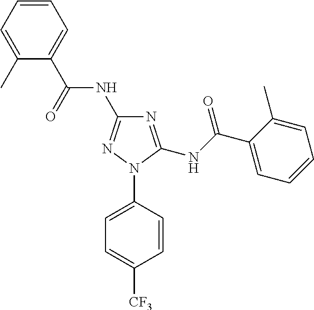 [SMILES: Cc1ccccc1C(=O)Nc1nc(NC(=O)c2ccccc2C)n(-c2ccc(C(F)(F)F)cc2)n1] |
| 26 | 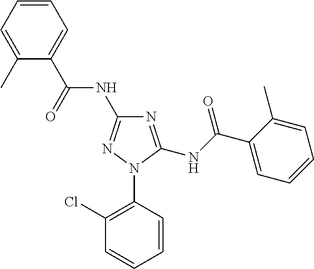 [SMILES: Cc1ccccc1C(=O)Nc1nc(NC(=O)c2ccccc2C)n(-c2ccccc2Cl)n1] |
| 27 | 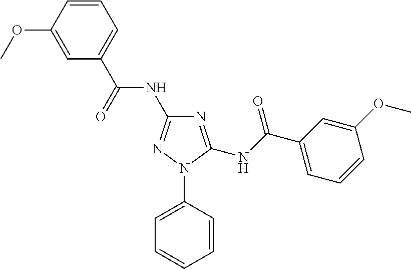 |
| 28 | 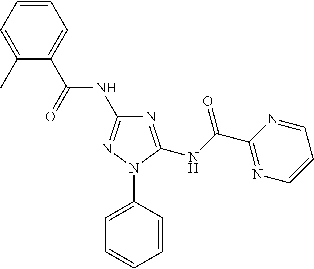 [SMILES: Cc1ccccc1C(=O)Nc1nc(NC(=O)c2ncccn2)n(-c2ccccc2)n1] |
| 29 | 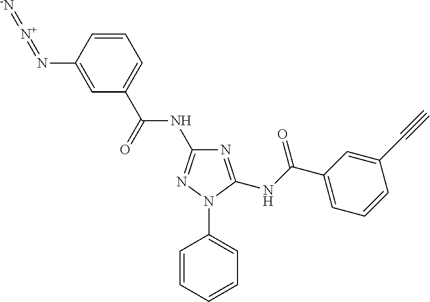 |
| 30 | 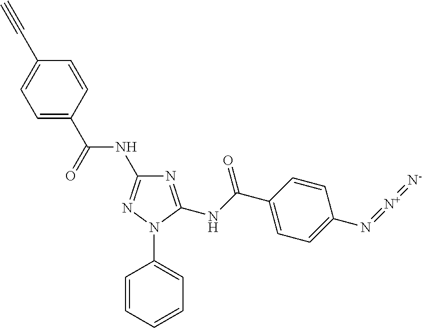 [SMILES: C#Cc1ccc(C(=O)Nc2nc(NC(=O)c3ccc(N=[N+]=[N-])cc3)n(-c3ccccc3)n2)cc1] |
| 31 | 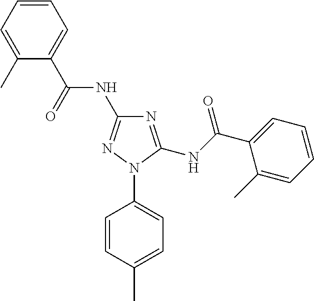 [SMILES: Cc1ccc(-n2nc(NC(=O)c3ccccc3C)nc2NC(=O)c2ccccc2C)cc1] |
| 32 | 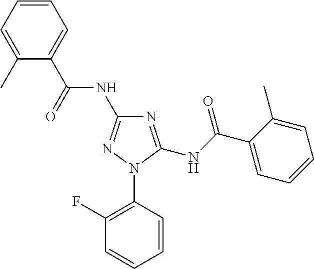 [SMILES: Cc1ccccc1C(=O)Nc1nc(NC(=O)c2ccccc2C)n(-c2ccccc2F)n1] |
| 33 | 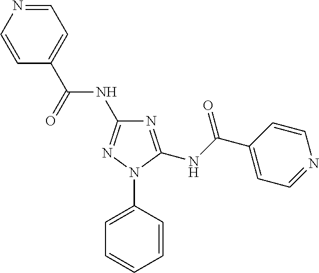 [SMILES: O=C(Nc1nc(NC(=O)c2ccncc2)n(-c2ccccc2)n1)c1ccncc1] |
| 34 | 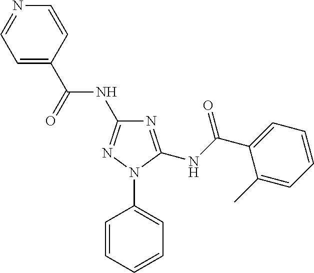 [SMILES: Cc1ccccc1C(=O)Nc1nc(NC(=O)c2ccncc2)nn1-c1ccccc1] |
| 35 | 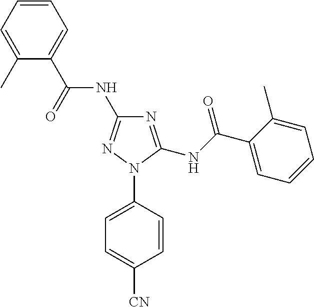 [SMILES: Cc1ccccc1C(=O)Nc1nc(NC(=O)c2ccccc2C)n(-c2ccc(C#N)cc2)n1] |
| 36 | 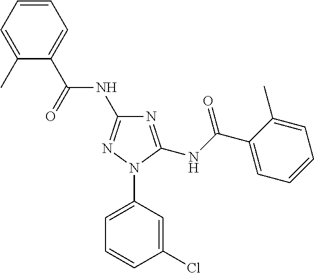 [SMILES: Cc1ccccc1C(=O)Nc1nc(NC(=O)c2ccccc2C)n(-c2cccc(Cl)c2)n1] |
| 37 | 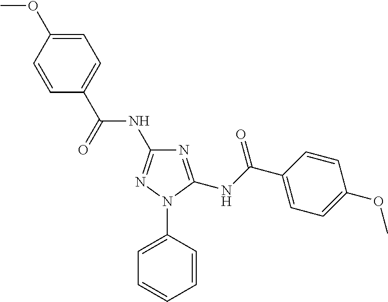 |
| 38 | 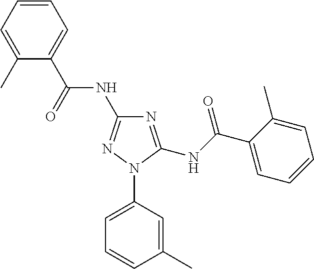 [SMILES: Cc1cccc(-n2nc(NC(=O)c3ccccc3C)nc2NC(=O)c2ccccc2C)c1] |
| 39 | 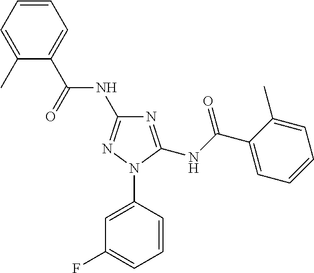 [SMILES: Cc1ccccc1C(=O)Nc1nc(NC(=O)c2ccccc2C)n(-c2cccc(F)c2)n1] |
| 40 | 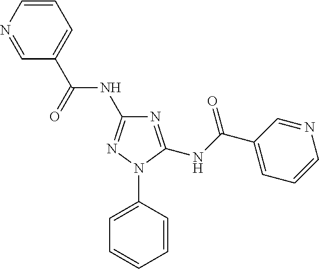 [SMILES: O=C(Nc1nc(NC(=O)c2cccnc2)n(-c2ccccc2)n1)c1cccnc1] |
| 41 | 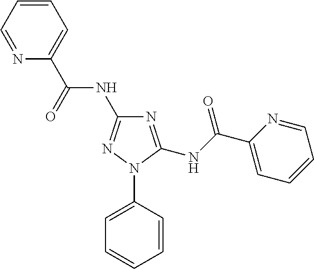 [SMILES: O=C(Nc1nc(NC(=O)c2ccccn2)n(-c2ccccc2)n1)c1ccccn1] |
| 42 | 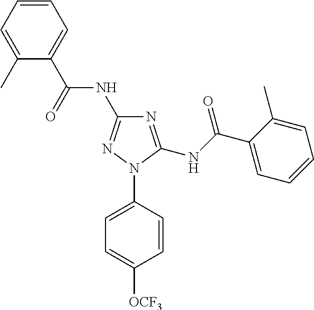 [SMILES: Cc1ccccc1C(=O)Nc1nc(NC(=O)c2ccccc2C)n(-c2ccc(OC(F)(F)F)cc2)n1] |
| 43 | 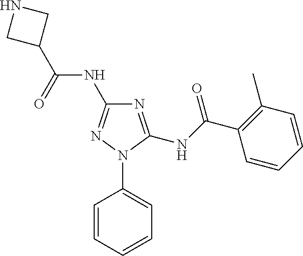 [SMILES: Cc1ccccc1C(=O)Nc1nc(NC(=O)C2CNC2)nn1-c1ccccc1] |
| 44 | 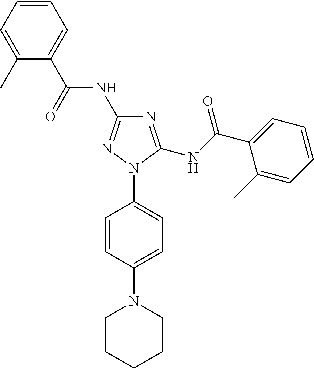 [SMILES: Cc1ccccc1C(=O)Nc1nc(NC(=O)c2ccccc2C)n(-c2ccc(N3CCCCC3)cc2)n1] |
| 45 | 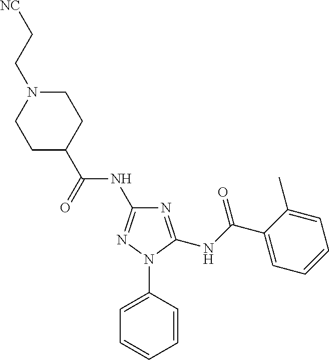 [SMILES: Cc1ccccc1C(=O)Nc1nc(NC(=O)C2CCN(CCC#N)CC2)nn1-c1ccccc1] |
| 46 | 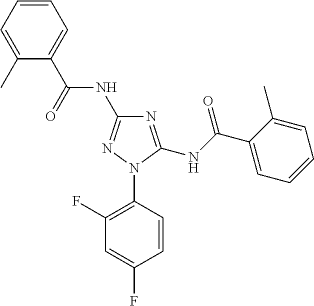 [SMILES: Cc1ccccc1C(=O)Nc1nc(NC(=O)c2ccccc2C)n(-c2ccc(F)cc2F)n1] |
| 47 | 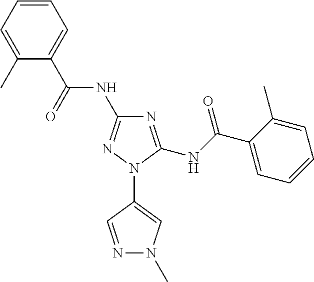 [SMILES: Cc1ccccc1C(=O)Nc1nc(NC(=O)c2ccccc2C)n(-c2cnn(C)c2)n1] |
| 48 | 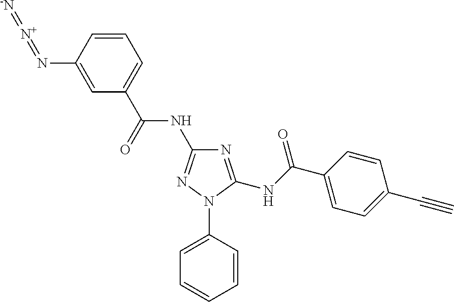 |
| 49 | 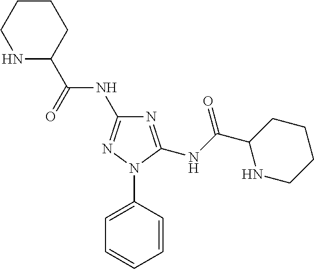 [SMILES: O=C(Nc1nc(NC(=O)C2CCCCN2)n(-c2ccccc2)n1)C1CCCCN1] |
| 50 | 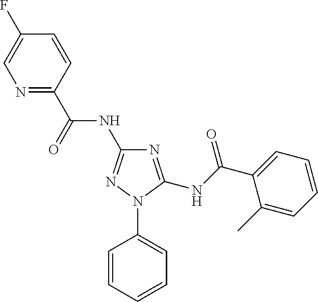 [SMILES: Cc1ccccc1C(=O)Nc1nc(NC(=O)c2ccc(F)cn2)nn1-c1ccccc1] |
| 51 | 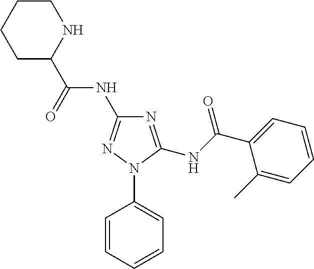 [SMILES: Cc1ccccc1C(=O)Nc1nc(NC(=O)C2CCCCN2)nn1-c1ccccc1] |
| 52 | 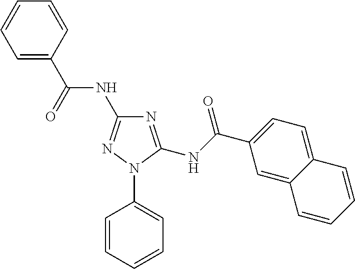 [SMILES: O=C(Nc1nc(NC(=O)c2ccc3ccccc3c2)n(-c2ccccc2)n1)c1ccccc1] |
| 53 | 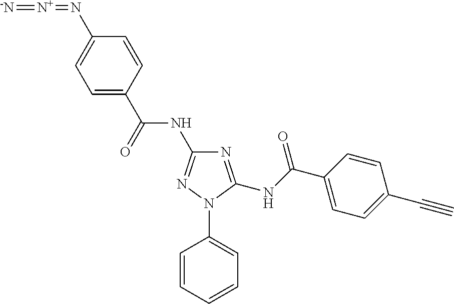 |
| 54 | 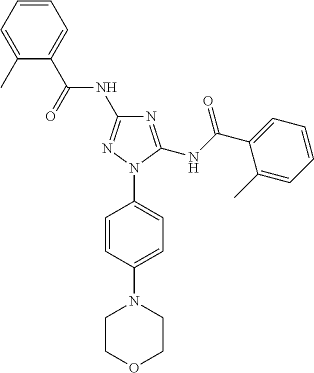 [SMILES: Cc1ccccc1C(=O)Nc1nc(NC(=O)c2ccccc2C)n(-c2ccc(N3CCOCC3)cc2)n1] |
| 55 | 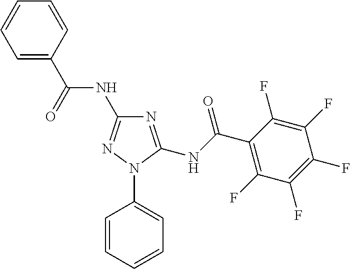 [SMILES: O=C(Nc1nc(NC(=O)c2c(F)c(F)c(F)c(F)c2F)n(-c2ccccc2)n1)c1ccccc1] |
| 56 | 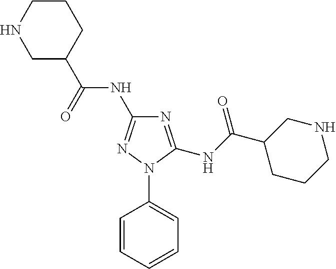 [SMILES: O=C(Nc1nc(NC(=O)C2CCCNC2)n(-c2ccccc2)n1)C1CCCNC1] |
| 57 | 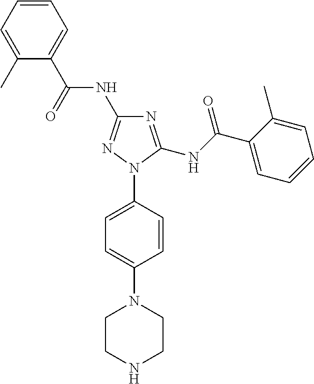 [SMILES: Cc1ccccc1C(=O)Nc1nc(NC(=O)c2ccccc2C)n(-c2ccc(N3CCNCC3)cc2)n1] |
| 58 | 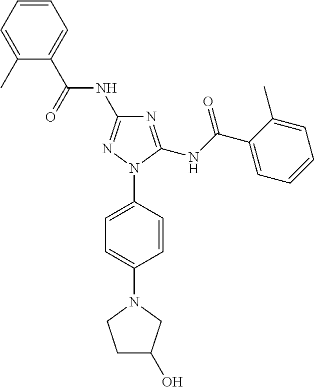 [SMILES: Cc1ccccc1C(=O)Nc1nc(NC(=O)c2ccccc2C)n(-c2ccc(N3CCC(O)C3)cc2)n1] |
| 59 | 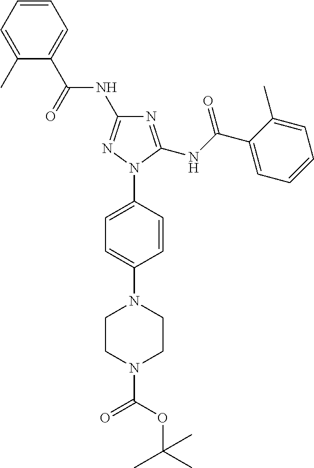 [SMILES: Cc1ccccc1C(=O)Nc1nc(NC(=O)c2ccccc2C)n(-c2ccc(N3CCN(C(=O)OC(C)(C)C)CC3)cc2)n1] |
| 60 | 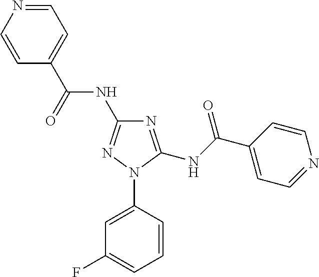 [SMILES: O=C(Nc1nc(NC(=O)c2ccncc2)n(-c2cccc(F)c2)n1)c1ccncc1] |
| 61 | 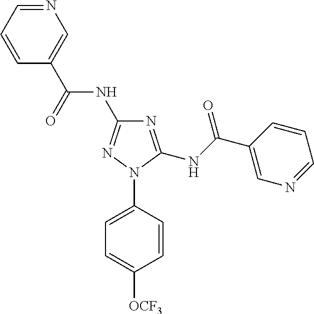 [SMILES: O=C(Nc1nc(NC(=O)c2cccnc2)n(-c2ccc(OC(F)(F)F)cc2)n1)c1cccnc1] |
| 62 | 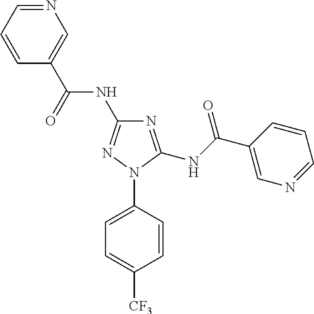 [SMILES: O=C(Nc1nc(NC(=O)c2cccnc2)n(-c2ccc(C(F)(F)F)cc2)n1)c1cccnc1] |
| 63 | 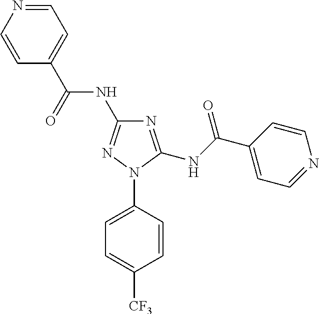 [SMILES: O=C(Nc1nc(NC(=O)c2ccncc2)n(-c2ccc(C(F)(F)F)cc2)n1)c1ccncc1] |
| 64 | 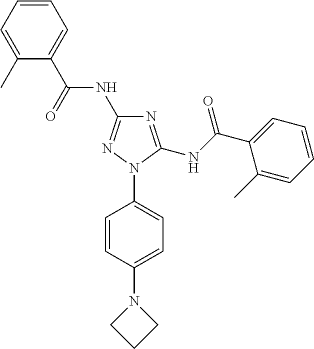 [SMILES: Cc1ccccc1C(=O)Nc1nc(NC(=O)c2ccccc2C)n(-c2ccc(N3CCC3)cc2)n1] |
| 65 | 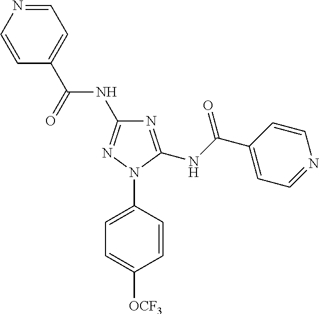 [SMILES: O=C(Nc1nc(NC(=O)c2ccncc2)n(-c2ccc(OC(F)(F)F)cc2)n1)c1ccncc1] |
| 66 | 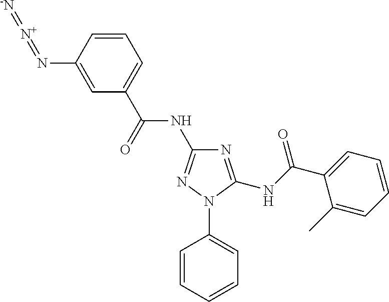 |
| 67 | 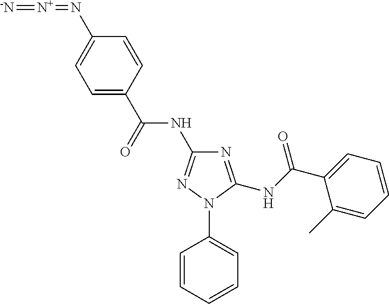 |
| 68 | 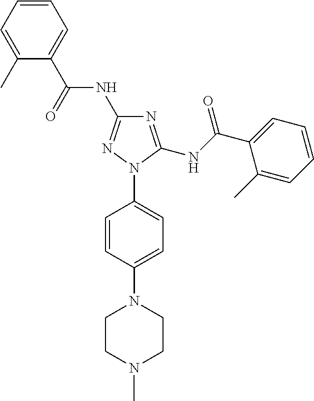 [SMILES: Cc1ccccc1C(=O)Nc1nc(NC(=O)c2ccccc2C)n(-c2ccc(N3CCN(C)CC3)cc2)n1] |
| 69 | 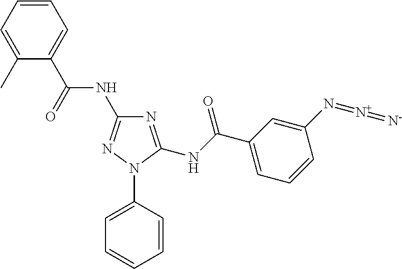 [SMILES: Cc1ccccc1C(=O)Nc1nc(NC(=O)c2cccc(N=[N+]=[N-])c2)n(-c2ccccc2)n1] |
| 70 | 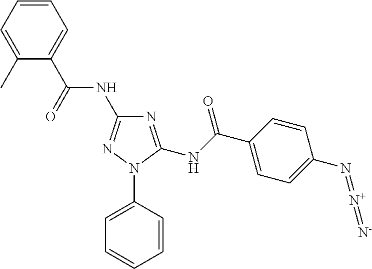 [SMILES: Cc1ccccc1C(=O)Nc1nc(NC(=O)c2ccc(N=[N+]=[N-])cc2)n(-c2ccccc2)n1] |
| 71 | 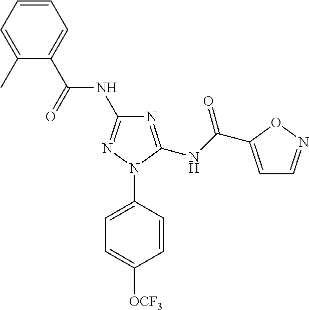 [SMILES: Cc1ccccc1C(=O)Nc1nc(NC(=O)c2ccno2)n(-c2ccc(OC(F)(F)F)cc2)n1] |
| 72 | 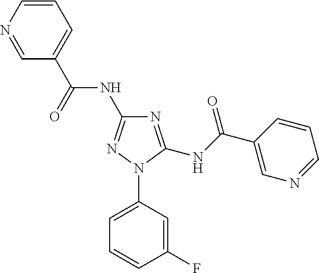 [SMILES: O=C(Nc1nc(NC(=O)c2cccnc2)n(-c2cccc(F)c2)n1)c1cccnc1] |
| 73 | 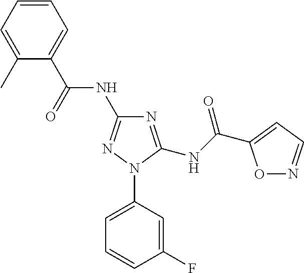 [SMILES: Cc1ccccc1C(=O)Nc1nc(NC(=O)c2ccno2)n(-c2cccc(F)c2)n1] |
| 74 | 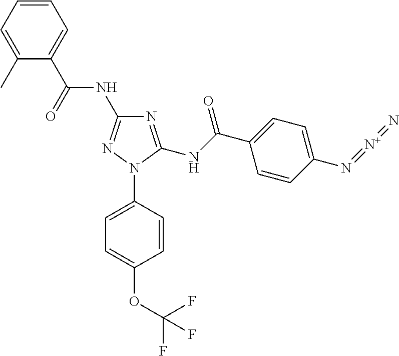 [SMILES: Cc1ccccc1C(=O)Nc1nc(NC(=O)c2ccc(N=[N+]=N)cc2)n(-c2ccc(OC(F)(F)F)cc2)n1] |
| 75 | 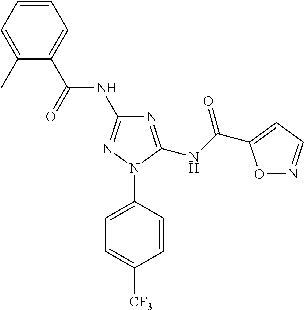 [SMILES: Cc1ccccc1C(=O)Nc1nc(NC(=O)c2ccno2)n(-c2ccc(C(F)(F)F)cc2)n1] |
| 76 | 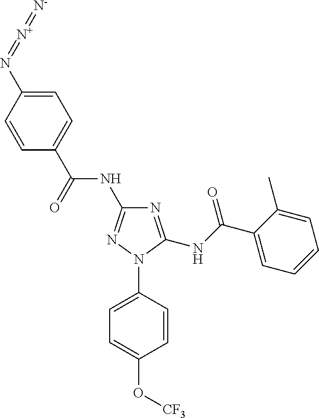 [SMILES: Cc1ccccc1C(=O)Nc1nc(NC(=O)c2ccc(N=[N+]=[N-])cc2)nn1-c1ccc(OC(F)(F)F)cc1] |
| 77 | 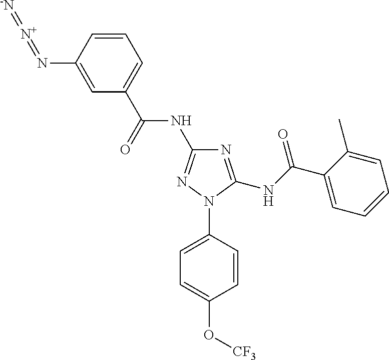 |
| 78 | 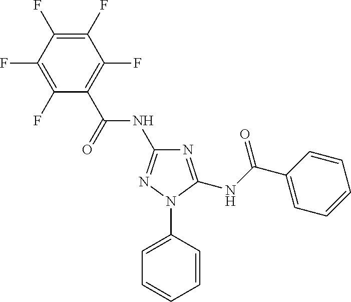 [SMILES: O=C(Nc1nc(NC(=O)c2c(F)c(F)c(F)c(F)c2F)nn1-c1ccccc1)c1ccccc1] |
| 79 | 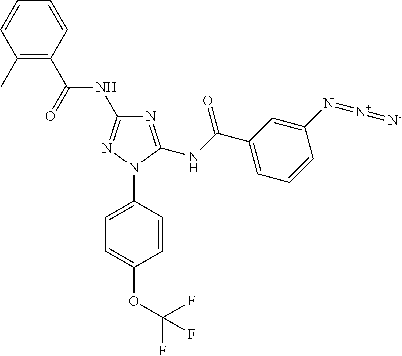 [SMILES: Cc1ccccc1C(=O)Nc1nc(NC(=O)c2cccc(N=[N+]=[N-])c2)n(-c2ccc(OC(F)(F)F)cc2)n1] |
| 80 | 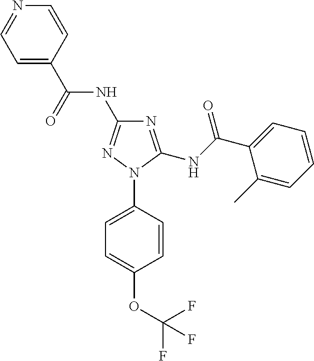 [SMILES: Cc1ccccc1C(=O)Nc1nc(NC(=O)c2ccncc2)nn1-c1ccc(OC(F)(F)F)cc1] |
| 81 | 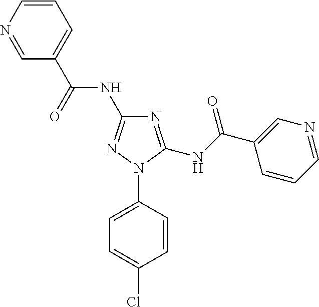 [SMILES: O=C(Nc1nc(NC(=O)c2cccnc2)n(-c2ccc(Cl)cc2)n1)c1cccnc1] |
| 82 | 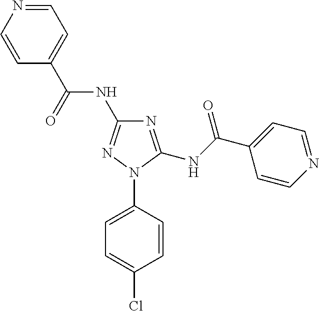 [SMILES: O=C(Nc1nc(NC(=O)c2ccncc2)n(-c2ccc(Cl)cc2)n1)c1ccncc1] |
| 83 | 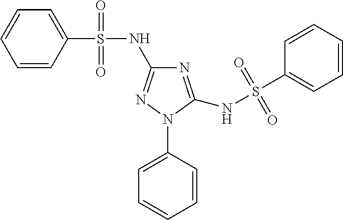 [SMILES: O=S(=O)(Nc1nc(NS(=O)(=O)c2ccccc2)n(-c2ccccc2)n1)c1ccccc1] |
| 84 | 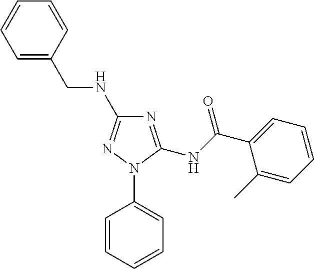 [SMILES: Cc1ccccc1C(=O)Nc1nc(NCc2ccccc2)nn1-c1ccccc1] |
| 85 | 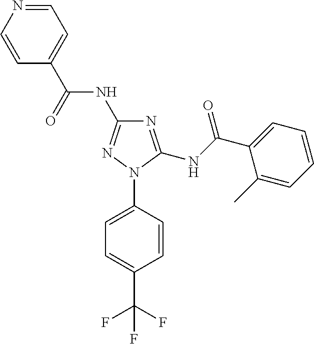 [SMILES: Cc1ccccc1C(=O)Nc1nc(NC(=O)c2ccncc2)nn1-c1ccc(C(F)(F)F)cc1] |
| 86 | 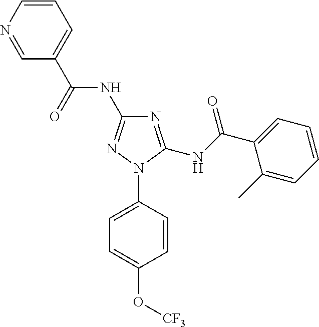 [SMILES: Cc1ccccc1C(=O)Nc1nc(NC(=O)c2cccnc2)nn1-c1ccc(OC(F)(F)F)cc1] |
| 87 | 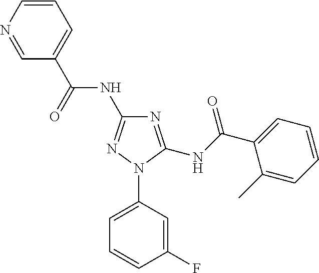 [SMILES: Cc1ccccc1C(=O)Nc1nc(NC(=O)c2cccnc2)nn1-c1cccc(F)c1] |
| 88 | 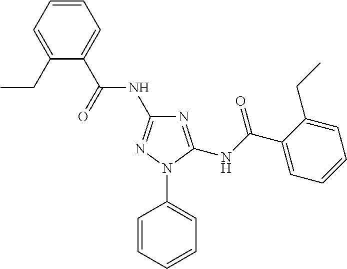 [SMILES: CCc1ccccc1C(=O)Nc1nc(NC(=O)c2ccccc2CC)n(-c2ccccc2)n1] |
| 89 | 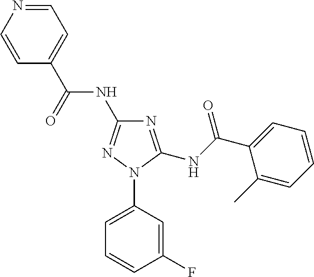 [SMILES: Cc1ccccc1C(=O)Nc1nc(NC(=O)c2ccncc2)nn1-c1cccc(F)c1] |
| 90 | 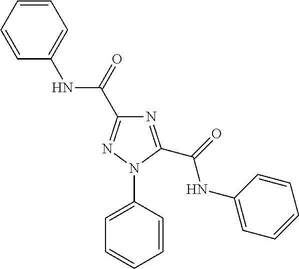 [SMILES: O=C(Nc1ccccc1)c1nc(C(=O)Nc2ccccc2)n(-c2ccccc2)n1] |
| 91 | 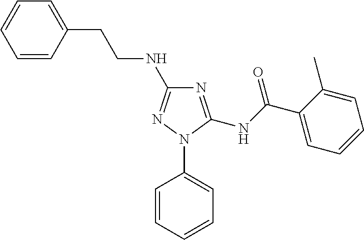 [SMILES: Cc1ccccc1C(=O)Nc1nc(NCCc2ccccc2)nn1-c1ccccc1] |
| 92 | 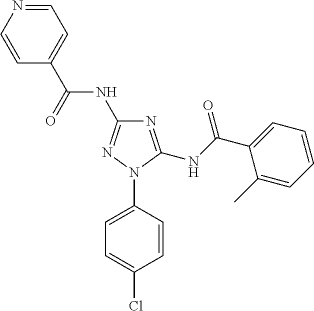 [SMILES: Cc1ccccc1C(=O)Nc1nc(NC(=O)c2ccncc2)nn1-c1ccc(Cl)cc1] |
| 93 | 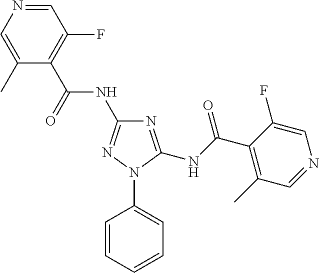 [SMILES: Cc1cncc(F)c1C(=O)Nc1nc(NC(=O)c2c(C)cncc2F)n(-c2ccccc2)n1] |
| 94 | 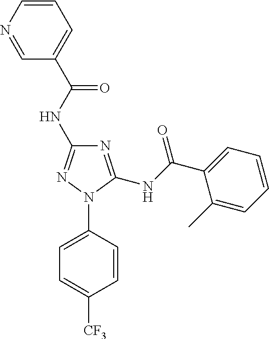 [SMILES: Cc1ccccc1C(=O)Nc1nc(NC(=O)c2cccnc2)nn1-c1ccc(C(F)(F)F)cc1] |
| 95 | 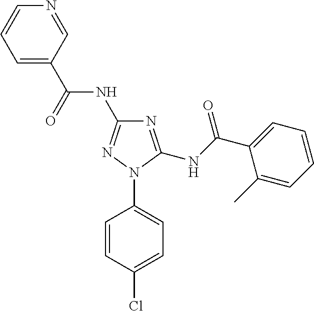 [SMILES: Cc1ccccc1C(=O)Nc1nc(NC(=O)c2cccnc2)nn1-c1ccc(Cl)cc1] |
| 96 | 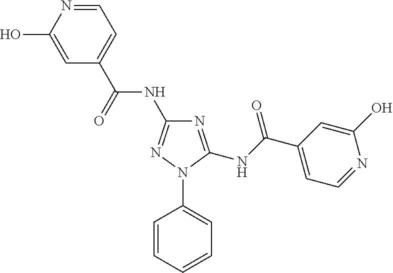 [SMILES: O=C(Nc1nc(NC(=O)c2ccnc(O)c2)n(-c2ccccc2)n1)c1ccnc(O)c1] |
| 97 | 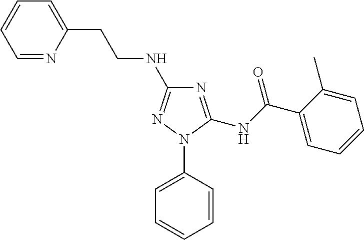 [SMILES: Cc1ccccc1C(=O)Nc1nc(NCCc2ccccn2)nn1-c1ccccc1] |
| 98 | 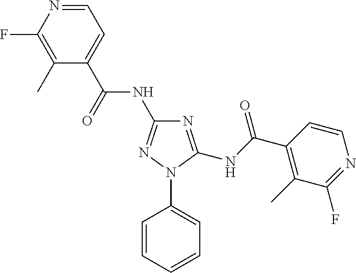 [SMILES: Cc1c(C(=O)Nc2nc(NC(=O)c3ccnc(F)c3C)n(-c3ccccc3)n2)ccnc1F] |
| 99 | 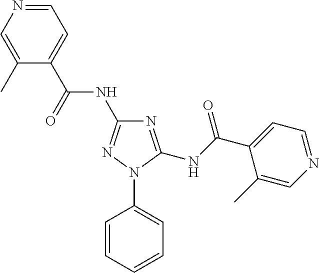 [SMILES: Cc1cnccc1C(=O)Nc1nc(NC(=O)c2ccncc2C)n(-c2ccccc2)n1] |
| 100 | 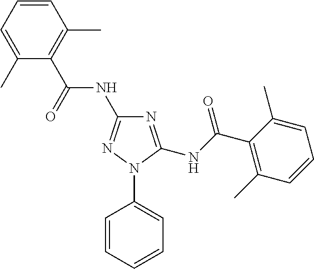 [SMILES: Cc1cccc(C)c1C(=O)Nc1nc(NC(=O)c2c(C)cccc2C)n(-c2ccccc2)n1] |
| 101 | 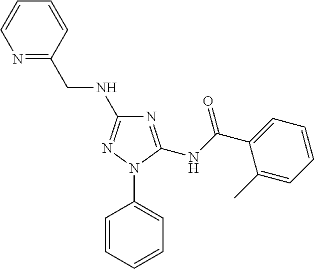 [SMILES: Cc1ccccc1C(=O)Nc1nc(NCc2ccccn2)nn1-c1ccccc1] |
| 102 | 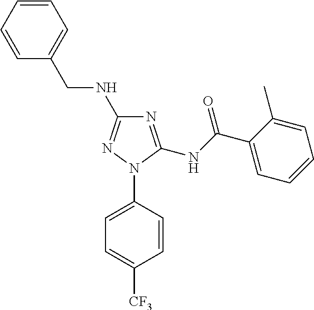 [SMILES: Cc1ccccc1C(=O)Nc1nc(NCc2ccccc2)nn1-c1ccc(C(F)(F)F)cc1] |
| 103 | 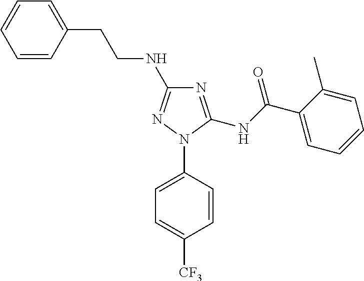 [SMILES: Cc1ccccc1C(=O)Nc1nc(NCCc2ccccc2)nn1-c1ccc(C(F)(F)F)cc1] |
| 104 | 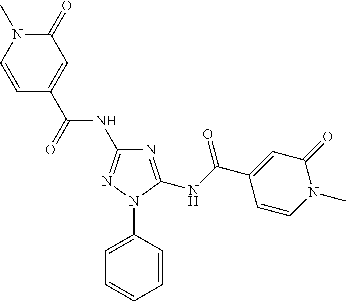 [SMILES: Cn1ccc(C(=O)Nc2nc(NC(=O)c3ccn(C)c(=O)c3)n(-c3ccccc3)n2)cc1=O] |
| 105 | 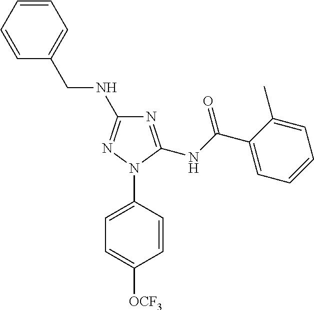 [SMILES: Cc1ccccc1C(=O)Nc1nc(NCc2ccccc2)nn1-c1ccc(OC(F)(F)F)cc1] |
| 106 | 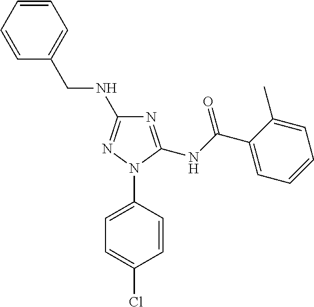 [SMILES: Cc1ccccc1C(=O)Nc1nc(NCc2ccccc2)nn1-c1ccc(Cl)cc1] |
| 107 | 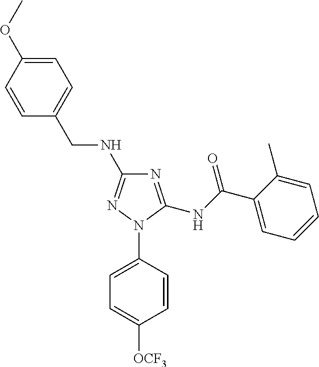 [SMILES: COc1ccc(CNc2nc(NC(=O)c3ccccc3C)n(-c3ccc(OC(F)(F)F)cc3)n2)cc1] |
| 108 | 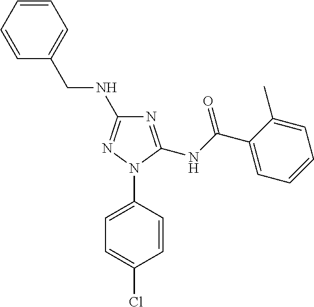 [SMILES: Cc1ccccc1C(=O)Nc1nc(NCc2ccccc2)nn1-c1ccc(Cl)cc1] |
| 109 | 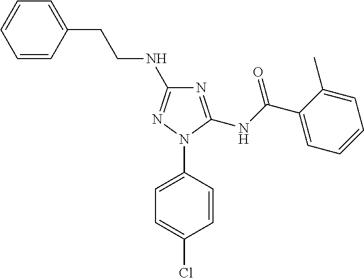 [SMILES: Cc1ccccc1C(=O)Nc1nc(NCCc2ccccc2)nn1-c1ccc(Cl)cc1] |
| 110 | 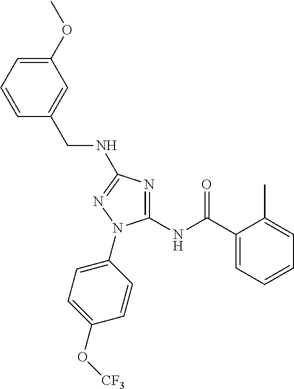 [SMILES: COc1cccc(CNc2nc(NC(=O)c3ccccc3C)n(-c3ccc(OC(F)(F)F)cc3)n2)c1] |
| 111 | 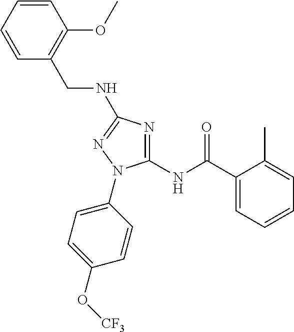 [SMILES: COc1ccccc1CNc1nc(NC(=O)c2ccccc2C)n(-c2ccc(OC(F)(F)F)cc2)n1] |
| 112 | 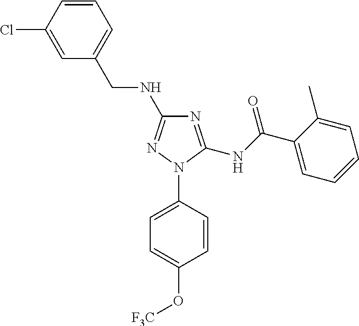 [SMILES: Cc1ccccc1C(=O)Nc1nc(NCc2cccc(Cl)c2)nn1-c1ccc(OC(F)(F)F)cc1] |
| 113 | 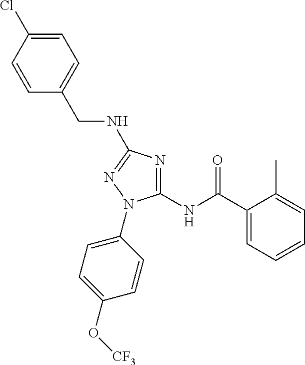 [SMILES: Cc1ccccc1C(=O)Nc1nc(NCc2ccc(Cl)cc2)nn1-c1ccc(OC(F)(F)F)cc1] |
| 114 | 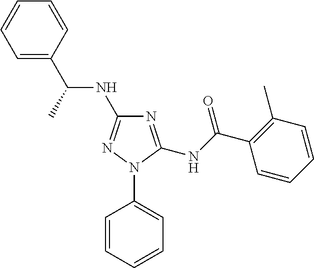 [SMILES: Cc1ccccc1C(=O)Nc1nc(N[C@H](C)c2ccccc2)nn1-c1ccccc1] |
| 115 | 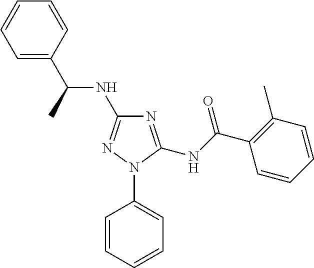 [SMILES: Cc1ccccc1C(=O)Nc1nc(N[C@@H](C)c2ccccc2)nn1-c1ccccc1] |
| 116 | 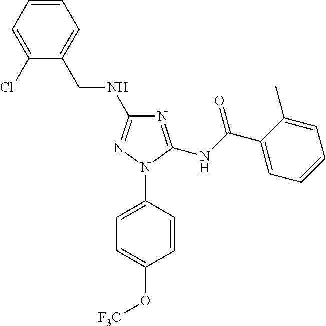 [SMILES: Cc1ccccc1C(=O)Nc1nc(NCc2ccccc2Cl)nn1-c1ccc(OC(F)(F)F)cc1] |
| 117 | 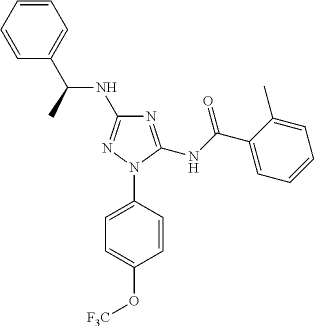 [SMILES: Cc1ccccc1C(=O)Nc1nc(N[C@@H](C)c2ccccc2)nn1-c1ccc(OC(F)(F)F)cc1] |
| 118 | 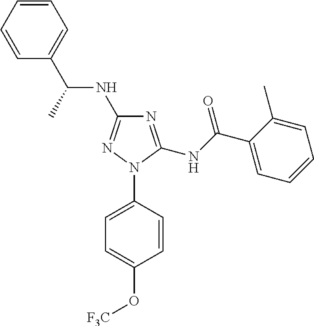 [SMILES: Cc1ccccc1C(=O)Nc1nc(N[C@H](C)c2ccccc2)nn1-c1ccc(OC(F)(F)F)cc1] |
| 119 | 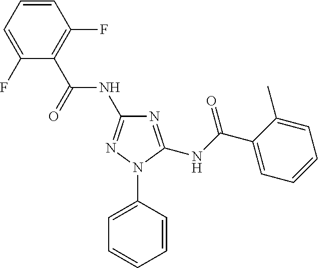 [SMILES: Cc1ccccc1C(=O)Nc1nc(NC(=O)c2c(F)cccc2F)nn1-c1ccccc1] |
| 120 | 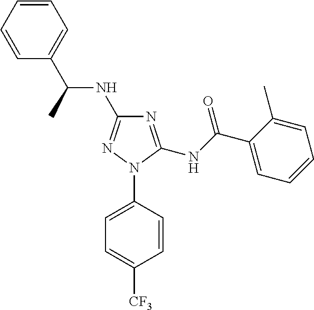 [SMILES: Cc1ccccc1C(=O)Nc1nc(N[C@@H](C)c2ccccc2)nn1-c1ccc(C(F)(F)F)cc1] |
| 121 | 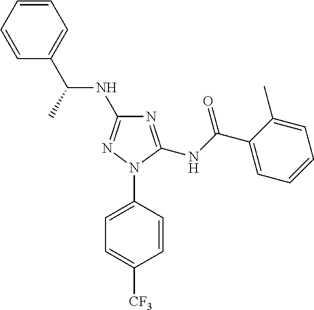 [SMILES: Cc1ccccc1C(=O)Nc1nc(N[C@H](C)c2ccccc2)nn1-c1ccc(C(F)(F)F)cc1] |
| 122 | 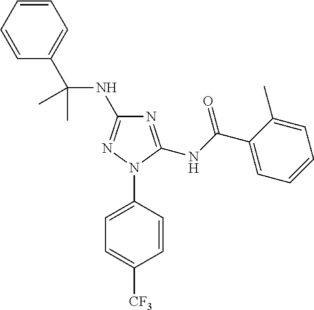 [SMILES: Cc1ccccc1C(=O)Nc1nc(NC(C)(C)c2ccccc2)nn1-c1ccc(C(F)(F)F)cc1] |
| 149 | 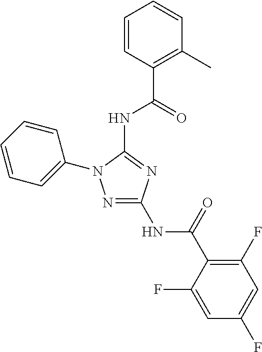 [SMILES: Cc1ccccc1C(=O)Nc1nc(NC(=O)c2c(F)cc(F)cc2F)nn1-c1ccccc1] |
| 150 | 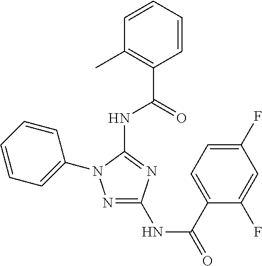 [SMILES: Cc1ccccc1C(=O)Nc1nc(NC(=O)c2ccc(F)cc2F)nn1-c1ccccc1] |
| 151 | 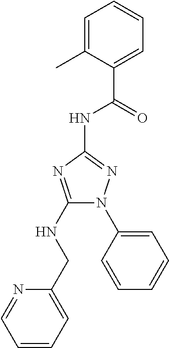 [SMILES: Cc1ccccc1C(=O)Nc1nc(NCc2ccccn2)n(-c2ccccc2)n1] |
| 152 | 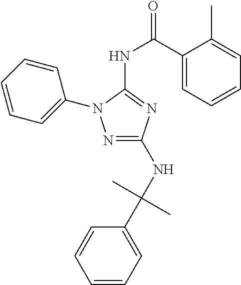 [SMILES: Cc1ccccc1C(=O)Nc1nc(NC(C)(C)c2ccccc2)nn1-c1ccccc1] |
| 153 | 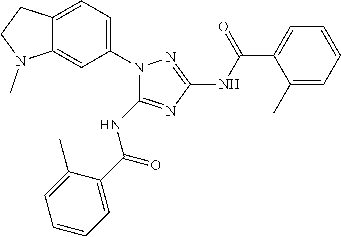 [SMILES: Cc1ccccc1C(=O)Nc1nc(NC(=O)c2ccccc2C)n(-c2ccc3c(c2)N(C)CC3)n1] |

In one embodiment, the compound of formula (I) or (IA′) is

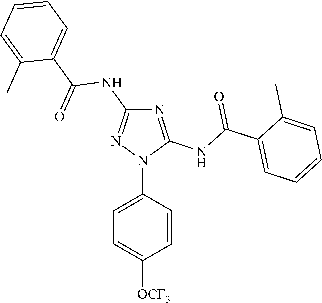 [SMILES: Cc1ccccc1C(=O)Nc1nc(NC(=O)c2ccccc2C)n(-c2ccc(OC(F)(F)F)cc2)n1]

or a pharmaceutically acceptable salt thereof.

In one embodiment, the compound of formula (I), (IA), (IB), (IC), (ID), (IE), (IF), or (IG) is selected from Table 1A below, or a pharmaceutically acceptable salt or solvate thereof.

TABLE 1A

| | |
|---|---|
| 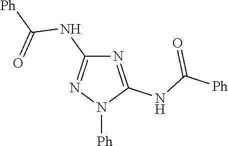 [SMILES: O=C(Nc1nc(NC(=O)c2ccccc2)n(-c2ccccc2)n1)c1ccccc1] | A |
| 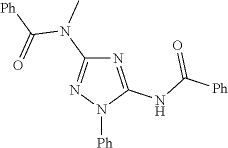 [SMILES: CN(C(=O)c1ccccc1)c1nc(NC(=O)c2ccccc2)n(-c2ccccc2)n1] | G |
| 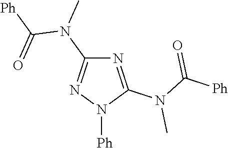 [SMILES: CN(C(=O)c1ccccc1)c1nc(N(C)C(=O)c2ccccc2)n(-c2ccccc2)n1] | J |
| 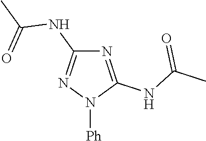 [SMILES: CC(=O)Nc1nc(NC(C)=O)n(-c2ccccc2)n1] | B |
| 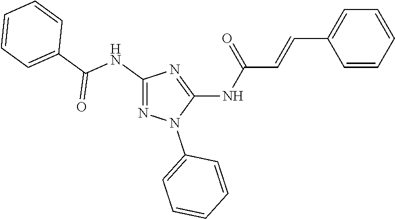 [SMILES: O=C(/C=C/c1ccccc1)Nc1nc(NC(=O)c2ccccc2)nn1-c1ccccc1] | C |
| 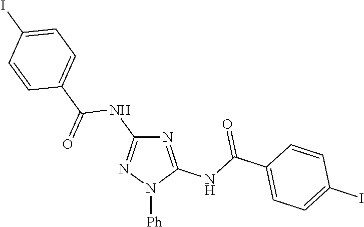 [SMILES: O=C(Nc1nc(NC(=O)c2ccc(I)cc2)n(-c2ccccc2)n1)c1ccc(I)cc1] | D |
| 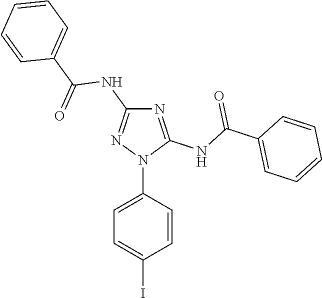 [SMILES: O=C(Nc1nc(NC(=O)c2ccccc2)n(-c2ccc(I)cc2)n1)c1ccccc1] | E |
| 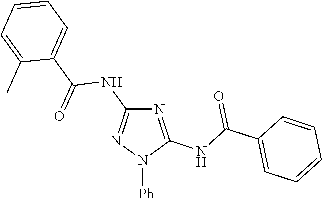 [SMILES: Cc1ccccc1C(=O)Nc1nc(NC(=O)c2ccccc2)n(-c2ccccc2)n1] | H |
| 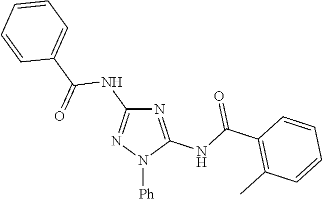 [SMILES: Cc1ccccc1C(=O)Nc1nc(NC(=O)c2ccccc2)nn1-c1ccccc1] | K |
| 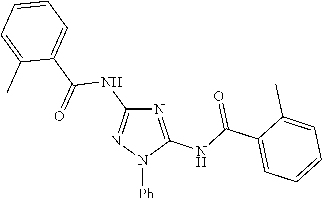 [SMILES: Cc1ccccc1C(=O)Nc1nc(NC(=O)c2ccccc2C)n(-c2ccccc2)n1] | F |
| 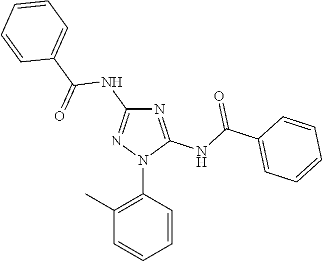 [SMILES: Cc1ccccc1-n1nc(NC(=O)c2ccccc2)nc1NC(=O)c1ccccc1] | M |
| 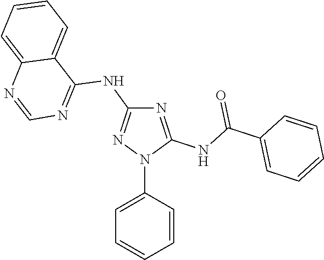 [SMILES: O=C(Nc1nc(Nc2ncnc3ccccc23)nn1-c1ccccc1)c1ccccc1] | I |
| 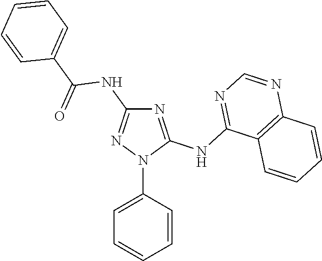 [SMILES: O=C(Nc1nc(Nc2ncnc3ccccc23)n(-c2ccccc2)n1)c1ccccc1] | L |
| 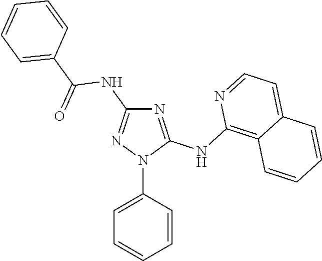 [SMILES: O=C(Nc1nc(Nc2nccc3ccccc23)n(-c2ccccc2)n1)c1ccccc1] | U |
| 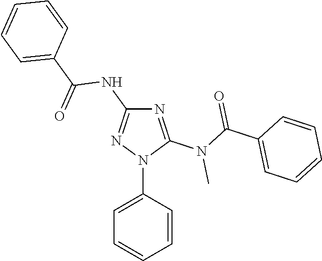 [SMILES: CN(C(=O)c1ccccc1)c1nc(NC(=O)c2ccccc2)nn1-c1ccccc1] | V |
| 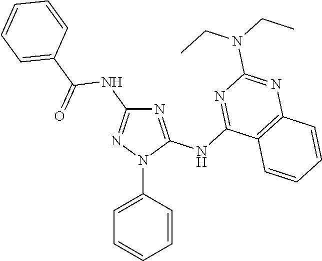 [SMILES: CCN(CC)c1nc(Nc2nc(NC(=O)c3ccccc3)nn2-c2ccccc2)c2ccccc2n1] | W |
| 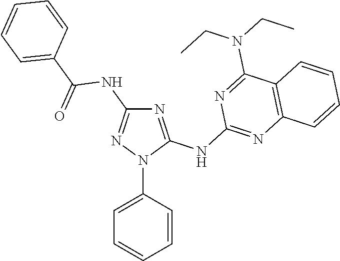 [SMILES: CCN(CC)c1nc(Nc2nc(NC(=O)c3ccccc3)nn2-c2ccccc2)nc2ccccc12] | X |
| 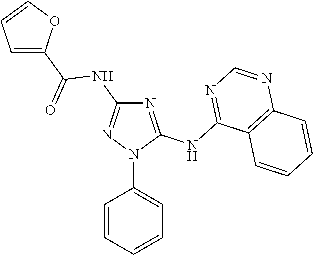 [SMILES: O=C(Nc1nc(Nc2ncnc3ccccc23)n(-c2ccccc2)n1)c1ccco1] | Z |
| 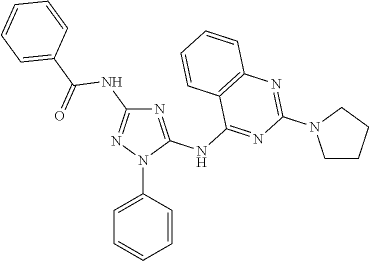 [SMILES: O=C(Nc1nc(Nc2nc(N3CCCC3)nc3ccccc23)n(-c2ccccc2)n1)c1ccccc1] | A1 |
| 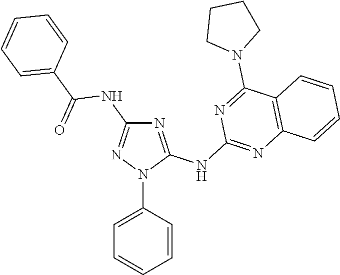 [SMILES: O=C(Nc1nc(Nc2nc(N3CCCC3)c3ccccc3n2)n(-c2ccccc2)n1)c1ccccc1] | B1 |
| 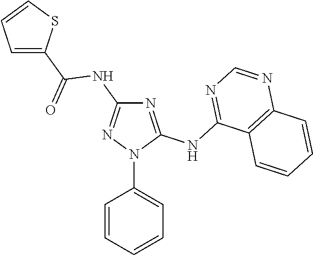 [SMILES: O=C(Nc1nc(Nc2ncnc3ccccc23)n(-c2ccccc2)n1)c1cccs1] | C1 |
| 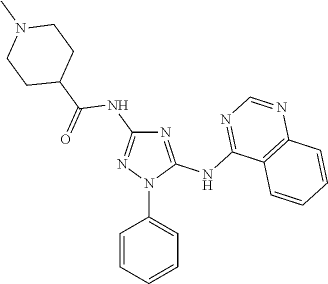 [SMILES: CN1CCC(C(=O)Nc2nc(Nc3ncnc4ccccc34)n(-c3ccccc3)n2)CC1] | D1 |
| 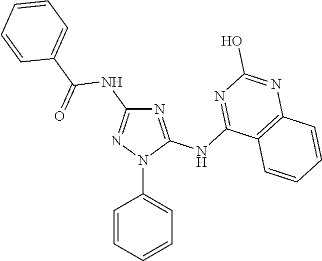 [SMILES: O=C(Nc1nc(Nc2nc(O)nc3ccccc23)n(-c2ccccc2)n1)c1ccccc1] | E1 |
| 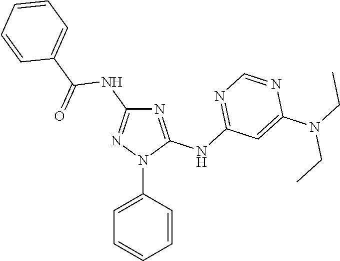 [SMILES: CCN(CC)c1cc(Nc2nc(NC(=O)c3ccccc3)nn2-c2ccccc2)ncn1] | F1 |
| 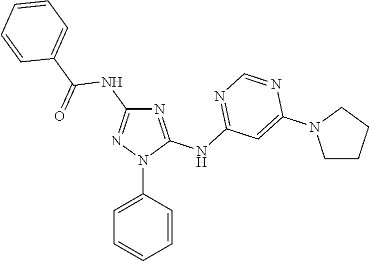 [SMILES: O=C(Nc1nc(Nc2cc(N3CCCC3)ncn2)n(-c2ccccc2)n1)c1ccccc1] | G1 |
| 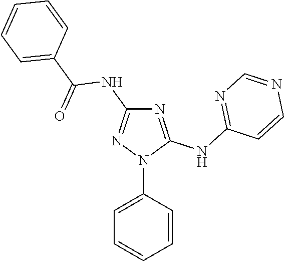 [SMILES: O=C(Nc1nc(Nc2ccncn2)n(-c2ccccc2)n1)c1ccccc1] | H1 |
| 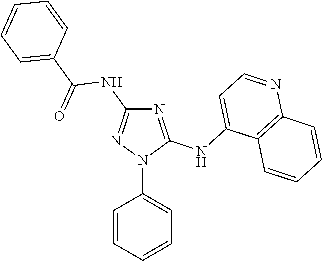 [SMILES: O=C(Nc1nc(Nc2ccnc3ccccc23)n(-c2ccccc2)n1)c1ccccc1] | I1 |
| 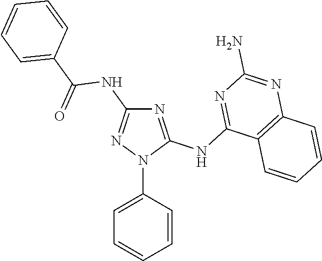 [SMILES: Nc1nc(Nc2nc(NC(=O)c3ccccc3)nn2-c2ccccc2)c2ccccc2n1] | J1 |
| 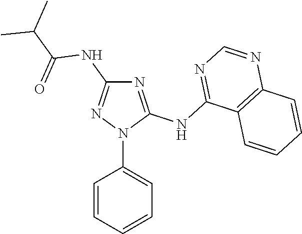 [SMILES: CC(C)C(=O)Nc1nc(Nc2ncnc3ccccc23)n(-c2ccccc2)n1] | K1 |
| 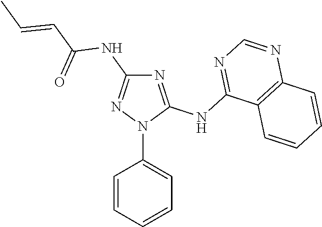 [SMILES: C/C=C/C(=O)Nc1nc(Nc2ncnc3ccccc23)n(-c2ccccc2)n1] | L1 |
| 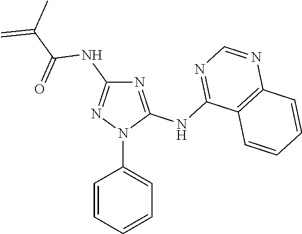 [SMILES: C=C(C)C(=O)Nc1nc(Nc2ncnc3ccccc23)n(-c2ccccc2)n1] | M1 |
| 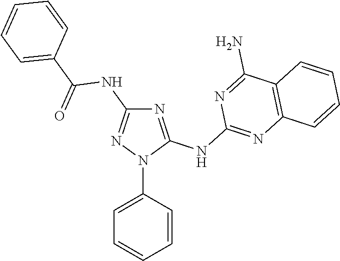 [SMILES: Nc1nc(Nc2nc(NC(=O)c3ccccc3)nn2-c2ccccc2)nc2ccccc12] | N1 |
| 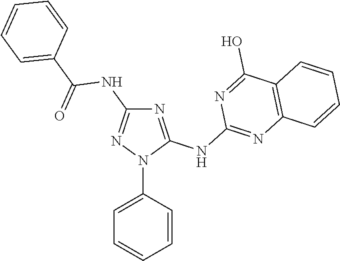 [SMILES: O=C(Nc1nc(Nc2nc(O)c3ccccc3n2)n(-c2ccccc2)n1)c1ccccc1] | O1 |
| 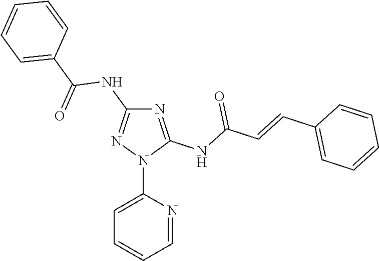 [SMILES: O=C(/C=C/c1ccccc1)Nc1nc(NC(=O)c2ccccc2)nn1-c1ccccn1] | U1 |
| 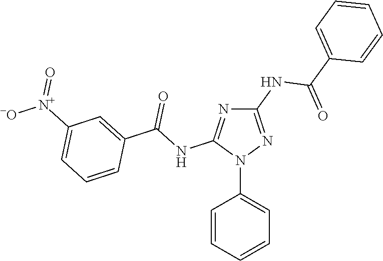 | V1 |
| 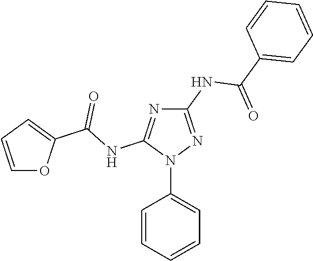 [SMILES: O=C(Nc1nc(NC(=O)c2ccco2)n(-c2ccccc2)n1)c1ccccc1] | W1 |
| 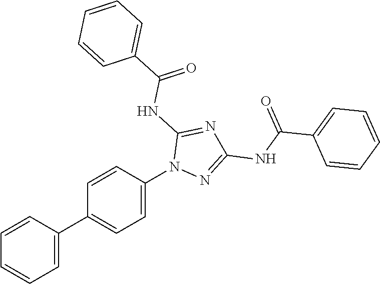 [SMILES: O=C(Nc1nc(NC(=O)c2ccccc2)n(-c2ccc(-c3ccccc3)cc2)n1)c1ccccc1] | X1 |
| 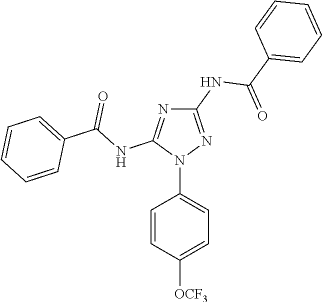 [SMILES: O=C(Nc1nc(NC(=O)c2ccccc2)n(-c2ccc(OC(F)(F)F)cc2)n1)c1ccccc1] | Z1 |
| 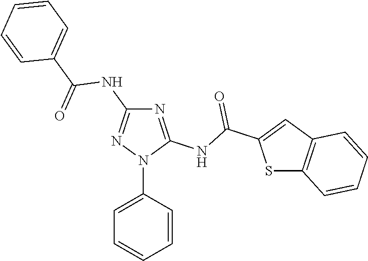 [SMILES: O=C(Nc1nc(NC(=O)c2cc3ccccc3s2)n(-c2ccccc2)n1)c1ccccc1] | A2 |
| 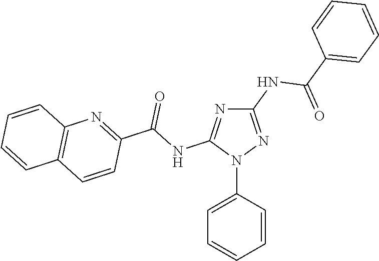 [SMILES: O=C(Nc1nc(NC(=O)c2ccc3ccccc3n2)n(-c2ccccc2)n1)c1ccccc1] | B2 |
| 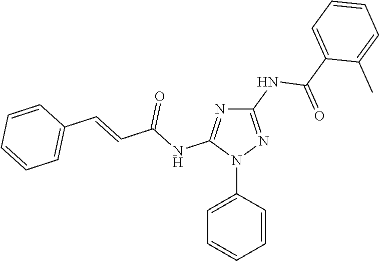 [SMILES: Cc1ccccc1C(=O)Nc1nc(NC(=O)/C=C/c2ccccc2)n(-c2ccccc2)n1] | C2 |
| 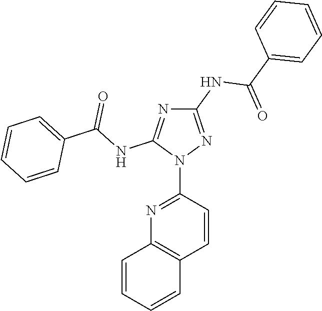 [SMILES: O=C(Nc1nc(NC(=O)c2ccccc2)n(-c2ccc3ccccc3n2)n1)c1ccccc1] | D2 |
| 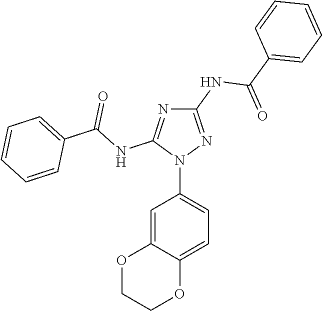 [SMILES: O=C(Nc1nc(NC(=O)c2ccccc2)n(-c2ccc3c(c2)OCCO3)n1)c1ccccc1] | E2 |
| 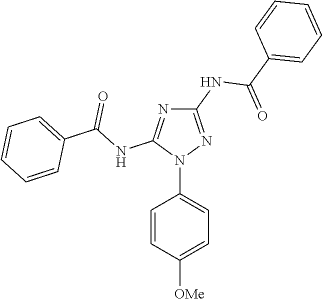 [SMILES: COc1ccc(-n2nc(NC(=O)c3ccccc3)nc2NC(=O)c2ccccc2)cc1] | F2 |
| 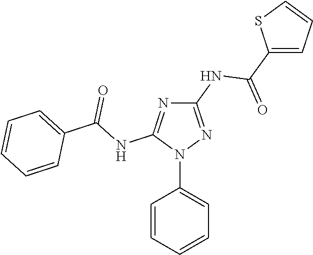 [SMILES: O=C(Nc1nc(NC(=O)c2cccs2)nn1-c1ccccc1)c1ccccc1] | G2 |
| 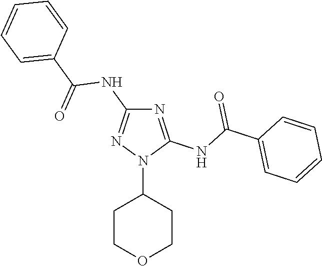 [SMILES: O=C(Nc1nc(NC(=O)c2ccccc2)n(C2CCOCC2)n1)c1ccccc1] | I2 |
| 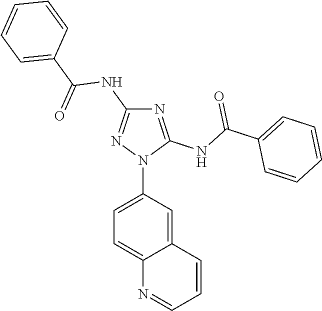 [SMILES: O=C(Nc1nc(NC(=O)c2ccccc2)n(-c2ccc3ncccc3c2)n1)c1ccccc1] | J2 |
| 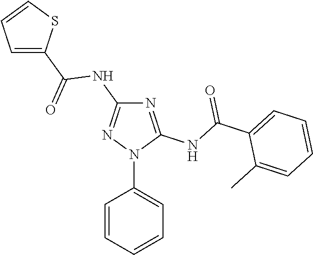 [SMILES: Cc1ccccc1C(=O)Nc1nc(NC(=O)c2cccs2)nn1-c1ccccc1] | K2 |
| 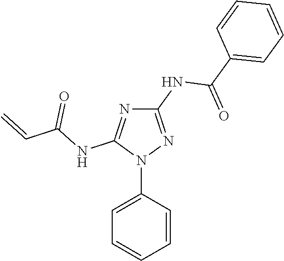 [SMILES: C=CC(=O)Nc1nc(NC(=O)c2ccccc2)nn1-c1ccccc1] | L2 |
| 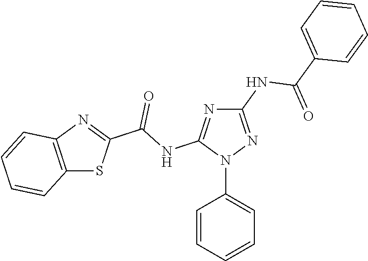 [SMILES: O=C(Nc1nc(NC(=O)c2nc3ccccc3s2)n(-c2ccccc2)n1)c1ccccc1] | M2 |
| 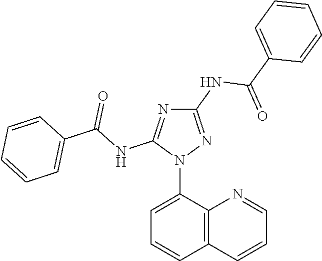 [SMILES: O=C(Nc1nc(NC(=O)c2ccccc2)n(-c2cccc3cccnc23)n1)c1ccccc1] | O2 |
| 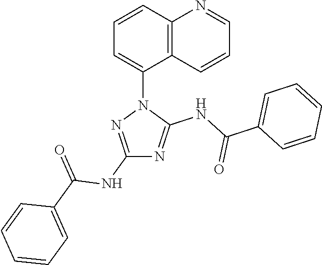 [SMILES: O=C(Nc1nc(NC(=O)c2ccccc2)n(-c2cccc3ncccc23)n1)c1ccccc1] | P2 |
| 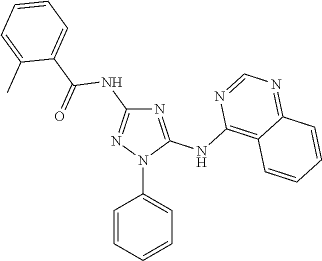 [SMILES: Cc1ccccc1C(=O)Nc1nc(Nc2ncnc3ccccc23)n(-c2ccccc2)n1] | R2 |
| 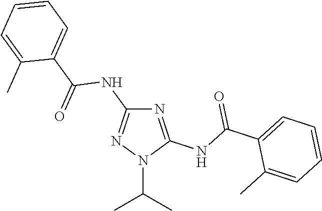 [SMILES: Cc1ccccc1C(=O)Nc1nc(NC(=O)c2ccccc2C)n(C(C)C)n1] | W2 |
| 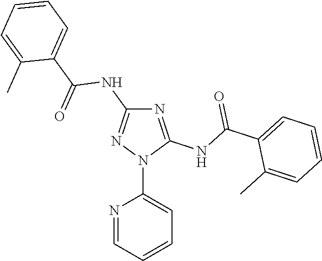 [SMILES: Cc1ccccc1C(=O)Nc1nc(NC(=O)c2ccccc2C)n(-c2ccccn2)n1] | X2 |
| 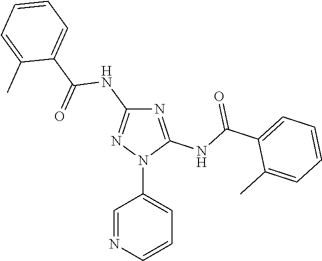 [SMILES: Cc1ccccc1C(=O)Nc1nc(NC(=O)c2ccccc2C)n(-c2cccnc2)n1] | Y2 |
| 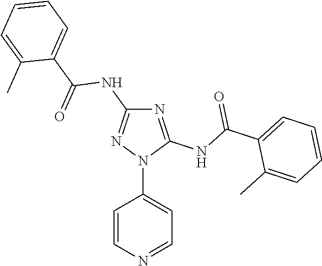 [SMILES: Cc1ccccc1C(=O)Nc1nc(NC(=O)c2ccccc2C)n(-c2ccncc2)n1] | B3 |
| 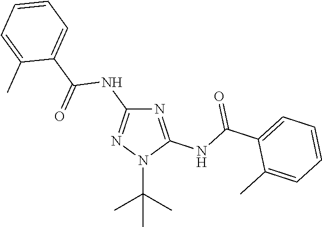 [SMILES: Cc1ccccc1C(=O)Nc1nc(NC(=O)c2ccccc2C)n(C(C)(C)C)n1] | D3 |
| 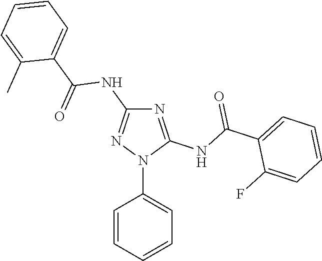 [SMILES: Cc1ccccc1C(=O)Nc1nc(NC(=O)c2ccccc2F)n(-c2ccccc2)n1] | E3 |
| 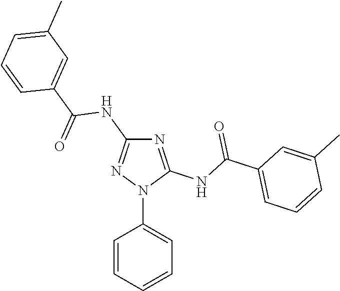 [SMILES: Cc1cccc(C(=O)Nc2nc(NC(=O)c3cccc(C)c3)n(-c3ccccc3)n2)c1] | F3 |
| 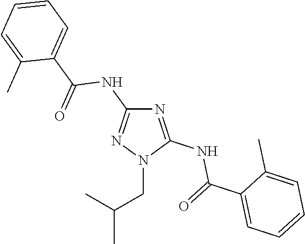 [SMILES: Cc1ccccc1C(=O)Nc1nc(NC(=O)c2ccccc2C)n(CC(C)C)n1] | I3 |
| 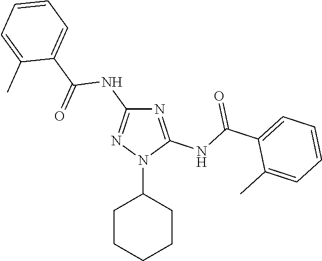 [SMILES: Cc1ccccc1C(=O)Nc1nc(NC(=O)c2ccccc2C)n(C2CCCCC2)n1] | J3 |
| 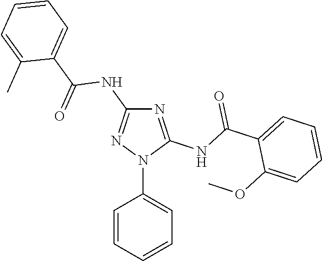 [SMILES: COc1ccccc1C(=O)Nc1nc(NC(=O)c2ccccc2C)nn1-c1ccccc1] | K3 |
| 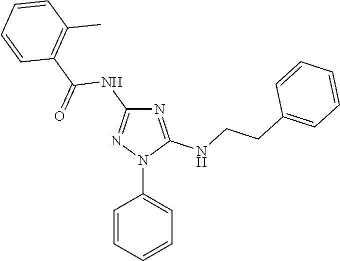 [SMILES: Cc1ccccc1C(=O)Nc1nc(NCCc2ccccc2)n(-c2ccccc2)n1] | L3 |
| 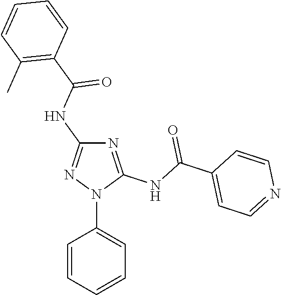 [SMILES: Cc1ccccc1C(=O)Nc1nc(NC(=O)c2ccncc2)n(-c2ccccc2)n1] | M3 |
| 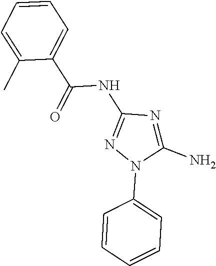 [SMILES: Cc1ccccc1C(=O)Nc1nc(N)n(-c2ccccc2)n1] | N3 |
| 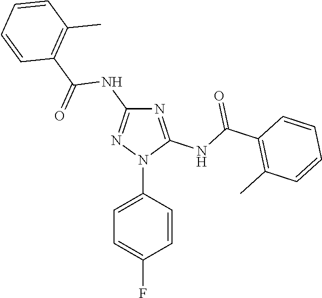 [SMILES: Cc1ccccc1C(=O)Nc1nc(NC(=O)c2ccccc2C)n(-c2ccc(F)cc2)n1] | O3 |
| 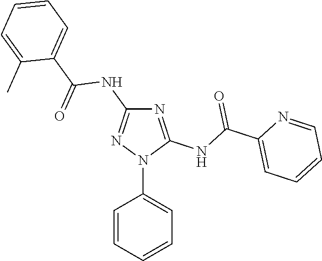 [SMILES: Cc1ccccc1C(=O)Nc1nc(NC(=O)c2ccccn2)n(-c2ccccc2)n1] | P3 |
| 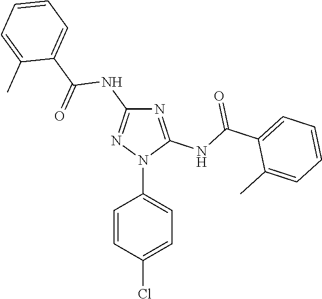 [SMILES: Cc1ccccc1C(=O)Nc1nc(NC(=O)c2ccccc2C)n(-c2ccc(Cl)cc2)n1] | Q3 |

In one embodiment, the compound of formula (I), (IA), (IB), (IC), (ID), (IE), (IF), or (IG) is

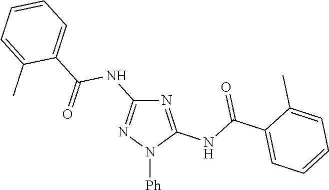 [SMILES: Cc1ccccc1C(=O)Nc1nc(NC(=O)c2ccccc2C)n(-c2ccccc2)n1]

or a pharmaceutically acceptable salt thereof.

In one embodiment, the compound of formula (I), (IA), (IB), (IC), (ID), (IE), (IF), and/or (IG) exclude

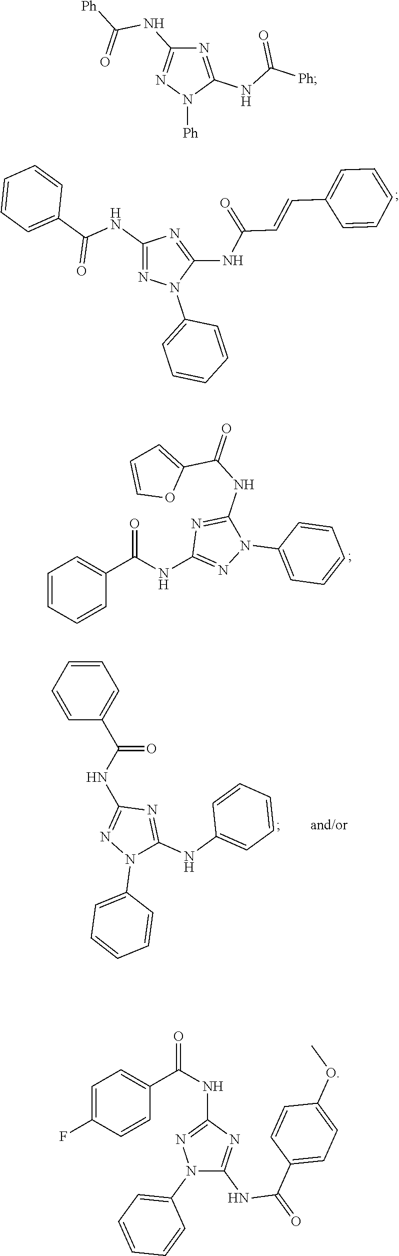

In one embodiment, the compound of the present disclosure is selected from Table 2, below, or a pharmaceutically acceptable salt or solvate thereof.

TABLE 2

| | |
|---|---|
| 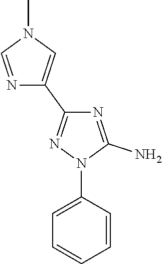 [SMILES: Cn1cnc(-c2nc(N)n(-c3ccccc3)n2)c1] | 147 |
| 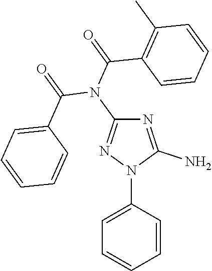 [SMILES: Cc1ccccc1C(=O)N(C(=O)c1ccccc1)c1nc(N)n(-c2ccccc2)n1] | 148 |

In one embodiment, the present disclosure provides a pharmaceutical composition comprising a pharmaceutically acceptable carrier or a pharmaceutically acceptable excipient and a compound of any one of formula (I), (I′), (IA′), (IA), (IB), (IC), (ID) (IE), (IF), and/or (IG).

In one embodiment, the pharmaceutical composition comprises

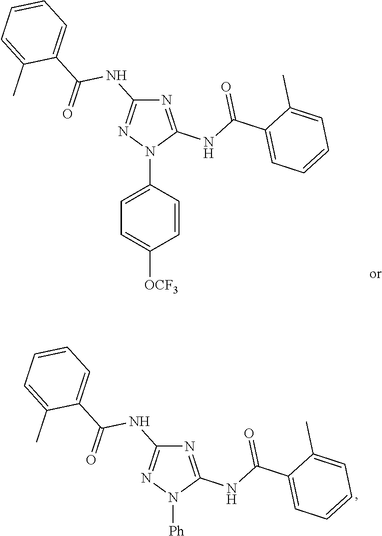 [SMILES: Cc1ccccc1C(=O)Nc1nc(NC(=O)c2ccccc2C)n(-c2ccc(OC(F)(F)F)cc2)n1 ; Cc1ccccc1C(=O)Nc1nc(NC(=O)c2ccccc2C)n(-c2ccccc2)n1]

or a pharmaceutically acceptable salt thereof.

In one embodiment, the present disclosure provides a pharmaceutical composition comprising a pharmaceutically acceptable carrier or a pharmaceutically acceptable excipient and a compound from Tables 1, 1A, or 2.

In one embodiment, the present disclosure provides compounds comprising the structure of formula (IIA):

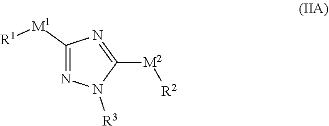

or a pharmaceutically acceptable salt or solvate thereof, wherein:

M¹ and M² are each independently selected from a bond, —NR⁴—, or —NR⁴C(O)—, —C(O)NR⁴—;

R¹ and R² are each independently selected from an alkyl, alkenyl, cycloalkyl, aryl, biphenyl, heterocyclyl heterocycloalkyl, heteroaryl, cycloalkylalkyl, arylalkyl, arylalkenyl, arylalkynyl, heterocyclylalkyl, heteroarylalkyl, heteroarylalkenyl, or heteroarylalkynyl, wherein each cycloalkyl, aryl, heteroaryl, and heterocyclyl portion is optionally substituted with one or more R⁵;

wherein at least one of M¹ and M² is a bond or —NR⁴—;

wherein when M¹ is —NR⁴—, then R¹ is cycloalkylalkyl, heterocyclylalkyl, arylalkyl, or heteroarylalkyl, wherein cycloalkyl, aryl, heteroaryl, and heterocyclyl portion is optionally substituted with one or more R⁵;

wherein when M² is —NR⁴—, then R² is cycloalkylalkyl, heterocyclylalkyl, arylalkyl, or heteroarylalkyl, wherein cycloalkyl, aryl, heteroaryl, and heterocyclyl portion is optionally substituted with one or more R⁵;

R³ is selected from an alkyl, alkenyl, cycloalkyl, aryl, biphenyl, heterocyclyl heterocycloalkyl, heteroaryl, cycloalkylalkyl, arylalkyl, arylalkenyl, arylalkynyl, heterocyclylalkyl, heteroarylalkyl, heteroarylalkenyl, or heteroarylalkynyl, wherein each cycloalkyl, aryl, heteroaryl, and heterocyclyl portion is optionally substituted with one or more R⁷;

R⁴ is each independently H, alkyl, wherein each alkyl is optionally substituted with one or more R⁵;

R⁵ is each independently I, Br, Cl, F, —CH₂F, —CHF₂, —CF₃, —OCF₃, —CN, -alkylene-CN, —CONH₂, —CONHR⁶, —CONR⁶R⁶, —COOH, —NH₂, -alkylene-NH², —NHR⁶, -alkylene-NHR⁶, —NO₂, —NR⁶R⁶, -alkylene-NR⁶R⁶, —N₃, —OH, OR⁶, —COOR⁶, —OSO₃R⁶, oxo, R⁶, —SH, —SO₂R⁶, —SO₃H, —SO₃R⁶, —SR⁶, heterocyclyl, or -alkylene-heterocyclyl, wherein heterocyclyl is optionally substituted with one or more R⁸;

R⁶ is each independently alkyl, alkenyl, alkynyl, haloalkyl, haloalkenyl, haloalkynyl; or alternatively two R⁶ on the same N atom can together form a 3-6 membered N-heterocyclyl;

R⁷ is each independently I, Br, Cl, F, —CH₂F, —CHF₂, —CF₃, —OCF₃, —CN, -alkylene-CN, —CONH₂, —CONHR⁶, —CONR⁶R⁶, —COOH, —NH₂, -alkylene-NH², —NHR⁶, -alkylene-NHR⁶, —NO₂, —NR⁶R⁶, -alkylene-NR⁶R⁶, —N₃, —OH, OR⁶, —COOR⁶, —OSO₃R⁶, oxo, R⁶, —SH, —SO₂R⁶, —SO₃H, —SO₃R⁶, —SR⁶, cycloalkyl, heterocyclyl, aryl, or heteroaryl, wherein each cycloalkyl, heterocyclyl, aryl, and heteroaryl is optionally substituted with one or more R⁵; and

wherein the compound is not N-benzyl-N-(5-(benzylamino)-1-phenyl-1H-1,2,4-triazol-3-yl)acetamide, N-(5-((2-chlorobenzyl)amino)-1-phenyl-1H-1,2,4-triazol-3-yl)-2-fluorobenzamide and N³,N⁵-bis(4-methylbenzyl)-1-phenyl-1H-1,2,4-triazole-3,5-diamine.

In one embodiment, the present disclosure provides compounds comprising the structure of formula (II):

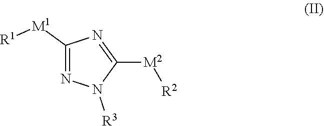

or a pharmaceutically acceptable salt or solvate thereof, wherein:

M¹ and M² are each independently selected from a bond, —NR⁴—, or —NR⁴C(O)—, —C(O)NR⁴—;

R¹ and R+ are each independently selected from an alkyl, alkenyl, cycloalkyl, aryl, biphenyl, heterocyclyl heterocycloalkyl, heteroaryl, cycloalkylalkyl, arylalkyl, arylalkenyl, arylalkynyl, heterocyclylalkyl, heteroarylalkyl, heteroarylalkenyl, or heteroarylalkynyl, wherein each cycloalkyl, aryl, heteroaryl, and heterocyclyl portion is optionally substituted with one or more R⁵;

wherein at least one of M¹ and M² is a bond or —NR⁴—;

wherein when M¹ is —NR⁴—, then R¹ is cycloalkylalkyl, heterocyclylalkyl, arylalkyl, or heteroarylalkyl, wherein cycloalkyl, aryl, heteroaryl, and heterocyclyl portion is optionally substituted with one or more R⁵;

wherein when M² is —NR⁴—, then R² is cycloalkylalkyl, heterocyclylalkyl, arylalkyl, or heteroarylalkyl, wherein cycloalkyl, aryl, heteroaryl, and heterocyclyl portion is optionally substituted with one or more R5;

R³ is selected from an alkyl, alkenyl, cycloalkyl, aryl, biphenyl, heterocyclyl heterocycloalkyl, heteroaryl, cycloalkylalkyl, arylalkyl, arylalkenyl, arylalkynyl, heterocyclylalkyl, heteroarylalkyl, heteroarylalkenyl, or heteroarylalkynyl, wherein each cycloalkyl, aryl, heteroaryl, and heterocyclyl portion is optionally substituted with one or more R7;

R⁴ is each independently H, alkyl, wherein each alkyl is optionally substituted with one or more R⁵;

R⁵ is each independently I, Br, Cl, F, —CH2F, —CHF2, —CF3, —OCF3, —CN, -alkyl-CN, —CONH2, —CONHR6, —CONR6R6, —COOH, —NH₂, —NHR⁶, —NO₂, —NR⁶R⁶, —N₃, —OH, OR⁶, —COOR⁶, —OSO₃R⁶, oxo, R⁶, —SH, —SO₂R⁶, —SO₃H, —SO₃R⁶, or —SR⁶;

R⁶ is each independently alkyl, alkenyl, alkynyl, haloalkyl, haloalkenyl, haloalkynyl; or alternatively two R⁶ on the same N atom can together form a 3-6 membered N-heterocyclyl; and

R⁷ is each independently I, Br, Cl, F, —CH₂F, —CHF₂, —CF₃, —OCF₃, —CN, -alkyl-CN, —CONH₂, —CONHR6, —CONR⁶R⁶, —COOH, —NH₂, —NHR⁶, —NO₂, —NR⁶R⁶, —N₃, —OH, OR⁶, —COOR⁶, —OSO₃R⁶, oxo, R⁶, —SH, —SO₂R⁶, —SO₃H, —SO₃R⁶, —SR⁶, cycloalkyl, heterocyclyl, aryl, or heteroaryl, wherein each cycloalkyl, heterocyclyl, aryl, and heteroaryl is optionally substituted with one or more R⁵;

wherein the compound is not N-benzyl-N-(5-(benzylamino)-1-phenyl-1H-1,2,4-triazol-3-yl)acetamide, N-(5-((2-chlorobenzyl)amino)-1-phenyl-1H-1,2,4-triazol-3-yl)-2-fluorobenzamide and N3,N5-bis(4-methylbenzyl)-1-phenyl-1H-1,2,4-triazole-3,5-diamine.

In one embodiment of the compound of formula (II) or (IIA), at least one of -M¹-R¹ and -M²-R² is —NR⁴—(C₁-C₃ alkylene)-cycloalkyl, —NR⁴—(C₁-C₃ alkylene)-heterocyclyl, —NR⁴—(C₁-C₃ alkylene)-aryl, or —NR⁴—(C₁-C₃ alkylene)-heteroaryl; wherein cycloalkyl, aryl, heteroaryl, and heterocyclyl is each optionally substituted with one or more R⁵.

In one embodiment of the compound of formula (II) or (IIA), at least one of -M¹-R¹ and -M²-R² is —NR⁴—(C₁-C₃ alkylene)-phenyl, or —NR⁴—(C₁-C₃ alkylene)-pyridyl, wherein phenyl and pyridyl is each optionally substituted with one or more R⁵. In other embodiments, at least one of -M¹-R¹ and -M²-R² is —NR⁴—CH₂-phenyl, —NR⁴—CH₂CH₂-phenyl, —NR⁴—CH₂-pyridyl, —NR⁴—CH₂CH₂-pyridyl,

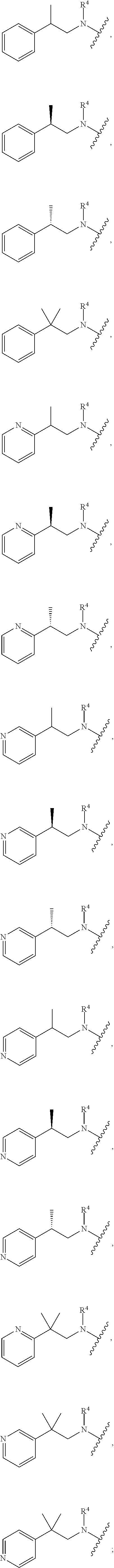

wherein phenyl and pyridyl is each optionally substituted with one or more R⁵. In some embodiments, -M¹-R¹ and -M²-R² are each selected from —NR⁴—CH₂-phenyl, —NR⁴—CH₂CH₂-phenyl, —NR⁴—CH₂-pyridyl, —NR⁴—CH₂CH₂-pyridyl,

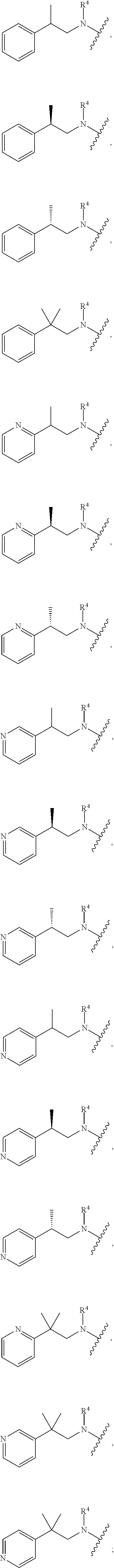

wherein phenyl and pyridyl is each optionally substituted with one or more R⁵.

In one embodiment of the compound of formula (II) or (IIA), -M¹-R¹ is —NR⁴C(O)R¹. In one embodiment of the compound of formula (II), -M²-R² is —NR⁴C(O)R².

In one embodiment of the compound of formula (II) or (IIA), R¹ and R² are each independently selected from phenyl or pyridyl, each optionally substituted with one or more R⁵.

In one embodiment of the compound of formula (II) or (IIA), one of M¹ and M² is a bond.

In one embodiment of the compound of formula (II) or (IIA), R¹ and R² are each independently selected from phenyl or 5-6 membered heteroaryl, each optionally substituted with one or more R⁵. In other embodiments, R¹ and R² are each independently selected from phenyl, azetidinyl, pyrrolidinyl, piperidinyl, imidazolyl, isoxazolyl, oxazolyl, thiazolyl, pyridyl, pyridazinyl, pyrazinyl, pyrimidinyl, pyridinoneor, or pyridine N-oxide, each optionally substituted with one or more R⁵.

In one embodiment of the compound of formula (II) or (IIA), M¹ is a bond and R¹ is pyridyl, optionally substituted with one or more R⁵.

In one embodiment of the compound of formula (II) or (IIA), M² is a bond and R² is pyridyl, optionally substituted with one or more R⁵.

In one embodiment of the compound of formula (II) or (IIA), M¹ is a bond and M² is —NR⁴— or —NR⁴C(O)—.

In one embodiment of the compound of formula (II) or (IIA), M² is a bond and M¹ is —NR⁴— or —NR⁴C(O)—.

In one embodiment of the compound of formula (II) or (IIA), R³ is phenyl, optionally substituted with one or more R⁷; and R⁷ is each independently I, Br, Cl, F, —CH₂F, —CHF₂, —CF₃, —OCF₃, —CN, —N₃, —OH, methyl, ethyl, propyl, —C≡CH; —CN, —NH₂, —NO₂, —N₃, —OH, —OCF₃, —OMe, —NMe₂, or —NEt₂.

In one embodiment of the compound of formula (II) or (IIA), R⁵ and R⁷ are each independently selected from I, Br, Cl, F, —CH₂F, —CHF₂, —CF₃, —OCF₃, —N₃, —CN, —OH, methyl, ethyl, propyl, —C≡CH; —CN, —NH₂, —NO₂, —N₃, —OH, —OCF₃, —OMe, —NMe₂, or —NEt₂.

In one embodiment of the compound of formula (II) or (IIA), at least one R⁵ is selected from -alkylene-NH₂, -alkylene-NHR⁶, or -alkylene-NR⁶R⁶.

In one embodiment of the compound of formula (II) or (IIA), at least one R⁷ is a 4-7 membered heterocyclyl, optionally substituted with one or more R⁵.

In one embodiment of the compound of formula (II) or (IIA), R⁴ is each independently H or C₁-C₃ alkyl.

In one embodiment of the compound of formula (II) or (IIA), the compound is selected from Table 3 below, or a pharmaceutically acceptable salt or solvate thereof.

TABLE 3

| | |
|---|---|
| 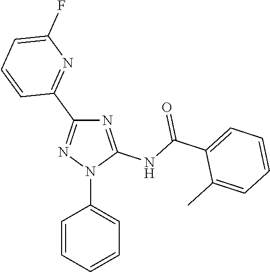 [SMILES: Cc1ccccc1C(=O)Nc1nc(-c2cccc(F)n2)nn1-c1ccccc1] | 123 |
| 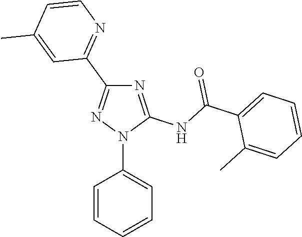 [SMILES: Cc1ccnc(-c2nc(NC(=O)c3ccccc3C)n(-c3ccccc3)n2)c1] | 124 |
| 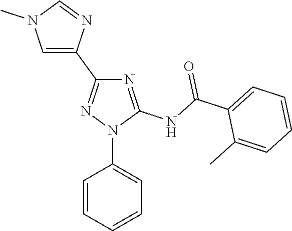 [SMILES: Cc1ccccc1C(=O)Nc1nc(-c2cn(C)cn2)nn1-c1ccccc1] | 125 |
| 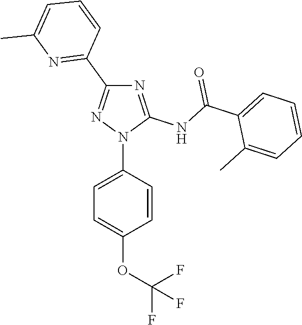 [SMILES: Cc1cccc(-c2nc(NC(=O)c3ccccc3C)n(-c3ccc(OC(F)(F)F)cc3)n2)n1] | 126 |
| 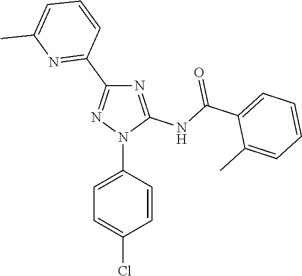 [SMILES: Cc1cccc(-c2nc(NC(=O)c3ccccc3C)n(-c3ccc(Cl)cc3)n2)n1] | 127 |
| 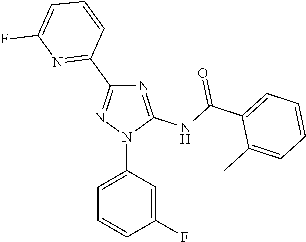 [SMILES: Cc1ccccc1C(=O)Nc1nc(-c2cccc(F)n2)nn1-c1cccc(F)c1] | 128 |
| 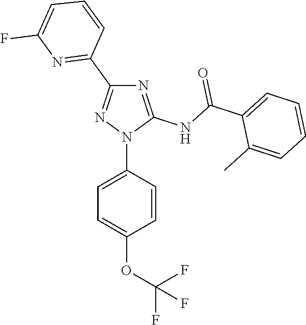 [SMILES: Cc1ccccc1C(=O)Nc1nc(-c2cccc(F)n2)nn1-c1ccc(OC(F)(F)F)cc1] | 129 |
| 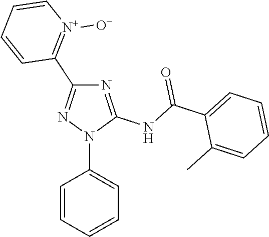 [SMILES: Cc1ccccc1C(=O)Nc1nc(-c2cccc[n+]2[O-])nn1-c1ccccc1] | 130 |
| 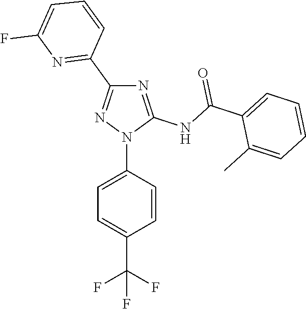 [SMILES: Cc1ccccc1C(=O)Nc1nc(-c2cccc(F)n2)nn1-c1ccc(C(F)(F)F)cc1] | 131 |
| 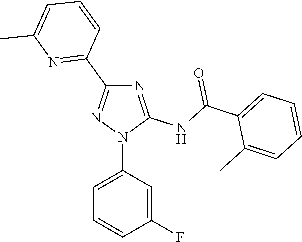 [SMILES: Cc1cccc(-c2nc(NC(=O)c3ccccc3C)n(-c3cccc(F)c3)n2)n1] | 132 |
| 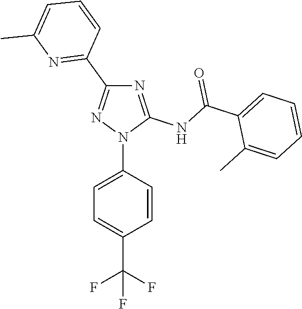 [SMILES: Cc1cccc(-c2nc(NC(=O)c3ccccc3C)n(-c3ccc(C(F)(F)F)cc3)n2)n1] | 133 |
| 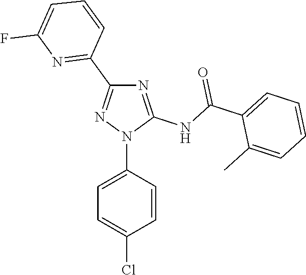 [SMILES: Cc1ccccc1C(=O)Nc1nc(-c2cccc(F)n2)nn1-c1ccc(Cl)cc1] | 134 |
| 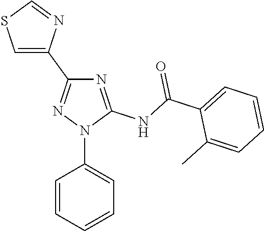 [SMILES: Cc1ccccc1C(=O)Nc1nc(-c2cscn2)nn1-c1ccccc1] | 135 |
| 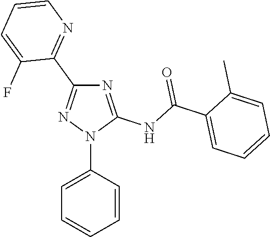 [SMILES: Cc1ccccc1C(=O)Nc1nc(-c2ncccc2F)nn1-c1ccccc1] | 136 |
| 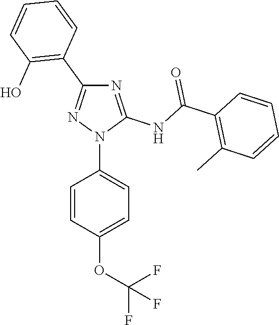 [SMILES: Cc1ccccc1C(=O)Nc1nc(-c2ccccc2O)nn1-c1ccc(OC(F)(F)F)cc1] | 137 |
| 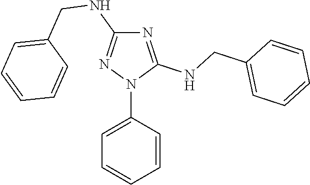 [SMILES: c1ccc(CNc2nc(NCc3ccccc3)n(-c3ccccc3)n2)cc1] | 138 |
| 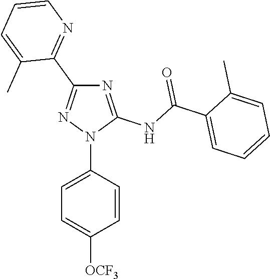 [SMILES: Cc1ccccc1C(=O)Nc1nc(-c2ncccc2C)nn1-c1ccc(OC(F)(F)F)cc1] | 139 |
| 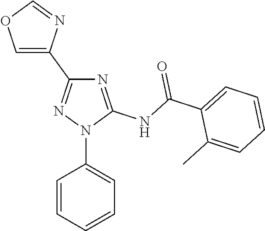 [SMILES: Cc1ccccc1C(=O)Nc1nc(-c2cocn2)nn1-c1ccccc1] | 140 |
| 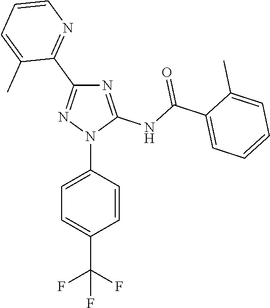 [SMILES: Cc1ccccc1C(=O)Nc1nc(-c2ncccc2C)nn1-c1ccc(C(F)(F)F)cc1] | 141 |
| 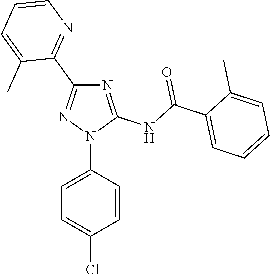 [SMILES: Cc1ccccc1C(=O)Nc1nc(-c2ncccc2C)nn1-c1ccc(Cl)cc1] | 142 |
| 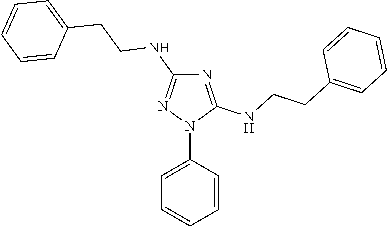 [SMILES: c1ccc(CCNc2nc(NCCc3ccccc3)n(-c3ccccc3)n2)cc1] | 143 |
| 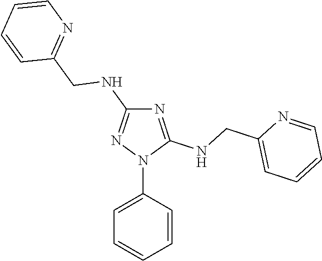 [SMILES: c1ccc(-n2nc(NCc3ccccn3)nc2NCc2ccccn2)cc1] | 144 |
| 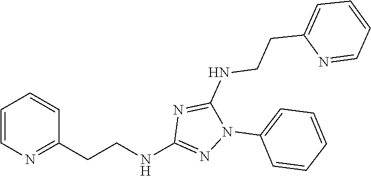 [SMILES: c1ccc(-n2nc(NCCc3ccccn3)nc2NCCc2ccccn2)cc1] | 145 |
| 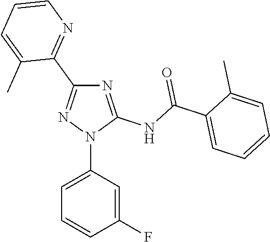 [SMILES: Cc1ccccc1C(=O)Nc1nc(-c2ncccc2C)nn1-c1cccc(F)c1] | 146 |
| 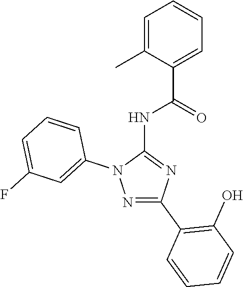 [SMILES: Cc1ccccc1C(=O)Nc1nc(-c2ccccc2O)nn1-c1cccc(F)c1] | |
| 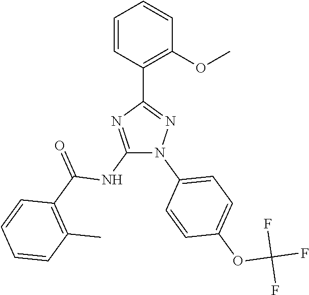 [SMILES: COc1ccccc1-c1nc(NC(=O)c2ccccc2C)n(-c2ccc(OC(F)(F)F)cc2)n1] | 154 |
| 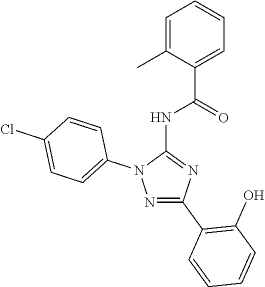 [SMILES: Cc1ccccc1C(=O)Nc1nc(-c2ccccc2O)nn1-c1ccc(Cl)cc1] | 155 |
| 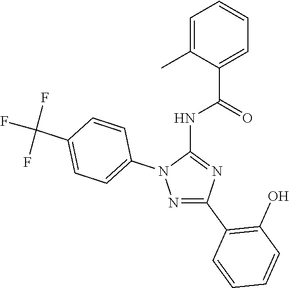 [SMILES: Cc1ccccc1C(=O)Nc1nc(-c2ccccc2O)nn1-c1ccc(C(F)(F)F)cc1] | 156 |
| 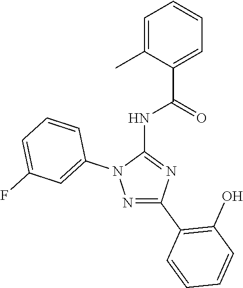 [SMILES: Cc1ccccc1C(=O)Nc1nc(-c2ccccc2O)nn1-c1cccc(F)c1] | 157 |
| 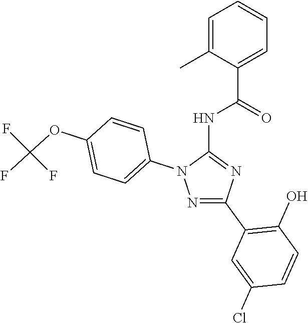 [SMILES: Cc1ccccc1C(=O)Nc1nc(-c2cc(Cl)ccc2O)nn1-c1ccc(OC(F)(F)F)cc1] | 158 |
| 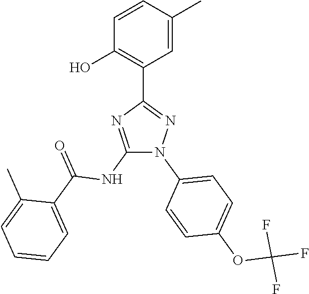 [SMILES: Cc1ccc(O)c(-c2nc(NC(=O)c3ccccc3C)n(-c3ccc(OC(F)(F)F)cc3)n2)c1] | 159 |

In one embodiment, the present disclosure provides compounds having the structure of formula (II′):

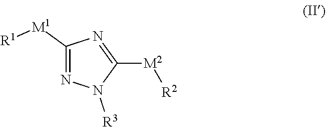

or a pharmaceutically acceptable salt or solvate thereof, wherein:

M¹ and M² are each independently selected tram a bond, —NR⁴—, —NR⁴C(O)—; —C(O)NR⁴—, provided that M¹ and M² are not both —NR⁴— or both a bond;

R¹ and R² are each independently selected from a cycloalkyl aryl, heterocyclyl or heteroaryl wherein each cycloalkyl, aryl, heteroaryl and heterocyclyl is optionally substituted with one or more R^{5a}, provided that R¹ and R² are not 1,3-dioxoisoindolin-2-yl;

R³ is selected from an alkyl, alkenyl, cycloalkyl, aryl, heterocyclyl, or heteroaryl, wherein each cycloalkyl, aryl, heteroaryl, and heterocyclyl is optionally substituted with one or more R^{5a};

R⁴ is each independently H or alkyl;

R^{5a} is each independently I, Br, Cl, F, CN, NH₂, NHR^{6a}, NO₂, NR^{6a}R^{6a}, OH, OR^{6a}, or R^{6a}; and

R^{6a} is each independently alkyl or haloalkyl; or alternatively two R^{6a} on the same N atom can together form a 3-6 membered N-heterocyclyl.

In one embodiment, M¹ in formula (II′) is a bond. In another embodiment, M² in formula (II′) is a bond. In one embodiment, M¹ in formula (II′) is a bond and M² is —NR⁴— or —NR⁴C(O)—. In one embodiment, M² in formula (II′) is a bond and M¹ is —NR⁴— or —NR⁴C(O)—.

In one embodiment, R⁴ at each occurrence in the definition of M¹ or M² is independently H or C1-C3 alkyl.

In one embodiment, M¹ and M² in formula (II′) are each independently selected from —NH—, —N(CH₃)—, —NHC(O)—, or —N(CH₃)C(O)—.

In one embodiment, R¹, R², and R³ in formula (II′) are each independently selected from phenyl, 5-10 membered heteroaryl, or 5-10 membered heterocyclyl, wherein each aryl, heteroaryl and heterocyclyl is optionally substituted with one or more R^{5a}.

In one embodiment of the compound of formula (II′), R¹ is an aryl, optionally substituted with one or more R^{5a}. In another embodiment, R¹ is a phenyl, optionally substituted with one or more R^{5a}. In one embodiment, R¹ is an unsubstituted phenyl. In some embodiments, R¹ is a heteroaryl, optionally substituted with one or more R^{5a}. In some embodiments, R¹ is a pyridyl, optionally substituted with one or more R^{5a}. In some embodiments, R¹ is a pyrimidinyl, optionally substituted with one or more R^{5a}. In one embodiment, R¹ is a bicyclic heteroaryl, optionally substituted with one or more R^{5a}. In another embodiment, R¹ is

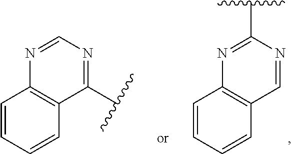 [SMILES: *c1ncnc2ccccc12 ; *c1ncc2ccccc2n1]

optionally substituted with one or more R^{5a}.

In some embodiments of the compound of formula (II′), R¹ is a heterocyclyl, optionally substituted with one or more R^{5a}. In some embodiments, R¹ is a tetrahydropyranyl, optionally substituted with one or more R^{5a}.

In one embodiment of the compound of formula (II′), R² is an aryl, optionally substituted with one or more R^{5a}. In another embodiment, R² is a phenyl, optionally substituted with one or more R^{5a}. In one embodiment, R² is an unsubstituted phenyl. In some embodiments, R² is a heteroaryl, optionally substituted with one or more R^{5a}. In some embodiments, R² is a pyridyl, optionally substituted with one or more R^{5a}. In some embodiments, R² is a pyrimidinyl, optionally substituted with one or more R^{5a}. In one embodiment, R² is a bicyclic heteroaryl, optionally substituted with one or more R^{5a}. In another embodiment, R² is

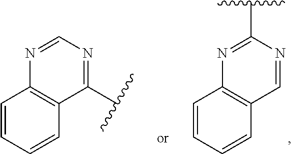 [SMILES: *c1ncnc2ccccc12 ; *c1ncc2ccccc2n1]

optionally substituted with one or more R^{5a}.

In some embodiments of the compound of formula (II′), R² is a heterocyclyl, optionally substituted with one or more R^{5a}. In some embodiments, R² is a tetrahydropyranyl, optionally substituted with one or more R^{5a}.

In one embodiment of the compound of formula (II′), R³ is an aryl, optionally substituted with one or more R^{5a}. In another embodiment, R³ is a phenyl, optionally substituted with one or more R^{5a}. In one embodiment, R³ is an unsubstituted phenyl. In some embodiments, R³ is a pyridyl, optionally substituted with one or more R^{5a}. In some embodiments, R³ is a pyrimidinyl, optionally substituted with one or more R^{5a}. In one embodiment, R³ is a bicyclic heteroaryl, optionally substituted with one or more R^{5a}. In another embodiment, R³ is

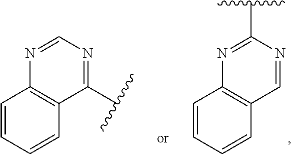 [SMILES: *c1ncnc2ccccc12 ; *c1ncc2ccccc2n1]

optionally substituted with one or more R^{5a}.

In some embodiments of the compound of formula (II′), at least one of R¹ or R² is a pyridyl, optionally substituted with one or more R^{5a}. In other embodiments, at least one of R¹ or R² is a 2-pyridyl, optionally substituted with one or more R^{5a}. In some embodiments, at least one of R¹ or R² is a pyridyl, substituted with at least one methyl and optionally with one or more R^{5a}. In one embodiment, R¹ is a pyridyl, optionally substituted with one or more R^{5a}. In one embodiment, R² is a pyridyl, optionally substituted with one or more R^{5a}.

In some embodiments of the compound of formula (II′), at least one of R¹ or R² is

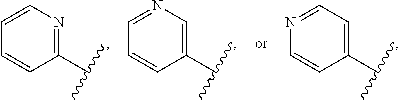

optionally substituted with one or more R^{5a}. In one embodiment, R^{5a} is C1-C3 alkyl. In another embodiment, R^{5a} is methyl.

In some embodiments of the compound of formula (II′), R¹ is

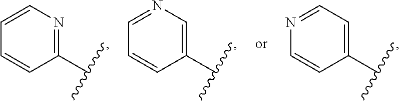

optionally substituted with one or more R^{5a}. In one embodiment, R^{5a} is C1-C3 alkyl. In another embodiment, R^{5a} is methyl. In some embodiments, R² is

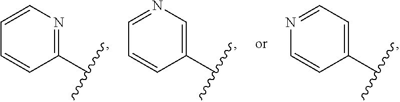

optionally substituted with one or more R^{5a}. In one embodiment, R^{5a} is C1-C3 alkyl. In another embodiment, R^{5a} is methyl.

In some embodiments of the compound of formula (II′), at least one of R¹ or R² is

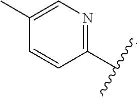 [SMILES: *c1ccc(C)cn1]

In some embodiments, R¹ is

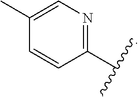 [SMILES: *c1ccc(C)cn1]

In some embodiments, R² is

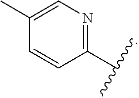 [SMILES: *c1ccc(C)cn1]

In some embodiments of the compound of formula (II′), R³ is a heterocyclyl, optionally substituted with one or more R^{5a}. In some embodiments, R³ is a tetrahydropyranyl, optionally substituted with one or more R^{5a}.

In one embodiment of the compound of formula (II′), R³ is alkyl. In one embodiment, R³ is C1-C6 alkyl. In another embodiment, R³ is methyl, ethyl, n-propyl, isopropyl, n-propyl, i-butyl, sec-butyl, or t-butyl. In one embodiment, R³ is cycloalkyl. In another embodiment, R³ is cyclohexyl.

In one embodiment of the compound of formula (II′), R^{5a} at each occurrence in the definition of R¹, R², or R³ is independently selected from I, Br, Cl, F, or C1-C6 alkyl. In another embodiment, R^{5a} at each occurrence in the definition of R¹, R², or R³ is independently selected from I, Br, Cl, F, or C1-C3 alkyl. In some embodiments, R^{5a} at each occurrence in the definition of R¹, R², or R³ is independently selected from I, Br, Cl, F, or methyl.

In one embodiment of the compound of formula (II′), R^{5a} is each independently —CH₃, I, Br, Cl, F, CN, NH₂, NO₂, OH, OCF₃, OMe, —NMe₂, —NEt₂, or

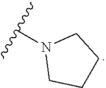 [SMILES: *N1CCCC1]

In one embodiment, the compound of formula (II′) is selected from Table 3A below, or a pharmaceutically acceptable salt or solvate thereof.

TABLE 3A

| | |
|---|---|
| 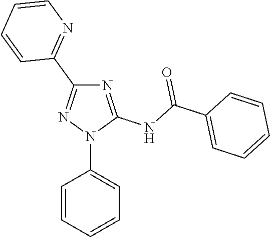 [SMILES: O=C(Nc1nc(-c2ccccn2)nn1-c1ccccc1)c1ccccc1] | T |
| 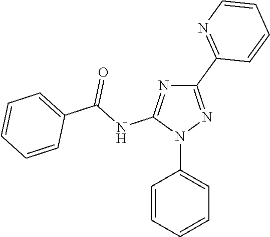 [SMILES: O=C(Nc1nc(-c2ccccn2)nn1-c1ccccc1)c1ccccc1] | Y1 |
| 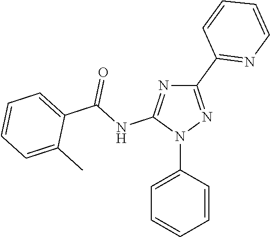 [SMILES: Cc1ccccc1C(=O)Nc1nc(-c2ccccn2)nn1-c1ccccc1] | Q2 |
| 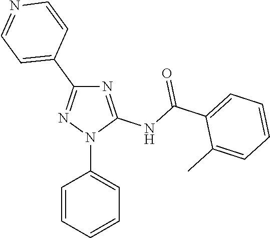 [SMILES: Cc1ccccc1C(=O)Nc1nc(-c2ccncc2)nn1-c1ccccc1] | S2 |
| 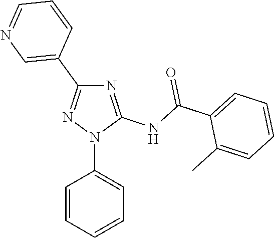 [SMILES: Cc1ccccc1C(=O)Nc1nc(-c2cccnc2)nn1-c1ccccc1] | T2 |
| 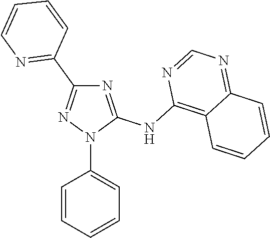 [SMILES: c1ccc(-n2nc(-c3ccccn3)nc2Nc2ncnc3ccccc23)cc1] | U2 |
| 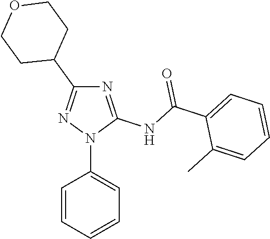 [SMILES: Cc1ccccc1C(=O)Nc1nc(C2CCOCC2)nn1-c1ccccc1] | V2 |
| 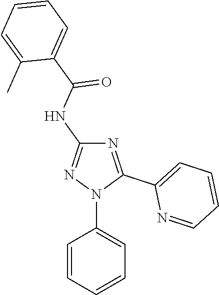 [SMILES: Cc1ccccc1C(=O)Nc1nc(-c2ccccn2)n(-c2ccccc2)n1] | A3 |
| 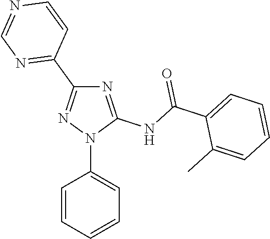 [SMILES: Cc1ccccc1C(=O)Nc1nc(-c2ccncn2)nn1-c1ccccc1] | G3 |
| 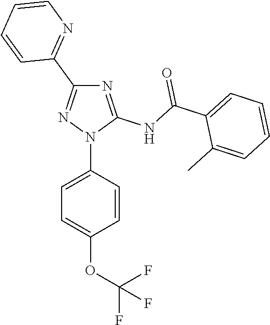 [SMILES: Cc1ccccc1C(=O)Nc1nc(-c2ccccn2)nn1-c1ccc(OC(F)(F)F)cc1] | H3 |
| 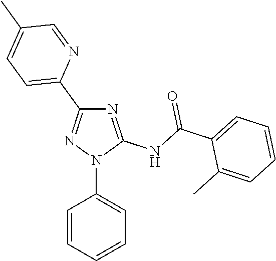 [SMILES: Cc1ccc(-c2nc(NC(=O)c3ccccc3C)n(-c3ccccc3)n2)nc1] | R3 |
| 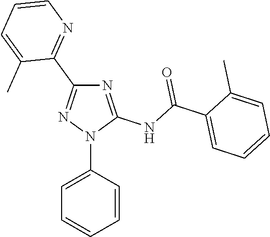 [SMILES: Cc1ccccc1C(=O)Nc1nc(-c2ncccc2C)nn1-c1ccccc1] | S3 |

In one embodiment, the present disclosure provides compounds having the structure of formula (IIB):

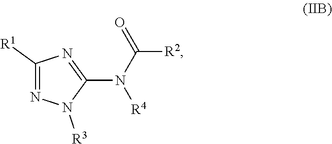

or a pharmaceutically acceptable salt or solvate thereof, wherein:
R¹ and R² are each independently selected from aryl or heteroaryl, each optionally substituted with one or more R⁵;
R³ is selected from aryl or heteroaryl, each optionally substituted with one or more R⁷;
R⁴ is each independently H or —C₁-C₃ alkyl;
R⁵ is I, Br, Cl, F, —CH₂F, —CHF₂, —CF₃, —C₁-C₆ alkyl, alkynyl, —CN, —(C₁-C₃ alkylene)-CN, —NH₂, —(C₁-C₃ alkylene)-NH₂, —(C₁-C₃ alkylene)-NHR⁶, —(C₁-C₃ alkylene)-NR⁶R⁶, —NO₂, —N₃, —OH, —OCF₃, —OMe, —NMe₂, —NEt₂, or —C(O)O(C₁-C₆ alkyl);
R⁶ is each independently alkyl, alkenyl, alkynyl, haloalkyl, haloalkenyl, haloalkynyl; or alternatively two R⁶ on the same N atom can together form a 3-6 membered N-heterocyclyl; and
R⁷ is each independently I, Br, Cl, F, —CH₂F, —CHF₂, —CF₃, —OCF₃, —CN, -alkylene-CN, —CONH₂, —CONHR⁶, —CONR⁶R⁶, —COOH, —NH₂, -alkylene-NH₂, —NHR⁶, -alkylene-NHR⁶, —NO₂, —NR⁶R⁶, -alkylene-NR⁶R⁶, —N₃, —OH, OR⁶, —COOR⁶, —OSO₃R⁶, oxo, R⁶, —SH, —SO₂R⁶, —SO₃H, —SO₃R⁶, —SR⁶, cycloalkyl, heterocyclyl, aryl, or heteroaryl, wherein each cycloalkyl, heterocyclyl, aryl, and heteroaryl is optionally substituted with one or more R⁵

In one embodiment of the compounds of formula (IIA) and/or (IIB), R¹ is phenyl or 5-6 membered heteroaryl, optionally substituted with one or more R⁵. In one embodiment, R¹ is phenyl or pyridyl, optionally substituted with one or more R⁵.

In one embodiment of the compounds of formula (IIA) and/or (IIB), R¹ is phenyl or pyridyl, optionally substituted with one substituent selected from I, Br, Cl, F, —CH₂F, —CHF₂, —CF₃, methyl, —CN, —CH₂—CN, —NH₂, —CH₂—NH₂, —NO₂, —N₃, —OH, —OCF₃, —OMe, —NMe₂, —NEt₂, or —C(O)OMe. In one embodiment, R¹ is

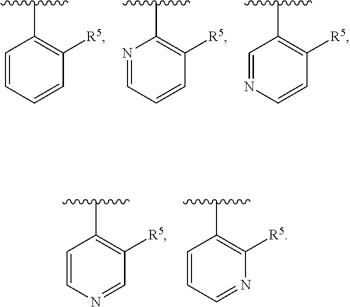

In one embodiment of the compounds of formula (IIA) and/or (IIB), at least one R⁵ is methyl or —OH. In one embodiment, at least one R⁵ substituent on R² is —CH₂—NH₂.

In one embodiment of the compounds of formula (IIA) and/or (IIB), R² is phenyl, optionally substituted with one or more R⁵. In one embodiment, R² is phenyl substituted with at least one R⁵ substituent on R² is I, Br, Cl, F, —CH₂F, —CHF₂, —CF₃, methyl, —CN, —CH₂—CN, —NH₂, —CH₂—NH₂, —NO₂, —N₃, —OH, —OCF₃, —OMe, —NMe₂, —NEt₂, or—C(O)OMe.

In one embodiment of the compounds of formula (IIA) and/or (IIB), R² is

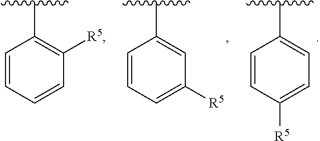

In one embodiment, R² is

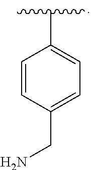 [SMILES: *c1ccc(CN)cc1]

In one embodiment of the compounds of formula (IIA) and/or (IIB),

R³ is

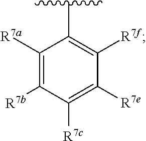

R^{7a}, R^{7b}, R^{7e}, and R^{7f} is each independently H, I, Br, Cl, F, —CH₂F, —CHF₂, —CF₃, —OCF₃, —N₃, —CN, —OH, methyl, ethyl, propyl, C₁-C₃ haloalkyl, C₁-C₃ alkoxy, or C₁-C₃ haloalkoxy;

R^{7c} is H, I, Br, Cl, F, —CH₂F, —CHF₂, —CF₃, —OCF₃, —N₃, —CN, —OH, —C₁-C₃ alkyl, —C₁-C₃ haloalkyl, —C₁-C₃ alkoxy, —C₁-C₃ haloalkoxy, 4-6 membered heterocyclyl, or 5-6 membered heteroaryl, wherein the heterocyclyl and heteroaryl is optionally substituted with one R^{5b};

R^{5b} is I, Br, Cl, F, —CH₂F, —CHF₂, —CF₃, —C₁-C₆ alkyl, alkynyl, —CN, —(C₁-C₃ alkylene)-CN, —NH₂, —(C₁-C₃ alkylene)-NH₂, —(C₁-C₃ alkylene)-NHR⁶, —(C₁-C₃ alkylene)-NR⁶R⁶, —NO₂. —N₃, —OH, —OCF₃, —OMe, —NMe₂, —NEt₂, or—C(O)O(C₁-C₆ alkyl); and

wherein at least one of R^{7a}, R^{7b}, R^{7c}, R^{7e}, and R^{7f} is not H.

In one embodiment of the compounds of formula (IIA) and/or (IIB), R³ is

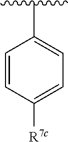

and R^{7c} is H, I, Br, Cl, F, —CH₂F, —CHF₂, —CF₃, —OCF₃, —N₃, —CN, —OH, —C₁-C₃ alkyl, —C₁-C₃ haloalkyl, —C₁-C₃ alkoxy, —C₁-C₃ haloalkoxy, or 4-6 membered heterocyclyl, wherein the heterocyclyl optionally substituted with one R^{5b}.

In one embodiment of the compounds of formula (IIA) and/or (IIB), R^{7c} is —OCF₃. In one embodiment, R^{7c} is 4-6 membered heterocyclyl, wherein the heterocyclyl optionally substituted with one R^{5b}. In one embodiment, R^{7c} is 6 membered heterocyclyl, wherein the heterocyclyl optionally substituted with one R^{5b}. In one embodiment, R^{7c} is piperazinyl, optionally substituted with one R^{5b}. In one embodiment, R^{7c} is

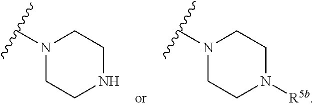

In one embodiment, the compound of formula (IIA) and/or (IIB) is selected from Table 3B below, or a pharmaceutically acceptable salt or solvate thereof.

TABLE 3B

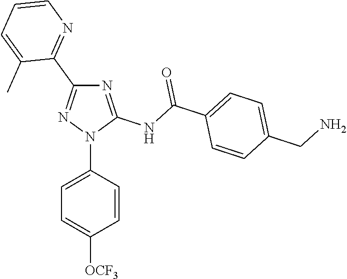 [SMILES: Cc1cccnc1-c1nc(NC(=O)c2ccc(CN)cc2)n(-c2ccc(OC(F)(F)F)cc2)n1]

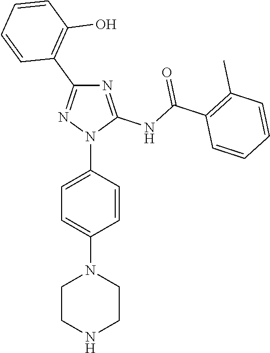 [SMILES: Cc1ccccc1C(=O)Nc1nc(-c2ccccc2O)nn1-c1ccc(N2CCNCC2)cc1]

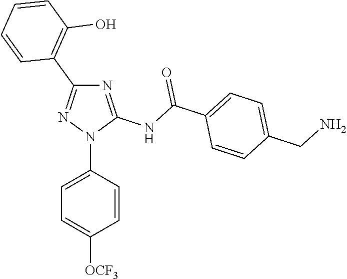 [SMILES: NCc1ccc(C(=O)Nc2nc(-c3ccccc3O)nn2-c2ccc(OC(F)(F)F)cc2)cc1]

In one embodiment, the present disclosure provides a pharmaceutical composition comprising a pharmaceutically acceptable carries or a pharmaceutically acceptable excipient and a compound of say one of formula (II), (II′), and/or (IIA)-(IIB).

In one embodiment the present disclosure provide compounds composing the structure of formula (III):

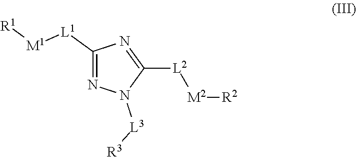

of a pharmaceutically acceptable salt or solvate thereof wherein:
L¹, L² and are each independently selected from a bond, alky lens, or alkenylene:
M¹ and M² are each, independently selected from —NR⁴—, —NR⁴C(O)—, —N(C(O)R¹)—, —C(O)NR⁴—, —NR⁴C(O)NR⁴—, —C(O)—, —C(═NR⁴)—, —C(═NOR⁴)—, —OC(O)—, —C(O)O—, —OC(O)O—, —OC(O)NR⁴—, —NR⁴C(O)O—, —S(O)ₘ—; —S(O)ₘNR⁴—, or —NR⁴S(O)ₘ—, provided that M¹ and M² and are not both —NR⁴—:
R¹ and R² are each, independently selected from an alkyl, alkenyl cycloalkyl, aryl, biphenyl, heterocyclyl heterocycloalkyl, heteroaryl, cycloalkylalkyl, arylalkyl, arylalkenyl, arylalkynyl, heterocyclylalkyl, heteroarylalkyl, heteroarylalkenyl, or heteroarylalkynyl, wherein each cycloalkyl, aryl, heteroaryl, and heterocyclyl portion is optionally substituted with one or more R⁵;
R³ is selected from an alkyl, alkenyl, cycloalkyl, aryl, biphenyl, heterocyclyl heterocycloalkyl, heteroaryl, cycloalkylalkyl, arylalkyl, arylalkenyl, arylalkynyl, heterocyclylalkyl, heteroarylalkyl, heteroarylalkenyl, or heteroarylalkynyl, wherein each cycloalkyl, aryl, heteroaryl, and heterocyclyl portion is optionally substituted with one or more R⁷;
R⁴ is each independently H, alkyl, wherein each alkyl is optionally substituted with one or more R⁵;
R⁵ is each independently I, Br, Cl, F, —CH₂F, —CHF₂, —CF₃, —OCF₃, —CN, -alkylene-CN, —CONH₂, —CONHR⁶, —CONR⁶R⁶, —COR⁶, —COOH, —NH₂, -alkylene-NH₂, —NHR⁶, -alkylene-NHR⁶, —NO₂, —NR⁶R⁶, -alkylene-NR⁶R⁶, —NR⁶COR⁶, -(alkylene)NR⁶COR⁶, —N₃, —OH, OR⁶, —COOR⁶, —OSO₃R⁶, oxo, R⁶, —SH, —SO₂R⁶, —SO₃H, —SO₃R⁶, —SR⁶, heterocyclyl, or -alkylene-heterocyclyl, wherein heterocyclyl is optionally substituted with one or more R⁸;
R⁶ is each independently alkyl, alkenyl, alkynyl, haloalkyl, haloalkenyl, haloalkynyl; or alternatively two R⁶ on the same N atom can together form a 3-6 membered N-heterocyclyl;
R⁷ is each independently I, Br, Cl, F, —CH₂F, —CHF₂, —CF₃, —OCF₃, —CN, -alkylene-CN, —CONH₂, —CONHR⁶, —CONR⁶R⁶, —COOH, —NH₂, -alkylene-NH₂, —NHR⁶, -alkylene-NHR⁶, —NO₂, —NR⁶R⁶, -alkylene-NR⁶R⁶, —N₃, —OH, OR⁶, —COOR⁶, —OSO₃R⁶, oxo, R⁶, —SH, —SO₂R⁶, —SO₃H, —SO₃R⁶, —SR⁶, cycloalkyl, heterocyclyl, aryl, or heteroaryl, wherein each cycloalkyl, heterocyclyl, aryl, and heteroaryl is optionally substituted with one or more R⁵;
R⁸ is each independently I, Br, Cl, F, —CH₂F, —CHF₂, —CF₃, —OCF₃, —CN, -alkyl-CN, —CONH₂, —CONHR⁶, —CONR⁶R⁶, —COOH, —NH₂, —NHR⁶, —NO₂, —NR⁶R⁶, —N₃, —OH, OR⁶, —COOR⁶, —OSO₃R⁶, OXO, R⁶, —SH, —SO₂R⁶, —SO₃H, —SO₃R⁶, or —SR⁶;
m is 0, 1, or 2; and
wherein the compound is not N,N′-(1-phenyl-1H-1,2,4-triazole-3,5-diyl)dibenzamide, N-(3-benzamido-1-phenyl-1H-1.2,4-triazol-5-yl)furan-2-carboxamide. N-(5-cinnamamido-1-phenyl-1H-1,2,4-triazol-3-yl)benzamide, N-(1-phenyl-5-(phenylamino)-1H-1,2,4-triazol-3-yl)benzamide, 4-fluoro-N-(5-(4-methoxybenzamido)-1-phenyl-1H-1,2,4-triazol-3-yl)benzamide, N,N′-(1-phenyl-1H-1.2,4-triazole-3,5-diyl)bis(4-methylbenzamide), N-(5-((2-chlorobenzyl)amino)-1-phenyl-1H-1,2,4-triazol-3-yl)-2-fluorobenzamide, N-(3-benzamido-1-phenyl-1H-1,2,4-triazol-5-yl)-4-fluorobenzamide, N-(3-benzamido-1-phenyl-1H-1,2,4-triazol-5-yl)-4-nitrobenzamide, N-(3-benzamido-1-phenyl-1H-1,2,4-triazol-5-yl)-3-nitrobenzamide, and 4-((3-benzamido-1-phenyl-1H-1,2,4-triazol-5-yl)carbamoyl)benzoic acid.

In one embodiment of the compound of formula (III), L¹, L² and L³ are each independently a bond.

In one embodiment of the compound of formula (III), L³ is a bond and R³ is an aryl or a heteroaryl, optionally substituted with one or more R⁷. In one embodiment, L³ is a bond and R³ is a phenyl, optionally substituted with one or more R⁷.

In one embodiment of the compound of formula (III), R³ is optionally substituted with one or more R⁷ selected from I, Br, Cl, F, —CH₃, —CH₂F, —CHF₂, —CF₃, —OCF₃, —CN, —NH₂, —(C₁-C₃ alkylene)-NH₂, —NMe₂, —(C₁-C₃ alkylene)-NMe₂, —NO₂, —N₃, —OH, OR⁶, R⁶, —SH, cycloalkyl, heterocyclyl, aryl, or heteroaryl, wherein each cycloalkyl, heterocyclyl, aryl, and heteroaryl is optionally substituted with one or more R⁵.

In one embodiment of the compound of formula (III), R³ is substituted with at least one R⁷ is a 4-6 membered heterocyclyl, optionally substituted with one or more R⁵.

In one embodiment, the compound of formula (III) has the structure of formula (IIIA):

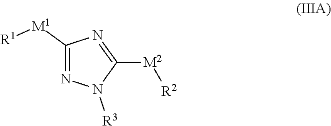

or a pharmaceutically acceptable salt or solvate thereof, wherein:
M¹ and M² are each independently selected from —NR⁴—, —NR⁴C(O)— or —C(O)NR⁴—, provided that M¹ and M² are not both —NR⁴—;
R¹ and R² are each independently phenyl, optionally substituted with one or more R⁵;
wherein at least one of R¹ or R² is substituted with —(C₁-C₆ alkylene)NHCO(C₁-C₁₀ alkyl) or —(C₁-C₆ alkylene)N(C₁-C₃ alkyl)CO(C₁-C₁₀ alkyl);
R³ is

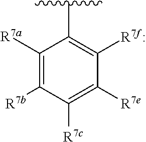

R⁴ is each independently H or C₁-C₃ alkyl;
R⁵ is each independently I, Br, Cl, F, —CH₂F, —CHF₂, —CF₃, —C₁-C₆ alkyl, alkynyl, —CN, —(C₁-C₃ alkylene)-CN, —NH₂, —NO₂, —N₃, —OH, —OCF₃, —OMe, —NMe₂, —NEt₂, —C(O)O(C₁-C₆ alkyl), —CO(C₁-C₁₀ alkyl), —NHCO(C₁-C₁₀ alkyl), —N(C₁-C₃ alky)CO(C₁—C₁₀ alkyl), —(C₁-C₆ alkylene)NHCO(C₁-C₁₀ alkyl), or —(C₁-C₆ alkylene)N(C₁-C₃ alkyl)CO(C₁-C₁₀ alkyl);
R^{7a}, R^{7b}, R^{7e}, and R^{7f} is each independently H, I, Br, Cl, F, —CH₂F, —CHF₂, —CF—₃, —OCF₃, —N₃, —CN, —OH, methyl, ethyl, propyl, C₁-C₃ haloalkyl, C₁-C₃ alkoxy, or C₁-C₃ haloalkoxy; and
R^{7c} is H, I, Br, Cl, F, —CH₂F, —CHF₂, —CF₃, —OCF₃, —N₃, —CN, —OH, C₁-C₃ alkyl, C₁-C₃ haloalkyl, C₁-C₃ alkoxy, C₁-C₃ haloalkoxy, 4-6 membered heterocyclyl, or 5-6 membered heteroaryl, wherein the heterocyclyl and heteroaryl is optionally substituted with one or more R⁵.

In one embodiment, the compound of formula (III) has the structure of formula (IIIB):

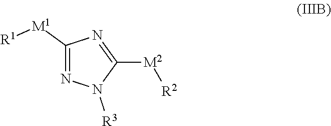

or a pharmaceutically acceptable salt or solvate thereof, wherein:
M¹ and M² are each independently selected from —NR⁴—, —NR⁴C(O)— or —C(O)NR⁴—, provided that M¹ and M² are not both —NR⁴—;
R¹ and R² are each independently phenyl, optionally substituted with one or more R⁵;
R³ is phenyl, substituted with one or more R⁷;
R⁴ is each independently H or C₁-C₃ alkyl;
R⁵ is each independently I, Br, Cl, F, —CH₂F, —CHF₂, —CF₃, —C₁-C₆, alkyl, alkynyl, —CN, —(C₁-C₃ alkylene)-CN, —NH₂, —NO₂, —N₃, —OH, —OCF₃, —OMe, —NMe₂, —NEt₂, —C(O)O(C₁-C₆ alkyl), —CO(C₁-C₁₀ alkyl), —NHCO(C₁-C₁₀ alkyl), —N(C₁-C₃ alkyl)CO(C₁-C₁₀ alkyl), —(C₁-C₆ alkylene)NHCO(C₁-C₁₀ alkyl), or —(C₁-C₆, alkylene)N(C₁-C₃ alkyl)CO(C₁-C₁₀ alkyl); and

wherein at least one R⁷ is heterocyclyl substituted with —CO(C₁-C₁₀ alkyl), which is optionally further substituted with one or more R⁵.

In one embodiment, the compound of formula (III) has the structure of formula (IIIC):

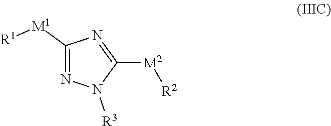

or a pharmaceutically acceptable salt or solvate thereof, wherein:
M¹ and M² are each independently selected from —NR⁴C(O)— or —C(O)NR⁴—;
R¹ and R² are each independently phenyl optionally substituted with one or more R⁵;
R³ is

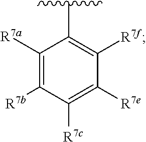

R⁴ is each independently H or C₁-C₃ alkyl; and
R^{7a}, R^{7b}, R^{7e}, and R^{7f} is each independently H, I, Br, Cl, F, —CH₂F, —CHF₂, —CF—₃, —OCF₃, —N₃, —CN, —OH, methyl, ethyl, propyl, C₁-C₃ haloalkyl, C₁-C₃ alkoxy, or C₁-C₃ haloalkoxy;
R^{7c} is H, I, Br, Cl, F, —CH₂F, —CHF₂, —CF₃, —OCF₃, —N₃, —CN, —OH, —C₁-C₃ alkyl, —C₁-C₃ haloalkyl, —C₁-C₃ alkoxy, —C₁-C₃ haloalkoxy, 4-6 membered heterocyclyl, or 5-6 membered heteroaryl, wherein the heterocyclyl and heteroaryl is optionally substituted with one or more R⁵;
R⁵ is I, Br, Cl, F, —CH₂F, —CHF₂, —CF₃, —C₁-C₆ alkyl, alkynyl, —CN, —(C₁-C₃ alkylene)-CN, —NH₂, —(C₁-C₃ alkylene)-NH₂, —(C₁-C₃ alkylene)-NHR⁶, —(C₁-C₃ alkylene)-NR⁶R⁶, —NO₂. —N₃, —OH, —OCF₃, —OMe, —NMe₂, —NEt₂, —C(O)O(C₃-C₆ alkyl), 4-6 membered heterocyclyl, or —(C₁-C₃ alkylene)-(4-6 membered heterocyclyl), wherein heterocyclyl is optionally substituted with one or more R⁸;
R⁶ is —C₁-C₃ alkyl;
R⁸ is I, Br, Cl, F, —CH₂F, —CHF₂, —CF₃, or —C₁-C₆ alkyl; and
wherein at least one of R^{7a}, R^{7b}, R^{7c}, R^{7e}, and R^{7f} is not H.

In one embodiment, the compound of formula (III) has the structure of formula (IIID):

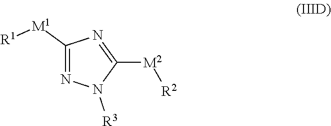

or a pharmaceutically acceptable salt or solvate thereof, wherein:
M¹ and M² are each independently selected from —NR⁴C(O)— or —C(O)NR⁴—;
R¹ is phenyl optionally substituted with one R^{5a};
R² is phenyl optionally substituted with one R^{5b};

R³ is

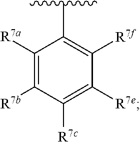

R⁴ is each independently H or C₁-C₃ alkyl; and
R^{7a}, R^{7b}, R^{7e}, and R^{7f} is each independently H, I, Br, Cl, F, —CH₂F, —CHF₂, —CF-3, —OCF₃, —N₃, —CN, —OH, methyl, ethyl, propyl, C₁-C₃ haloalkyl, C₁-C₃ alkoxy, or C₁-C₃ haloalkoxy;
R^{7c} is H, I, Br, Cl, F, —CH₂F, —CHF₂, —CF₃, —OCF₃, —N₃, —CN, —OH, —C₁-C₃ alkyl, —C₁-C₃ haloalkyl, —C₁-C₃ alkoxy, —C₁-C₃ haloalkoxy, 4-6 membered heterocyclyl, or 5-6 membered heteroaryl, wherein the heterocyclyl and heteroaryl is optionally substituted with one R^{5b};
R^{5a} is —(C₁-C₃ alkylene)-(4-6 membered heterocyclyl), wherein heterocyclyl is optionally substituted with one R⁸;

R^{5b} is I, Br, Cl, F, —CH₂F, —CHF₂, —CF₃, —C₁-C₆, alkyl, alkynyl, —CN, —(C₁-C₃ alkylene)-CN, —NH₂, —(C₁-C₃ alkylene)-NH₂, —(C₁-C₃ alkylene)-NHR⁶, —(C₁-C₃ alkylene)-NR⁶R⁶, —NO₂, —N₃, —OH, —OCF₃, —OMe, —NMe₂, -NEC, or —C(O)O(C₁-C₆ alkyl);
R⁶ is —C₁-C₃ alkyl;
R⁸ is —O—C₃ alkyl; and
wherein at least one of R^{7a}, R^{7b}, R^{7c}, R^{7e}, and R^{7f} is not H.

In one embodiment, the compound of formula (III) has the structure of formula (IIIE):

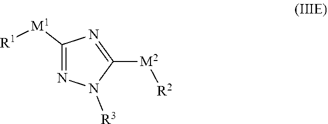

or a pharmaceutically acceptable salt or solvate thereof, wherein:
M¹ and M² are each independently selected from —NR⁴C(O)— or —C(O)NR⁴—;
R¹ is phenyl optionally substituted with —(C₁-C₃ alkylene)-(5-6 membered heterocyclyl), wherein heterocyclyl is optionally substituted with one R⁸;
R² is phenyl optionally substituted with one R^{5b};
R³ is

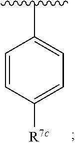

R⁴ is each independently H or C₁-C₃ alkyl;
R^{7a}, R^{7b}, R^{7e}, and R^{7f} is each independently H, I, Br, Cl, F, —CH₂F, —CHF₂, —CF—₃, —OCF₃, —N₃, —CN, —OH, methyl, ethyl, propyl, C₁-C₃ haloalkyl, C₁-C₃ alkoxy, or C₁-C₃ haloalkoxy;
R^{7c} is H, I, Br, Cl, F, —CH₂F, —CHF₂, —CF₃, —OCF₃, —N₃, —CN, —OH, —C₁-C₃ alkyl, —C₁-C₃ haloalkyl, —C₁-C₃ alkoxy, —C₁-C₃ haloalkoxy, 4-6 membered heterocyclyl, or 5-6 membered heteroaryl, wherein the heterocyclyl and heteroaryl is optionally substituted with one R^{5b};
R^{5b} is I, Br, Cl, F, —CH₂F, —CHF₂, —CF₃, —C₁-C₆ alkyl, alkynyl, —CN, —(C₁-C₃ alkylene)-CN, —NH₂, —(C₁-C₃ alkylene)-NH₂, —(C₁-C₃ alkylene)-NHR⁶, —(C₁-C₃ alkylene)-NR⁶R⁶, —NO₂, —N₃, —OH, —OCF₃, —OMe, —NMe₂, —NEt₂, or —C(O)O(C₁-C₆ alkyl);
R⁶ is —C₁-C₃ alkyl; and
R⁸ is —C₁-C₃ alkyl.

In some embodiment of the compounds of formula (III) and/or (IIIA)-(IIIB), M¹ and M² are each independently selected from —NR⁴—, —NR⁴C(O)— or —C(O)NR⁴—.

In some embodiment of the compounds of formula (III) and/or (IIIA)-(IIIE), M¹ and M² are each —NR⁴C(O)— or —C(O)NR⁴—. In one embodiment, M¹ and M² are each —NHC(O)— or —C(O)NH—.

In some embodiment of the compounds of formula (III) and/or (IIIA)-(IIIE), R¹ and R² are each independently selected from phenyl or 5-10 membered heteroaryl, wherein each phenyl and heteroaryl is optionally substituted with one or more R⁵.

In some embodiment of the compounds of formula (III) and/or (IIIA)-(IIIE), at least one of R¹ or R² is substituted with —(C₁-C₃ alkylene)NHCO(C₁-C₈ alkyl) or —(C₁-C₃ alkylene)N(C₁-C₃ alkyl)CO(C₁-C₈ alkyl). In some embodiments, at least one of R¹ or R² is substituted with —CH₂NHCO(C₁-C₈ alkyl) or —CH₂N(C₁-C₃ alkyl)CO(C₁-C₈ alkyl).

In some embodiment of the compounds of formula (III) and/or (IIIA)-(IIIE), R¹ is phenyl substituted with —(C₁-C₃ alkylene)NHCO(C₁-C₈ alkyl) or —(C₁-C₃ alkylene)N(C₁-C₃ alkyl)CO(C₁-C₈ alkyl). In some embodiments, R¹ is phenyl substituted with —CH₂NHCO(C₁-C₈ alkyl) or —CH₂N(C₁-C₃ alkyl)CO(C₁-C₈ alkyl). In some embodiments, R¹ is phenyl substituted with CH₂NHCO(C₄-C₈ alkyl) or —CH₂N(C₁-C₃ alkyl)CO(C₄-C₈ alkyl).

In some embodiment of the compounds of formula (III) and/or (IIIA)-(IIIE), R¹ is

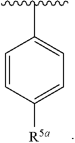

In some embodiment of the compounds of formula (III) and/or (IIIA)-(IIIE), R² is phenyl substituted with —(C₁-C₃ alkylene)NHCO(C₁-C₈ alkyl) or —(C₁-C₃ alkylene)N(C₁-C₃ alkyl)CO(C₁-C₈ alkyl). In some embodiments, R² is phenyl substituted with —CH₂NHCO(C₁-C₈ alkyl) or —CH₂N(C₁-C₃ alkyl)CO(C₁-C₈ alkyl). In some embodiments, R² is phenyl substituted with CH₂NHCO(C₄-C₈ alkyl) or —CH₂N(C₁-C₃ alkyl)CO(C₄-C₈ alkyl).

In some embodiment of the compounds of formula (III) and/or (IIIA)-(IIIE), R² is

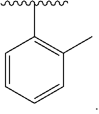 [SMILES: *c1ccccc1C]

In some embodiment of the compounds of formula (III) and/or (IIIA)-(IIIE), R¹ and R² are each independently phenyl, optionally substituted with one or more substituent selected from I, Br, Cl, F, —CH₂F, —CHF₂, —CF₃, —OCF₃, —N₃, —CN, —OH, methyl, ethyl, propyl, —C≡CH; —CN, —NH₂, —NO₂, —N₃, —OH, —OCF₃, —OMe, —NMe₂, or —NEt₂.

In some embodiment of the compounds of formula (III) and/or (IIIA)-(IIIE), at least one of R¹ and R² is phenyl substituted with —CH₂—NH₂, —CH₂—NHMe, or —CH₂—NMe₂. In one embodiment, at least one of R¹ or R² is substituted with —(C₁-C₃ alkylene)NHCO(C₁-C₈ alkyl) or —(C₁-C₃ alkylene)N(C₁-C₃ alkyl)CO(C₁-C₈ alkyl). In one embodiment, at least one of R¹ or R² is substituted with —CH₂NHCO(C₁-C₈ alkyl) or —CH₂N(C₁-C₃ alkyl)CO(C₁-C₈ alkyl).

In some embodiment of the compounds of formula (III) and/or (IIIA)-(IIIE), at least one of R¹ or R² is substituted with —(C₁-C₃ alkylene)NHCO(C₁-C₈ alkyl) or —(C₁-C₃ alkylene)N(C₁-C₃ alkyl)CO(C₁-C₈ alkyl) and R⁷ is each independently, I, Br, Cl, F, —CH₃, —CH₂F, —CHF₂, —CF₃, —OCF₃, —CN, —NH₂, —(C₁-C₃ alkylene)-NH₂, —NMe₂, —(C₁-C₃ alkylene)-NMe₂, —NO₂, —N₃, —OH, OR⁶, R⁶, —SH, cycloalkyl, heterocyclyl, aryl, or heteroaryl, wherein each cycloalkyl, heterocyclyl, aryl, and heteroaryl is optionally substituted with one or more R⁵. In one embodiment, at least one of R¹ or R² is substituted with —CH₂NHCO(C₁-C₈ alkyl) or —CH₂N(C₁-C₃ alkyl)CO(C₁-C₈ alkyl) and R⁷ is each independently, I, Br, Cl, F, —CH₃, —CH₂F, —CHF₂, —CF₃, —OCF₃, —CN, —NH₂, —(C₁-C₃ alkylene)-NH₂, —NMe₂, —(C₁-C₃ alkylene)-NMe₂, —NO₂, —N₃, —OH, OR⁶, R⁶, —SH, cycloalkyl, heterocyclyl, aryl, or heteroaryl, wherein each cycloalkyl, heterocyclyl, aryl, and heteroaryl is optionally substituted with one or more R⁵.

In some embodiment of the compounds of formula (III) and/or (IIIA)-(IIIE), R³ is phenyl, substituted with 6-membered heterocyclyl and wherein the 6-membered heterocyclyl is substituted with —CO(C₁-C₁₀ alkyl). In one embodiment, R³ is phenyl substituted with a R³ is phenyl, substituted with 6-membered heterocyclyl comprising one or two heteroatoms selected from O, N, and S, and wherein the 6-membered heterocyclyl is substituted with —CO(C₁-C₁₀ alkyl).

In some embodiment of the compounds of formula (III) and/or (IIIA)-(IIIE), R³ is phenyl substituted with a piperidine or a piperazine, wherein the piperidine or the piperazine is substituted with —CO(C₁-C₁₀ alkyl). In one embodiment, R³ is phenyl substituted with a piperidine or a piperazine, wherein the piperidine or the piperazine is substituted with —CO(C₄-C₁₀ alkyl).

In some embodiment of the compounds of formula (III) and/or (IIIA)-(IIIE), R^{7a}, R^{b}. R^{7c}, R^{7e}, and R^{7f} is each independently H, I, Br, Cl, F, —CH₂F, —CHF₂, —CF₃, —OCF₃, —N₃, —CN, —OH, methyl, ethyl, propyl, —C≡CH; —CN, —NH₂, —NO₂, —N₃, —OH, —OCF₃, —OMe, —NMe₂, or —NEt₂. In some embodiments, three of R^{7a}, R^{7b}, R^{7c}, R^{7e}, and R^{7f} is H. In some embodiments, four of R^{7a}, R^{7b}, R^{7c}, R^{7e}, and R^{7f} is H. In some embodiments, R^{7a}, R^{7b}. R^{7e}, and R^{7f} is each independently H.

In some embodiment of the compounds of formula (III) and/or (IIIA)-(IIIE), R³ is

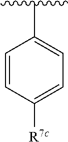

and R^{7c} is H, I, Br, Cl, F, —CH₂F, —CHF₂, —CF₃, —OCF₃, —N₃, —CN, —OH, —C₁-C₃ alkyl, —C₁-C₃ haloalkyl, —C₁-C₃ alkoxy, or —C₁-C₃ haloalkoxy. In some embodiments, R³ is

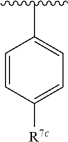

and R^{7c} is —OCF₃.

In some embodiment of the compounds of formula (III) and/or (IIIA)-(IIIE), at least one of R¹, R², and R³ is phenyl substituted with —(C₁-C₃ alkylene)-(4-6 membered heterocyclyl), wherein heterocyclyl is optionally substituted with one R⁸. In one embodiment, at least one of R¹, R², and R³ is phenyl and substituted with at least one of methyl, ethyl, —C≡CH, I, Br, Cl, F, —CF₃, —CN, —CH₂CN, —CH₂CH₂CN, —NH₂, —CH₂—NH₂, —NO₂, —N₃, —OH, —OCF₃, —OMe, —NMe₂ or —CH₂—NMe.

In some embodiments of the compounds of formula (III) and/or (IIIA)-(IIIE), R⁴ at each occurrence is independently H or C₁-C₃ alkyl.

In some embodiment of the compounds of formula (III) and/or (IIIA)-(IIIE), R⁵ is selected from I, Br, Cl, F, —CH₂F, —CF₃, —C₁-C₅ alkyl, alkynyl, —CN, —(C₁-C₃ alkylene)—, CN, —NH₂, —(C₁-C₃ alkylene)-NH₂, —C₁-C₃ alkylene)-NHR⁶, —(C₁-C₃ alkylene)-NR⁶R⁶, —NO₂, —N₃, —OH, —OCF₃, —OMe, —NMe₂, —NEt₂, —C(O)O(C₁-C₆ alkyl), 4-6 membered heterocyclyl, or —(C₁-C₃ alkylene)-(4-6 membered heterocyclyl), wherein heterocyclyl is optionally substituted with one or more R⁸.

In some embodiment of the compounds of formula (III) and/or (IIIC), R⁵ is —(C₁-C₃ alkylene)-(4-6 membered heterocyclyl), wherein heterocyclyl is optionally substituted with one R⁸. In one embodiment, R⁵ is —CH₂—NH₂, —CH₂—NHMe, or —CH₂—NMe₂.

In some embodiment of the compounds of formula (III) and/or (IIID), R^{5a} is —CH₂-(6 membered heterocyclyl), wherein heterocyclyl is optionally substituted with one R⁸. In one embodiment, R^{5a} is —CH₂-(piperazinyl), wherein piperazinyl is optionally substituted with one R⁸. In one embodiment R^{5a} is

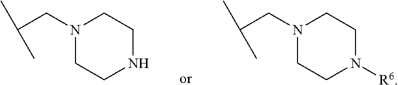

In one embodiment, the compound of formula (III) and/or (IIIA)-(IIIE), excludes compounds of Table A. In one embodiment, the compound of formula (III) and/or (IIIA)-(IIIE), exclude compounds of Table B.

In some embodiments, various embodiments disclosed herein for formula (I), (I′), (IA), (IB), (IB′), (IC), (ID), (IE), and/or (IF) cm be applied to the compounds of formula (III) and/or (IIIA)-(IIIE).

In one embodiment of the compounds of formula (III) and or (IIIA)-(IIIE), the compound is selected from Table 3C below, or a pharmaceutically acceptable salt or solvate thereof.

TABLE 3C

| | |
|---|---|
| 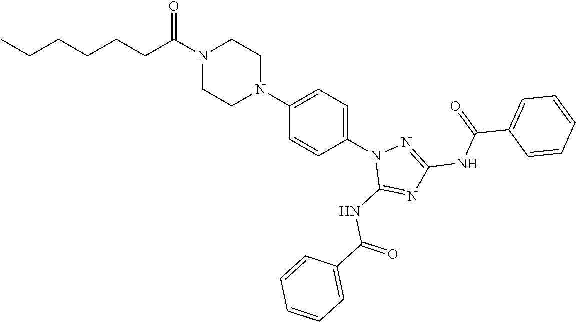 [SMILES: CCCCCCC(=O)N1CCN(c2ccc(-n3nc(NC(=O)c4ccccc4)nc3NC(=O)c3ccccc3)cc2)CC1] | 160 |
| 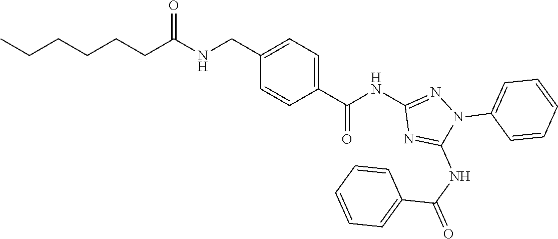 [SMILES: CCCCCCC(=O)NCc1ccc(C(=O)Nc2nc(NC(=O)c3ccccc3)n(-c3ccccc3)n2)cc1] | 161 |
| 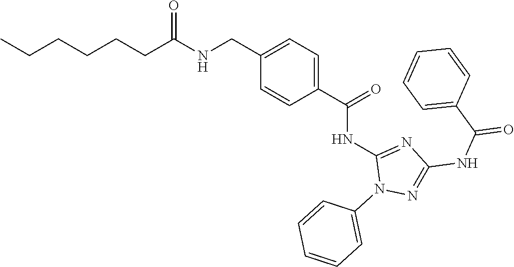 [SMILES: CCCCCCC(=O)NCc1ccc(C(=O)Nc2nc(NC(=O)c3ccccc3)nn2-c2ccccc2)cc1] | 162 |

In one embodiment, the present disclosure provides a pharmaceutical composition comprising any one of the compounds of formula (III) and/or (IIIA)-(IIIE), or a pharmaceutically acceptable salt thereof, and a pharmaceutically acceptable carrier. In one embodiment, the pharmaceutical composition comprises a compound selected from Table 3C, or a pharmaceutically acceptable salt thereof, and a pharmaceutically acceptable carrier.

In one embodiment, the present disclosure provides a comprising a pharmaceutically acceptable carrier or a pharmaceutically acceptable excipient and a compound from Tables 3, 3A, 3B, 3C or 3D.

R5

In one embodiment, the present disclosure provides compounds comprising the structure of formula (IV):

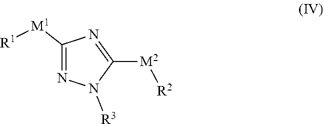

or a pharmaceutically acceptable salt or solvate thereof, wherein:
M¹ and M² are each independently selected from a bond, —NR⁴C(O)— or —C(O)NR⁴—, provided that M¹ and M² are not both a bond;
R¹ and R² are each independently phenyl or pyridyl, optionally substituted with one or more R⁵;
R³ is

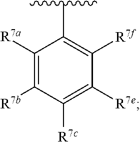

R⁴ is each independently H or C₁-C₃ alkyl; and
R^{7a}, R^{7b}, R^{7e}, and R^{7f} is each independently H, I, Br, Cl, F, —CH₂F, —CHF₂, —CF—₃, —OCF₃, —N₃, —CN, —OH, methyl, ethyl, propyl, C₁-C₃ haloalkyl, C₁-C₃ alkoxy, or C₁-C₃ haloalkoxy;
R^{7c} is H, I, Br, Cl, F, —CH₂F, —CHF₂, —CF₃, —OCF₃, —N₃, —CN, —OH, —C₁-C₃ alkyl, —C₁-C₃ haloalkyl, —C₁-C₃ alkoxy, —C₁-C₃ haloalkoxy, 4-6 membered heterocyclyl, or 5-6 membered heteroaryl, wherein the heterocyclyl and heteroaryl is optionally substituted with one or more R⁵;
R⁵ is I, Br, Cl, F, —CH₂F, —CHF₂, —CF₃, —C₁-C₆ alkyl, alkynyl, —CN, —(C₁-C₃ alkylene)-CN, —NH₂, —(C₁-C₃ alkylene)-NH₂, —(C₁-C₃ alkylene)-NHR⁶, —(C₁-C₃ alkylene)-NR⁶R⁶, —NO₂, —N₃, —OH, —OCF₃, —OMe, —NMe₂, —NEt₂, —C(O)O(C₁-C₆ alkyl), —(C₁-C₃ alkylene)-NHCO(4-6 membered heterocyclyl), —(C₁-C₃ alkylene)-NR⁶CO(4-6 membered heterocyclyl), 4-6 membered heterocyclyl, or —(C₁-C₃ alkylene)-(4-6 membered heterocyclyl), wherein heterocyclyl is optionally substituted with one or more R⁸;
wherein at least one R⁵ is present and selected from —CH₂NH₂, —CH₂NHR⁶, —CH₂NR⁶R⁶, —CH₂NHCO(heterocyclyl), —CH₂NR⁶CO(heterocyclyl), or —CH₂-heterocyclyl, wherein heterocyclyl is optionally substituted with one or more R⁸;
R⁶ is —C₁-C₃ alkyl;
R⁸ is I, Br, Cl, F, —CH₂F, —CHF₂, —CF₃, or —C₁-C₆ alkyl; and
wherein at least one of R^{7a}, R^{7b}, R^{7c}, R^{7e}, and R^{7f} is not H.

In one embodiment of the compound of formula (IV), R¹ and R² are each phenyl, optionally substituted with one or more R⁵. In one embodiment of the compound of formula (IV), R² is phenyl, optionally substituted with one or more R⁵.

In one embodiment of the compound of formula (IV), M¹ and M² are each —NHC(O)— or —C(O)NH—.

In one embodiment of the compound of formula (IV), R¹ is 2-pyridyl, optionally substituted with one or more R⁵.

In one embodiment of the compound of formula (IV), R¹ is

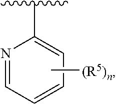

wherein n is 0, 1, 2, or 3.

In one embodiment of the compound of formula (IV), R¹ is

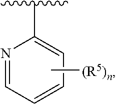

wherein n is 0, 1, or 2, and R² is phenyl, optionally substituted with one or more R⁵.

In one embodiment of the compound of formula (IV), R¹ is phenol, optionally substituted with one or more R⁵. In one embodiment, R¹ is

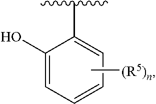

wherein n is 0, 1, 2, or 3. In one embodiment, R¹ is

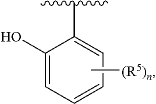

wherein n is 0, 1 or 2. In one embodiment, R¹ is

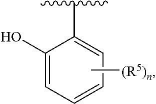

wherein n is 0 or 1.

In one embodiment of the compound of formula (IV), R² is phenyl, optionally substituted with one or more R⁵.

In one embodiment of the compound of formula (IV), R¹ is

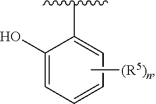

wherein n is 0, 1, 2, or 3, and R² is phenyl, optionally substituted with one or more R⁵. In one embodiment of the compound of formula (IV), R¹ is

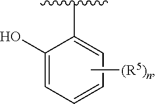

wherein n is 0 or 1, and R² is phenyl, optionally substituted with one or more R⁵.

In one embodiment of the compound of formula (IV), R² is

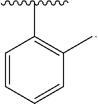 [SMILES: *c1ccccc1C]

In one embodiment of the compound of formula (IV), -M²-R² is

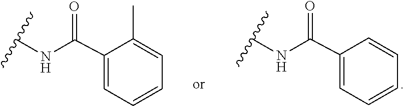 [SMILES: *NC(=O)c1ccccc1C ; *NC(=O)c1ccccc1]

In one embodiment of the compound of formula (IV), R³ is R³ is phenyl,

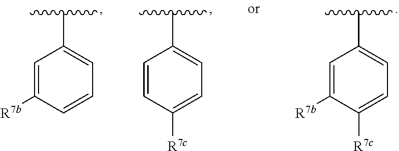

In one embodiment, R³ is

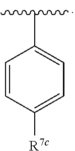

In one embodiment of the compound of formula (IV), R^{7b} and R^{7c} are each independently H, I, Br, Cl, F, —CH₃, —CH₂F, —CHF₂, —CF₃, —OCF₃, —CN, —NH₂, —(C₁-C₃ alkylene)-NH₂, —NMe₂, —(C₁-C₃ alkylene)-NMe₂, —NO₂, —N₃, —OH, OR⁶, R⁶, —SH, cycloalkyl, heterocyclyl, aryl, or heteroaryl, wherein each cycloalkyl, heterocyclyl, aryl, and heteroaryl is optionally substituted with one or more R⁵. In one embodiment, R^{7b} and R^{7c} are each independently H, I, Br, Cl, F, —CH₃, —CH₂F, —CHF₂, —CF₃, or —OCF₃. In one embodiment, R^{b} and R^{7c} are each independently H, F, or —OCF₃.

In one embodiment of the compound of formula (IV), wherein at least one R⁷ is a 4-6 membered heterocyclyl, optionally substituted with one or more R⁵.

In one embodiment of the compound of formula (IV), at least one R⁵ is present and selected from —CH₂NH₂, —CH₂NHCH₃, —CH₂NHCH₂CH₃, —CH₂NHCH₂CH₂CH₃, —CH₂NHCH(CH₃)₂, —CH₂NHCO(C₆ alkyl), —CH₂-morpholinyl, —CH₂-piperazinyl, or —CH₂NHCO(pyrrolidonyl), wherein, morpholinyl, piperazinyl, and pyrrolidonyl are each optionally substituted with I, Br, Cl, F, —CH₂F, —CHF₂, —CF₃, or —C₁-C₆ alkyl.

In one embodiment of the compound of formula (IV), R¹ is substituted with at least one group selected from —CH₂NH₂, —CH₂NHCH₃, —CH₂NHCH₂CH₃, —CH₂NHCH₂CH₂CH₃, —CH₂NHCH(CH₃)₂, —CH₂NHCO(C₆ alkyl), —CH₂-morpholinyl, —CH₂-piperazinyl, or —CH₂NHCO(pyrrolidonyl), wherein, morpholinyl, piperazinyl, and pyrrolidonyl are each optionally substituted with I, Br, Cl, F, —CH₂F, —CHF₂, —CF₃, or —C₁-C₆ alkyl, wherein R¹ is further optionally substituted with one or more R⁵. In one embodiment, R¹ is substituted with at least one R⁵, wherein at least one R⁵ is selected from —CH₂NH₂, —CH₂NHCH₃, —CH₂NHCH₂CH₃, —CH₂NHCH₂CH₂CH₃, —CH₂NHCH(CH₃)₂, —CH₂NHCO(Cₑ alkyl),

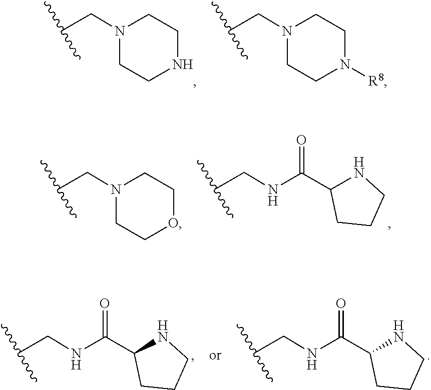

In one embodiment, R¹ is substituted with at least one R⁵, wherein at least one R⁵ is —CH₂NH₂.

In one embodiment of the compound of formula (IV), R² is substituted with at least one group selected from —CH₂NH₂, —CH₂NHCH₃, —CH₂NHCH₂CH₃, —CH₂NHCH₂CH₂CH₃, —CH₂NHCH(CH₃)₂, —CH₂NHCO(C₆ alkyl), —CH₂-morpholinyl, —CH₂-piperazinyl, or —CH₂NHCO(pyrrolidonyl), wherein, morpholinyl, piperazinyl, and pyrrolidonyl are each optionally substituted with I, Br, Cl, F, —CH₂F, —CHF₂, —CF₃, or —C₁-C₆ alkyl, wherein R² is further optionally substituted with one or more R⁵. In one embodiment, R² is substituted with at least one R⁵, wherein at least one R⁵ is selected from —CH₂NH₂, —CH₂NHCH₃, —CH₂NHCH₂CH₃, —CH₂NHCH₂CH₂CH₃, —CH₂NHCH(CH₃)₂, —CH₂NHCO(C₆ alkyl),

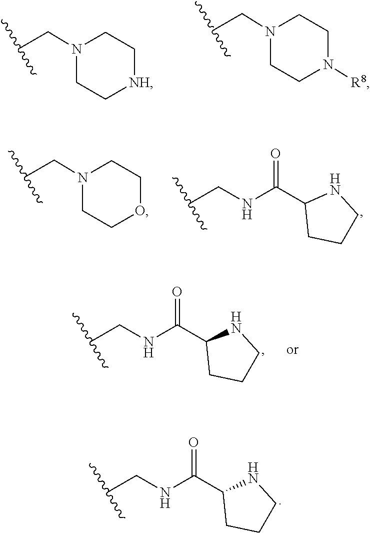

In one embodiment, R² is substituted with at least one R⁵, wherein at least one R⁵ is —CH₂NH₂.

In one embodiment of, the compound is selected from Table 3D below, or a pharmaceutically acceptable salt or solvate thereof.

TABLE 3D

| Compd | |
|---|---|
| ID | Structure |
| 1x | 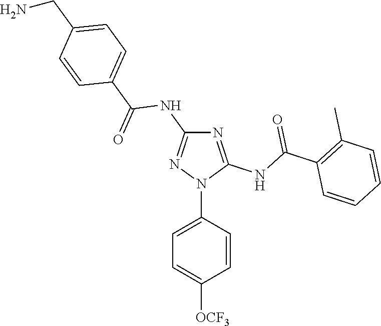 [SMILES: Cc1ccccc1C(=O)Nc1nc(NC(=O)c2ccc(CN)cc2)nn1-c1ccc(OC(F)(F)F)cc1] |
| 2x | 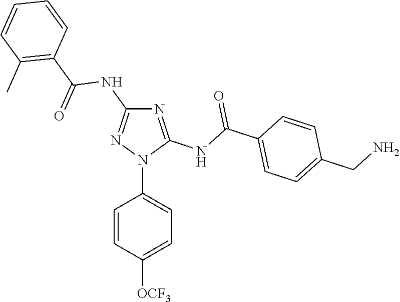 [SMILES: Cc1ccccc1C(=O)Nc1nc(NC(=O)c2ccc(CN)cc2)n(-c2ccc(OC(F)(F)F)cc2)n1] |
| 3x | 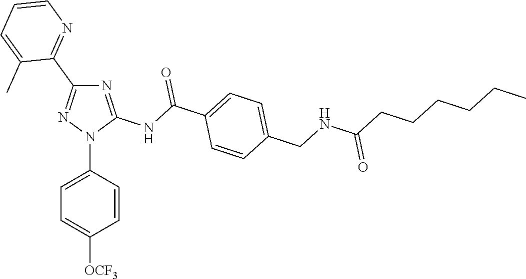 [SMILES: CCCCCCC(=O)NCc1ccc(C(=O)Nc2nc(-c3ncccc3C)nn2-c2ccc(OC(F)(F)F)cc2)cc1] |
| 4x | 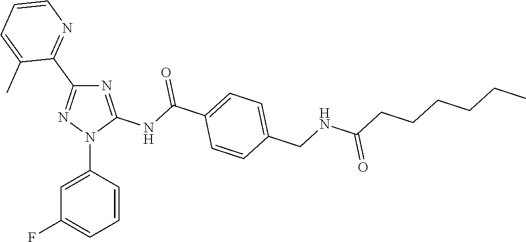 [SMILES: CCCCCCC(=O)NCc1ccc(C(=O)Nc2nc(-c3ncccc3C)nn2-c2cccc(F)c2)cc1] |
| 5x | 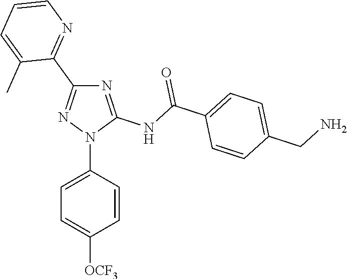 [SMILES: Cc1cccnc1-c1nc(NC(=O)c2ccc(CN)cc2)n(-c2ccc(OC(F)(F)F)cc2)n1] |
| 6x | 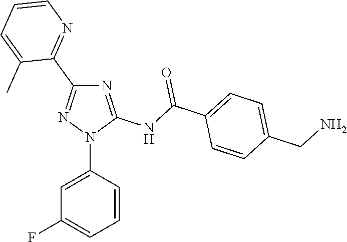 [SMILES: Cc1cccnc1-c1nc(NC(=O)c2ccc(CN)cc2)n(-c2cccc(F)c2)n1] |
| 7x | 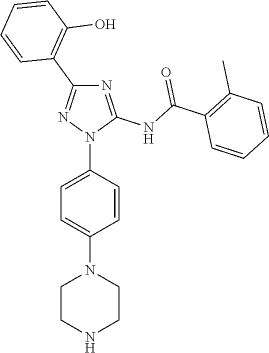 [SMILES: Cc1ccccc1C(=O)Nc1nc(-c2ccccc2O)nn1-c1ccc(N2CCNCC2)cc1] |
| 8x | 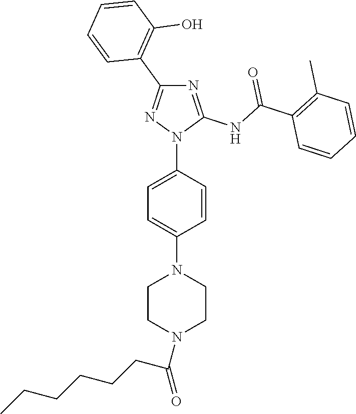 [SMILES: CCCCCCC(=O)N1CCN(c2ccc(-n3nc(-c4ccccc4O)nc3NC(=O)c3ccccc3C)cc2)CC1] |
| 9x | 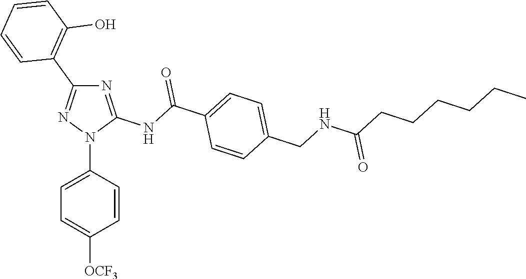 [SMILES: CCCCCCC(=O)NCc1ccc(C(=O)Nc2nc(-c3ccccc3O)nn2-c2ccc(OC(F)(F)F)cc2)cc1] |
| 10x | 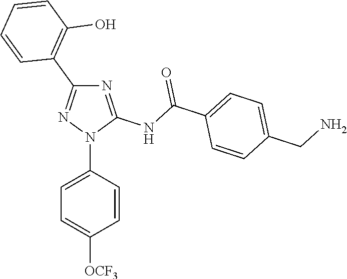 [SMILES: NCc1ccc(C(=O)Nc2nc(-c3ccccc3O)nn2-c2ccc(OC(F)(F)F)cc2)cc1] |
| 11x | 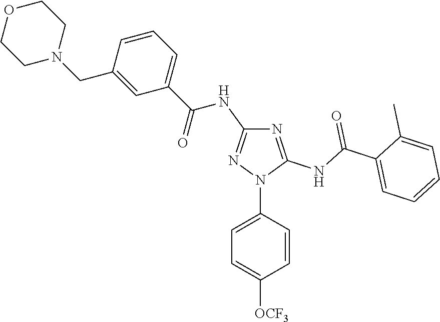 [SMILES: Cc1ccccc1C(=O)Nc1nc(NC(=O)c2cccc(CN3CCOCC3)c2)nn1-c1ccc(OC(F)(F)F)cc1] |
| 12x | 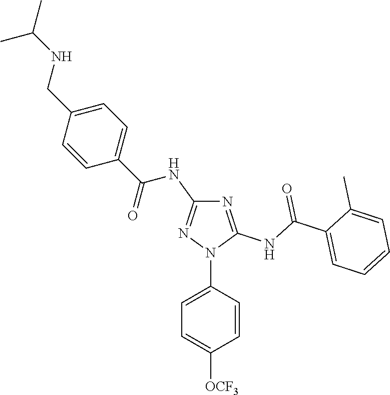 [SMILES: Cc1ccccc1C(=O)Nc1nc(NC(=O)c2ccc(CNC(C)C)cc2)nn1-c1ccc(OC(F)(F)F)cc1] |
| 13x | 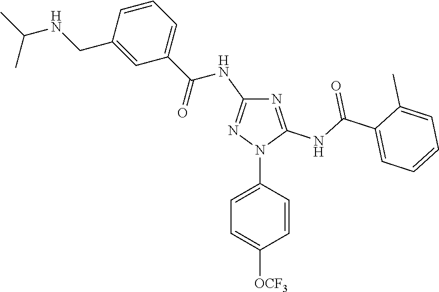 [SMILES: Cc1ccccc1C(=O)Nc1nc(NC(=O)c2cccc(CNC(C)C)c2)nn1-c1ccc(OC(F)(F)F)cc1] |
| 14x | 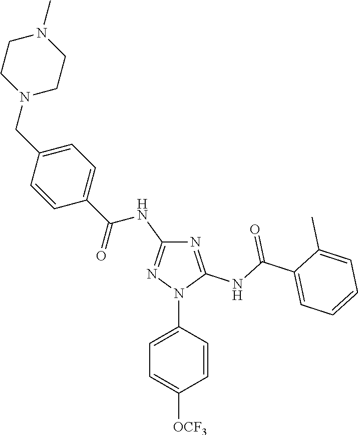 [SMILES: Cc1ccccc1C(=O)Nc1nc(NC(=O)c2ccc(CN3CCN(C)CC3)cc2)nn1-c1ccc(OC(F)(F)F)cc1] |
| 15x | 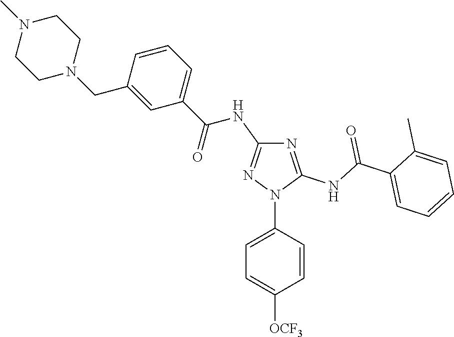 [SMILES: Cc1ccccc1C(=O)Nc1nc(NC(=O)c2cccc(CN3CCN(C)CC3)c2)nn1-c1ccc(OC(F)(F)F)cc1] |
| 16x | 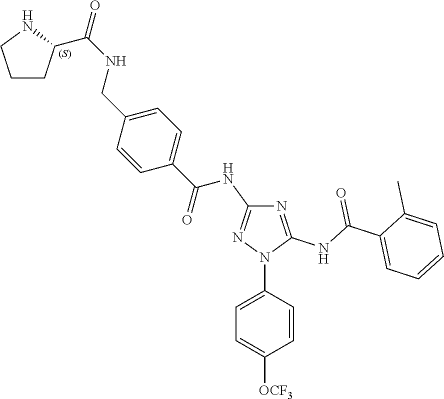 |
| 17x | 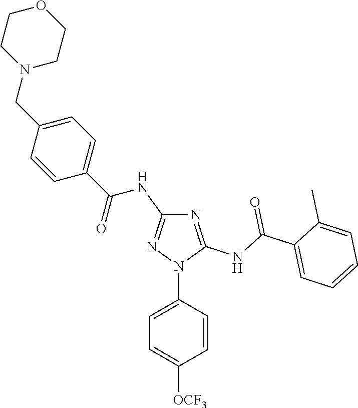 [SMILES: Cc1ccccc1C(=O)Nc1nc(NC(=O)c2ccc(CN3CCOCC3)cc2)nn1-c1ccc(OC(F)(F)F)cc1] |

In one embodiment, the present disclosure provides a pharmaceutical composition comprising any one of the compounds of formula (IV), or a pharmaceutically acceptable salt thereof, and a pharmaceutically acceptable carrier.

In one embodiment, the present disclosure provides a pharmaceutical composition comprising any one of the compounds of Table 3D, or a pharmaceutically acceptable salt, thereof, and a pharmaceutically acceptable carrier.

In one embodiment, the present disclosure provides a pharmaceutical composition as disclosed herein comprises one additional therapeutically active agent.

In some embodiments, the Parkin ligase activator of the invention excludes one or more compounds selected from

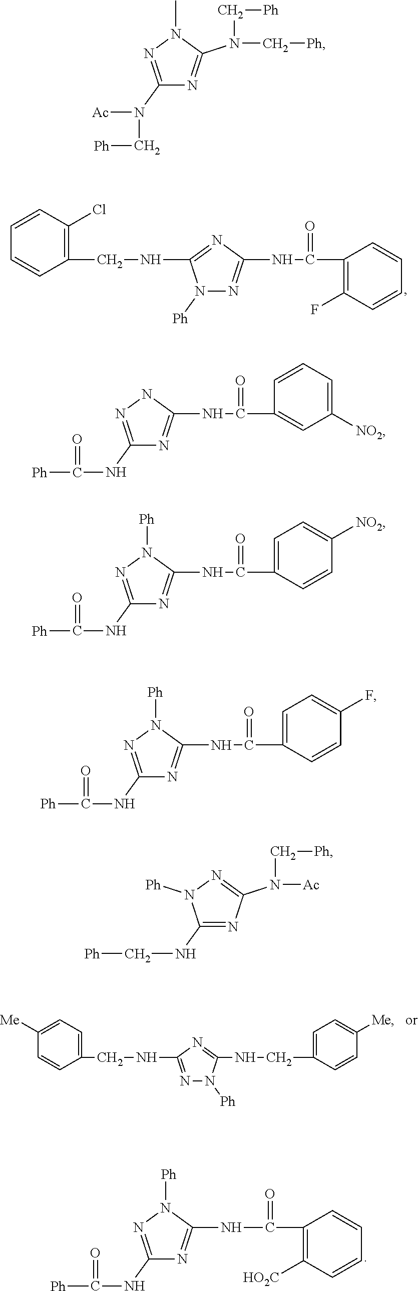

In one embodiment, the Parkin ligase activator of the invention excludes compounds in Table A.

TABLE A

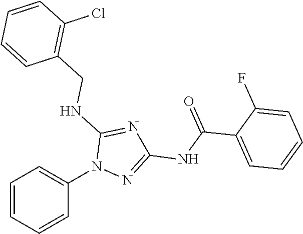 [SMILES: O=C(Nc1nc(NCc2ccccc2Cl)n(-c2ccccc2)n1)c1ccccc1F]

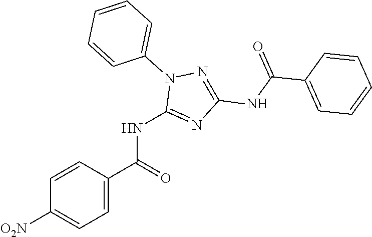 [SMILES: O=C(Nc1nc(NC(=O)c2ccc([N+](=O)[O-])cc2)n(-c2ccccc2)n1)c1ccccc1]

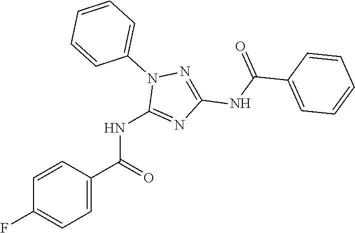 [SMILES: O=C(Nc1nc(NC(=O)c2ccc(F)cc2)n(-c2ccccc2)n1)c1ccccc1]

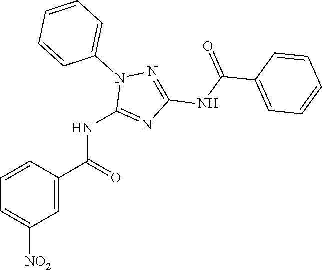 [SMILES: O=C(Nc1nc(NC(=O)c2cccc([N+](=O)[O-])c2)n(-c2ccccc2)n1)c1ccccc1]

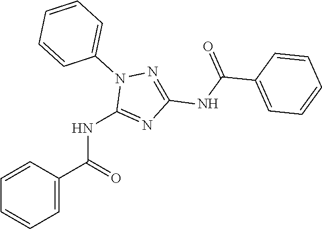 [SMILES: O=C(Nc1nc(NC(=O)c2ccccc2)n(-c2ccccc2)n1)c1ccccc1]

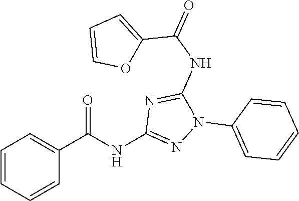 [SMILES: O=C(Nc1nc(NC(=O)c2ccco2)n(-c2ccccc2)n1)c1ccccc1]

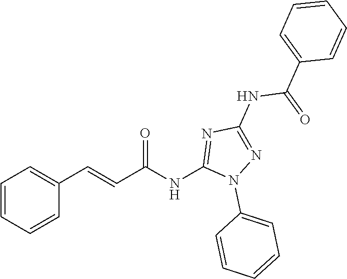 [SMILES: O=C(/C=C/c1ccccc1)Nc1nc(NC(=O)c2ccccc2)nn1-c1ccccc1]

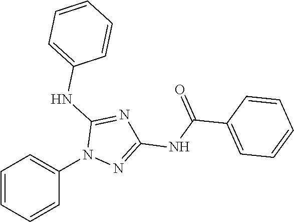 [SMILES: O=C(Nc1nc(Nc2ccccc2)n(-c2ccccc2)n1)c1ccccc1]

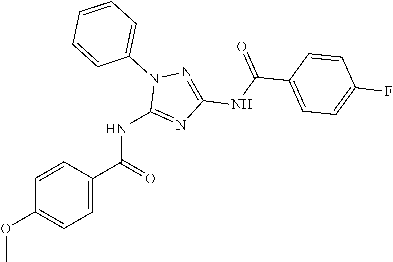 [SMILES: COc1ccc(C(=O)Nc2nc(NC(=O)c3ccc(F)cc3)nn2-c2ccccc2)cc1]

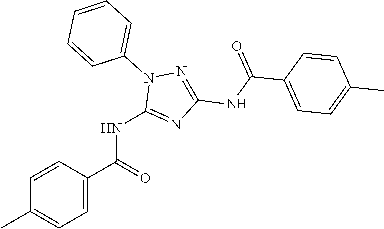

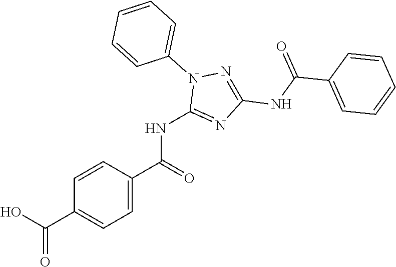 [SMILES: O=C(O)c1ccc(C(=O)Nc2nc(NC(=O)c3ccccc3)nn2-c2ccccc2)cc1]

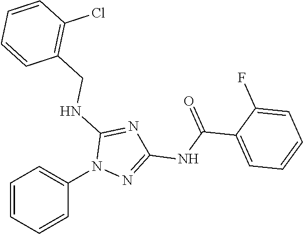 [SMILES: O=C(Nc1nc(NCc2ccccc2Cl)n(-c2ccccc2)n1)c1ccccc1F]

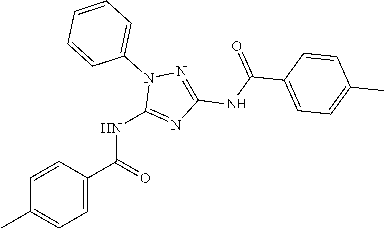

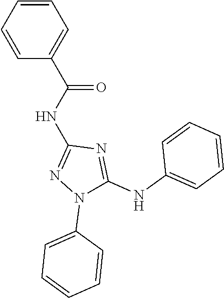 [SMILES: O=C(Nc1nc(Nc2ccccc2)n(-c2ccccc2)n1)c1ccccc1]

In another embodiment, the compounds described above may have particular functional characteristics. In one embodiment, the compound may have an oral bioavailability of about 10% to about 70% in a patient. In another embodiment, the compound may have an oral bioavailability of about 10% to about 50%. In another embodiment, the compound may have an oral bioavailability of about 10% to about 30%. In another embodiment the compound may have as oral bioavailability greater than shout 20%.

In another embodiment, when administered, orally, the compound may have a Tmax of about 0.2 hrs to about 2 hrs in a patient. In another embodiment, the compound may have a Tmax of about 0.3 hrs to about 1 hr in a patient. In another embodiment, the compound may have a Tmax of about 0.4 hrs to about 0.6 hr in a patient.

In another embodiment, when administered orally, the compound may have a Cmax of shout 100 ng/mL to about 1,000 ng/mL in a patient. In another embodiment, when administered orally, the compound may have a Cmax of about 150 ng/mL to about 500 ng/mL in a patient. In another embodiment when administered orally, the compound may have a Cmax of about 200 ng/mL to about 400 ng/mL in a patient.

In another embodiment, the compound may have a half-life in human liver microsomes greater than about 100 minutes. In another embodiment the compound may have: a half-life in human liver microsomes greater than about 300 minutes. In another embodiment the compound may have a half-life in human liver microsomes greater than about 500 minutes.

In another embodiment, the compound may have half-life in human liver microsomes of about 100 minutes to about 1,000 minutes. In another embodiment, the compound may have half-life in human liver microsomes of about 200 minutes to about 800 minutes. In another embodiment, the compound may have half-life in human liver microsomes of about 500 minutes to about 700 minutes.

In another embodiment, the compound may have a half-life in rat liver microsomes greater than about 100 minutes. In another embodiment, the compound may have a half-life in rat liver microsomes greater than about 300 minutes. In another embodiment, the compound may have a half-life in rat liver microsomes greater than about 500 minutes.

In another embodiment, the compound may have half-life in rat liver microsomes of about 100 minutes to about 1,000 minutes. In another embodiment, the compound may have half-life in rat liver microsomes of about 200 minutes to about 800 minutes. In another embodiment, the compound may have half-life in rat liver microsomes of about 500 minutes to about 700 minutes.

In a specific embodiment, the compound with any of the functional characteristics as described above may be a compound of formula (I), (I′), (IA′), (IA), (IB), (IC), (ID), (IE), (IF), (IG), (II), (II′), (IIA), (IIB), (III), (IIIA), (IIIB), (IIIC), (IIID), and/or (IIIE), or a pharmaceutically acceptable salt or solvate thereof. In a specific embodiment, the compound with the functional characteristics as described above may be from Table 1, Table IA, Table 2, Table 3 Table 3A, Tabled 3B, Table 3C, and/or Table 3D.

### Methods

Many cancers or tumorigenesis relate to genetic changes that regulate the G1/S cell cycle transition. While there are many genetic alterations that contribute to human cancers, a large number of mutations are in genes that encode proteins that regulate progression through the G1 phase of the cell cycle and in particular, the exit from G1. A potential regulator of the G1/S cell cycle switch is parkin ligase. Parkin ligase appears to degrade at least Cyclin D1, and thus impacts the binding of cyclin D1 with CDK4/6, a key regulator in the G1/S switch.

Many cancers specifically harbor mutations in the protein present in the Rb checkpoint pathway that controls progression of the cell cycle further downstream. For example, mutations in p16, p15 and/or p21 proteins or amplifications of the Cyclin D gene. Such mutations can favor the formation of the G1 cyclin D-CDK4/6 dimer complex which could increase the phosphorylation of Rb and increase the release of E2F, facilitating cells from the G1 phase to the S phase. Parkin ligase activators can be useful in degrading cyclin D and/or cyclin E, thereby minimizing the G1 cyclin D-CDK4/6 dimer complex formation and causing cell arrest or senescence. Thus, Parkin ligase activators as disclosed herein can be useful in treating subjects who have abnormally high expression levels of specific proteins (e.g., Cyclin D, Cyclin E) in the Rb checkpoint pathway or subjects who harbor mutations (e.g., p15, p16, p21) in the protein present in the Rb checkpoint pathway. Some cancers have chromosomal translocation of specific proteins in the Rb checkpoint pathway, which can cause amplification of one or more genes associated with the Rb checkpoint pathway (See Body, S. et al. Scientific Reports 2017, 7, 13946; which is hereby incorporated by reference in its entirety).

Furthermore, it appears that the increased activation of Parkin ligase, such as by the administration of a Parkin ligase activator disclosed herein, are particularly sensitive to patients/cancer cells with a mutation to any protein associated with the G1/S switch and/or senescence. Accordingly, in one embodiment of the methods herein, the Parkin ligase in a subject is wild type, or still has its function resulting in the degradation of cyclin D.

In one embodiment, the present disclosure provides a method of treating cancer in a subject in need thereof, comprising administering to the subject a Parkin ligase activator or a pharmaceutically acceptable salt thereof, wherein the Parkin ligase activator induces or prolongs senescence in a cell. In another embodiment, the subject has as least one mutation in a protein associated with the G1/S switch and/or senescence. In another embodiment, the subject has as least one mutation in a protein associated with the G1/S switch and/or senescence, wherein he Parkin ligase in a subject is wild type, or still has its function resulting in the degradation of cyclin D. In one embodiment, the mutated protein is selected from one or more of the group consisting of CDK4, CDK6, E2F, R_{b}, pRb, cyclin D, cyclin D1, cyclin E, cyclin E1, Ki67, INK4, p21, p27, p14, p15, and p16. In one embodiment, the mutated protein is selected from one or more of the group consisting of CDK4, CDK6, E2F, R_{b}, pRb, cyclin D, cyclin D1, cyclin E and cyclin E1. In one embodiment, the mutated protein is p53.

In another embodiment, the expression levels of a protein or co-factor associated with the G1/S switch and/or senescence is determined in a subject. In another embodiment, the expression levels of and/or activities of CDK4, CDK6, E2F, R_{b}, pRb, cyclin D, cyclin D1, cyclin E, cyclin E1, Ki67, INK4, p53, p21, p27, p14, p15, and/or p16 is determined in a subject. In another embodiment, the expression levels of and/or activities of CDK4, CDK6, E2F, R_{b}, pRb, cyclin D, cyclin D1, cyclin E, cyclin E1, Ki67, INK4, p53, p21, p27, p14, p15, parkin ligase, and/or p16 is determined in a cancer cell of a subject.

In another embodiment, the genotype of genes associated with the G1/S switch and/or senescence is determined in a patient. In a specific embodiment, the protein coding genes of CDK4, CDK6, E2F, Rb, pRb, cyclin D, cyclin D1, cyclin E, cyclin E1, Ki67, INK4, p53, p21, p27, p14, p15, and/or p16 is determined in a subject. In another specific embodiment, the protein coding genes are determined in a cancer cell of the subject. In another embodiment, the expression level, activity and/or genotype related to proteins associated with the G1/S switch and/or senescence in a patient are determined to establish a particular cancer treatment regimen for a subject. In another embodiment, the proteins are CDK4, CDK6, E2F, Rb, pRb, cyclin D, cyclin D1, cyclin E, cyclin E1, Ki67, INK4, p53, p21, p27, p14, p15, parkin ligase, and/or p16. In another embodiment, the expression level, activity and/or genotype is determined from a cancer cell of a patient. In another embodiment, after the expression level, activity and/or genotype are determined, the subject is administered a regimen that includes a parkin ligase activator. In another embodiment, the parkin ligase activator is a triazole compound or a pharmaceutically acceptable salt thereof. In another embodiment, the parkin ligase activator is a compound or a pharmaceutically acceptable salt thereof disclosed herein.

In one embodiment, the present disclosure provides a method of treating cancer in a subject in need thereof, comprising administering to the subject a Parkin ligase activator or a pharmaceutically acceptable salt thereof, wherein the subject has a mutant form of a protein in the R_{b} checkpoint pathway.

In one embodiment, the present disclosure provides a method of treating cancer in a subject in need thereof, comprising administering to the subject a Parkin ligase activator or a pharmaceutically acceptable salt thereof, wherein the subject has a chromosomal translocation of one or more genes associated with the Rb checkpoint pathway and/or with the G1/S switch and/or senescence. In one embodiment, the one or more genes where a chromosomal translocation occurs is selected from protein coding genes of CDK4, CDK6, E2F, Rb, pRb, cyclin D, cyclin D1, cyclin E, cyclin E1, Ki67, INK4, p53, p21, p27, p14, p15, and/or p16. In one embodiment, the chromosomal translocation occurs in protein coding genes of cyclin D. In one embodiment, a chromosomal translocation of cyclin D results in cyclin D amplification.

In one embodiment, the present disclosure provides a method of inhibiting or reducing abnormal (e.g., overexpressed) wild-type or mutated cyclin D activity in a subject in need thereof, comprising administering a Parkin ligase activator or a pharmaceutically acceptable salt thereof. In one embodiment, the present disclosure provides a method of inhibiting or reducing abnormal (e.g., overexpressed) wild-type or mutated cyclin D1 activity in a subject in need thereof, comprising administering a Parkin ligase activator or a pharmaceutically acceptable salt thereof.

In one embodiment, the present disclosure provides a method of inhibiting or reducing abnormal (e.g., overexpressed) wild-type or mutated cyclin D activity or expression in human cells, comprising contacting a Parkin ligase activator or a pharmaceutically acceptable salt thereof with the human cells. In one embodiment, the present disclosure provides a method of inhibiting or reducing abnormal (e.g., overexpressed) wild-type or mutated cyclin D1 activity or expression in human cells, comprising contacting a Parkin ligase activator or a pharmaceutically acceptable salt thereof with the human cells.

In one embodiment, the present disclosure provides a method of inducing cell cycle arrest or senescence in a subject in need thereof, comprising administering a Parkin ligase activator or a pharmaceutically acceptable salt thereof.

In one embodiment of any of the methods disclosed herein, the cancer is selected from one or more of Acute Lymphoblastic Leukemia, Acute Myeloid Leukemia, Adrenocortical Carcinoma, AIDS-Related Cancers, Kaposi Sarcoma, liposarcoma, soft tissue sarcoma, Lymphoma, Anal Cancer, Appendix Cancer, Astrocytomas, Childhood Atypical Teratoid/Rhabdoid Tumor, Basal Cell Carcinoma, Skin Cancer (Nonmelanoma), Childhood Bile Duct Cancer, Extrahepatic Bladder Cancer, Bone Cancer, Ewing Sarcoma Family of Tumors, Osteosarcoma and Malignant Fibrous Histiocytoma, Brain Stem Glioma, Brain Tumors, Embryonal Tumors, Germ Cell Tumors, Craniopharyngioma, Ependymoma, Bronchial Tumors, Burkitt Lymphoma (Non-Hodgkin Lymphoma), Carcinoid Tumor, Gastrointestinal Carcinoma of Unknown Primary, Cardiac (Heart) Tumors, Lymphoma, Primary, Cervical Cancer, Childhood Cancers, Chordoma, Chronic Lymphocytic Leukemia, Chronic Myelogenous Leukemia, Chronic Myeloproliferative Neoplasms Colon Cancer, Colorectal Cancer, Cutaneous T-Cell Lymphoma, Ductal Carcinoma In Situ, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Intraocular Melanoma, Retinoblastoma, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors, Extragonadal Cancer, Ovarian Cancer, Testicular Cancer, Gestational Trophoblastic Disease, Glioma, Brain Stem Cancer, Hairy Cell Leukemia, Head and Neck Cancer, Heart Cancer, Hepatocellular (Liver) Cancer, Histiocytosis, Langerhans Cell Cancer, Hodgkin Lymphoma, Hypopharyngeal Cancer, Intraocular Melanoma, Islet Cell Tumors, Pancreatic Neuroendocrine Tumors, Kidney Cancer, Renal Cell Cancer, Wilms Tumor and Other Childhood Kidney Tumors, Langerhans Cell Histiocytosis, Laryngeal Cancer, Leukemia, Chronic Lymphocytic Cancer, Chronic Myelogenous Cancer, Hairy Cell Cancer, Lip and Oral Cavity Cancer, Liver Cancer (Primary), Lobular Carcinoma In Situ (LCIS), Lung Cancer, Non-Small Cell Cancer, Small Cell Cancer, Lymphoma, Mantle cell lymphoma, Cutaneous T-Cell (Mycosis Fungoides and Sézary Syndrome), Hodgkin Cancer, Non-Hodgkin Cancer, Macroglobulinemia, Waldenström, Male Breast Cancer, Malignant Fibrous Histiocytoma of Bone and Osteosarcoma, Melanoma, Intraocular (Eye) Cancer, Merkel Cell Carcinoma, Mesothelioma, Malignant, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic, Myeloid Leukemia, Acute, Myeloma Multiple, Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and Oropharyngeal Cancer, Osteosarcoma and Malignant Fibrous Histiocytoma of Bone, Epithelial Cancer, Low Malignant Potential Tumor, Pancreatic Cancer, Pancreatic Neuroendocrine Tumors (Islet Cell Tumors), Papillomatosis, Paraganglioma, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System Lymphoma, Rectal Cancer, Renal Cell (Kidney) Cancer, Retinoblastoma, Rhabdomyosarcoma, Salivary Gland Cancer, Sarcoma, Ewing Cancer, Kaposi Cancer, Osteosarcoma (Bone Cancer), Soft Tissue Cancer, Uterine Cancer, Sézary Syndrome, Skin Cancer, Childhood Melanoma, Merkel Cell Carcinoma, Nonmelanoma, Small Cell Lung Cancer, Small Intestine Cancer, Soft Tissue Sarcoma, Squamous Cell Carcinoma, Skin Cancer (Nonmelanoma), Childhood Squamous Neck Cancer with Occult Primary, Metastatic Cancer, Stomach (Gastric) Cancer, T-Cell Lymphoma, Cutaneous Cancer, Testicular Cancer, Throat Cancer, Thymoma and Thymic Carcinoma, Thyroid Cancer, Transitional Cell Cancer of the Renal Pelvis and Ureter, Unknown Primary, Carcinoma of Childhood, Unusual Cancers of Childhood, Urethral Cancer, Uterine Cancer, Endometrial Cancer, Uterine Sarcoma, Vaginal Cancer, Vulvar Cancer, Waldenström Macroglobulinemia, Wilms Tumor, and/or Women's Cancers

In one embodiment, the cancer is sarcoma, lymphoma, colon cancer, lung cancer, or ovarian cancer.

In one embodiment of any of the methods disclosed herein, the mutated protein is selected from one or more of the group consisting of CDK4, CDK6, E2F, Rb, pRb, cyclin D, cyclin D1, cyclin E, cyclin E1, Ki67, INK4, p53, p14, p21, p27, p15, and p16. In one embodiment, the mutated protein is selected from one or more of the group consisting of R_{b}, pRb, cyclin D1 and cyclin E. In one embodiment, the mutated protein is selected from one or more of the group consisting of cyclin D1, p14, p15, p16, p21, Rb, and/or p53. In some embodiments, the mutated protein is cyclin D1.

In another embodiment, the subject has a loss of p16 or diminished activity relative to wildtype p16. In a specific embodiment, the loss or diminished activity is due to a mutation of p16. In another embodiment, the mutation is a point mutation. In another embodiment, the loss of activity is due to deletion. In another embodiment, the deletion may be either homozygous or heterozygous.

In one embodiment, the mutated protein is encoded by one or more gene selected from CCNDf CDKN2A, CDKN2B, CDKN1A, RB, and/or TP53.

In one embodiment of any of the methods disclosed herein, the mutated protein is from a point mutation. In another embodiment, cyclin D1 has at least one point mutation. In one embodiment, the mutated cyclin D1 comprises at least one point mutation on R260H. In some embodiments, the point mutation of cyclin D1 is T286I.

In one embodiment of any of the methods disclosed herein, the mutated protein is cyclin D1. In one embodiment, mutated cyclin D1 has a copy number variation (CNV) of 1 or greater than 2. In one embodiment, mutated cyclin D1 has a CNV of greater than 2. In some embodiments, the mutated cyclin D1 has CNV3, CNV4, CNV5, or CNV6.

In one embodiment, the mutated cyclin D1 is due to one or more genomic deletion in CCND1.

In one embodiment of any of the methods disclosed herein, the at least one point mutation is on p14, p15, p16, and/or p21. In one embodiment, the mutated p14, p15, p16, and/or p21 comprises at least one point mutation on H83Y, D84Y, D84V, R19H, or R67L. In one embodiment, the mutated p21 comprises at least one stop codon (*) at R90* or G23*.

In one embodiment of any of the methods disclosed herein, the mutated protein is pi 4, p15, p16, and/or p21. In one embodiment, mutated p21 has a CNV of 1 or greater than 2. In some embodiments, the mutated p21 has CNV1, CNV3, CNV4, CNV5, or CNV6.

In one embodiment, the mutated p14, p15, p16, and/or p21 is due to one or more genomic deletion in CDKN1A, CDKN2A, and/or CDKN2B.

In one embodiment of any of the methods disclosed herein, the at least one point mutation is on Rb. In one embodiment, the mutated Rb comprises at least one stop codon (*) at K715*.

In one embodiment of any of the methods disclosed herein, the mutated protein is Rb. In one embodiment, mutated Rb has a CNV of 1 or greater than 2. In some embodiments, the mutated Rb has CNV1, CNV3, CNV4, CNV5, CNV6, or CNV7

In one embodiment, the mutated Rb is due to one or more genomic deletion in RB. In one embodiment, Rb is wild type.

In one embodiment of any of the methods disclosed herein, the at least one point mutation is on p53. In one embodiment, the mutated p53 comprises at least one point mutation on R175H, R43H, M237I, R273H, C176W, R280K, L52R, L145R, R248W, and/or L130V. In one embodiment, the mutated p53 comprises at least one stop codon (*) at R306*, V73*, S90*, P153*, R196*, Y103*, or T118*.

In one embodiment of any of the methods disclosed herein, the mutated protein is p53. In one embodiment, mutated p53 has a CNV of 1 or greater than 2.

In one embodiment, the mutated p53 is due to one or more genomic deletion in TP53.

In another embodiment, the list of possible mutated proteins described above are wild type, wherein not all proteins selected from one or more of the group consisting of CDK4, CDK6, E2F, Rb, pRb, cyclin D, cyclin D1, cyclin E, cyclin E1, Ki67, INK4, p53, p14, p21, p27, p15, and p16 are mutated. In a specific embodiment, parkin ligase is wild type parkin ligase. In a specific embodiment, proteins selected from one or more of the group consisting of CDK4, CDK6, E2F, Rb, pRb, cyclin D, cyclin D1, cyclin E, cyclin E1, Ki67, INK4, p53, p14, p21, p27, p15, and p16 are wild type. In one embodiment, the wild type protein is selected from one or more of the group consisting of Rb, pRb, cyclin D1 and cyclin E. In one embodiment, the wild type protein is selected from one or more of the group consisting of cyclin D1, p14, p15, p16, p21, Rb, and/or p53. In a specific embodiment, Rb and/or pRb is a wild type protein. In a specific embodiment, the wild type/mutation pattern of CDK4, CDK6, E2F, Rb, pRb, cyclin D, cyclin D1, cyclin E, cyclin E1, Ki67, INK4, p53, p14, p21, p27, p15, and p16 is as in specific cancerous cell lines. In one embodiment, the mutated protein comprises mutations and wild type proteins as observed in the cell lines as shown below in Table 4,

TABLE 4
Cyclin D Pathway Mutations

| Cell Line | CCND1 | CDKN2A/CDKN2B | CDKN1A | RB | TP53 | Others |
|---|---|---|---|---|---|---|
| MOLT-4 | wt | lossA/CNV4B | wt | wt | R306* | |
| DOHH2 | wt | lossA/lossB | wt | wt | wt | |
| KM12 | wt | wt | R19H | wt | V73* | |
| CCRF-CEM | R260H | loss | wt | wt | R175H | |
| A-427 | CNV5 | wtA/lossB | CNV3 | wt | wt | |
| NCl-H292 | CNV3 | loss | wt | wt | wt | |
| LNCaP | CNV4 | CNV4 | CNV4 | wt | wt | |
| SK-OV-3 | CNV5 | loss (p21) | Cnv4 | wt | S90* | |
| SK-CO-1 | CNV4 | CNV6/CNV6 | CNV3 | wt | wt | |
| TOV-112D | wt | wt | wt | wt | R43H | |
| TOV-21G | wt | wt | wt | wt | wt | |
| HGC-27 | CNV3 | CNV4 | CNV4 | CNV3 | P153* | |
| Calu-3 | CNV3 | H83Y | R67L | wt | M237I | |
| Hutu 80 | wt | wt | wt | wt | | |
| HL-60 | wt | R80* | wt | wt | wt | |
| DU 145 | CNV3 | D84Y | CNV3 | K715* | P153* | |
| A-204 | WT | wt | wt | wt | wt | |
| NCl-H1703 | wt | D84V(A)/CNV1(B) | CNV3 | wt | wt | |
| OVCAR8 | CNV3 | wt | wt | wt | wt | |
| Calu-6 | CNV3 | wt | wt | wt | R196* | |
| COV504 | wt | wt | wt | wt | point mutant | |
| A431 | CNV8 | CNV4(A)/loss (B) | CNV3 | wt | R273H | |
| HCT 116 | wt | G23* | wt | wt | wt | |
| A2780 | wt | wt | wt | wt | wt | |
| SHP-77 | CNV3 | wt | wt | CNV1 | C176W | |
| MSTO-211H | CNV3 | CNV3(A)/CNV1(B) | CNV3 | CNV3 | wt | |
| MCF-7 | CNV3 | loss | CNV3 | | wt | |
| HT-29 | CNV4 | CNV3 | CNV3 | wt | R273H | |
| COLO 205 | CNV3 | CNV3 | | CNV7 | Y103* | |
| MDA-MB-231 | CNV4 | | CNV6 | | R280K | |
| OVISE | wt | CNV3 | CNV3 | wt | wt | WT CDKs |
| SK-MEL-28 | CNV6 | CNV3 | CNV4 | CNV3 | L52R/L145R | R24C CDK4 |
| SW837 | wt | wt | CNV1 | CNV1 | R248W | |
| SW948 | CNV3 | wt | CNV3 | CNV6 | T118* | |
| JIMT-1 | CNV3 | wt | CNV3 | wt | R248W | |
| T98G | CNV3 | lossA/wtB | CNV3 | CNV3 | M237I | R392* pkn |
| U-87 MG | wt | lossA/lossB | CNV1 | CNV1 | | |
| OVTOKO | wt | loss | | | wt | |
| OVCAR4 | CNV3 | wt | CNV3 | wt | L130V | CDK6 |
| | | | | | | CNV5/CNV5 CCNE |

*Indicates a stop codon (protein not expressed)

In one embodiment of any of the methods disclosed herein, the mutated protein has a copy number variation (CNV) of 1 or greater than 2. In some embodiments, the mutated protein has CNV1, CNV3, CNV4, CNV5, CNV6, CNV7, CNV8, CNV9, or CNV10.

In one embodiment of any of the methods disclosed herein, the point mutation is on only one allele. In another embodiment, the point mutation is on two alleles.

In one embodiment of any of the methods disclosed herein, the subject harbors any of the mutation as disclosed herein.

In one embodiment of any of the methods disclosed herein, the subject harbors wildtype Rb. In one embodiment, the subject harbors wildtype Rb and loss ofp 14, p15, p16, and/or p21.

In one embodiment, the subject harbors wildtype Rb and loss p21. In one embodiment, the subject harbors wildtype Rb, loss of p14, p15, p16, and/or p21, and cyclin D1 with a CNV of greater than 2. In one embodiment, the subject harbors wildtype Rb, loss p21, and cyclin D1 with a CNV of greater than 2.

In one embodiment of any of the methods disclosed herein, the subject harbors loss of p14, p15, p16, and/or p21. In one embodiment, the subject harbors loss of p21. In one embodiment of any of the methods disclosed herein, the subject harbors loss of p14, p15, p16, and/or p21 and cyclin D1 with a CNV of greater than 2. In one embodiment, the subject harbors loss of p21 and cyclin D1 with a CNV of greater than 2.

In one embodiment of any of the methods disclosed herein, the subject harbors cyclin D1 with a CNV of greater than 2.

In one embodiment, the mutated protein comprises mutation observed in the cell lines as shown below in Table 5.

TABLE 5
Cyclin D Pathway Mutations

| | Mutations | | | | | | |
|---|---|---|---|---|---|---|---|
| Cell Line | p16 | CDK4 | CDK6 | Cyclin D | Parkin | RB | p53 |
| Colon (HCT-116) | ✓ | | | | | | |
| Lung (A549) | ✓ | | | | | | |
| Mantle Cell (Jeko-1) | | ✓ | | ✓ | | | ✓ |
| Ovarian (A2780) | | | | | | | |
| Ovarian (ES-2) | | | | ✓ | | | ✓ |
| Ovarian (OAW28) | | | | ✓ | | ✓ | ✓ |
| Ovarian (OAW42) | | ✓ | ✓ | | | | |
| Ovarian (SKOV3) | ✓ | ✓ | | ✓ | | | ✓ |
| Ovarian (SW626) | ✓ | | | | | | ✓ |
| Ovarian (TOV112D) | | ✓ | ✓ | | | | ✓ |
| Ovarian (TOV21G) | | | | | | | |

✓ Means Mutations Found

In some instances, mutated protein can promote gene amplification and/or overexpression (see, Williams, M. E. et al. Cancer Research (Supply 1992, 52, 5541, which is hereby incorporated in its entirety). In some cancers, cyclin D genes have been shown to be amplified (see Jiang, W., et. al. Cancer Research 1992, 52, 2980; Herman, J. G., et. al. Cancer Research, 1995, 55, 4525; Nair B. C., et. al. Gene Ther. Mol. Biol. 2008, 12, 395; which are hereby incorporated in their entireties).

In one embodiment, the mutated protein promotes gene amplification. In one embodiment, the mutated protein promotes amplification of one or more genes associated with the Rb checkpoint pathway. In one embodiment, the mutated protein promotes amplifications of the Cyclin D gene. In another embodiment, the mutated proteins are selected from pi 6, p15 and/or p21 proteins. In some embodiments, mutated p16, p15 and/or p21 proteins promotes amplifications of the Cyclin D gene.

In one embodiment of any of the methods disclosed herein, the mutated protein promotes chromosome translocation.

In one embodiment of any of the methods disclosed herein, the mutation provides overexpression of one or more genes associated with the R_{b} checkpoint pathway. In one embodiment of any of the methods disclosed herein, the mutation provides overexpression of cyclin D, cyclin D1, and/or cyclin E. In one embodiment of any of the methods disclosed herein, the mutation provides overexpression of cyclin D1. In a specific embodiment, the mutation is on a protein, wherein the mutation provides overexpression of cyclin D, cyclin D1, and/or cyclin E. In a specific embodiment, the overexpressed protein is cyclin D1. In another embodiment, the gene of cyclin D, cyclin D1, and/or cyclin E is overexpressed. In a specific embodiment, the overexpressed gene is cyclin D1.

In one embodiment of any of the methods disclosed herein, the subject is human. In one embodiment, the subject harbors any one of the mutations as disclosed herein.

In one embodiment, the Parkin ligase activators as used in any of the method disclosed herein is selected from a compound of formula (I), (I′), (IA′), (IA), (IB), (IC), (ID), (IE), (IF), (IG), (II), (II′), (IIA), (IIB), (III), (IIIA), (IIIB), (IIIC), (IIID), and/or (IIIE), or a pharmaceutically acceptable salt or solvate thereof. In one embodiment, the parkin activators may be a compound from Table 1, Table IA, Table 2, Table 3 Table 3A, Tabled 3B, Table 3C, and/or Table 3D or a pharmaceutically acceptable salt or solvate thereof. In one embodiment, the Parkin ligase activators as used in any of the method disclosed herein is selected from a compound from Tables 3, and 3A to 3D or a pharmaceutically acceptable salt or solvate thereof.

In one embodiment, the Parkin ligase activators as used in any of the method disclosed herein is

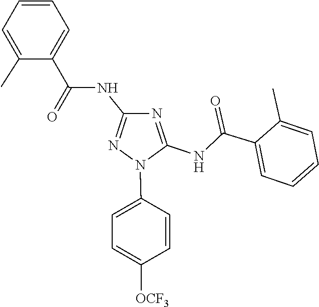 [SMILES: Cc1ccccc1C(=O)Nc1nc(NC(=O)c2ccccc2C)n(-c2ccc(OC(F)(F)F)cc2)n1]

(Compound 42) or a pharmaceutically acceptable salt thereof. In one embodiment, the Parkin ligase activators as used in any of the method disclosed herein is

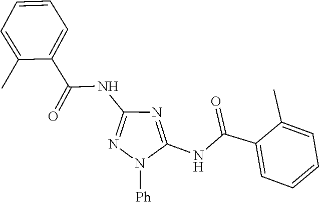 [SMILES: Cc1ccccc1C(=O)Nc1nc(NC(=O)c2ccccc2C)n(-c2ccccc2)n1]

(Compound F), or a pharmaceutically acceptable salt thereof.

In one embodiment, a method of treating cancer in a subject in need thereof, comprising administering to the subject a therapeutically effective amount of Compound 42 or Compound F, or a pharmaceutically acceptable salt thereof, wherein the subject has a mutant form of a protein in the Rb checkpoint pathway is disclosed. In one embodiment, a method of treating cancer in a subject in need thereof, comprising administering to the subject a therapeutically effective amount of Compound 42 or Compound F, or a pharmaceutically acceptable salt thereof, wherein the subject has a mutant form of a protein selected from cyclin D1, p14, p15, p16, p21, Rb, and/or p53 is disclosed. In other embodiments, a method of treating cancer in a subject in need thereof, comprising administering to the subject a therapeutically effective amount of Compound 42 or Compound F, or a pharmaceutically acceptable salt thereof, wherein the subject has a mutant form of a protein selected from cyclin D1, p21, Rb, and/or p53 is disclosed. In one embodiment, the mutant form of protein is in at least one of the forms selected from point mutation, copy number variation number of greater than 2, gene amplification, and/or chromosome translocation, is disclosed. In one embodiment, a method of treating sarcoma, lymphoma, colon cancer, lung cancer, or ovarian cancer in a subject in need thereof, comprising administering to the subject a therapeutically effective amount of Compound 42 or Compound F, or a pharmaceutically acceptable salt thereof, wherein the subject has a mutant form of a protein in the Rb checkpoint pathway is disclosed.

In one embodiment, a method of inhibiting or reducing abnormal (e.g., overexpressed) wild-type or mutated cyclin D1, p21, Rb, and/or p53 in a subject in need thereof, comprising administering to the subject a therapeutically effective amount of Compound 42 or Compound F, or a pharmaceutically acceptable salt thereof is disclosed. In one embodiment, a method of inhibiting or reducing abnormal (e.g., overexpressed) wild-type or mutated cyclin D1 in a subject in need thereof, comprising administering to the subject a therapeutically effective amount of Compound 42 or Compound F, or a pharmaceutically acceptable salt thereof is disclosed.

In one embodiment, a method of inducing cell cycle arrest or senescence in a subject in need thereof, comprising administering to the subject a therapeutically effective amount of Compound 42 or Compound F, or a pharmaceutically acceptable salt thereof, wherein the subject has a mutant form of a protein in the Rb checkpoint pathway is disclosed. In one embodiment, a method of inducing cell cycle arrest or senescence in a subject in need thereof, comprising administering to the subject a therapeutically effective amount of Compound 42 or Compound F, or a pharmaceutically acceptable salt thereof, wherein the subject has a mutant form of a protein selected from cyclin D1, p14, p15, p16, p21, Rb, and/or p53 is disclosed. In other embodiments, a method of inducing cell cycle arrest or senescence in a subject in need thereof, comprising administering to the subject a therapeutically effective amount of Compound 42 or Compound F, or a pharmaceutically acceptable salt thereof, wherein the subject has a mutant form of a protein selected from cyclin D1, p21, Rb, and/or p53 is disclosed.

In one embodiment, a method of inhibiting or reducing abnormal (e.g., overexpressed) wild-type or mutated cyclin D1, p21, Rb, and/or p53 activity or expression in human cells, comprising contacting Compound 42 or Compound F, or a pharmaceutically acceptable salt thereof with the human cells is disclosed. In one embodiment, a method of inhibiting or reducing abnormal (e.g., overexpressed) wild-type or mutated cyclin D1 activity or expression in human cells, comprising contacting Compound 42 or Compound F, or a pharmaceutically acceptable salt thereof with the human cells is disclosed. In one embodiment human cells is selected from cells of any cancer as disclosed herein. In one embodiment, the human cells harbor any one of the mutations as disclosed herein.

In one embodiment of any one of the methods herein, cancer has developed resistant to one or more anticancer treatment. In one embodiment of any one of the methods herein, cancer is resistant to one or more CDK4/6 inhibitors. In one embodiment of any one of the methods herein, cancer is resistant to palbociclib. In one embodiment of any one of the methods herein, cancer is platinum-resistant.

In a specific embodiment, the methods include treating cancer in a subject in need thereof, comprising administering to the subject a Parkin ligase activator or a pharmaceutically acceptable salt thereof, wherein the subject has a mutant form of a protein in the Rb checkpoint pathway and/or mutant form of p53, wherein the Parkin ligase activator is selected from a compound of formula (I), (I′), (IA′), (IA), (IB), (IC), (ID), (IE), (IF), (IG), (II), (II′), (IIA), (IIB), (III), (IIIA), (IIIB), (IIIC), (IIID), and/or (IIIE), or a pharmaceutically acceptable salt or solvate thereof. In one embodiment, the Parkin ligase activator may be a compound from Table 1, Table IA, Table 2, Table 3 Table 3A, Tabled 3B, Table 3C, and/or Table 3D or a pharmaceutically acceptable salt or solvate thereof. In a specific embodiment, the Parkin ligase activator is Compound 42 or Compound F or a pharmaceutically acceptable salt or solvate thereof.

In another specific embodiment, the methods include treating cancer in a subject in need thereof, comprising administering to the subject a Parkin ligase activator or a pharmaceutically acceptable salt thereof, wherein the subject has a defect in the regulation of Cyclin D expression or activity. In another specific embodiment, the Parkin ligase activator is selected from a compound of formula (I), (I′), (IA′), (IA), (IB), (IC), (ID), (IE), (IF), (IG), (II), (II′), (IIA), (IIB), (III), (IIIA), (IIIB), (IIIC), (IIID), and/or (IIIE), or a pharmaceutically acceptable salt or solvate thereof. In one embodiment, the Parkin ligase activator may be a compound from Table 1, Table IA, Table 2, Table 3 Table 3A, Tabled 3B, Table 3C, and/or Table 3D or a pharmaceutically acceptable salt or solvate thereof. In a specific embodiment, the Parkin ligase activator is Compound 42 or Compound F or a pharmaceutically acceptable salt or solvate thereof.

In another specific embodiment, the methods include inhibiting or reducing overexpressed wild-type or mutated cyclin D1 activity in a subject in need thereof, comprising administering a Parkin ligase activator or a pharmaceutically acceptable salt thereof, wherein the Parkin ligase activator is selected from a compound of formula (I), (I′), (IA′), (IA), (IB), (IC), (ID), (IE), (IF), (IG), (II), (II′), (IIA), (IIB), (III), (IIIA), (IIIB), (IIIC), (IIID), and/or (IIIE), or a pharmaceutically acceptable salt or solvate thereof. In one embodiment, the Parkin ligase activator may be a compound from Table 1, Table 1A, Table 2, Table 3 Table 3A, Tabled 3B, Table 3C, and/or Table 3D or a pharmaceutically acceptable salt or solvate thereof. In a specific embodiment, the Parkin ligase activator is Compound 42 or Compound F or a pharmaceutically acceptable salt or solvate thereof. In a specific embodiment, the overexpressed wild-type or mutated cyclin D1 activity is associated with a mutation on a protein, or the mutation provides overexpression, amplification, or deletion of one or more protein coding genes of the Rb checkpoint pathway or p53. In a specific embodiment, the mutation provides overexpression of the cyclin D1 gene. In a specific embodiment, the overexpression or mutated cyclin D1 activity is in human cells. In a specific embodiment, the human cells are cancer cells.

In another embodiment, the subject harbors a mutation in an upstream signaling pathway that leads to increased expression of cyclin D. In some embodiments, the signaling pathways that increase expression by increasing transcription of cyclin D1 include MAP kinases, phosphoinositide 3-kinase/protein kinase B (PI3K/Akt) signaling, IKK/IκB/NF-κB pathway, Wnt/β-catenin signaling, STAT signaling, and nuclear hormone receptors. Consistent with these, the cyclin D1 promoter has consensus sequences for Ets, Fos/Jun, NF-κB, STAT, TCF, E2F, Sp1, EGR, and ERα.

In another specific embodiment, the methods include inducing cell cycle arrest or senescence in a subject in need thereof, comprising administering a Parkin ligase activator or a pharmaceutically acceptable salt thereof, wherein the Parkin ligase activator is selected from a compound of formula (I), (I′), (IA′), (IA), (IB), (IC), (ID), (IE), (IF), (IG), (II), (II′), (IIA), (IIB), (III), (IIIA), (IIIB), (IIIC), (IIID), and/or (IIIE), or a pharmaceutically acceptable salt or solvate thereof. In one embodiment, the Parkin ligase activator may be a compound from Table 1, Table 1A, Table 2, Table 3 Table 3A, Tabled 3B, Table 3C, and/or Table 3D or a pharmaceutically acceptable salt or solvate thereof. In a specific embodiment, the Parkin ligase activator is Compound 42 or Compound F or a pharmaceutically acceptable salt or solvate thereof

In another embodiment, the methods of the present invention include treating a subject having a dysregulated Rb checkpoint pathway that results in increased cell growth, comprising administering to the subject a Parkin ligase activator or a pharmaceutically acceptable salt thereof. In a specific embodiment, the Parkin ligase activator is selected from a compound of formula (I), (I′), (IA′), (IA), (IB), (IC), (ID), (IE), (IF), (IG), (II), (II′), (IIA), (IIB), (III), (IIIA), (IIIB), (IIIC), (IIID), and/or (IIIE), or a pharmaceutically acceptable salt or solvate thereof. In one embodiment, the Parkin ligase activator may be a compound from Table 1, Table 1A, Table 2, Table 3 Table 3A, Tabled 3B, Table 3C, and/or Table 3D or a pharmaceutically acceptable salt or solvate thereof. In a specific embodiment, the Parkin ligase activator is Compound 42 or Compound F or a pharmaceutically acceptable salt or solvate thereof. In another specific embodiment, the pathway is dysregulated by increased or decreased expression of a wild type protein in the pathway. In another embodiment, the increased or decreased expression results from a mutation of regulatory elements that control protein expression. In another embodiment, the mutation of regulatory elements may be through alteration of enhancers or promoters, or that control transcription and translation of a protein in the pathway, or microRNAs that control transcription and degradation of mRNAs. In another embodiment, the pathway is dysregulated by a mutation of a protein in the Rb checkpoint pathway. In another embodiment, there is altered expression of any one of proteins CDK4, CDK6, E2F, Rb, pRb, cyclin D, cyclin D1, cyclin E, cyclin E1, Ki67, INK4, p53, p21, p27, p14, p15, and/or p16. In another embodiment, the protein is selected from Rb, cyclin D1, p53, p16, p15 and/or p21. In another embodiment, the pathway is dysregulated by a mutated protein. In another embodiment, the mutated protein is selected from CDK4, CDK6, E2F, Rb, pRb, cyclin D, cyclin D1, cyclin E, cyclin E1, Ki67, INK4, p53, p21, p27, p14, p15, and/or p16. In a specific embodiment, the mutated protein is selected from Rb, cyclin D1, p53, p16, p15 and/or p21. In another embodiment, the dysregulated Rb checkpoint pathway is associated with cancer.

In another specific embodiment, the cancer is selected from Acute Lymphoblastic Leukemia, Acute Myeloid Leukemia, Adrenocortical Carcinoma, AIDS-Related Cancers, Kaposi Sarcoma, liposarcoma, soft tissue sarcoma, Lymphoma, Anal Cancer, Appendix Cancer, Astrocytomas, Childhood Atypical Teratoid/Rhabdoid Tumor, Basal Cell Carcinoma, Skin Cancer (Nonmelanoma), Childhood Bile Duct Cancer, Extrahepatic Bladder Cancer, Bone Cancer, Ewing Sarcoma Family of Tumors, Osteosarcoma and Malignant Fibrous Histiocytoma, Brain Stem Glioma, Brain Tumors, Embryonal Tumors, Germ Cell Tumors, Craniopharyngioma, Ependymoma, Bronchial Tumors, Burkitt Lymphoma (Non-Hodgkin Lymphoma), Carcinoid Tumor, Gastrointestinal Carcinoma of Unknown Primary, Cardiac (Heart) Tumors, Lymphoma, Primary, Cervical Cancer, Childhood Cancers, Chordoma, Chronic Lymphocytic Leukemia, Chronic Myelogenous Leukemia, Chronic Myeloproliferative Neoplasms Colon Cancer, Colorectal Cancer, Cutaneous T-Cell Lymphoma, Ductal Carcinoma In Situ, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Intraocular Melanoma, Retinoblastoma, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors, Extragonadal Cancer, Ovarian Cancer, Testicular Cancer, Gestational Trophoblastic Disease, Glioma, Brain Stem Cancer, Hairy Cell Leukemia, Head and Neck Cancer, Heart Cancer, Hepatocellular (Liver) Cancer, Histiocytosis, Langerhans Cell Cancer, Hodgkin Lymphoma, Hypopharyngeal Cancer, Intraocular Melanoma, Islet Cell Tumors, Pancreatic Neuroendocrine Tumors, Kidney Cancer, Renal Cell Cancer, Wilms Tumor and Other Childhood Kidney Tumors, Langerhans Cell Histiocytosis, Laryngeal Cancer, Leukemia, Chronic Lymphocytic Cancer, Chronic Myelogenous Cancer, Hairy Cell Cancer, Lip and Oral Cavity Cancer, Liver Cancer (Primary), Lobular Carcinoma In Situ (LCIS), Lung Cancer, Non-Small Cell Cancer, Small Cell Cancer, Lymphoma, Mantle cell lymphoma, Cutaneous T-Cell (Mycosis Fungoides and Sézary Syndrome), Hodgkin Cancer, Non-Hodgkin Cancer, Macroglobulinemia, Waldenström, Male Breast Cancer, Malignant Fibrous Histiocytoma of Bone and Osteosarcoma, Melanoma, Intraocular (Eye) Cancer, Merkel Cell Carcinoma, Mesothelioma, Malignant, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic, Myeloid Leukemia, Acute, Myeloma Multiple, Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and Oropharyngeal Cancer, Osteosarcoma and Malignant Fibrous Histiocytoma of Bone, Epithelial Cancer, Low Malignant Potential Tumor, Pancreatic Cancer, Pancreatic Neuroendocrine Tumors (Islet Cell Tumors), Papillomatosis, Paraganglioma, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System Lymphoma, Rectal Cancer, Renal Cell (Kidney) Cancer, Retinoblastoma, Rhabdomyosarcoma, Salivary Gland Cancer, Sarcoma, Ewing Cancer, Kaposi Cancer, Osteosarcoma (Bone Cancer), Soft Tissue Cancer, Uterine Cancer, Sëzary Syndrome, Skin Cancer, Childhood Melanoma, Merkel Cell Carcinoma, Nonmelanoma, Small Cell Lung Cancer, Small Intestine Cancer, Soft Tissue Sarcoma, Squamous Cell Carcinoma, Skin Cancer (Nonmelanoma), Childhood Squamous Neck Cancer with Occult Primary, Metastatic Cancer, Stomach (Gastric) Cancer, T-Cell Lymphoma, Cutaneous Cancer, Testicular Cancer, Throat Cancer, Thymoma and Thymic Carcinoma, Thyroid Cancer, Transitional Cell Cancer of the Renal Pelvis and Ureter, Unknown Primary, Carcinoma of Childhood, Unusual Cancers of Childhood, Urethral Cancer, Uterine Cancer, Endometrial Cancer, Uterine Sarcoma, Vaginal Cancer, Vulvar Cancer, Waldenström Macroglobulinemia, Wilms Tumor, and/or Women's Cancers. In another embodiment, the cancer is selected from breast cancer, sarcoma, lymphoma, colon cancer, lung cancer, or ovarian cancer.

### Pharmaceutical Compositions and Formulations

The present disclosure also includes pharmaceutical compositions useful any of the method disclosed herein. In one embodiment, a pharmaceutical composition comprises one or more compounds of formula (I), (I′), (IA′), (IA), (IB), (IC), (ID), (IE), (IF), (IG), (II), (II′), (IIA), (IIB), (III), (IIIA), (IIIB), (IIIC), (IIID), and/or (IIIE), or a pharmaceutically acceptable salt or solvate thereof.

In one embodiment of the present disclosure, a pharmaceutical composition comprises a therapeutically effective amounts of one or more compounds of formula (I), (I′), (IA′), (IA), (IB), (IC), (ID), (IE), (IF), (IG), (II), (II′), (IIA), (IIB), (III), (IIIA), (IIIB), (IIIC), (IIID), and/or (IIIE), or a pharmaceutically acceptable salt or solvate thereof.

In a specific embodiment, a pharmaceutical composition, as described herein, comprises one or more compounds selected from Table 1 or Table IA, or a pharmaceutically acceptable salt or solvate thereof. In one embodiment, a pharmaceutical composition as described herein comprise one or more compounds selected from Table 2, or a pharmaceutically acceptable salt or solvate thereof. In one embodiment, a pharmaceutical composition as described herein comprise one or more compounds selected from Table 3 or Table 3A, or a pharmaceutically acceptable salt or solvate thereof.

In one embodiment, a pharmaceutical composition described herein does not contain a compound disclosed in Table A.

In certain embodiments, the pharmaceutical compositions of the present disclosure may additionally contain other adjunct components conventionally found in pharmaceutical compositions, at their art-established usage levels. Thus, for example, the pharmaceutical compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The compounds disclosed herein can be formulated in accordance with the routine procedures adapted for desired administration route. Accordingly, the compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa.

In certain embodiments, a pharmaceutical composition of the present disclosure is prepared using known techniques, including, but not limited to mixing, dissolving, granulating, dragee-making, levigating, emulsifying, encapsulating, entrapping or tableting processes.

Having now generally described the invention, the same will be more readily understood through reference to the following examples, which are provided by way of illustration and are not intended to be limiting of the present invention.

### EXAMPLES

Representative Synthesis of Parkin Ligase Modulators

Below examples demonstrate general methods in which one skilled in the art could use to synthesize the compounds of the disclosures. One skilled in the art would readily understand that below examples provide guidance for the synthesis and one skilled in the art would understand that by changing starting materials or intermediates, various asymmetric triazole compounds of the present disclosure can be obtained.

### Example 1: Synthesis of N-[5-benzamido-1-(4-iodophenyl)-1,2,4-triazol-3-yl]benzamide

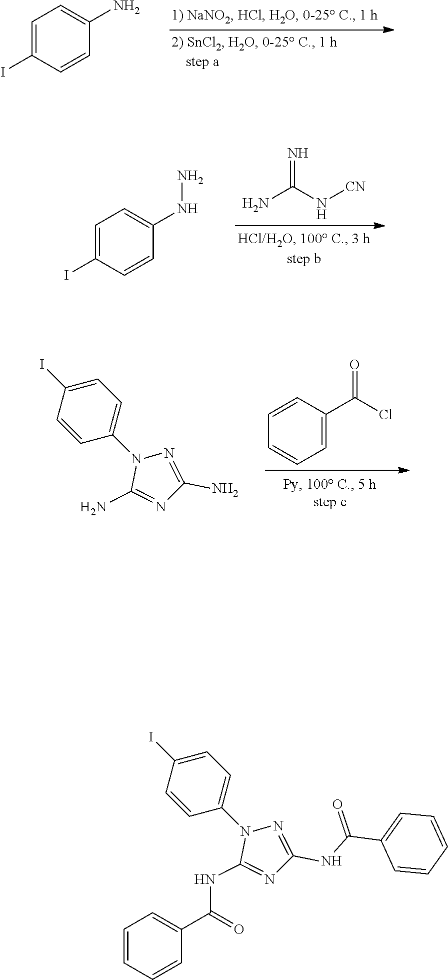 [SMILES: Nc1ccc(I)cc1 ; NNc1ccc(I)cc1 ; N#CNC(=N)N ; Nc1nc(N)n(-c2ccc(I)cc2)n1 ; O=C(Cl)c1ccccc1 ; O=C(Nc1nc(NC(=O)c2ccccc2)n(-c2ccc(I)cc2)n1)c1ccccc1]

### Step a: Synthesis of (4-iodophenyl)hydrazine

To a suspension of 4-iodoaniline (10.00 g, 45.66 mmol, 1.00 eq) in HCl (12 M, 30 mL) at 0° C. was added dropwise a solution of NaNO₂ (3.15 g, 45.66 mmol, 1.00 eq) in H₂O (15 mL), and the resulting mixture was stirred at 25° C. for 1 h. Then a solution of SnCl₂ (30.91 g, 136.98 mmol, 3.00 eq) in HCl (12 M, 20 mL) was added dropwise at 0° C. The reaction mixture was stirred at 25° C. for 1 h. TLC (DCM/MeOH=10:1) indicated the starting material was consumed completely and a new spot formed. The reaction mixture was filtered and the filter cake was dried under reduced pressure to afford (4-iodophenyl)hydrazine (9.50 g, HCl salt, crude) as a purplish red solid. ¹H NMR (400 MHz, DMSO-d₆) δ ppm: 10.39 (br, 3H), 8.50 (br, 1H), 7.59 (d, J=8.8 Hz, 2H), 6.86 (d, J=8.8 Hz, 2H).

### Step b: Synthesis of l-(4-iodophenyl)-1,2,4-triazole-3,5-diamine

To a solution of (4-iodophenyl)hydrazine hydrochloride (6.50 g, 24.03 mmol, 1.00 eq) in H₂O (15 mL) was added 1-cyanoguanidine (2.02 g, 24.03 mmol, 1.00 eq) and HCl (12 M, 5 mL). The reaction mixture was stirred at 100° C. for 3 h. TLC (DCM/MeOH=8/1) indicated the starting material was consumed completely and new spots formed. The reaction was basified to pH=8 with aq. NaOH solution (40%, w/v). After removal of the solvent under vacuum, hexane (100 mL) was added and the resulting mixture was stirred at 25° C. for 15 min. The mixture was filtered and the filter cake was washed with DCM (150 mL). The solids were collected and dried in vacuo to afford 1-(4-iodophenyl)-1,2,4-triazole-3,5-diamine (1.00 g, crude) as a brown solid. LC-MS (ESI): m/z 301.8 (M+H)⁺.

### Step c: Synthesis of N-[5-benzamido-1-(4-iodophenyl)-1,2,4-triazol-3-yl]benzamide

To a mixture of 1-(4-iodophenyl)-1,2,4-triazole-3,5-diamine (500 mg, 1.66 mmol, 1.00 eq) in pyridine (20 mL) was added benzoyl chloride (934 mg, 6.64 mmol, 771.69 μL, 4.00 eq), then the mixture was stirred at 100° C. for 5 h. LC-MS indicated the desired product was detected. Ethyl acetate (60 mL) was added and the resulting mixture was washed with HCl (1 M, 40 mL×3). The organic layer was dried over anhydrous Na₂SO₄, concentrated to afford the crude product, which was purified by prep-HPLC (column: Phenomenex Synergi C18 150×25×10 μm; mobile phase: [water (0.05% HCl)-ACN]; B %: 50%-70%, 10.5 min) to afford N-[5-benzamido-1-(4-iodophenyl)-1,2,4-triazol-3-yl]benzamide (21.80 mg, 42.80 μmol. 3% yield, 99+% purity) as a white solid. ¹H NMR (400 MHz, DMSO-d₆) δ ppm: 11.27 (br, 1H), 11.01 (br, 1H), 8.10-8.00 (m, 2H), 8.00-7.80 (m, 4H), 7.67-7.60 (m, 2H), 7.56-7.36 (m, 6H); ¹³C NMR (75 MHz, DMSO-d₆) δ ppm: 166.8, 165.8, 155.5, 146.1, 138.7, 137.2, 134.0, 133.3, 132.6, 129.2, 129.0, 128.4, 125.1, 94.6. LC-MS (ESI): m/z 510.0 (M+H)⁺.

### Example 2: Synthesis of 2-methyl-N-[5-[(2-methylbenzoyl)amino]-1-phenyl-1,2,4-triazol-3-yl]benzamide (Compound F)

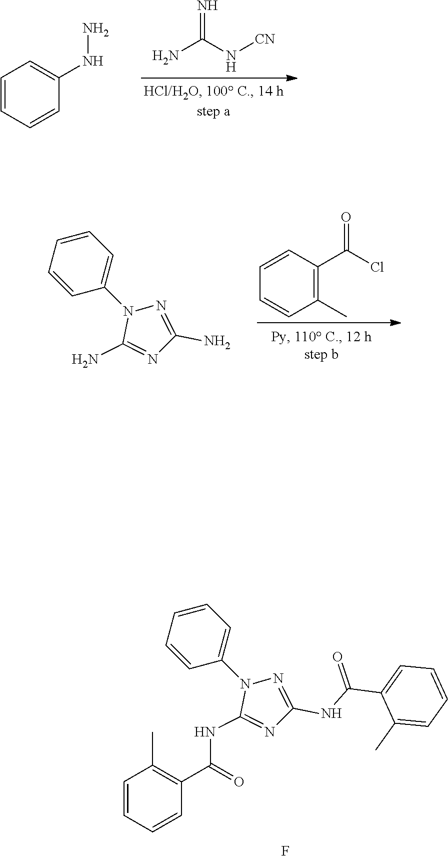

### Step a: Synthesis of 1-phenyl-1,2,4-triazole-3,5-diamine

To a solution of 1-cyanoguanidine (10.00 g, 118.93 mmol, 1.00 eq) and phenylhydrazine (12.86 g, 118.93 mmol, 11.69 mL, 1.00 eq) in H₂O (40 mL) was added hydrochloric acid (12 M in water, 8 mL). Then the reaction mixture was stirred at 100° C. for 14 h. The reaction mixture was basified pH to 8 with 40% sodium hydroxide aqueous solution. After removal of the solvent, the residue was slurried with hexane (150 mL) and followed by DCM (200 mL). The mixture was filtered and the filter cake was collected to give 1-phenyl-1,2,4-triazole-3,5-diamine (36.00 g, 197.27 mmol, 83% yield) as a yellow solid. ¹H NMR (400 MHz, DMSO-d₆) δ 7.48-7.40 (m, 4H), 7.23-7.19 (m, 1H), 6.70 (brs, 2H), 6.23 (brs, 2H).

### Step b: Synthesis of 2-methyl-N-[5-[(2-methylbenzoyl)amino]-1-phenyl-1,2,4-triazol-3-yl]benzamide

To a solution of 1-phenyl-1,2,4-triazole-3,5-diamine (800 mg, 4.57 mmol, 1.00 eq) in pyridine (10 mL) was added 2-methylbenzoyl chloride (2.12 g, 13.70 mmol, 1.78 mL, 3.00 eq). The reaction mixture was heated to 110° C. and stirred for 12 h. LC-MS indicated the starting material was consumed completely and desired compound was detected. The reaction mixture was quenched by saturated aqueous NH₄Cl (80 mL), and then extracted with ethyl acetate (100 mL×3). The organic layers were combined, washed with sat. NH₄Cl (50 mL), and sat. brine (50 mL), then dried over anhydrous Na₂SO₄, filtered and concentrated under reduced pressure to give a residue, which was purified by column chromatography on silica gel (3-50% ethyl acetate/petroleum ether) to afford the crude product. It was further purified by prep-HPLC (column: Phenomenex Synergi C18 150*25*10/an; mobile phase:[water (0.05% HCl)-ACN]; B %: 37%-57%, 10.5 min) to afford 2-methyl-N-[5-[(2-methylbenzoyl) amino]-1-phenyl-1,2,4-triazol-3-yl]benzamide (22.0 mg, 52.40 μmol. 98% purity) as a white solid. ¹H NMR (400 MHz, CD₃OD) δ 7.61-7.54 (m, 2H), 7.52-7.50 (m, 4H), 7.42-7.39 (m, 3H), 7.31-7.25 (m, 4H), 2.48 (s, 3H), 2.29 (s, 3H); ¹³C NMR (100 MHz, CD₃OD) δ 169.9, 169.6, 154.1, 145.4, 136.7, 136.2, 130.8, 130.7, 129.2, 127.2, 127.1, 125.5, 125.5, 125.4, 18.4. LC-MS (ESI): m/z 412.1 (M+H)⁺.

### Example 3: Synthesis of N,N′-(1-(4-(trifluoromethoxy)phenyl)-1H-1,2,4-triazole-3,5-diyl)bis(2-methylbenzamide) (Compound 42)

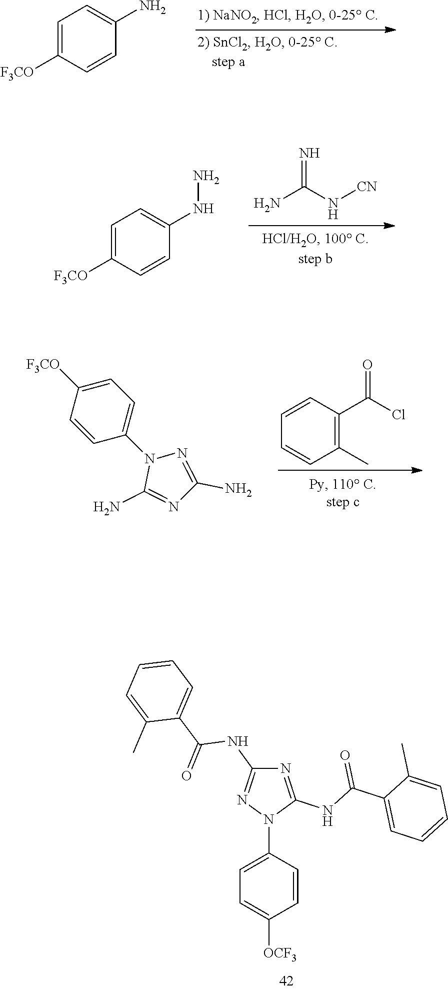

Compound 42 can be synthesized according to Example 2. (4-(Trifluoromethoxy)phenyl)hydrazine can be prepared starting from 4-(trifluoromethoxy)aniline instead of 4-iodoaniline according to Example 1, step a. Compound 42 can then be prepared in two steps according to Example 2 starting with (4-(Trifluoromethoxy)phenyl)hydrazine instead of phenylhydrazine.

### Example 4: Effects of Parkin Ligase Activators on Cellular Biomarkers

Senescence was quantified in ovarian cell lines A2780, OAW42, and ES-2. Cell lines A2780 has no cyclin D pathway mutations and has wt Rb and Parkin expression. Cell line OAW42 has increased CDK4 and increased CDK6 with wt Rb and Parkin expression. Cell line ES-2 has increased cyclin D and increased mutant p52 with wt Rb and Parkin expression.

Day 1: 96 well plate was seeded with 4000 cells/well and incubated for 24 hours at 37° C.

Day 2: Compound 42 (50, 100, or 150 nM) or palbociclib (1 μM) as positive control were added to the wells and the plate was incubated for 96 hours at 37° C.

Day 6: Senescence cells histochemical Staining Kit (Sigma) was used. The growth medium form the cells were aspirated then washed twice with fresh PBX. The entire wash was carefully removed so that cells do not detach. 100 μL filtered Fixation Buffer was added per well and the plate was incubated for 6-7 minutes at room temperature. Then, the cells were rinsed 3 times with PBS. 50 μL of the Staining Mixture was added per well and incubated at 37° C. overnight without CO₂ (staining is pH dependent). After staining, the Staining Mixture was removed, PBS and Hoechst dye (1 μg/mL) were added. Ten images were taken with Epifluorescence Microscope using the blue filter and 10 images were taken in the brightfield. The images were used to count the total cell number—the positive blue cells were counted in the bright field. The percentage of cells expressing β-galactosidase was calculated.

Method 1: Mammalian β-Galactosidase Assay Kit (Thermo Scientific) was used. The β-Galactosidase Assay Reagent was slowly thawed and equilibrated at to room temperature (do not heat). The cells were washed once with PBS. 100 μL of the β-Galactosidase Assay Reagent was added to each well and the plate was covered and incubated for 30 minutes at 37° C. without CO₂. The absorbance was measured at 405 nm. If no change was observed, the plate was incubated for an extra 30 minutes.

Method 2: Assay of a portion of the lysate. The cells were washed once with PBS. 50 μL of the M-PER Reagent was added to each well and the plate was incubated at room temperature for minutes. 25 μL of lysate was removed and use it for Protein Quantification (BCA). The date was normalize by protein content.

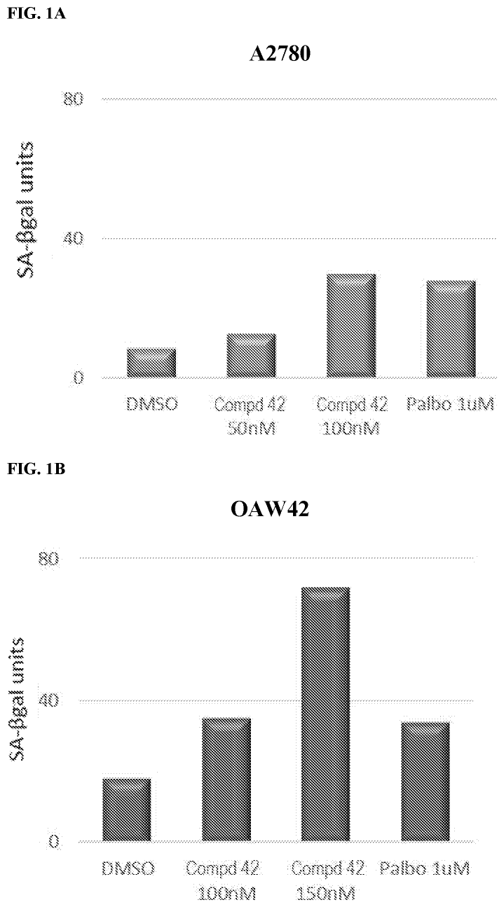

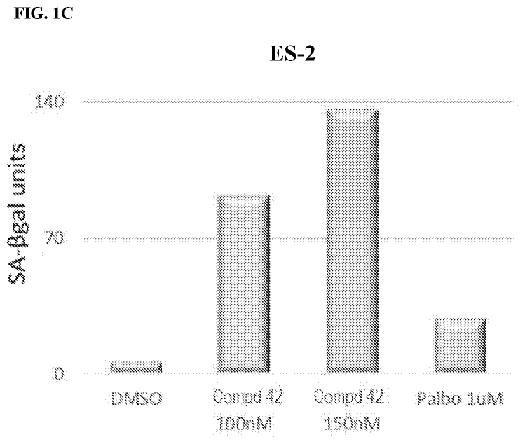

As shown in FIGS. 1A-1C, Compound 42 induced senescence as indicated by increased levels of SA-βGal (senescence-associated beta-galactosidase) compared to the control (DMSO).

Various cancer cells lines were treated with Compound 42 or palbociclib (control) to measure the expression of cyclin D, cyclin E, pRb, and R_{b}.

Day 1: 6 well plate was seeded with 3×10⁵ cells/well with a final volume of 3 mL and incubated for 24 hours at 37° C.

Day 2: The media was removed and Compound 42 (see below for dose) or palbociclib (1 μM) as positive control were added to the wells the desired concentration in cell culture media, giving a final volume of 3 mL per well. The plate was incubated for 24 hours at 37° C. (or desired period).

Day 3: The media was removed from wells and washed ×3 with PBS. 500 μl of trypsin was added to the wells and incubate until cells detach. 500 μl media was added to neutralise trypsin, transferred to 2 ml Eppendorfs, and centrifuged (1500 RPM for 5 minutes). While spinning, the wells were washed with 1 mL PBS to collect any remaining cells. This wash was used to centrifuged cell pellet. After the first spin, the supernatant was discarded and the pellet was washed with PBS collected from the previous step. The centrifugation step was repeated and the supernatant was discarded and the pellet wash again with fresh PBS. The centrifugation and wash with PBS was repeated one more time. RIPA cell lysate buffer was prepared and Protease Inhibitor Cocktail was included. The supernatant was discarded and 75 pi RIPA cell lysate buffer was added to the pellet. The protein concentration was determined using the BCA assay kit.

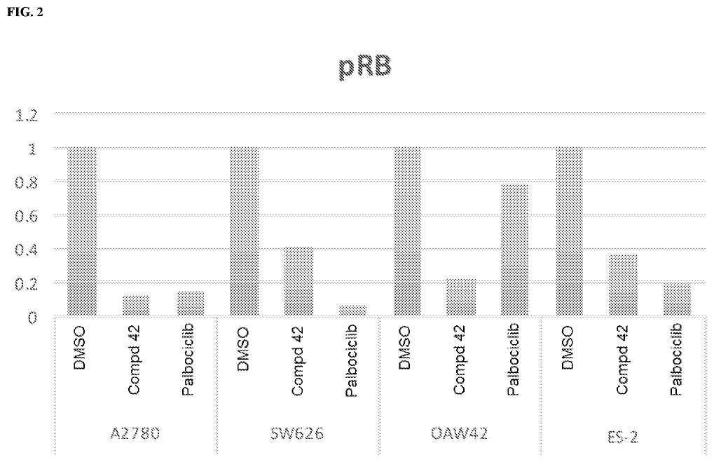

Day 4: Western blot analysis was performed. 4-12% Bis-Tris gel and MOPS running buffer was used. The running buffer was made to sufficient volume of running buffer to 1× concentration. 20 μg protein (v/v loading & reducing buffer) of each sample was loaded into wells. Some proteins may require more, or less, protein loaded. The gel was ran at standard settings 200 V for 45 minutes. Once gel has run, the gel was transferred for 7 minutes using the iBlot Blotting System. The membrane was blocked using 5% non-fat powdered milk* made up in 1×TBS containing 0.1% Tween (TBS-T and incubated for 1 hr with gentle rocking. The observation from the Western blot analysis is quantified in FIGS. 2-7D.

Cell lines A2780 (ovarian cell line with no cyclin D pathway mutations), ES-2 (ovarian cell line with cyclin D amplification, p53 mutation), OAW42, and SW626 were treated with DMSO (control), 250 nM of Compound 42, or 1 μM of Palbociclib. Treated cells were incubated for 24 hours and data represented in FIGS. 2-5 represents n=3.

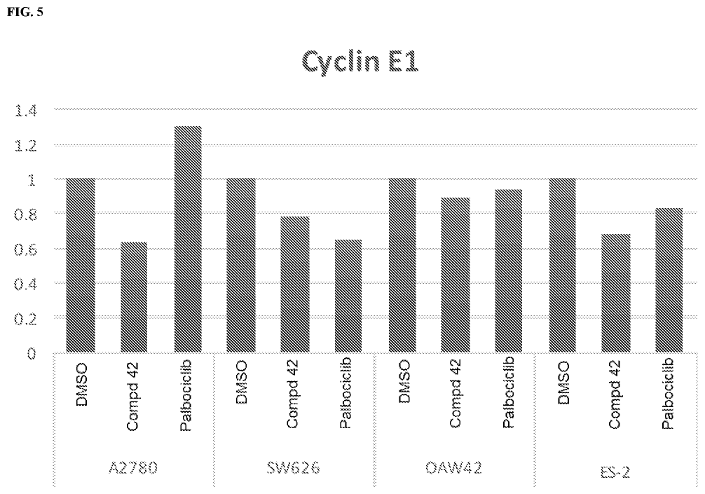

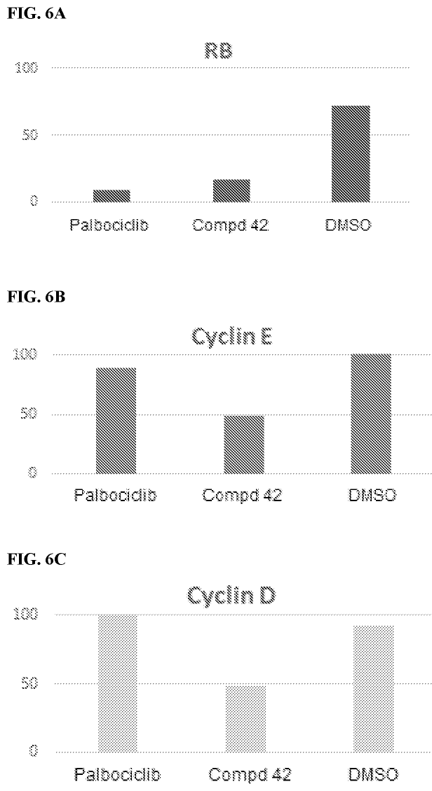

Cell line JEKO-1 (mantle cell line with cyclin D chromosome translocation, amplification of cyclin D) was treated with DMSO (control), 500 nM of Compound 42, or 1 μM of Palbociclib. Treated cells were incubated for 48 hours and data represented in FIGS. 6A-6C represents n=1.

Compound 42 demonstrated reduction of pRb expression in cell line OAW42 when compared to palbociclib (FIG. 2). Compound 42 demonstrated some reduction of pRb expression in cell line A2780 when compared to palbociclib.

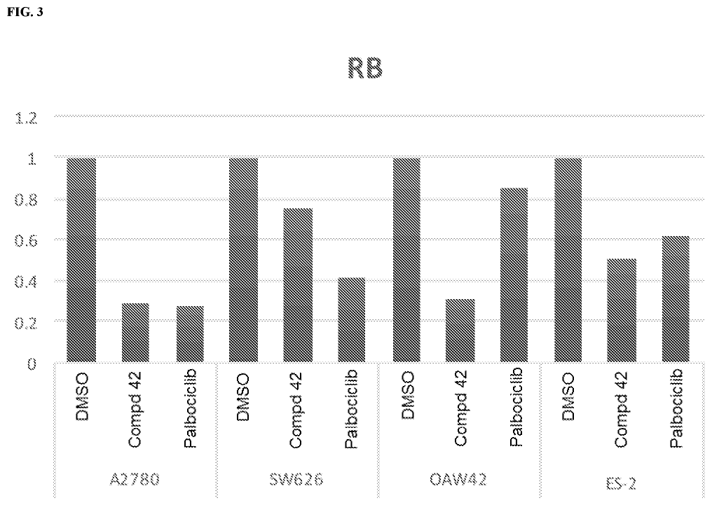

Compound 42 demonstrated reduction of Rb expression in cell lines OAW42 and ES-2 when compared to palbociclib (FIG. 3).

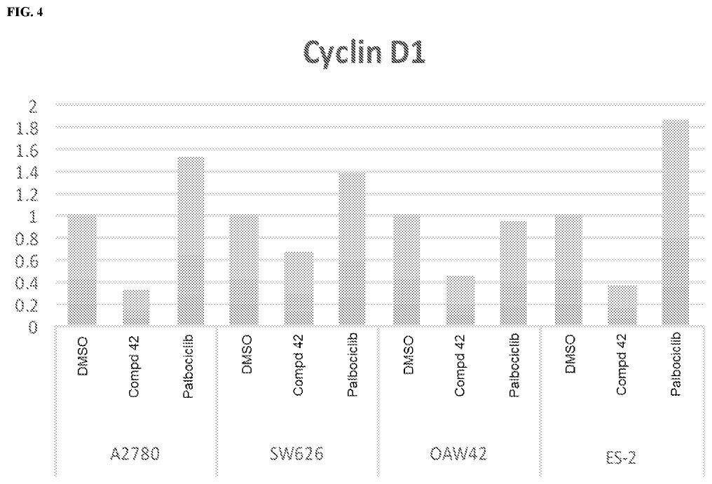

Compound 42 demonstrated reduction of cyclin D1 expressions in cell lines A2780, SW626 (mutation in p16 and pRb), OAW42, and ES-2 when compared to palbociclib (FIG. 4). Notably, palbociclib did not reduce the expressions of cyclin D1 but in most cell lines increased the expression of cyclin D1.

Compound 42 demonstrated reduction of cyclin E1 expressions in cell lines A2780 and ES-2 when compared to palbociclib (FIG. 5). Compound 42 demonstrated some reduction of cyclin E1 expression in cell line OAW42 when compared to palbociclib.

Compound 42 demonstrated reduction of cyclin E and cyclin D expression in JEKO-1 when compared to palbociclib (FIGS. 6B-6C).

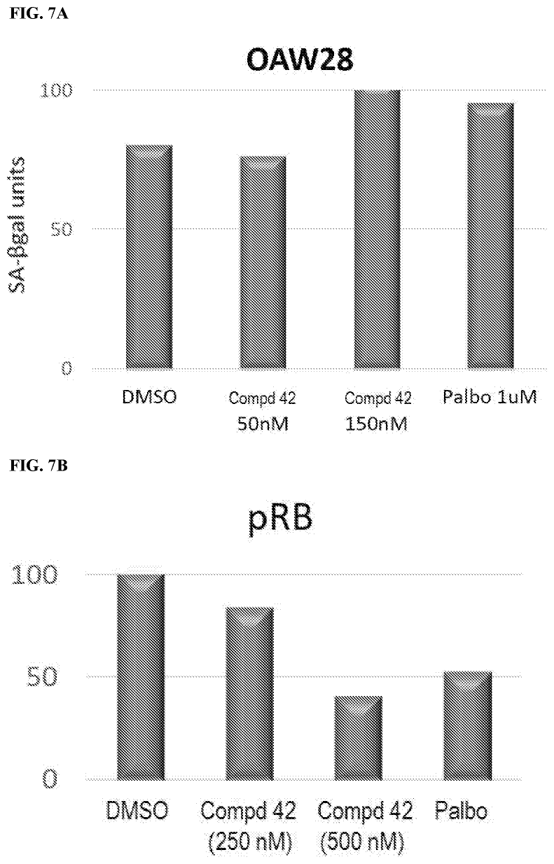

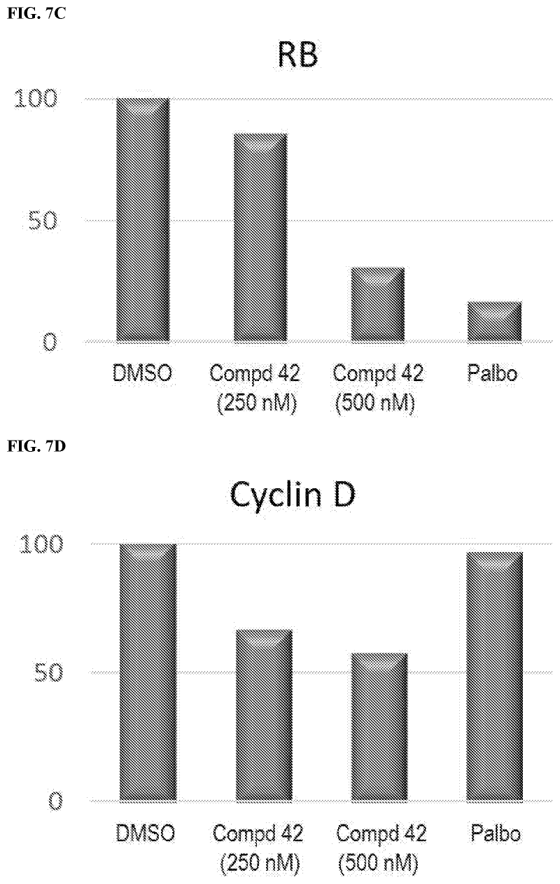

In comparison, cell senescence was not observed in OAW28, ovarian cell line with increased cyclin D and increased mutant p53 but with loss of Rb expression (FIG. 7A). The effect of Compound 42 on OAW28 with respect to pRb, Rb, and cyclin D is shown in FIGS. 7B-7D.

In summary, Compound 42 showed no senescence with OAW28 which has a loss of Rb expression, even though cyclin D expression was decreased with Compound 42. In contrast, Compound 42 demonstrated senescence and decrease in cyclin D expression for cells lines with wt Rb. These finding supports that Compound 42 (Parkin ligase modulator) decreases pRB protein through loss of cyclin D while Palbociclib decreases pRb protein without loss of cyclin D.

### Example 5: Cell Proliferation Assay

Compounds C and F were tested in a cell proliferation assay as shown in Table 6. Measurement of the inhibitive effect of compounds on cancer cell proliferation was performed. Various cell lines as indicated in Table 6 were tested. The assays were performed under the following conditions: Cells are harvested at a concentration of 4×10⁴ cells/ml in media. Volumes of 100 μL/well of these cell suspensions were added to a 96 well plate using a multichannel pipette. Plates were gently agitated to ensure an even dispersion of cells over a given plate. Cells were then incubated at 37° C., 5% CO₂ overnight. Following this, 100 μL of compound at varying concentrations was added to wells in triplicate. Control wells are those with 100 μL media containing 0.33% DMSO added to cell suspension (this is the equivalent volume of DMSO found in the highest concentration of drug). Plates were then gently agitated, as above, and incubated at 37° C., 5% CO₂ for 72 hrs (control wells have reached 80-90% confluency). Assessment of cell proliferation in the presence of Compounds C and F was determined by the acid phosphatase assay.

Following the incubation period of 72 hrs, media was removed from the plates. Each well on the plate was washed twice with 100 μL PBS. This was then removed and 100 μL of freshly prepared phosphatase substrate (10 mM p-nitrophenol phosphate in 0.1M sodium acetate, 0.1% triton X-100, pH 5.5) was added to each well. The plates were then incubated in the dark at 37° C. for 2 hours. Color development was monitored during this time. The enzymatic reaction was stopped by the addition of 50 μL of 1N NaOH. The plate was read in a dual beam plate reader at 405 nm with a reference wavelength of 620 nm. The absorbance reading of the latter is subtracted from the former, and the effect of the drug on cell proliferation was then measured as a percentage against the control cells (DMSO), which is taken as 0% inhibition. Tables 6 and 6A below shows IC₅₀ values.

TABLE 6
Cell Proliferation Assay (IC₅₀) for Compounds C and F

| | Cell | | | Cell | |
|---|---|---|---|---|---|
| | Viability: | | | Viability: | |
| Cell Line¹ | IC50 (μM) | Compound | Cell Line | IC50 (μM) | Compound |
| MOLT-4 | 0.26 | Compound C | SW480 | 3.49 | Compound F |
| DOHH2 | 0.3 | Compound C | NCl-N87 | 3.56 | Compound C |
| KM12 | 0.65 | Compound C | MKN-45 | 3.6 | Compound C |
| CCRF-CEM | 0.8 | Compound C | NCl-H226 | 3.63 | Compound C |
| A-427 | 0.86 | Compound C | NCl-H226 | 7.33 | Compound F |
| NCl-H292 | 2.01 | Compound C | HCC1954 | 3.69 | Compound F |
| NCl-H292 | 0.95 | Compound F | A-673 | 3.84 | Compound C |
| LNCaP | 0.95 | Compound C | KPL-1 | 3.86 | Compound F |
| SK-OV-3 | 0.96 | Compound C | ZR-75-1 | 3.87 | Compound F |
| SK-CO-1 | 0.98 | Compound F | SK-UT-1 | 4.04 | Compound C |
| TOV-112D | 0.99 | Compound F | RKO | 4.12 | Compound F |
| TOV-21G | 1.16 | Compound F | K562 | 4.14 | Compound C |
| HGC-27 | 1.22 | Compound C | PC-3 | 4.15 | Compound C |
| Calu-3 | 1.38 | Compound C | T-47D | 4.32 | Compound C |
| Hutu 80 | 1.63 | Compound F | T-47D | 7.37 | Compound F |
| HL-60 | 1.7 | Compound C | SW982 | 4.42 | Compound C |
| DU 145 | 1.82 | Compound C | MIA PaCa-2 | 4.48 | Compound C |
| A-204 | 1.85 | Compound C | HCT-8 | 4.74 | Compound F |
| NCl-H1703 | 1.85 | Compound F | NCl-H1975 | 5.37 | Compound C |
| OVCAR8 | 1.85 | Compound C | MD | 5.53 | Compound C |
| Calu-6 | 1.89 | Compound F | LOVO | 5.64 | Compound F |
| COV504 | 2 | Compound F | SK-LU-1 | 5.73 | Compound F |
| A431 | 2 | Compound C | HCC38 | 5.77 | Compound F |
| HCT 116 | 2 | Compound F | MDA-MB-157 | 5.82 | Compound F |
| A2780 | 2.02 | Compound C | A549 | 6.2 | Compound C |
| SHP-77 | 2.23 | Compound C | A549 | 7.54 | Compound F |
| MSTO-211H | 2.31 | Compound F | A549 | 7.54 | Compound F |
| LS174T | 2.37 | Compound F | NCl-H1395 | 7.6 | Compound F |
| 5637 | 2.46 | Compound C | SK-MEL-2 | 6.3 | Compound C |
| MCF-7 | 2.54 | Compound C | OVSAHO | 6.53 | Compound F |
| DLD-1 | 2.58 | Compound C | MDA-MB-436 | 6.55 | Compound F |
| DLD-1 | 7.49 | Compound F | SW527 | 6.69 | Compound F |
| NC-H196 | 2.83 | Compound F | SN-48 | 6.91 | Compound C |
| HT-29 | 2.96 | Compound F | MDA-MB-231 | 7.48 | Compound C |
| 22RV1 | 2.99 | Compound C | MDA-MB-231 | 6.98 | Compound F |
| SK-MEL-5 | 3.01 | Compound C | OVISE | 7.33 | Compound F |
| BT-474 | 3.03 | Compound F | SK-MEL-28 | 7.6 | Compound C |
| MDA-MB-453 | 3.04 | Compound F | SW837 | 7.66 | Compound C |
| SK-BR-3 | 3.05 | Compound F | SW948 | 8.37 | Compound F |
| COLO 205 | 3.07 | Compound C | JIMT-1 | 8.49 | Compound F |
| CAL 27 | 3.11 | Compound C | T98G | >10.0 | Compound C |
| BT-549 | 3.11 | Compound C | U-87 MG | >10.0 | Compound C |
| MDA-MB-468 | 3.18 | Compound F | OVTOKO | >10.0 | Compound F |
| ACHN | 3.33 | Compound C | Calu-1 | 43.3 | Compound F |
| HCC-78 | 3.35 | Compound C | Caco-2 | >10 | Compound F |
| MDA-MB-435S | 3.47 | Compound C | OVCAR4 | >10 | Compound C |

¹Cell line characteristics corresponds to those disclosed in Table 4.

TABLE 6A
Cell Proliferation Assay (IC₅₀) and
Cyclic D Degradation for Compound F

Compound F

| | Proliferation | Cyclin D |
|---|---|---|
| Cell Line | IC50 (nM) | degradation |

| | | | |
|---|---|---|---|
| Ovarian | A2780 | 1300 | Y |
| | ES-2*^{∧} | Not Tested | Y |
| | OAW28*^{∧}+ | Not Tested | Not Tested |
| | OAW42 | >2500 | Y |
| | OVCAR-3^{∧} | Not Tested | Not Tested |
| | TOV21G | 1500 | Y |
| | TOV112D^{∧} | 850 | Y |
| CRC | HCT-116^{∧} | 1300 | Y |
| Mantle Cell | Jeko-1* | >2500 | Y |
| | Mino* | 884 | Y |

*= overexpression of Cyclin D
^{∧}= loss of p16 (mutant p53)
+= weak senescence due to loss of RB

Compounds 42 and 1x-16x were tested in cell proliferation assays as shown in Tables 7, 7a, and 7b. These compounds displayed activity across a variety of cell lines, including those that overexpress Cyclin D or have a loss of p16 (mutant p53). Furthermore, Compound 42 was found to be effective in degrading Cyclin D.

TABLE 7
Cell Proliferation Assay (IC₅₀) for compound 42

Compound 42

| | Proliferation | Cyclin D |
|---|---|---|
| Cell Line | IC50 (nM) | degradation |

| | | | |
|---|---|---|---|
| Ovarian | A2780 | 84 | Y |
| | ES-2*^{∧} | 57 | Y |
| | OAW28*^{∧}+ | 140 | Y |
| | OAW42 | 166 | Y |
| | OVCAR-3^{∧} | 140 | Y |
| | TOV21G | 109 | Y |
| | TOV112D^{∧} | 103 | Y |
| CRC | HCT-116^{∧} | 87 | Y |
| Mantle Cell | Jeko-1* | 154 | Y |
| | Mino* | 345 | Y |

*= overexpression of Cyclin D
^{∧}= loss of p16 (mutant p53)
+= weak senescence due to loss of RB

TABLE 7a
Cell Proliferation Assay (EC₅₀) for compound 42

| Cell Line | EC50 (nM) |
|---|---|
| Colon (HCT-116) | 87 |
| Lung (A549) | 72 |
| Mantle Cell (Jeko-1) | 154 |
| Ovarian (A2780) | 58 |
| Ovarian (ES-2) | 42 |
| Ovarian (OAW28) | 114 |
| Ovarian (OAW42) | 166 |
| Ovarian (SKOV3) | 376 |
| Ovarian (SW626) | 334 |
| Ovarian (TOV112D) | 98 |
| Ovarian (TOV21G) | 74 |

TABLE 7b
Cell Proliferation Assay (EC₅₀) for compounds 1x-16x

| Compound # | 1x | 2x | 3x | 4x | 5x | 6x | 7x | 8x | 9x | 10x |
|---|---|---|---|---|---|---|---|---|---|---|
| HCT-116 | 972 nM | 3 μM | 484 nM | 295 nM | 133 nM | 2 μM | 406 nM | 246 nM | 121 nM | 108 nM |
| TOV-112D | 350 nM | 4 μM | 349 nM | 227 nM | 114 nM | 2 μM | 285 nM | 124 nM | 82 nM | 71 nM |

| Compound # | 11x | 12x | 13x | 14x | 15x | 16x |
|---|---|---|---|---|---|---|
| HCT-116 | 929 nM | 755 nM | 426 nM | 412 nM | 1 μM | 412 nM |
| TOV-112D | 774 nM | 183 nM | 231 nM | 655 nM | 3 μM | 659 nM |

### Example 6A: Effects of Parkin Ligase Activators is p16 Mutated Colon Cancer Cells

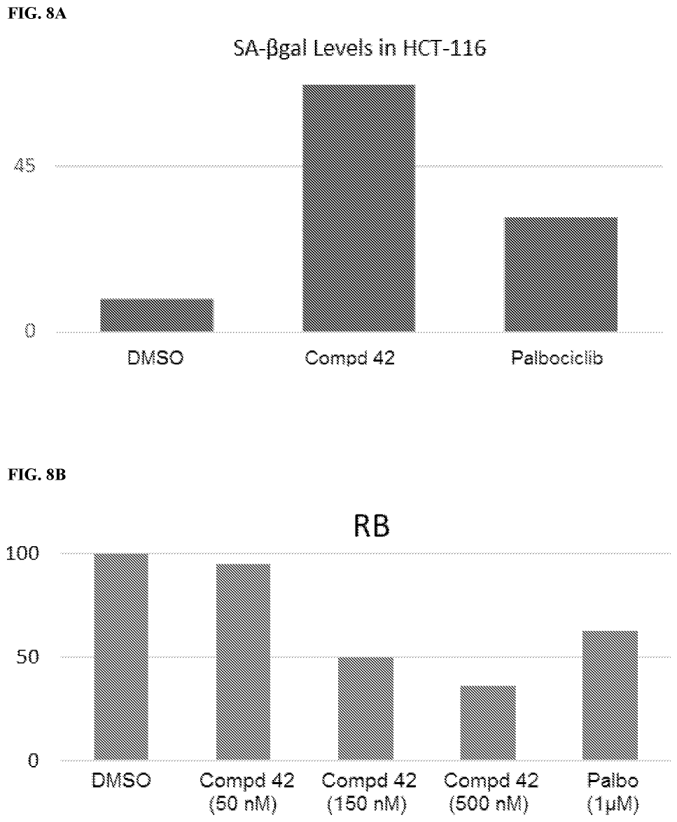

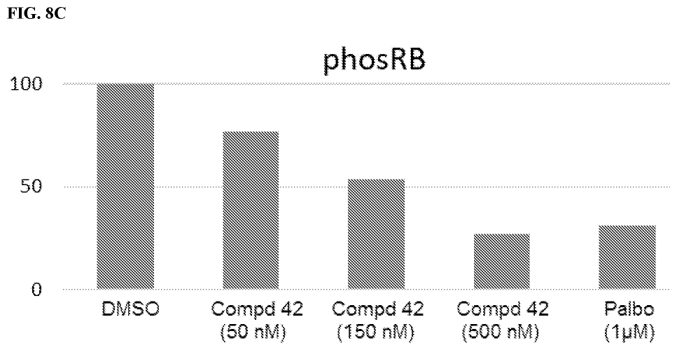

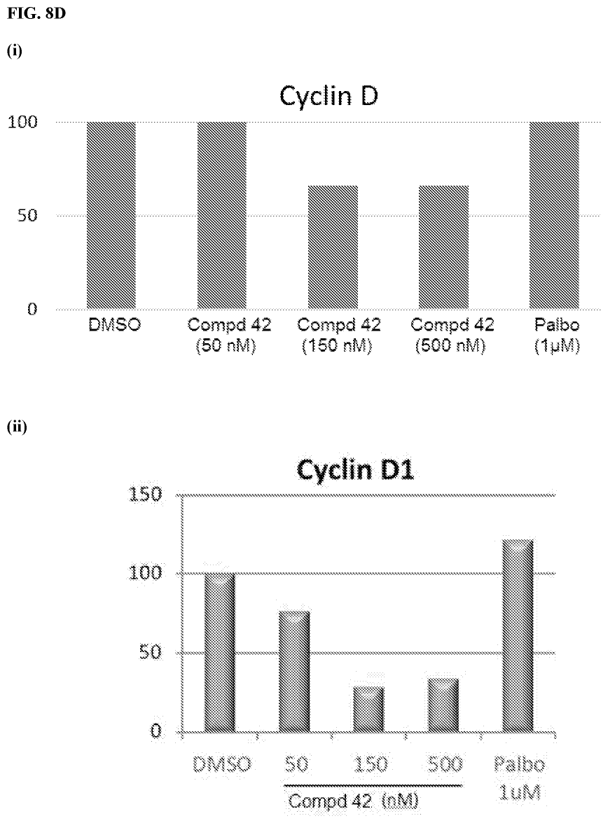

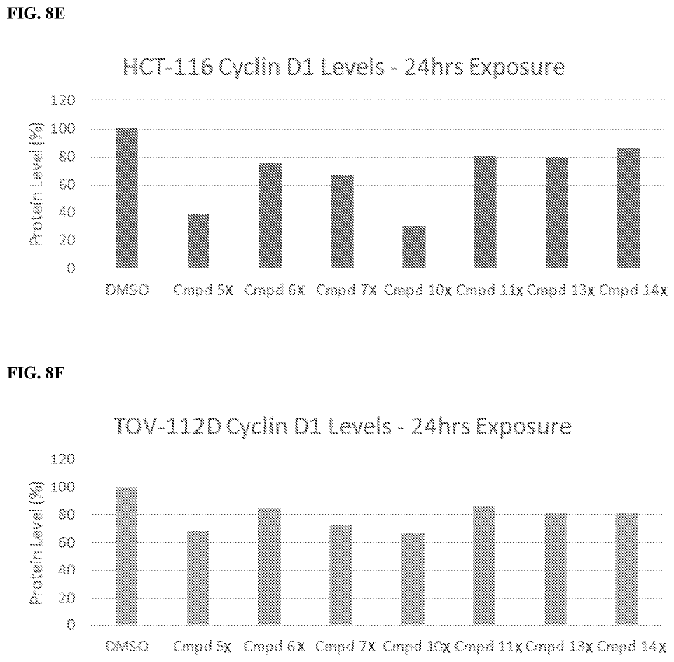

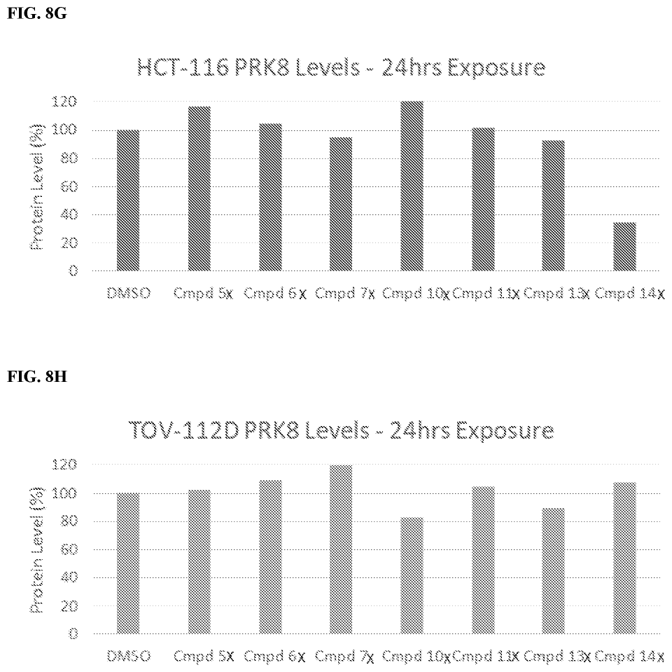

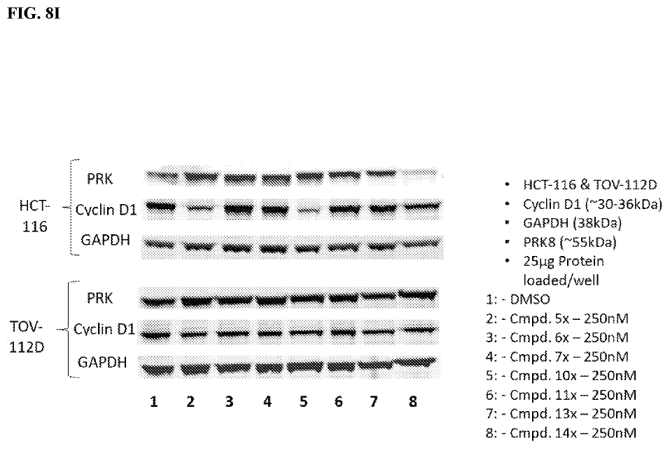

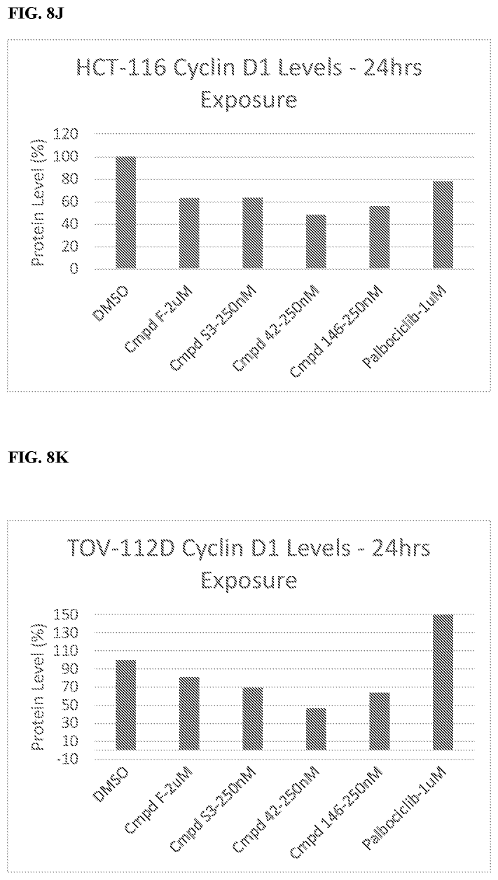

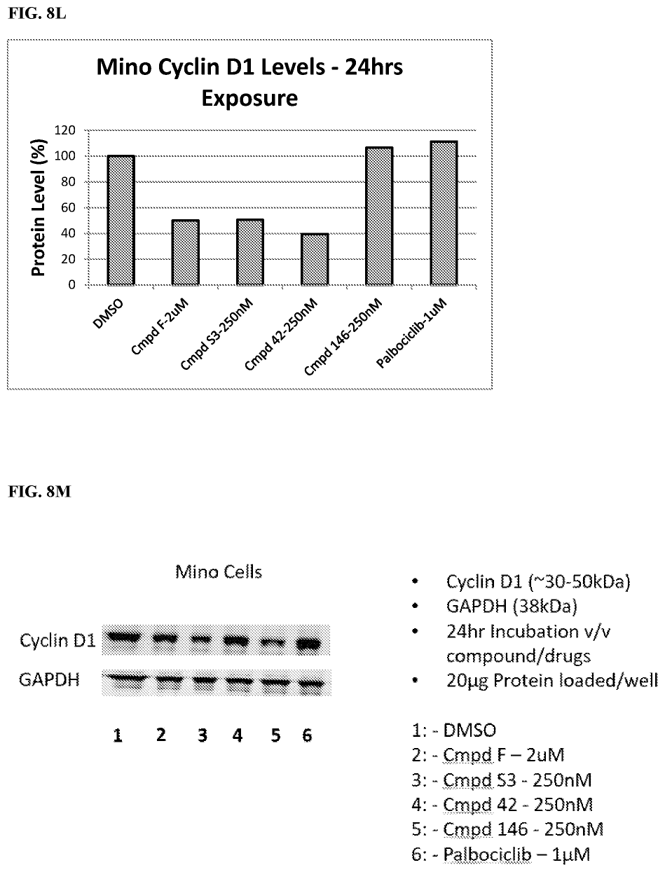

Senescence was quantified according to Example 4. Cell line HCT-116, colon cancer cell line with p16 mutation, results in increased cyclin D activity. As shown in FIG. 8A, Compound 42 (250 nM), and to a lesser extent CDK4/6 inhibitor Palbociclib (1 μM), normalized cyclic D and induced senescence as indicated by increased levels of SA-βGal (senescence-associated: beta-galactosidase) compared to tide control (DMSO). No apoptosis or other cell death was observed.

Rb, pRb, and cyclic D expressions in HT116 cell were measured according to Example 4. Compound 42 demonstrated reduction of Rb (FIG. 8B), pRb (FIG. 8C), cyclin D (FIG. 8B-(A)), and cyclifi D1 (FIG. 8B-(B)) expression m HT116 with increasing concentration.

The efficacy of Compound 42, Compound F, and Compound Q3 were evaluated in the treatment of subcutaneous HCT-116 xenograft model in nude mice.

Cell Culture: HCT-116 tumor cell lines were maintained in vitro as a monolayer culture in DMEM medium supplemented with 10% fetal bovine serum at 37° C. in an atmosphere of 5% CO₂ in air. The tumor cells were routinely subcultured twice weekly by trypsin-EDTA treatment (0.05% Trypsin-EDTA) and 1:4 split: The cells in an exponential growth phase were harvested and counted for tumor inoculation.

Tumor Inoculation: Each, mouse was inoculated subcutaneously at the flank region with HCT-1.16 cells (1.0×106) in 0.1 ml of 1×PBS mixed with Matrigel (1:1) for tumor development. The day Xenotransplantation was performed is denoted as day 0.

Thirty (30) animals with approx 100-150 mm³ tumors were selected for HCT-116 follow up experiment and randomly placed Into Groups 1, 2, 5, 4, 5, and 6 as shown in Table 8.

TABLE 8
Study Design for Xenograft Model

| Group | N | Treatment | Dose (mg/kg) | Dosing Route | Schedule |
|---|---|---|---|---|---|
| 1 | 5 | Vehicle Control | — | IP | — |
| 2 | 5 | Compound 42 | 1 | IP | Daily for 20 days |
| 3 | 5 | Compound 42 | 5 | IP | Daily for 20 days |
| 4 | 5 | Compound Q3 | 5 | IP | Daily for 20 days |
| 5 | 5 | Compound Q3 | 15 | IP | Daily for 20 days |
| 6 | 5 | Compound F | 25 | IP | Daily for 20 days |

Group Assignment: Before grouping and treatment, all animals were weighed and fee tumor volumes were confirmed (100-150 mm³) using electronic caliper. Since the tumor volume can affect fee effectiveness of any given treatment mice into groups using randomized block design as following: First the experimental animals were divided into homogeneous blocks based on their tumor volume. Secondly, within each block, randomization of experimental animals to different groups By using randomized block design to assign experimental animals, we ensure that each animal has the same probability of being assigned to any given treatment groups and therefore systematic error is minimized.

Treatments were initiated when turner volume reached 100-150 mm³. Treatments started on Day 5. Test compounds were administered intraperitoneally (II′) in 10 mg/mL solution comprising 10% N,N-dimethyl acetamide, 15% macrogol 15 hydroxy stearate, polyethylene glycol-15-hydroxystearate, and 75% aqueous hydroxypropyl-beta-cyclodextrin.

Observation and data collection: After tumor cells inoculation, the animals were checked daily for morbidity and mortality. At the time of routine monitoring, the animals were checked for any adverse effects of tumor growth and treatments on normal behavior such as mobility, visual estimation of food and water consumption, body weight gain/loss, eye/hair matting and any other abnormal effects, Death and observed clinical signs were recorded, per Alleges Labs IACUC. Tumor volumes were measured every 3-4 days in two dimensions using an electronic caliper, sad the volume data are expressed in, mm3 using the formula: V=0.5 a×b² where a and b are fee long and short diameters of the tumor, respectively. Dosing and tumor volume measurement procedures were conducted in a Laminar Flow Cabinet according to Altogen Labs IACUC regulations.

Body weight loss daring fee study: Sacrifice fee mouse when one measurement of body weight loss (BWL)>20% (only for control animals). Animal research associated wife this study was conducted under Altogen Labs IACUC regulatory space. Altogen Labs IACUC guidelines were followed in case of BWL and any other conditions.

Sample Collection: At the end of the study tissues were isolated and stored in 10% NBF formalin. On Days 1, 7, 21, and 28 blood from tail vein (50-100 ul) were collected into K2EDTA (BD) microtubes and serum isolation performed. Samples were processed to serum within 30-60 min of collection and stored at −80° C. Samples were labeled with the following information: Group ID, Sample ID, Study ID.

Statistics: Summary statistics, the mean and the standard error of the mean (SEM), are provided for the tumor volume of each group at each time point. Statistical analysis of difference in tumor volume among the groups and the analysis of drug interaction were conducted on the data obtained after the final dose.

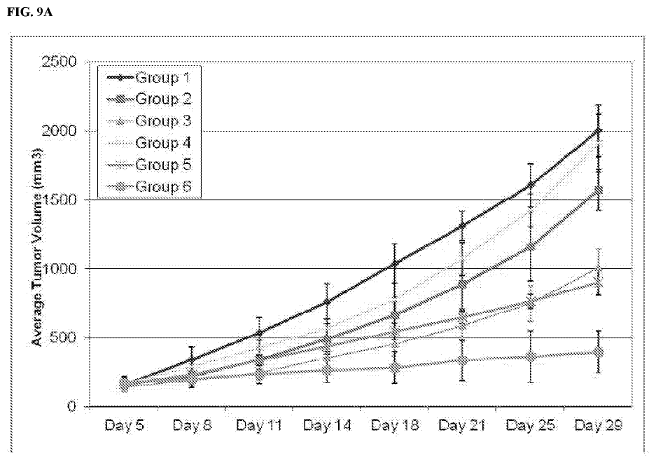

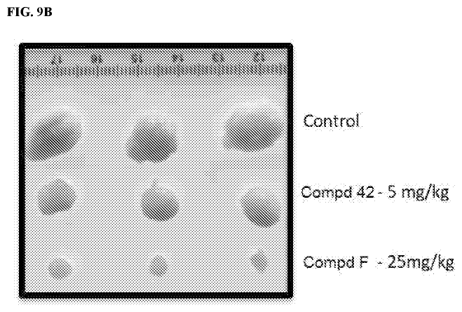

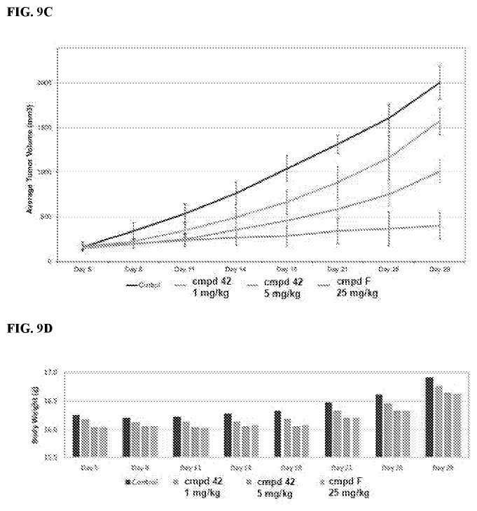

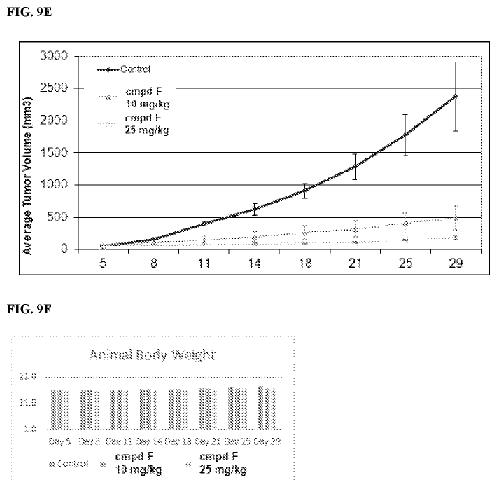

FIG. 9A shows change in tumor volume in a mouse xenograft model over 28 days with treatment of Compound 42 (Groups 2 and 3), Compound F (Group 6), and Compound Q3 (Groups 4 and 5). Compound 42, Compound F, and Compound Q3 all reduce the tumor growth when compared to the control (see also FIG. 9B). No weight loss or any adverse events were observed.

FIG. 9C shows change in tumor volume in a HCT116 mouse xenograft model over 28 days with treatment of 1 mg/kg of Compound 42, 5 mg/kg of Compound 42, or 25 mg/kg of Compound F. A point mutation in p16 removes negative regulation of Cyclin D. As shown by the data, all doses reduce tumor growth when compared to control. However, tumor volume is most substantially reduced after treatment with 25 mg/kg of Compound F. The mice in this study maintained body weight throughout the duration of the study (FIG. 9D).

The effect of reducing the dose of Compound F was also evaluated. FIG. 9E shows change in tumor volume in a HCT116 mouse xenograft model over 28 days with treatment of 10 mg/kg or 25 mg/kg of Compound F. According to the data, both doses substantially reduce tumor growth when compared to control, with the tumor reduction at 10 mg/kg nearly as effective as the reduction observed at 25 mg/kg. As before, the mice in this study maintained body weight throughout the duration of the study (FIG. 9F).

### Example 6B: Effects of Parkin Ligase Activators in Mutated Colon Cancer Cells and Mutated Ovarian Cancer Cells

Cells were plated at a density of 3×10⁵ cells/well for 24 hrs. Representative compounds or DMSO alone was then added to the wells for 24 h at 250 nM (note: Compound F was administered at a concentration of 2 μM). Cells were trypsinised, pelleted, washed ×3 in PBS, and lysed using a 2% SDS lysis solution (Sigma—05030).

Western blot analysis was then carried out to look at Cyclin D1 (Abeam—ab134175), PRK8 (Sigma—P6248), Rb (Abeam—ab134175) and GAPDH (Sigma—G9545) levels. See FIG. 8I.

As shown in FIGS. 8E and 8F, Compounds 5x, 6x, 7x, 10x, 11x, 13x, and 14x demonstrated reduction of Cyclin D1 expression in HCT-116 and TOV-112 lines compared to the control (DMSO).

FIGS. 8G and 8H show PRK8 protein levels in HCT-116 and TOV-112 cell lines after being treated with Compounds 5x, 6x, 7x, 10x, 11x, 13x, and 14x.

As shown in FIGS. 8J and 8K, Compounds F, S3, 42, and 146 demonstrated reduction of Cyclin D1 expression in HCT-116 and TOV-112 lines compared to the control (DMSO).

### Example 6C: Effects of Parkin Ligase Activators in Mutated Mantle Cell Lymphoma Cancer Cells

Mino cells have a chromosomal translocation of the Cyclin D gene that increases expression of cyclin D1. Western blot analysis was then carried out to look at Cyclin D1 (Abeam—ab134175) and GAPDH (Sigma—G9545) levels after treatment with Compounds F, S3, 42, and 146. See FIG. 8M.

FIG. 8L shows that Compounds S3 and 146 are able to appreciably reduce Cyclin D expression in this type of cells compared to control (DMSO).

### Example 7. Mechanistic Study of Parkin Degradation of Cyclin D

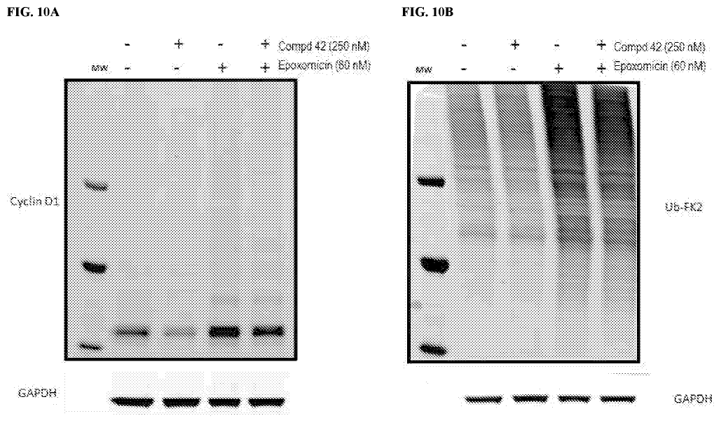

In order to establish that the decrease in Cyclin D seen after Compound 42 treatment is due to proteasomal degradation, ES-2, ovarian cancer cell line was treated with Compound 42 (250 nM) in the presence or absence of epoxomicin, a specific inhibitor of the proteasome (60 nM) for 18 hours. Following the treatment, western blotting for Cyclin D (FIG. 10A), Ub (FK-2) (FIG. 10B), and GAPDH control (bottom blot, below FIGS. 10A and 10B) was performed. Epoxomicin is a specific inhibitor of the proteasome.

As demonstrated in FIG. 10A, Compound 42 alone induced cyclin D levels to decrease while Compound 42 in the presence of epoxomicin returned cyclin D levels to normal (or slightly above). This data demonstrates that activation of Parkin leads to cyclin degradation by the proteasome. A control western blot (FIG. 10B) showed the effect of the epoxomicin inhibiting the proteasome, as evidenced by the overall increase of ubiquitinated proteins (using FK-2 antibody on the blot) in the lanes with epoxomicin.

The publications discussed herein are provided solely for their disclosure prior to the filing date of the present application. Nothing herein is to be construed as an admission that the present invention is not entitled to antedate such publication by virtue of prior invention.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### Example 8. HCT-116 Senescence Assay

HCT-116 were plated at a concentration of 2000 cells/well in a 6 well plate. 24 hrs later, drug treatments were added. Plates were incubated for 120 hrs.

Cells were stained using the Senescence Cells Histochemical Staining Kit from Sigma (CS0030). Briefly, the cells are washed ×3 with PBS, and Fixed for 6-7 minutes using the Fixation Buffer. Cells are then re-washed ×3 with PBS, and stained with a SA-β-Galactosidase Stain (which stains senescent cells) for 2 hrs-ovemight. Senescent cells are then counted visually.

Taking 10 random fields in the well, cells are counted with the aid of a grid reticule in the microscope eyepiece. This gives an average cell number for each well. In the compound-treated wells, virtually all the cells are senescent and remain at very low density, whereas the control cells have proliferated dramatically over the 120 hours.

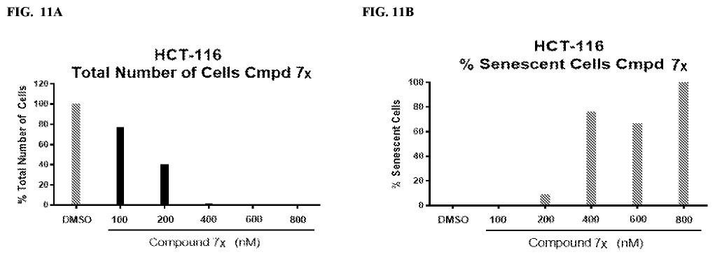

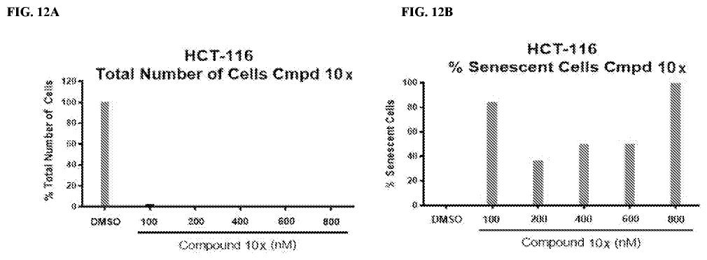

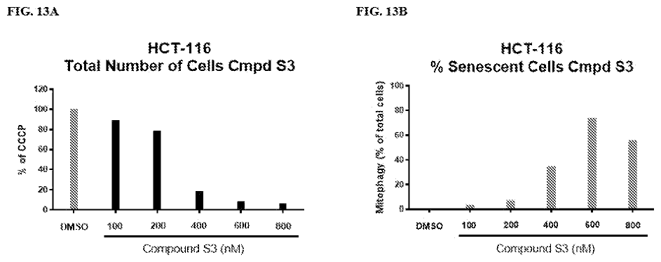

FIGS. 11A and 12A shows cell viability results and FIGS. 11B and 12B shows % senescent for cells treated with Compound 7x and Compound 10x, respectively. FIGS. 13A and 13B show % senescent for cells treated with Compound S3.

Below examples demonstrate general methods in which one skilled in the art could use to synthesize the compounds of the disclosures. One skilled in the art would readily understand that below examples provide guidance for the synthesis and one skilled in the art would understand that by changing starting materials or intermediates, various asymmetric triazole compounds of the present disclosure can be obtained.

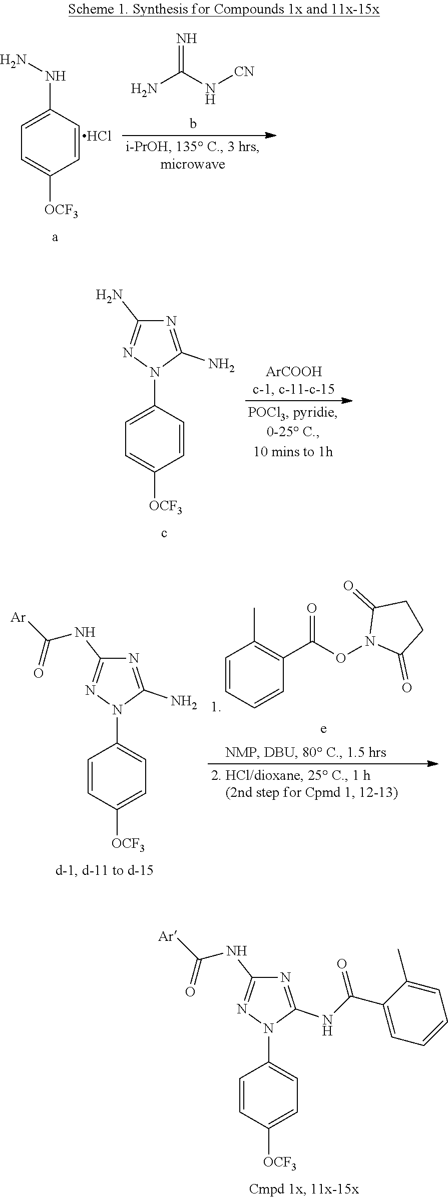

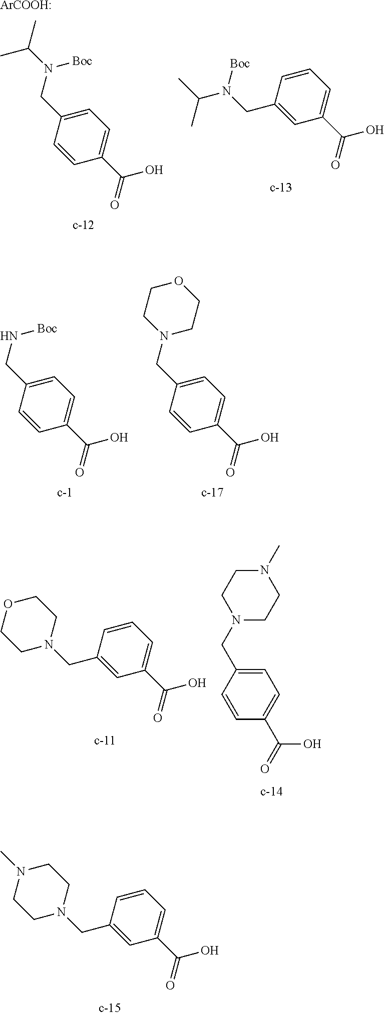

### Example 9: Synthesis of N-(3-(4-((isopropylamino)methyl)benzamido)-1-(4-(trifluoromethoxy)phenyl)-1H-1,2,4-triazol-5-yl)-2-methylbenzamide (Compound 12x)

### Step 1. Synthesis of Intermediate c

The mixture of compound a (4.00 g, 17.5 mmol, 1.00 eq, HCl) and compound b (1.54 g, 18.4 mmol, 1.05 eq) in i-PrOH (13 mL) was stirred for 3 hours at 135° C. under microwave.

The mixture was worked up without monitoring. The mixture was cooled and diluted with Ethyl acetate (100 mL), then washed with water (30 mL×3), brine (30 mL), dried with Na₂SO₄, concentrated in vacuo to give compound c (16.0 g, 55.8 mmol, 63.7% yield, 90.3% purity) as yellow solid, which was confirmed by LCMS (EW14597-1-P1B, RT=0.625 min, m/z+1=260.5). LC-MS (ESI): m/z (M+H) 260.5.

### Step 2. Synthesis of Intermediate c-12

A mixture of methyl 4-(bromomethyl)benzoate (10.0 g, 43.7 mmol, 1.00 eq) and compound isopropyl amine (25.8 g, 437 mmol, 37.5 mL, 10.0 eq) in 2-methyltetrahydrofuran (80 mL) was stirred at 100° C. for 8 hrs. LCMS showed the material was consumed completely and the desired MS was detected. The reaction mixture was poured into water (100 mL) and extracted with ethyl acetate (200 mL×3). The combined organic phase was dried over Na₂SO₄, filtered and concentrated in vacuum to give methyl 4-((isopropylamino)methyl)benzoate (8.90 g, 41.7 mmol, 95.4% yield, 97.0% purity) was obtained as a colorless oil. LC-MS (ESI): m/z (M+H) 208.2.

To a solution of methyl 4-((isopropylamino)methyl)benzoate (4.50 g, 21.7 mmol, 1.00 eq) in DCM (20 mL) was added Boc₂O (9.48 g, 43.4 mmol, 9.98 mL, 2.00 eq) and TEA (6.59 g, 65.1 mmol, 9.07 mL, 3.00 eq). The mixture was stirred at 25° C. for 2 hours. TLC (Petroleum ether:Ethyl acetate=5:1) indicated Reactant 1 (R_{f}=0.56) was consumed completely and one new spot (R_{f}=0.32) formed. The reaction mixture was diluted with dichloromethane (20 xL×2). The combined organic layers were washed with HCl (20 mL, 1M in water) then water (20 mL). The combined organic layers were washed with brine (20 mL×2), dried over Na₂SO₄, filtered and concentrated under reduced pressure to give methyl 4-(((tert-butoxycarbonyl)(isopropyl)amino)methyl)benzoate (6.00 g, 19.5 mmol, 89.9% yield) as a yellow oil which was confirmed by ¹H NMR. ¹H NMR: (400 MHz CDCl₃) δ=8.11-8.09 (m, 1H), 7.96-7.84 (m, 1H), 7.49-7.44 (m, 1H), 7.24-7.21 (m, 1H), 4.46-4.37 (m, 3H), 3.91 (s, 3H), 1.56-1.48 (m, 9H), 1.25-1.23 (m, 3H), 1.22-1.02 (m, 3H).

The mixture of methyl 4-(((tert-butoxycarbonyl)(isopropyl)amino)methyl)benzoate (6.00 g, 19.5 mmol, 1.00 eq) in THL (50 mL) and H₂O (50 mL) was added LiOH*H₂O (1.64 g, 39.0 mmol, 2.00 eq) at 0° C. Then the mixture was stirred at 25° C. for 2 hours. The desired mass (RT=0.942 min, m/z-55=238.2) was detected by LCMS (EW14597-9-P1A). TLC (Petroleum ether:Ethyl acetate=5:1) indicated reactant methyl 4-(((tert-butoxycarbonyl)(isopropyl)amino)methyl)benzoate (R_{f}=0.36) was consumed completely and one new spot (R_{f}=0.02) formed. Then the mixture was adjusted to pH=5 with HCl (1M in water). The reaction mixture was extracted with Ethyl acetate (30 mL*2). The combined organic layers were washed with brine (20 mL*2), dried over Na₂SO₄, filtered and concentrated under reduced pressure to give compound c-12 (1.50 g, 5.11 mmol, 26.2% yield) was obtained as a white solid, which was confirmed by ¹H NMR. LC-MS (ESI): m/z (M+H) 238.2; ¹H NMR (400 MHz, CDCl₃) δ=8.07-8.05 (m, 2H), 7.36-7.34 (m, 2H), 4.39 (s, 3H), 1.34-1.26 (m, 6H), 1.13-1.11 (m, 9H).

### Step 3. Synthesis of Intermediate d-12

To a mixture of compound c (4.00 g, 15.4 mmol, 1.00 eq) and compound c-12 (3.62 g, 12.4 mmol, 0.800 eq) in pyridie (40 mL) was added POCb (2.37 g, 15.4 mmol, 1.43 mL, 1.00 eq) at 0° C. The mixture was stirred at 25° C. for 10 mins. LCMS showed compound c was consumed, and 22.1% of desired MS (RT=0.974 min) was detected. The mixture was quenched by H₂O (10 mL) and concentrated under reduced pressure to give oil. The oil was purification by prep-HPLC (column: Phenomenex Synergi Max-RP 250*80 mm*10 um; mobile phase: [water (0.225% PA)—ACN]; B %: 40%-70%, 33 MIN, 40% min) to give Compound d-12 (1.47 g, 1.98 mmol, 12.8% yield, 72.1% purity) as yellow solid, which was confirmed by LCMS (RT=0.981 min), HPLC (RT=2.228 min) and ¹H NMR. LC-MS (ESI): m/z (M+H) 238.2; ¹H NMR (400 MHz, CDCl₃) δ=10.6 (s, 1H), 7.92-7.89 (m, 2H), 7.74-7.68 (m, 2H), 7.59-7.53 (m, 2H), 7.44-7.37 (m, 1H), 7.32-7.24 (m, 2H), 6.72 (s, 1H), 4.44 (s, 2H), 3.68 (s, 1H), 1.28-1.26 (m, 9H), 1.07-1.05 (m, 6H).

### Step 4. Synthesis of Compound 12x

To a solution of compound d-12 (400 mg, 748 umol, 1.00 eq) in NMP (2 mL) was added DBU (569 mg, 3.74 mmol, 564 uL, 5.00 eq) and compound 5 (873 mg, 3.74 mmol, 5.00 eq). The mixture was stirred at 80° C. for 1.5 hrs. LCMS (EW14611-25-P1A) showed reactant d-12 was consumed, desired m/z=653.1 (RT=1.056 min) was detected. The crude product was purified by reversed-phase HPLC (0.1% FA condition) to give tert-butyl isopropyl(4-((5-(2-methylbenzamido)-1-(4-(trifluoromethoxy)phenyl)-1H-1,2,4-triazol-3-yl)carbamoyl)benzyl)carbamate (120 mg, 183 umol, 24.6% yield) as a gray solid. LC-MS (ESI): m/z (M+H) 653.1.

A mixture of tert-butyl isopropyl (4-((5-(2-methylbenzamido)-1-(4-(trifluoromethoxy)phenyl)-1H-1,2,4-triazol-3-yl)carbamoyl)benzyl)carbamate (120 mg, 184 umol, 1.00 eq) and HCl/dioxane (4.00 M, 2.40 mL, 52.2 eq) was strried at 25° C. for 1 hr. LCMS (EW14611-26-P1A) showed starting material was consumed, desired m/z=553.1 (RT=0.827 min) was detected. The reaction mixture was concentrated under reduced pressure to give a residue. The crude product was purified by reversed-phase HPLC (0.1% HCl condition) to give Compound 12x (103.99 mg, 175 umol, 95.4% yield, 99.3% purity, HCl) as a brown solid. LC-MS (ESI): m/z (M+H) 553.1; ¹H NMR (400 MHz, DMSO) 5=11.2 (s, 1H), 11.1 (s, 1H), 9.04 (s, 2H), 8.08-8.06 (m, 2H), 7.76-7.70 (m, 4H), 7.61-7.59 (m, 2H), 7.43-7.42 (m, 1H), 7.33-7.31 (m, 1H), 7.30-7.29 (m, 2H), 4.25-4.22 (m, 2H), 3.65 (s, 1H), 3.38 (s, 1H), 2.19 (s, 3H), 1.32-1.31 (m, 6H).

### Example 10: Synthesis of N-(3-(3-((isopropylamino)methyl)benzamido)-1-(4-(trifluoromethoxy)phenyl)-1H-1,2,4-triazol-5-yl)-2-methylbenzamide (Compound 13x)

Compound 13x was synthesized according to Example 9 and Scheme 1. Obtained as yellow solid (34.24 mg, 57.3 umol, 37.4% yield, 98.5% purity, HCl salt). LC-MS (ESI): m/z (M+H) 553.1; ¹H NMR: (400 MHz DMSO) δ=11.2 (s, 1H), 11.1 (s, 1H), 9.17 (s, 2H), 8.19-8.17 (m, 1H), 8.05-8.03 (m, 1H), 7.76-7.74 (m, 1H), 7.61-7.60 (m, 2H), 7.59-7.49 (m, 4H), 7.48-7.47 (m, 1H), 7.43-7.42 (m, 1H), 7.31-7.29 (m, 2H), 4.23 (s, 2H), 3.36-3.30 (m, 2H), 2.19 (s, 3H), 1.33-1.31 (m, 6H).

### Example 11: Synthesis of N-(3-(4-(aminomethyl)benzamido)-1-(4-(trifluoromethoxy)phenyl)-1H-1,2,4-triazol-5-yl)-2-methylbenzamide (Compound 1x)

Compound 1x was synthesized according to Example 9 and Scheme 1. Obtained as white solid (99 mg, 180.07 umol, 91.92% yield, 99.48% purity, HCl salt). LC-MS (ESI): m/z (M+H) 511.4; ¹H NMR: (400 MHz DMSO) δ=11.2 (s, 1H), 11.1 (s, 1H), 8.46-8.44 (m, 3H), 8.05-8.03 (m, 2H), 7.76-7.74 (m, 2H), 7.64-7.63 (m, 4H), 7.61-7.58 (m, 1H), 7.49-7.47 (m, 1H), 7.31-7.29 (m, 2H), 4.12 (s, 2H), 2.19 (s, 3H).

### Example 12: Synthesis of 2-methyl-N-(3-(4-(morpholinomethyl)benzamido)-1-(4-(trifluoromethoxy)phenyl)-1H-1,2,4-triazol-5-yl)benzamide (Compound 17x)

### Step 1. Synthesis of Intermediate c-17

To a mixture of methyl 4-(bromomethyl)benzoate (10.0 g, 43.7 mmol, 1.00 eq) and K₂CO₃ (10.3 g, 74.2 mmol, 1.70 eq) in MeCN (50 mL) was added morpholine (4.18 g, 48.0 mmol, 4.23 mL, 1.10 eq). Then the mixture was stirred at 25° C. for 8 hrs. LCMS showed the material was consumed completely and the desired MS was detected. TLC (Petroleum ether: Ethyl acetate=5:1 material R_{f}=0.8 product R_{f}=0.4) showed the material was consumed completely and a new spot formed. The reaction mixture was filtered to remove the insoluble. The filter liquor was concentrated in vacuo to give the methyl 4-(morpholinomethyl)benzoate (10.0 g, 40.0 mmol, 91.5% yield, 94.0% purity) as a colorless oil. ¹H NMR: (400 MHz DMSO) δ=7.97-7.88 (m, 2H), 7.46-7.38 (m, 2H), 3.75 (s, 3H), 3.68-3.56 (m, 4H), 3.49-3.44 (m, 2H), 2.45-2.38 (m, 4H).

To a solution of methyl 4-(morpholinomethyl)benzoate (10.0 g, 42.5 mmol, 1.00 eq) in H₂O (30 mL) and THF (30 mL) was added LiOH.H₂O (3.57 g, 85.0 mmol, 2.00 eq). The mixture was stirred at 25° C. for 12 hrs. TLC (Petroleum ether:Ethyl acetate=1:1) showed methyl 4-(morpholinomethyl)benzoate (R_{f}=0.5) was consumed, and new spot (R_{f}=0.01) formed. The reaction mixture was concentrated under reduced pressure to remove THF. The residue was adjusted to pH=5-6 with HCl (1M), and concentrated under reduced pressure to give compound c-17 (10.0 g, crude) as a white solid. ¹H NMR: (400 MHz DMSO) δ=7.90-7.88 (m, 2H), 7.42-7.40 (m, 2H), 3.58-3.55 (m, 4H), 3.51 (s, 2H), 2.35-2.33 (m, 4H).

### Step 2. Synthesis of Intermediate d-17

To a solution of compound c-17 (2.05 g, 9.26 mmol, 0.800 eq) in pyridine (2 mL) was added POCl₃ (1.42 g, 9.25 mmol, 860 uL, 0.800 eq). The mixture was stirred at 0° C. for 0.5 hr. Then compound c (3.00 g, 11.5 mmol, 1.00 eq) in pyridine (2 mL) was added at 0° C., The mixture was stirred at 25° C. for 0.5 hr. Then POCl₃ (1.42 g, 9.25 mmol, 860 uL, 0.800 eq) was added, the mixture was stirred at 25° C. for 0.5 hr. LCMS showed Compound c was consumed, desired m/z=463.2 (RT=0.741 min) was detected. The reaction mixture was partitioned between water 50 mL and Ethyl acetate 100 mL. The organic phase was separated, washed with water 60 mL (30 mL×2), dried over Na₂SO₄, filtered and concentrated under reduced pressure to give a residue. The crude product was purified by reversed-phase HPLC (PA condition) to give compound d-17 (100 mg, 216 umol, 1.87% yield) as a gray solid.

### Step 3. Synthesis of Compound 17x

To a solution of compound d-17 (100 mg, 216 umol, 1.00 eq) in NMP (2 mL) was added DBU (165 mg, 1.08 mmol, 163 uL, 5.00 eq) and compound 5 (252 mg, 1.08 mmol, 5.00 eq). The mixture was stirred at 80° C. for 0.5 hr. LCMS (EW14611-33-P1A) showed reactant d-17 was consumed, desired m/z=581.5 (RT=0.828 min) was detected. The crude product was purified by reversed-phase HPLC (0.1% HCl condition) to give Compound 17x (19.18 mg, 29.7 umol, 13.7% yield, 95.6% purity, HCl) as a yellow gum. LC-MS (ESI): m/z (M+H) 581.2; ¹H NMR: (400 MHz DMSO) δ=11.2 (s, 1H), 11.1 (s, 1H), 11.0 (s, 1H), 8.08-8.06 (m, 2H), 7.75-7.73 (m, 4H), 7.60-7.57 (m, 2H), 7.48-7.46 (m, 1H), 7.43-7.41 (m, 1H), 7.30-7.29 (m, 2H), 4.41 (s, 2H), 3.95-3.92 (m, 2H), 3.79-3.76 (m, 2H), 3.26-3.23 (m, 2H), 3.21-3.12 (m, 2H), 2.18 (s, 3H).

### Example 13: Synthesis of 2-methyl-N-(3-(3-(morpholinomethyl)benzamido)-1-(4-(trifluoromethoxy)phenyl)-1H-1,2,4-triazol-5-yl)benzamide (Compound 11x)

Compound 11x was synthesized according to Example 12 and Scheme 1. Obtained as white solid (27.63 mg, 38.1 umol, 6.22% yield, 95.7% purity, TLA salt). LC-MS (ESI): m/z (M+H) 581.3; ¹H NMR: (400 MHz DMSO) δ=11.2 (s, 1H), 11.1 (s, 1H), 10.0 (s, 1H), 7.65-7.61 (m, 2H), 7.59-7.49 (m, 3H), 7.47-7.42 (m, 3H), 7.33-7.31 (m, 1H), 7.30-7.29 (m, 1H), 7.29-7.28 (m, 2H), 4.41 (s, 2H), 3.99-3.97 (m, 2H), 3.96-3.86 (m, 2H), 3.38-3.36 (m, 2H), 3.32-3.30 (m, 2H), 2.19 (s, 3H).

### Example 14: Synthesis of 2-methyl-N-(3-(4-((4-methylpiperazin-1-yl)methyl)benzamido)-1-(4-(trifluoromethoxy)phenyl)-1H-1,2,4-triazol-5-yl)benzamide (Compound 14x)

Compound 14x was synthesized according to Example 12 and Scheme 1. Obtained as yellow solid (29.36 mg, 45.5 umol, 10.8% yield, 97.6% purity, HCl salt). LC-MS (ESI): m/z (M+H) 594.5; ¹H NMR: (400 MHz DMSO) δ=11.2 (s, 1H), 11.1 (s, 1H), 8.09-8.07 (m, 2H), 7.82-7.80 (m, 4H), 7.77-7.75 (m, 2H), 7.61-7.59 (m, 1H), 7.48-7.42 (m, 1H), 7.30-7.28 (m, 2H), 4.44 (s, 2H), 3.62-3.35 (m, 8H), 2.82 (s, 3H), 2.19 (s, 3H).

### Example 15: Synthesis of 2-methyl-N-(3-(3-((4-methylpiperazin-1-yl)methyl)benzamido)-1-(4-(trifluoromethoxy)phenyl)-1H-1,2,4-triazol-5-yl)benzamide (Compound 15x)

Compound 15x was synthesized according to Example 12 and Scheme 1. Obtained as yellow solid (29.53 mg, 45.8 umol, 10.9% yield, 97.7% purity, HCl salt). LC-MS (ESI): m/z (M+H) 594.5; ¹H NMR: (400 MHz DMSO) δ=11.2 (s, 1H), 11.1 (s, 1H), 8.17-8.16 (m, 1H), 8.09-8.07 (m, 1H), 7.82-7.80 (m, 2H), 7.77-7.75 (m, 3H), 7.61-7.59 (m, 1H), 7.48-7.42 (m, 1H), 7.30-7.28 (m, 2H), 4.41 (s, 2H), 3.61-3.39 (m, 8H), 2.82 (s, 3H), 2.19 (s, 3H).

### Example 16: Synthesis of (S)—N-(4-((5-(2-methylbenzamido)-1-(4-(trifluoromethoxy)phenyl)-1H-1,2,4-triazol-3-yl)carbamoyl)benzyl)pyrrolidine-2-carboxamide (Compound 16x)

### Step 1. Synthesis of 2,5-dioxopyrrolidin-1-yl 2-methylbenzoate (g)

The mixture of l-hydroxypyrrolidine-2,5-dione (30.0 g, 261 mmol, 1.00 eq). 2-methylbenzoic acid (33.7 g, 247 mmol, 31.8 mL, 0.950 eq) and DCC (64.5 g, 313 mmol, 63.3 mL, 1.20 eq) in DMF (300 mL) was stirred at 25° C. for 3 hours. TLC (Petroleum ether: Ethyl acetate=5:1, (UV 254 nm)) indicated l-hydroxypyrrolidine-2,5-dione (R_{f}=0.42) was consumed completely and one new spot (R_{f}=0.28) formed. The mixture was filtered and the filter cake was collected. The crude product was triturated with MeOH (200 mL) at 25° C. for 30 min to give 2,5-dioxopyrrolidin-1-yl 2-methylbenzoate (20.0 g, 85.7 mmol, 32.9% yield) as a white solid. Tl NMR: (400 MHz, CDCl₃) δ=7.35-7.31 (m, 1H), 4.09-4.07 (m, 2H), 3.52-3.47 (m, 2H), 2.97-2.90 (m, 1H), 2.64-2.63 (m, 1H), 1.97-1.94 (m, 4H), 1.73-1.70 (m, 4H), 1.39-1.36 (m, 4H), 1.15-1.09 (m, 6H).

### Step 2. Synthesis of Intermediate h

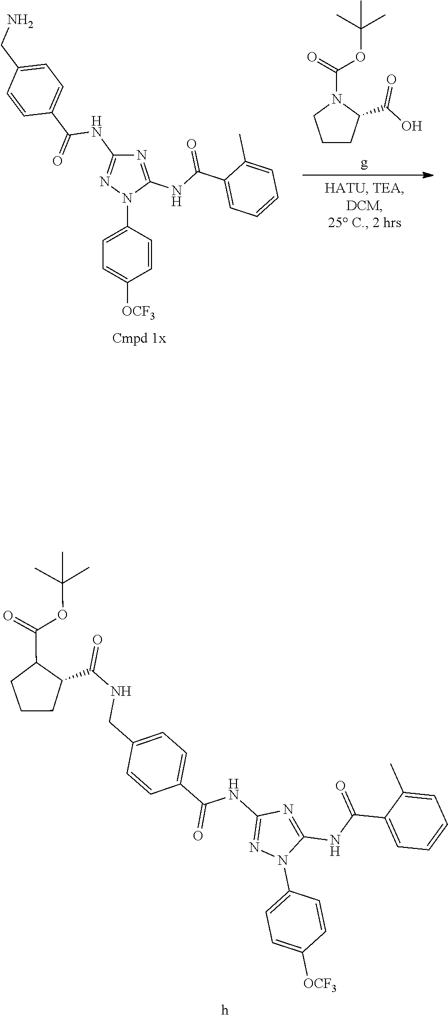 [SMILES: Cc1ccccc1C(=O)Nc1nc(NC(=O)c2ccc(CN)cc2)nn1-c1ccc(OC(F)(F)F)cc1 ; CC(C)(C)OC(=O)N1CCC[C@H]1C(=O)O ; Cc1ccccc1C(=O)Nc1nc(NC(=O)c2ccc(CNC(=O)[C@@H]3CCCC3C(=O)OC(C)(C)C)cc2)nn1-c1ccc(OC(F)(F)F)cc1]

To a solution of compound g (101 mg, 469 umol, 1.20 eq), Compound 1x (200 mg, 391 umol, 1.00 eq) in DCM (5 mL) was added TEA (48.0 mg, 474 umol, 66.0 uL, 1.21 eq) and then was added HATU (179 mg, 471 umol, 1.20 eq). The mixture was stirred at 25° C. for 2 hours. The desired mass (RT=0.992 min, m/z+1=708.2) was detected by LCMS. The mixture was diluted with water (15 mL), then extracted with dichloromethane (5 mL×3), dried with Na₂SO₄, concentrated in vacuo to give compound h (260 mg, crude) was obtained as a brown solid. LC-MS (ESI): m/z (M+H) 708.3.

### Step 3. Synthesis of Compound 16x

The mixture of compound h (260 mg, 367 umol, 1.00 eq) in HCl/dioxane (5 mL) was stirred at 25° C. for 1 h. The desired mass (RT=0.834 min, m/z+1=608.4) was detected by LCMS. The mixture concentrated in vacuo to give crude product. The residue was purified by prep-HPLC (HCl condition; column: Phenomenex Synergi C18 150×25×10 um; mobile phase: [water (0.05% HCl)—ACN]; B %: 23%-43%, 9 min) to give Compound 16×(66 mg, 101 umol, 27.4% yield, 98.4% purity, HCl) as a yellow solid. LC-MS (ESI): m/z (M+H) 608.6; ¹H NMR: (400 MHz CDCl₃) δ=11.2 (s, 1H), 11.1 (s, 1H), 9.78-9.76 (m, 1H), 9.21-9.18 (m, 1H), 8.59-8.58 (m, 1H), 8.00-7.97 (m, 2H), 7.76-7.74 (m, 2H), 7.61-7.58 (m, 2H), 7.56-7.45 (m, 1H), 7.44-7.42 (m, 3H), 7.30-7.29 (m, 2H), 4.44 (s, 2H), 4.26-4.23 (m, 1H), 3.32-3.23 (m, 2H), 2.32-2.23 (m, 1H), 2.19 (s, 3H), 1.98-1.89 (m, 3H).

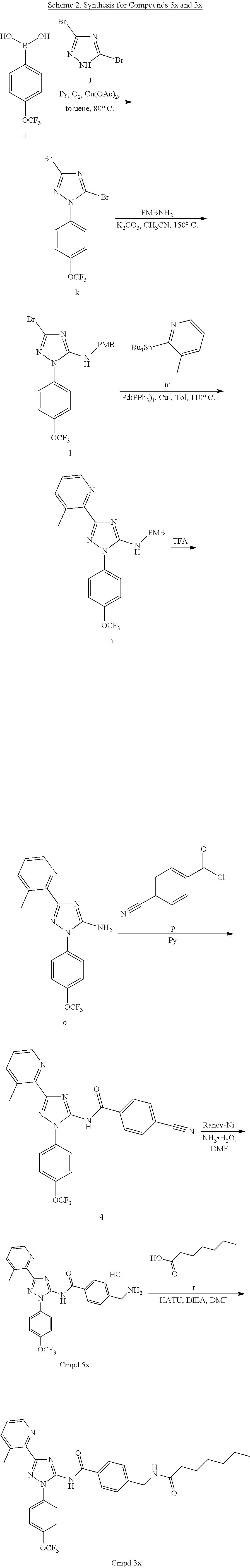

### Example 17: Synthesis of 4-(aminomethyl)-N-(3-(3-methylpyridin-2-yl)-1-(4-(trifluoromethoxy)phenyl)-1H-1,2,4-triazol-5-yl)benzamide (Compound 5x) and Synthesis of 4-(heptanamidomethyl)-N-(3-(3-methylpyridin-2-yl)-1-(4-(trifluoromethoxy)phenyl)-1H-1,2,4-triazol-5-yl)benzamide (Compound 3x)

### Step 1 (see Scheme 2). Synthesis of Intermediate k

A mixture of compound j (5.00 g, 22.0 mmol, 1.00 eq), compound i (5.45 g, 26.5 mmol, 1.20 eq), Cu(OAC)₂ (6.00 g, 33.1 mmol, 1.50 eq), Py (5.23 g, 66.1 mmol, 5.34 mL, 3.00 eq) and 4A MS (3.00 g, 22.0 mmol, 1.00 eq) in toluene (75 mL) was degassed and purged with O₂ for 3 times, and then the mixture was stirred at 90° C. for 6 hr under O₂ atmosphere. The resulting mixture was cooled, then filtered with celite, washed with ethyl acetate (50 mL), then the solution was concentrated under reduced pressure. The residue was purified by column chromatography (SiO₂, Petroleum ether/Ethyl acetate=100/1 to 5:1). Compound k (4.97 g, 97.15% purity) was obtained as a yellow oil and confirmed by LCMS (EW12195-115-P1C), ¹H NMR. LC-MS (ESI): m/z (M+H) 387.7; ¹H NMR (400 MHz, CDCl₃) δ=7.65-7.60 (m, 2H), 7.41-7.37 (m, 2H).

### Step 2. Synthesis of Intermediate 1

To a solution of compound k (3.00 g, 7.75 mmol, 1.00 eq) in NMP (20 mL) was added K₂CO₃ (1.29 g, 9.33 mmol, 1.20 eq) and (4-methoxyphenyl)methanamine (1.17 g, 8.53 mmol, 1.11 mL, 1.10 eq). The mixture was stirred at 150° C. for 1 hr under microwave. LCMS (EW14613-1-P1A) showed desired mass (RT=1.032 min, m/z (M+H)=444.8) was detected. The reaction mixture was diluted with H₂O 50 mL and extracted with ethyl acetate 60 mL. The combined organic layers were washed with brine 20 mL, dried over Na₂SO₄, filtered and concentrated under reduced pressure to give a residue. The residue was purified by column chromatography (SiO₂, Petroleum ether/Ethyl acetate=20/1 to 3:1). Intermediate 1 (8.00 g, 18.1 mmol, 58.2% yield) was obtained as a light yellow solid and confirmed by next step. LC-MS (ESI): m/z (M+H) 444.8.

### Step 3. Synthesis of Intermediate n

A mixture of Intermediate 1 (3.00 g, 6.77 mmol, 1.00 eq), compound m (7.76 g, 20.3 mmol, 3.00 eq), Pd(PPh₃)₄ (156 mg, 135 umol, 0.02 eq), CuI (257 mg, 1.35 mmol, 0.20 eq) and LiCl (57.4 mg, 1.35 mmol, 27.7 uL, 0.20 eq) in DMF (10 mL) was degassed and purged with N₂ for 3 times, and then the mixture was stirred at 110° C. for 2 hrs under N₂ atmosphere. LCMS (EW14634-43-P1B) showed desired mass (RT=0.950 min, m/z (M+H)=456.2) was detected. The reaction mixture was diluted with H₂O 30 mL and extracted with ethyl acetate 50 mL. The combined organic layers were washed with brine 50 mL, dried over Na₂SO₄, filtered and concentrated under reduced pressure to give a residue. The residue was purified by column chromatography (SiO₂, Petroleum ether/Ethyl acetate=10/1 to 0:1). Compound n (1.30 g, 2.85 mmol, 42.2% yield) was obtained as a brown solid and confirmed by next step. LC-MS (ESI): m/z (M+H) 456.2.

### Step 4. Synthesis of Intermediate o

A solution of compound n (1.20 g, 2.63 mmol, 1.00 eq) in TFA (22.2 g, 194 mmol, 14.4 mL, 73.8 eg) was stirred at 50° C. for 2 hours. The mixture was concentrated and the residue was suspended in water (50 mL) and the aqueous mixture was basified with solid NaHCO₃ to pH=7-8. The mixture was extracted with EtOAc (50 mL×3). The combined organic layer was washed with brine (50 mL), dried over anhydrous Na₂SO₄, filtered and concentrated in vacuo. Compound o (1.00 g, crude) was obtained as yellow solid and confirmed with next step.

### Step 5. Synthesis of Intermediate q

To a solution of compound o (1.00 g, 2.98 mmol, 1.00 eq) in PYRIDINE (9.80 g, 124 mmol, 10 mL, 41.5 eq) was added 4-cyanobenzoyl chloride (1.20 g, 7.25 mmol, 2.43 eq) at 20° C. Then the mixture was stirred at 90° C. for 12 h. To the mixture was added a solution of LiOH.H₂O (700 mg, 16.7 mmol, 5.59 eq) in MeOH (5 mL) and H₂O (5 mL) at 5° C. with an ice-water bath and the mixture was stirred at 25° C. for 1 h. Water (20 mL) was added into the mixture, filtered, collected the solid and concentrated. The residue was used directly in the next without further purification. Compound q (1.10 g, 2.37 mmol, 79.4% yield) was obtained as yellow solid and confirmed with next step.

### Step 6. Synthesis of Compound 5x

A mixture of compound q (1.10 g, 2.37 mmol, 1.00 eq), NH₃.H₂O (2.01 g, 57.3 mmol, 2.20 mL, 24.2 eq) in DMF (20 mL) was degassed and purged with H₂ for 3 times, and Raney-Ni (882 mg, 10.3 mmol, 4.35 eq) was added in the mixture, then the mixture was stirred at 25° C. for 1 hour under H₂ (15 psi). The mixture was filtered, and the filtrate was concentrated. ⅓ of the residue was purified with pre-HPLC (column: Phenomenex luna C18 250*80 mm*10 um; mobile phase: [water (0.05% HCl)-ACN]; B %: 32acn %-62acn %, 32 min, 22% min). ⅔ of the residue used directly in the next step. Compound 5x (31 mg, 61.17 umol, 2.58% yield, 99.62% purity, HCl) was obtained as an off-white solid. Compound 5x (300 mg, crude) was obtained as yellow solid and confirmed with next step. LC-MS (ESI): m/z (M+H) 469.1; HPLC: RT=1.629 min; ¹H NMR (400 MHz, MeOD) δ=8.79 (d, J=5.6 Hz, 1H), 8.67 (d, J=7.8 Hz, 1H), 8.10-8.10 (m, 3H), 7.88 (d, J=8.8 Hz, 2H), 7.65 (d, J=8.2 Hz, 2H), 7.50 (d, J=8.8 Hz, 2H), 4.24 (s, 2H), 3.00 (s, 3H).

### Step 7. Synthesis of Compound 3x

To a solution of Compound 5x (150 mg, 320 umol, 1.00 eq) and heptanoic acid (75.0 mg, 576 umol, 81.8 uL, 1.80 eq) in DMF (3 mL) was added HATU (301 mg, 791 umol, 2.47 eq) and DIEA (120 mg, 931 umol, 162 uL, 2.91 eq), the mixture was stirred at 20° C. for 1 hour. The mixture was filtered. The residue was purified with pre-HPLC (column: Phenomenex Synergi C18 150*30 mm*4 um; mobile phase: [water (0.05% HCl)-ACN]; B %: 40%-60%, 11 min). Compound 3x (30 mg, 49.9 umol, 15.6% yield, 96.6% purity) was obtained as yellow solid. LC-MS (ESI): m/z (M+H) 581.2; HPLC: RT=2.224 min; ¹H NMR (400 MHz, MeOD) δ=8.78 (d, J=5.6 Hz, 1H), 8.65 (d, J=7.8 Hz, 1H), 8.07-8.04 (m, 1H), 7.88 (t, J=9.2 Hz, 4H), 7.57-7.39 (m, 5H), 4.52-4.38 (m, 2H), 3.00 (s, 3H), 2.26 (s, 2H), 1.71-1.55 (m, 2H), 1.32 (s, 6H), 0.90 (s, 3H).

### Example 18: Synthesis of 4-(aminomethyl)-N-(1-(3-fluorophenyl)-3-(3-methylpyridin-2-yl)-1H-1,2,4-triazol-5-yl)benzamide (Compound 6x) and Synthesis of N-(1-(3-fluorophenyl)-3-(3-methylpyridin-2-yl)-1H-1,2,4-triazol-5-yl)-4-(heptanamidomethyl)benzamide (Compound 4x)

Compound 6x and Compound 4x was synthesized according to Example 17 and Scheme 2, starting with (3-fluorophenyl)boronic acid instead of compound i.

Step 1. 3,5-dibromo-1-(3-fluorophenyl)-1H-1,2,4-triazole was obtained as awhite solid (23.4 g, 71.6 mmol, 64.8% yield, 98.0% purity). LC-MS (ESI): m/z (M+H) 321.7; HPLC: RT=2.861 min; ¹H NMR (400 MHz, CDCl₃) δ=7.51 (m, 1H), 7.40 (s, 1H), 7.39 (m, 1H), 7.25 (m, 1H).

Step 2. 3-bromo-1-(3-fluorophenyl)-N-(4-methoxybenzyl)-1H-1,2,4-triazol-5-amine was obtained as a white solid (9.50 g, 23.9 mmol, 76.6% yield) and confirmed by next step. LC-MS (ESI): m/z (M+H) 376.8.

Step 3. 1-(3-fluorophenyl)-N-(4-methoxybenzyl)-3-(3-methylpyridin-2-yl)-1H-1,2,4-triazol-5-amine was obtained as black brown oil (1.50 g, 3.85 mmol, 29.1% yield) and confirmed by next step. LC-MS (ESI): m/z (M+H) 390.2.

Step 4. 1-(3-fluorophenyl)-3-(3-methylpyridin-2-yl)-1H-1,2,4-triazol-5-amine was obtained as a black brown solid (900 mg, 3.34 mmol, 92.9% yield) and confirmed by next step.

Step 5. 4-(aminomethyl)-N-(1-(3-fluorophenyl)-3-(3-methylpyridin-2-yl)-1H-1,2,4-triazol-5-yl)benzamide was obtained as a yellow solid (800 mg, 2.01 mmol, 60.1% yield) and confirmed by next step. LC-MS (ESI): m/z (M+H) 398.9.

### Step 6. Synthesis of Compound 6x

A mixture of 4-(aminomethyl)-N-(1-(3-fluorophenyl)-3-(3-methylpyridin-2-yl)-1H-1,2,4-triazol-5-yl)benzamide (800 mg, 2.01 mmol, 1.00 eq), NH₃.H₂O (2.73 g, 19.5 mmol, 3 mL, 25% purity, 9.70 eq) in DMF (20 mL) was degassed and purged with H₂ for 3 times, and Raney-Ni (235 mg, 2.75 mmol, 1.37 eq) was added in the mixture, then the mixture was stirred at 25° C. for 2 hrs under H₂ (15 psi). The residue was purified by prep-HPLC (FA condition; column: Phenomenex Synergi Max-RP 150×50 mm×10 um; mobile phase: [water (0.225% FA)-ACN]; B %: 5%-35%, 11 min). Compound 6x (26.42 mg, 58.30 umol, 2.90% yield, 98.96% purity, Formic acid salt) was obtained as a light yellow solid. LC-MS (ESI): m/z (M+H) 403.1; HPLC: RT=1.201 min; Tl NMR (400 MHz, D₂O-d₆) δ=8.50 (d, J=4.6 Hz, 1H), 8.37 (s, 1H), 7.98 (d, J=7.8 Hz, 1H), 7.86 (d, J=7.8 Hz, 2H), 7.59-7.44 (m, 6H), 7.25 (t, J=8.4 Hz, 1H), 4.25 (s, 2H), 2.58 (s, 3H).

### Step 7. Synthesis of Compound 4x

A mixture of Compound 6x (200 mg, 497 umol, 1.00 eq), compound r (116 mg, 895 umol, 127 uL, 1.80 eq), HATU (467 mg, 1.23 mmol, 2.47 eq), DIEA (187 mg, 1.45 mmol, 252 uL, 2.91 eq) in DMF (5 mL) was stirred at 25° C. for 1 hr under N₂ atmosphere. The reaction mixture was purified by prep-HPLC (HCl condition; column: Phenomenex Synergi C18 150*30 mm*4 um; mobile phase: [water (0.05% HCl)-ACN]; B %: 33%-53%, 11 min). Compound 4x (26.8 mg, 51.4 umol, 10.3% yield, 98.7% purity) was obtained as a pink solid. LC-MS (ESI): m/z (M+H) 515.2; ¹H NMR (400 MHz, MeOD-r/e) 5=8.79 (d, J=5.5 Hz, 1H), 8.67 (d, J=7.8 Hz, 1H), 8.10-8.04 (m, 1H), 7.90 (d, J=8.2 Hz, 2H), 7.62-7.53 (m, 3H), 7.45 (d, J=8.2 Hz, 2H), 7.31-7.23 (m, 1H), 4.45 (s, 2H), 3.00 (s, 3H), 2.27 (t, J=7.5 Hz, 2H), 1.68-1.59 (m, 2H), 1.31 (s, 6H), 0.93-0.86 (m, 3H).

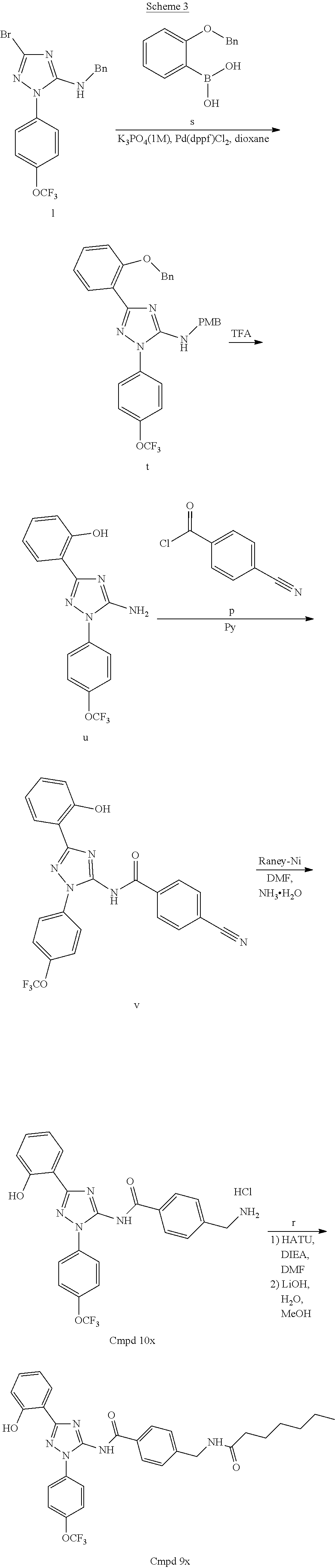

### Example 19: Synthesis of 4-(aminomethyl)-N-(3-(2-hydroxyphenyl)-1-(4-(trifluoromethoxy)phenyl)-1H-1,2,4-triazol-5-yl)benzamide (Compound 10x) and Synthesis of 4-(heptanamidomethyl)-N-(3-(2-hydroxyphenyl)-1-(4-(trifluoromethoxy)phenyl)-1H-1,2,4-triazol-5-yl)benzamide (Compound 9x)

### Step 1 (see Scheme 3). Synthesis of Intermediate t

A mixture of Intermediate 1 (4.00 g, 9.02 mmol, 1.00 eq), compound s (2.26 g, 9.92 mmol, 1.10 eq), K₃PO₄ (2 M, 13.5 mL, 3.00 eq), Pd(dppf)Cl₂ (330 mg, 451 umol, 0.05 eq) in dioxane (20 mL) was degassed and purged with N₂ for 3 times, and then the mixture was stirred at 100° C. for 16 hours under N₂ atmosphere. The reaction mixture was extracted with ethyl acetate (50 mL*3) and brine (50 mL*3), the aqueous phase was extracted with ethyl acetate (50 mL*1) again. The combined organic phase was dried with anhydrous Na₂SO₄, filtered and concentrated in vacuum. The residue was purified by prep-HPLC (FA condition). Compound t (3.20 g, 5.39 mmol, 59.7% yield, 91.9% purity) was obtained as a red oil. LCMS (RT=1.021 min), HPLC (RT=2.623 min), HNMR (CDCl3, 400 MHz) indicated the compound. MS (M+H⁺): 547.5. HPLC: RT=2.623 min, 91.9% purity. ¹H NMR (400 MHz, CDCl₃) 8.02 (d, J=2.0 Hz, 1H), 7.60 (d, J=6.0 Hz, 2H), 7.57 (d, J=8.8 Hz, 2H), 7.30-7.26 (m, 8H), 7.06-7.04 (m, 2H), 6.84 (d, J=8.4 Hz, 2H), 5.20 (s, 2H), 4.60 (s, 2H), 3.75 (s, 3H).

### Step 2. Synthesis of Intermediate u

A solution of compound t (2.00 g, 3.66 mmol, 1.00 eq) in TFA (30.8 g, 270 mmol, 20.0 mL, 73.8 eq) was stirred at 50° C. for 18 hours. The mixture was concentrated and the residue was suspended in water (50 mL) and the aqueous mixture was basified with solid NaHCO₃ to pH=7-8. The mixture was extracted with EtOAc (50 mL×3). The combined organic layer was washed with brine (50 mL), dried over anhydrous Na₂SO₄, filtered and concentrated in vacuo. Compound u (1.6 g, crude) was obtained as yellow oil and confirmed with next step.

### Step 3. Synthesis of Intermediate v

To a solution of compound u (2.00 g, 5.95 mmol, 1.00 eq) in PYRIDINE (17.8 g, 225 mmol, 18.2 mL, 37.9 eq) was added compound p (2.80 g, 16.9 mmol, 2.84 eq) at 20° C. Then the mixture was stirred at 90° C. for 12 h. To the mixture was added a solution of LiOH.H₂O (5 g) in H₂O/MeOH (1:1, 50 mL) at 5° C. with an ice-water bath and the mixture was stirred at 25° C. for 3 h. LCMS (EW14592-28-P1E) showed desired mass (RT=1.013 min, m/z=466.2) was detected. The mixture was evaporated under reduced pressure to remove methanol. The water phase was extracted with EtOAc (100 mL×3). The combined organic layer was washed with saturated aqueous NaHCO₃ (100 mL×3) and brine (100 mL), dried over Na₂SO₄, filtered and concentrated in vacuo. The residue was used directly in the next without further purification. Compound v (3.00 g, crude) was obtained as yellow oil and confirmed with next step. LCMS: RT=1.013 min, MS (M+H⁺): 466.2.

### Step 4. Synthesis of Compound 10x

A mixture of compound v (2.50 g, 5.37 mmol, 1.00 eq), NH₃.H₂O (4.55 g, 130 mmol, 5 mL, 24.2 eq) in DMF (20 mL) was degassed and purged with H₂ for 3 times, and Raney-Ni (2.00 g, 23.3 mmol, 4.35 eq) was added in the mixture, then the mixture was stirred at 25° C. for 12 hour under H₂ (15 psi). The mixture was filtered, and the filtrate was concentrated. The residue was purified with pre-HPLC (column: Phenomenex luna C18 250*80 mm*10 um; mobile phase: [water (0.05% HCl)-ACN]; B %: 32acn %-62acn %, 32 min, 22% min). Compound 10x (300 mg, 570 umol, 10.6% yield, 96.1% purity, HCl) was obtained as yellow solid. LC-MS (ESI): m/z (M+H) 470.0; ¹H NMR (400 MHz, DMSO) δ=11.66 (s, 1H), 10.33 (s, 1H), 8.52-8.46 (m, 3H), 7.98 (s, 3H), 7.86 (d, J=8.2 Hz, 2H), 7.65-7.55 (m, 4H), 7.38 (t, J=13 Hz, 1H), 7.08-6.99 (m, 2H), 4.12 (d, J=5.5 Hz, 2H).

### Step 5. Synthesis of Compound 9x

To a solution of Compound 10x (100 mg, 197.68 umol, 1 eq, HCl) and compound r (46.40 mg, 356.44 umol, 50.60 uL, 1.80 eq) in DML (3 mL) was added HATU (185.58 mg, 488.09 umol, 2.47 eq) and DIEA (74.24 mg, 574.47 umol, 100.06 uL, 2.91 eq), the mixture was stirred at 20° C. for 1 hour. The LiOH.H₂O (92.79 mg, 2.21 mmol, 11.19 eq) in H₂O (1 mL) and MeOH (1 mL) was added to the reaction mixture and the mixture was stirred at 20° C. for 1 hour. The residue was purified with pre-HPLC (column: Phenomenex Synergi C18 150*30 mm*4 um; mobile phase: [water (0.05% HCl)-ACN]; B %: 82%-98%, 11 min). Compound 9x (33 mg, 56.74 umol, 28.70% yield, 100% purity) was obtained as an off-white solid. LC-MS (ESI): m/z (M+H) 482.3; ¹H NMR (400 MHz, DMSO) δ=11.46 (s, 1H), 10.36 (s, 1H), 8.38 (t, J=5.7 Hz, 1H), 7.98 (d, J=7.8 Hz, 1H), 7.88-7.82 (m, 4H), 7.55 (d, J=7.8 Hz, 2H), 7.38 (d, J=8.1 Hz, 3H), 7.03-6.99 (m, 2H), 4.33 (d, J=5.8 Hz, 2H), 2.15 (t, J=7.4 Hz, 2H), 1.51 (d, J=6.8 Hz, 2H), 1.25 (s, 6H), 0.87-0.84 (m, 3H).

### Example 20: Synthesis of N-(3-(2-hydroxyphenyl)-1-(4-(piperazin-1-yl)phenyl)-1H-1,2,4-triazol-5-yl)-2-methylbenzamide (Compound 7x) and Synthesis of N-(1-(4-(4-heptanoylpiperazin-1-yl)phenyl)-3-(2-hydroxyphenyl)-1H-1,2,4-triazol-5-yl)-2-methylbenzamide (Compound 8x)

Compound 7x and Compound 8x was synthesized according to Examples 17 and 19, Schemes 2 and 3, using (4-(4-(tert-butoxycarbonyl)piperazin-1-yl)phenyl)boronic acid instead of compound i (Scheme 2) (4-(4-(tert-Butoxycarbonyl)piperazin-1-yl)phenyl)boronic acid was prepared in two steps as follows.

Step a. A mixture of l-bromo-4-iodobenzene (100 g, 353 mmol, 1.00 eq), tert-butyl piperazine-1-carboxylate (74.3 g, 399 mmol, 1.13 eq), CS₂CO₃ (320 g, 982 mmol, 2.78 eq), Xantphos (14.3 g, 24.7 mmol, 0.07 eq) and Pd₂(dba)₃ (8.09 g, 8.84 mmol, 0.025 eq) in dioxane (1.50 L) was degassed and purged with N₂ for 3 times, and then the mixture was stirred at 110° C. for 16 hrs under N₂ atmosphere. The mixture was filtered by diatomite, the reaction mixture was concentrated under reduced pressure to remove dioxane. The residue was triturated with (Petroleum ether: Ethyl acetate=4:1, 100.0 mL, 1 h), tert-Butyl 4-(4-bromophenyl)piperazine-1-carboxylate (69.0 g, 190.6 mmol, 53.9% yield, 94.3% purity) was obtained as a white solid and confirmed by LCM, HPLC and HNMR. LC-MS (ESI): m/z (M+H) 340.9; HPLC: RT=2.639 minl; ¹H NMR: (400 MHz CDCl₃) δ=7.37 (s, 1H), 7.34 (s, 1H), 6.80 (s, 1H), 6.78 (s, 1H), 3.59-3.56 (m, 4H), 3.11-3.09 (m, 4H), 1.48 (s, 9H).

Step b. To a solution of tert-Butyl 4-(4-bromophenyl)piperazine-1-carboxylate (30.0 g, 87.9 mmol, 1.00 eq) in THF (180 mL), the solution was cooled to −70° C., followed by dropwise addition of n-BuLi (2.5 M, 76.7 mL, 2.18 eq) while maintaining the temperature below −60° C. The mixture was aged at −70° C. for 30 min followed by the addition of triisopropyl borate (36.4 g, 193 mmol, 44.5 mL, 2.20 eq). The mixture was warmed to 0° C. and stirred at 0° C. for 1 hour. The reaction was quenched with saturated aqueous NH₄Cl (100 mL) and water (100 mL). Phosphoric acid (12.0 g) was added and the mixture agitated for 30 min, then extracted with ethyl acetate 200 mL. The combined organic layers were washed with brine (200 mL), dried overanhydrous Na₂SO₄, filtered and concentrated under reduced pressure to give a residue. The residue was purified by prep-HPLC (TFA condition). (4-(4-(tert-Butoxycarbonyl)piperazin-1-yl)phenyl)boronic acid (6.00 g, 19.6 mmol, 22.3% yield) was obtained as pink solid, which was confirmed by ¹H NMR: (400 MHz MeOD) 5=7.75-7.52 (m, 2H), 7.03-6.85 (m, 2H), 3.58 (s, 4H), 3.20 (s, 4H), 1.50 (s, 9H).

Step 1. tert-butyl 4-(4-(3,5-dibromo-1H-1,2,4-triazol-1-yl)phenyl)piperazine-1-carboxylate was obtained according to Example 17, step 1, as a white solid 4.00 g). LC-MS (ESI): m/z (M+H) 431.7; HPLC: RT=2.603 min; ¹H NMR: (400 MHz CDCl₃-d₆) δ=7.41-7.32 (m, 2H), 7.00-6.85 (m, 2H), 3.68-3.60 (m, 4H), 3.30-3.22 (m, 4H), 1.50 (s, 9H).

Step 2. tert-butyl 4-(4-(3-bromo-5-((4-methoxybenzyl)amino)-1H-1,2,4-triazol-1-yl)phenyl)piperazine-1-carboxylate was obtained according to Example 17, step 2, as colorless oil (2.00 g, 11.0 mmol, 71.7% yield). LC-MS (ESI): m/z (M+H) 545.2.

Step 3. tert-butyl 4-(4-(3-(2-(benzyloxy)phenyl)-5-((4-methoxybenzyl)amino)-1H-1,2,4-triazol-1-yl)phenyl)piperazine-1-carboxylate was obtained according to Example 19, step 1, as a yellow solid (2.20 g, 3.40 mmol, 61.6% yield). LC-MS (ESI): m/z (M+H) 647.2.

Step 4. 2-(5-amino-1-(4-(piperazin-1-yl)phenyl)-1H-1,2,4-triazol-3-yl)phenol was obtained according to Example 19, step 2, as a brown solid (1.10 g, 3.27 mmol, 96.1% yield). LC-MS (ESI): m/z (M+H) 336.9.

Step 5. tert-butyl 4-(4-(5-amino-3-(2-hydroxyphenyl)-1H-1,2,4-triazol-1-yl)phenyl)piperazine-1-carboxylate by Boc protection of 2-(5-amino-1-(4-(piperazin-1-yl)phenyl)-1H-1,2,4-triazol-3-yl)phenol.

A mixture of 2-(5-amino-1-(4-(piperazin-1-yl)phenyl)-1H-1,2,4-triazol-3-yl)phenol (1.14 g, 3.39 mmol, 1.00 eq) and B0C₂O (739 mg, 3.39 mmol, 778 uL, 1.00 eq) in DMF (10 mL) was stirred at 25° C. for 2 hrs. LCMS (EW14634-44-P1P) showed desired mass (RT=0.963 min, m/z (M+H)=437.3) was detected. The reaction mixture was extracted with ethyl acetate (50 mL×3) and brine (50 mL×3), The aqueous phase was extracted with ethyl acetate (50 mL×1) again. The combined organic phase was dried with anhydrous Na₂SO₄, filtered and concentrated in vacuum. The crude product was purified by reversed-phase HPLC (0.1% FA condition), tert-Butyl 4-(4-(5-amino-3-(2-hydroxyphenyl)-1H-1,2,4-triazol-1-yl)phenyl)piperazine-1-carboxylate (400 mg, 916 umol, 27.0% yield) was obtained as a white solid and confirmed by next step. LC-MS (ESI): m/z (M+H) 437.3.

Step 6. tert-butyl 4-(4-(3-(2-hydroxyphenyl)-5-(2-methylbenzamido)-1H-1,2,4-triazol-1-yl)phenyl)piperazine-1-carboxylate was obtained according to Example 19, step 3, using 2-methylbenzoyl chloride instead of compound p and obtained as an yellow oil (440 mg, 793 umol, 98.9% yield). LCMS: RT=0.913 min, MS (M+H⁺): 555.1

### Step 7. Synthesis of Compound 7x

A solution of tert-butyl 4-(4-(3-(2-hydroxyphenyl)-5-(2-methylbenzamido)-1H-1,2,4-triazol-1-yl)phenyl)piperazine-1-carboxylate (440 mg, 793.31 umol, 1 eq) in HCl/EtOAc (4 M, 20.00 mL, 100.84 eq) was stirred at 25° C. for 10 min. The mixture was concentrated. ⅓ of the residue was purified with pre-HPLC (column: Phenomenex Synergi C18 150*25*10 um; mobile phase: [water (0.05% HCl)-ACN]; B %: 15%-45%, 10 min). ⅔ of the residue used directly in the next step. Compound 7x (30 mg, 60.88 umol, 7.67% yield, 99.64% purity, HCl) was obtained as yellow solid. Compound 7x (240 mg, crude, HCl) was obtained as yellow solid and concentrated with next step. LC-MS (ESI): m/z (M+H) 455.1; ¹H NMR (400 MHz, D₂O) δ=7.55 (s, 1H), 7.16 (s, 6H), 7.00 (s, 2H), 6.80 (s, 2H), 6.65 (s, 1H), 3.12 (s, 8H), 1.97 (s, 3H).

### Step 7. Synthesis of Compound 8x

To a solution of compound r (100 mg, 768.14 umol, 109.05 uL, 3.14 eq) and Compound 7x (120 mg, 244.41 umol, 1 eq, HCl) in DMF (1 mL) was added DIEA (120.00 mg, 928.48 umol, 161.73 uL, 3.80 eq) and HATU (240 mg, 631 umol, 2.58 eq), the mixture was stirred at 20° C. for 1 hour. Then LiOH.H₂O (103 mg, 2.44 mmol, 10 eq) in H₂O (0.5 mL) and MeOH (0.5 mL) was added into the mixture, and the mixture was stirred at 20° C. for 1 hour. The mixture was filtered. The residue was purified with pre-HPLC (column: Phenomenex Synergi C18 150*25*10 um; mobile phase: [water (0.05% HCl)-ACN]; B %: 55%-85%, 10 min). Compound 8x (30 mg, 51.6 umol, 21.1% yield, 97.4% purity) was obtained as light yellow solid. LC-MS (ESI): m/z (M+H) 567.2; ¹H NMR (400 MHz, MeOD) δ=8.05-8.02 (m, 1H), 7.54-7.48 (m, 3H), 7.40-7.26 (m, 4H), 7.14 (d, J=9.0 Hz, 2H), 7.00-6.90 (m, 2H), 3.76-3.71 (m, 4H), 3.28-3.26 (m, 2H), 2.70 (s, 2H), 2.44 (t, J=7.6 Hz, 2H), 2.31 (s, 3H), 1.66-1.59 (m, 2H), 1.42-1.34 (m, 6H), 0.92 (t, J=7.2 Hz, 3H).

The publications discussed herein are provided solely for their disclosure prior to the filing date of the present application. Nothing herein is to be construed as an admission that the present invention is not entitled to antedate such publication by virtue of prior invention.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A method of treating cancer in a subject in need thereof, comprising administering to the subject a Parkin ligase activator or a pharmaceutically acceptable salt thereof, wherein the subject has a mutant form of a protein in the Rb checkpoint pathway and/or mutant form of p53.

2. The method of claim 1, wherein the cancer one or more selected from Acute Lymphoblastic Leukemia, Acute Myeloid Leukemia, Adrenocortical Carcinoma, AIDS-Related Cancers, Kaposi Sarcoma, liposarcoma, soft tissue sarcoma, Lymphoma, Anal Cancer, Appendix Cancer, Astrocytomas, Childhood Atypical Teratoid/Rhabdoid Tumor, Basal Cell Carcinoma, Skin Cancer (Nonmelanoma), Childhood Bile Duct Cancer, Extrahepatic Bladder Cancer, Bone Cancer, Ewing Sarcoma Lamily of Tumors, Osteosarcoma and Malignant Librous Histiocytoma, Brain Stem Glioma, Brain Tumors, Embryonal Tumors, Germ Cell Tumors, Craniopharyngioma, Ependymoma, Bronchial Tumors, Burkitt Lymphoma (Non-Hodgkin Lymphoma), Carcinoid Tumor, Gastrointestinal Carcinoma of Unknown Primary, Cardiac (Heart) Tumors, Lymphoma, Primary, Cervical Cancer, Childhood Cancers, Chordoma, Chronic Lymphocytic Leukemia, Chronic Myelogenous Leukemia, Chronic Myeloproliferative Neoplasms Colon Cancer, Colorectal Cancer, Cutaneous T-Cell Lymphoma, Ductal Carcinoma In Situ, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Intraocular Melanoma, Retinoblastoma, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors, Extragonadal Cancer, Ovarian Cancer, Testicular Cancer, Gestational Trophoblastic Disease, Glioma, Brain Stem Cancer, Hairy Cell Leukemia, Head and Neck Cancer, Heart Cancer, Hepatocellular (Liver) Cancer, Histiocytosis, Langerhans Cell Cancer, Hodgkin Lymphoma, Hypopharyngeal Cancer, Intraocular Melanoma, Islet Cell Tumors, Pancreatic Neuroendocrine Tumors, Kidney Cancer, Renal Cell Cancer, Wilms Tumor and Other Childhood Kidney Tumors, Langerhans Cell Histiocytosis, Laryngeal Cancer, Leukemia, Chronic Lymphocytic Cancer, Chronic Myelogenous Cancer, Hairy Cell Cancer, Lip and Oral Cavity Cancer, Liver Cancer (Primary), Lobular Carcinoma In Situ (LCIS), Lung Cancer, Non-Small Cell Cancer, Small Cell Cancer, Lymphoma, Mantle cell lymphoma, Cutaneous T-Cell (Mycosis Fungoides and Sézary Syndrome), Hodgkin Cancer, Non-Hodgkin Cancer, Macroglobulinemia, Waldenström, Male Breast Cancer, Malignant Fibrous Histiocytoma of Bone and Osteosarcoma, Melanoma, Intraocular (Eye) Cancer, Merkel Cell Carcinoma, Mesothelioma, Malignant, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic, Myeloid Leukemia, Acute, Myeloma Multiple, Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and Oropharyngeal Cancer, Osteosarcoma and Malignant Fibrous Histiocytoma of Bone, Epithelial Cancer, Low Malignant Potential Tumor, Pancreatic Cancer, Pancreatic Neuroendocrine Tumors (Islet Cell Tumors), Papillomatosis, Paraganglioma, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System Lymphoma, Rectal Cancer, Renal Cell (Kidney) Cancer, Retinoblastoma, Rhabdomyosarcoma, Salivary Gland Cancer, Sarcoma, Ewing Cancer, Kaposi Cancer, Osteosarcoma (Bone Cancer), Soft Tissue Cancer, Uterine Cancer, Sézary Syndrome, Skin Cancer, Childhood Melanoma, Merkel Cell Carcinoma, Nonmelanoma, Small Cell Lung Cancer, Small Intestine Cancer, Soft Tissue Sarcoma, Squamous Cell Carcinoma, Skin Cancer (Nonmelanoma), Childhood Squamous Neck Cancer with Occult Primary, Metastatic Cancer, Stomach (Gastric) Cancer, T-Cell Lymphoma, Cutaneous Cancer, Testicular Cancer, Throat Cancer, Thymoma and Thymic Carcinoma, Thyroid Cancer, Transitional Cell Cancer of the Renal Pelvis and Ureter, Unknown Primary, Carcinoma of Childhood, Unusual Cancers of Childhood, Urethral Cancer, Uterine Cancer, Endometrial Cancer, Uterine Sarcoma, Vaginal Cancer, Vulvar Cancer, Waldenström Macroglobulinemia, Wilms Tumor, or Women's Cancers.

3. The method of claim 2, wherein the cancer is one or more selected from sarcoma, lymphoma, colon cancer, lung cancer, or ovarian cancer.

4. The method of any one of claims 1-3, wherein the mutated protein is selected from one or more of the group consisting of CDK4, CDK6, E2F, R_{b}, pRb, cyclin D, cyclin D1, cyclin E, cyclin E1, Ki67, INK4, p53, p21, p27, p14, p15, and p16.

5. The method of any one of claims 1-4, wherein the mutated protein is selected from one or more of the group consisting of p21, p53, R_{b}, pRb, cyclin D1 and cyclin E.

6. The method of any one of claims 1-5, wherein the mutated protein is from a point mutation.

7. The method of any one of claims 1-6, wherein the mutated protein is a cyclin D1 having at least one point mutation.

8. The method of claim 8, wherein the point mutation of cyclin D1 is on R260H.

9. The method of any one of claims 1-6, wherein the subject has cyclin D1 with a copy number variation (CNV) greater than 2.

10. The method of any one of claims 1-6, wherein the mutated protein is a p14, p15, p16, and/or p21 having at least one point mutation.

11. The method of claim 10, wherein the point mutation of p14, p15, p16, and/or p21 is on at least one of H83Y, D84Y, D84V, R19H, and/or R67.

12. The method of any one of claims 1-11, wherein the subject has a loss of p21.

13. The method of any one of claims 1-12, wherein the subject has a wildtype Rb.

14. The method of any one of claims 1-6, wherein the mutated protein is a p53 having at least one point mutation.

15. The method of claim 14, wherein the point mutation of p53 is on at least one of R175H, R43H, M237I, R273H, C176W, R280K, L52R, L145R, R248W, and/or L130V.

16. The method of claim 6, wherein the point mutation on is on only one allele.

17. The method of claim 6, wherein the point mutation is on two alleles.

18. The method of any one of claims 1-3, wherein the mutation is on a protein, wherein the mutation provides overexpression, amplification, or deletion of one or more protein coding genes of the Rb checkpoint pathway and/or deletion of p53.

19. The method of claim 18, wherein the one or more protein coding genes is selected from a protein coding gene of CDK4, CDK6, E2F, Rb, pRb, cyclin D, cyclin D1, cyclin E, cyclin E1, Ki67, INK4, p53, p21, p27, p14, p15, and/or p16.

20. The method of claim 18 or 19, wherein the mutation is on a protein selected from Rb, cyclin D1, p53, p16, p15 and/or p21.

21. The method of claim 18-20, wherein the gene is selected from CCND1, CDKN2A, CDKN2B, CDKN1A, RB, and/or TP53.

22. The method of any one of claims 18-20, wherein the mutation provides overexpression or amplification of cyclin D or cyclin E genes.

23. The method of claim 22, wherein the mutation is on a protein, wherein the mutation provides overexpression of cyclin D1 gene.

24. The method of any one of claims 1-3, wherein the mutant form of the protein is provided by chromosome translocation of one or more protein coding genes of the Rb checkpoint pathway or p53.

25. The method of claim 24, wherein the one or more protein coding genes is selected from a protein coding gene of CDK4, CDK6, E2F, Rb, pRb, cyclin D, cyclin D1, cyclin E, cyclin E1, Ki67, INK4, p53, p21, p27, p14, p15, and/or p16.

26. The method of any one of claims 1-3, wherein the mutant form of the protein has a copy number variation (CNV) greater than 2.

27. The method of claim 26, wherein the mutant form of the protein comprises CNV3, CNV4, CNV5, CNV6, CNV7, CNV8, CNV9, or CNV10.

28. A method of inhibiting or reducing abnormal (e.g., overexpressed) wild-type or mutated cyclin D1 activity in a subject in need thereof, comprising administering a Parkin ligase activator or a pharmaceutically acceptable salt thereof.

29. A method of inducing cell cycle arrest or senescence in a subject in need thereof, comprising administering a Parkin ligase activator or a pharmaceutically acceptable salt thereof.

30. The method of claim 28 or 29, wherein the subject is human, wherein Parkin ligase in the subject is wild type, or still has its function resulting in the degradation of cyclin D.

31. The method of claim 30, wherein the subject has cancer.

32. The method of claim 31, wherein the cancer is one or more selected from Acute Lymphoblastic Leukemia, Acute Myeloid Leukemia, Adrenocortical Carcinoma, AIDS-Related Cancers, Kaposi Sarcoma, liposarcoma, soft tissue sarcoma, Lymphoma, Anal Cancer, Appendix Cancer, Astrocytomas, Childhood Atypical Teratoid/Rhabdoid Tumor, Basal Cell Carcinoma, Skin Cancer (Nonmelanoma), Childhood Bile Duct Cancer, Extrahepatic Bladder Cancer, Bone Cancer, Ewing Sarcoma Family of Tumors, Osteosarcoma and Malignant Fibrous Histiocytoma, Brain Stem Glioma, Brain Tumors, Embryonal Tumors, Germ Cell Tumors, Craniopharyngioma, Ependymoma, Bronchial Tumors, Burkitt Lymphoma (Non-Hodgkin Lymphoma), Carcinoid Tumor, Gastrointestinal Carcinoma of Unknown Primary, Cardiac (Heart) Tumors, Lymphoma, Primary, Cervical Cancer, Childhood Cancers, Chordoma, Chronic Lymphocytic Leukemia, Chronic Myelogenous Leukemia, Chronic Myeloproliferative Neoplasms Colon Cancer, Colorectal Cancer, Cutaneous T-Cell Lymphoma, Ductal Carcinoma In Situ, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Intraocular Melanoma, Retinoblastoma, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors, Extragonadal Cancer, Ovarian Cancer, Testicular Cancer, Gestational Trophoblastic Disease, Glioma, Brain Stem Cancer, Hairy Cell Leukemia, Head and Neck Cancer, Heart Cancer, Hepatocellular (Liver) Cancer, Histiocytosis, Langerhans Cell Cancer, Hodgkin Lymphoma, Hypopharyngeal Cancer, Intraocular Melanoma, Islet Cell Tumors, Pancreatic Neuroendocrine Tumors, Kidney Cancer, Renal Cell Cancer, Wilms Tumor and Other Childhood Kidney Tumors, Langerhans Cell Histiocytosis, Laryngeal Cancer, Leukemia, Chronic Lymphocytic Cancer, Chronic Myelogenous Cancer, Hairy Cell Cancer, Lip and Oral Cavity Cancer, Liver Cancer (Primary), Lobular Carcinoma In Situ (LCIS), Lung Cancer, Non-Small Cell Cancer, Small Cell Cancer, Lymphoma, Mantle cell lymphoma, Cutaneous T-Cell (Mycosis Fungoides and Sëzary Syndrome), Hodgkin Cancer, Non-Hodgkin Cancer, Macroglobulinemia, Waldenström, Male Breast Cancer, Malignant Fibrous Histiocytoma of Bone and Osteosarcoma, Melanoma, Intraocular (Eye) Cancer, Merkel Cell Carcinoma, Mesothelioma, Malignant, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic, Myeloid Leukemia, Acute, Myeloma Multiple, Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and Oropharyngeal Cancer, Osteosarcoma and Malignant Fibrous Histiocytoma of Bone, Epithelial Cancer, Low Malignant Potential Tumor, Pancreatic Cancer, Pancreatic Neuroendocrine Tumors (Islet Cell Tumors), Papillomatosis, Paraganglioma, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System Lymphoma, Rectal Cancer, Renal Cell (Kidney) Cancer, Retinoblastoma, Rhabdomyosarcoma, Salivary Gland Cancer, Sarcoma, Ewing Cancer, Kaposi Cancer, Osteosarcoma (Bone Cancer), Soft Tissue Cancer, Uterine Cancer, Sézary Syndrome, Skin Cancer, Childhood Melanoma, Merkel Cell Carcinoma, Nonmelanoma, Small Cell Lung Cancer, Small Intestine Cancer, Soft Tissue Sarcoma, Squamous Cell Carcinoma, Skin Cancer (Nonmelanoma), Childhood Squamous Neck Cancer with Occult Primary, Metastatic Cancer, Stomach (Gastric) Cancer, T-Cell Lymphoma, Cutaneous Cancer, Testicular Cancer, Throat Cancer, Thymoma and Thymic Carcinoma, Thyroid Cancer, Transitional Cell Cancer of the Renal Pelvis and Ureter, Unknown Primary, Carcinoma of Childhood, Unusual Cancers of Childhood, Urethral Cancer, Uterine Cancer, Endometrial Cancer, Uterine Sarcoma, Vaginal Cancer, Vulvar Cancer, Waldenström Macroglobulinemia, Wilms Tumor, or Women's Cancers.

33. The method of claim 31, wherein the cancer is one or more selected from sarcoma, lymphoma, colon cancer, lung cancer, or ovarian cancer.

34. The method of any one of claims 28-33, wherein the subject harbors a mutated protein selected from one or more of the group consisting of CDK4, CDK6, E2F, R_{b}, pRb, cyclin D, cyclin D1, cyclin E, cyclin E1, Ki67, INK4, p53, p21, p27, p15, and p16.

35. The method of any one of claims 28-35, wherein the mutated protein is selected from one or more of the group consisting of p21, p53, R_{b}, pRb, cyclin D1 and cyclin E.

36. The method of any one of claims 28-35, wherein the mutated protein is from a point mutation.

37. The method of any on one of claims 28-35, wherein the mutated protein is a cyclin D1 having at least one point mutation.

38. The method of claim 37, wherein the point mutation of cyclin D1 is on R260H.

39. The method of any one of claims 28-35, wherein the subject has cyclin D1 with a copy number variation (CNV) greater than 2.

40. The method of any one of claims 28-35, wherein the mutated protein is a p14, p15, p16, and/or p21 having at least one point mutation.

41. The method of claim 40, wherein the point mutation of p14, p15, p16, and/or p21 is on at least one of H83Y, D84Y, D84V, R19H, and/or R67.

42. The method of any one of claims 28-41, wherein the subject has a loss of p21.

43. The method of any one of claims 28-42, wherein the subject has a wildtype Rb.

44. The method of any on one of claims 28-35, wherein the mutated protein is a p53 having at least one point mutation.

45. The method of claim 44, wherein the point mutation of p53 is on at least one of R175H, R43H, M237I, R273H, C176W, R280K, L52R, L145R, R248W, and/or L130V.

46. The method of claim 36, wherein the point mutation on is on only one allele.

47. The method of claim 36, wherein the point mutation is on two alleles.

48. The method of any one of claims 28-33, wherein the mutation is on a protein, or the mutation provides overexpression, amplification, or deletion of one or more protein coding genes of the Rb checkpoint pathway or p53.

49. The method of claim 48, wherein the one or more protein coding genes is selected from a protein coding gene of CDK4, CDK6, E2F, Rb, pRb, cyclin D, cyclin D1, cyclin E, cyclin E1, Ki67, INK4, p53, p21, p27, p14, p15, and/or p16.

50. The method of claim 48 or 49, wherein the mutation is on a protein selected from Rb, cyclin D1, p53, p16, p15 and/or p21.

51. The method of claim 48-50, wherein the gene is selected from CCND1, CDKN2A, CDKN2B, CDKN1A, RB, and/or TP53.

52. The method of any one of claims 48-50, wherein the mutation provides overexpression or amplification of cyclin D or cyclin E genes.

53. The method of claim 52, wherein the mutation provides overexpression of cyclin D1 gene.

54. The method of any one of claims 28-33, wherein the mutant form of the protein is provided by chromosome translocation of one or more protein coding genes of the Rb checkpoint pathway or p53.

55. The method of claim 54, wherein the one or more protein coding genes is selected from a protein coding gene of CDK4, CDK6, E2F, Rb, pRb, cyclin D, cyclin D1, cyclin E, cyclin E1, Ki67, INK4, p53, p21, p27, p14, p15, and/or p16.

56. The method of any one of claims 28-33, wherein the mutant form of the protein has a copy number variation (CNV) greater than 2.

57. The method of claim 56, wherein the mutant form of the protein comprises CNV3, CNV4, CNV5, CNV6, CNV7, CNV8, CNV9, or CNV10.

58. A method of inhibiting or reducing abnormal (e.g., overexpressed) wild-type or mutated cyclin D1 activity or expression in human cells, comprising contacting a Parkin ligase activator or a pharmaceutically acceptable salt thereof with the human cells.

59. The method of claim 58, wherein the human cells are cancer cells.

60. The method of claim 59, wherein the cancer cells are one or more cells selected from Acute Lymphoblastic Leukemia, Acute Myeloid Leukemia, Adrenocortical Carcinoma, AIDS-Related Cancers, Kaposi Sarcoma, liposarcoma, soft tissue sarcoma, Lymphoma, Anal Cancer, Appendix Cancer, Astrocytomas, Childhood Atypical Teratoid/Rhabdoid Tumor, Basal Cell Carcinoma, Skin Cancer (Nonmelanoma), Childhood Bile Duct Cancer, Extrahepatic Bladder Cancer, Bone Cancer, Ewing Sarcoma Family of Tumors, Osteosarcoma and Malignant Fibrous Histiocytoma, Brain Stem Glioma, Brain Tumors, Embryonal Tumors, Germ Cell Tumors, Craniopharyngioma, Ependymoma, Bronchial Tumors, Burkitt Lymphoma (Non-Hodgkin Lymphoma), Carcinoid Tumor, Gastrointestinal Carcinoma of Unknown Primary, Cardiac (Heart) Tumors, Lymphoma, Primary, Cervical Cancer, Childhood Cancers, Chordoma, Chronic Lymphocytic Leukemia, Chronic Myelogenous Leukemia, Chronic Myeloproliferative Neoplasms Colon Cancer, Colorectal Cancer, Cutaneous T-Cell Lymphoma, Ductal Carcinoma In Situ, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Intraocular Melanoma, Retinoblastoma, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors, Extragonadal Cancer, Ovarian Cancer, Testicular Cancer, Gestational Trophoblastic Disease, Glioma, Brain Stem Cancer, Hairy Cell Leukemia, Head and Neck Cancer, Heart Cancer, Hepatocellular (Liver) Cancer, Histiocytosis, Langerhans Cell Cancer, Hodgkin Lymphoma, Hypopharyngeal Cancer, Intraocular Melanoma, Islet Cell Tumors, Pancreatic Neuroendocrine Tumors, Kidney Cancer, Renal Cell Cancer, Wilms Tumor and Other Childhood Kidney Tumors, Langerhans Cell Histiocytosis, Laryngeal Cancer, Leukemia, Chronic Lymphocytic Cancer, Chronic Myelogenous Cancer, Hairy Cell Cancer, Lip and Oral Cavity Cancer, Liver Cancer (Primary), Lobular Carcinoma In Situ (LCIS), Lung Cancer, Non-Small Cell Cancer, Small Cell Cancer, Lymphoma, Mantle cell lymphoma, Cutaneous T-Cell (Mycosis Fungoides and Sézary Syndrome), Hodgkin Cancer, Non-Hodgkin Cancer, Macroglobulinemia, Waldenström, Male Breast Cancer, Malignant Fibrous Histiocytoma of Bone and Osteosarcoma, Melanoma, Intraocular (Eye) Cancer, Merkel Cell Carcinoma, Mesothelioma, Malignant, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic, Myeloid Leukemia, Acute, Myeloma Multiple, Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and Oropharyngeal Cancer, Osteosarcoma and Malignant Fibrous Histiocytoma of Bone, Epithelial Cancer, Low Malignant Potential Tumor, Pancreatic Cancer, Pancreatic Neuroendocrine Tumors (Islet Cell Tumors), Papillomatosis, Paraganglioma, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System Lymphoma, Rectal Cancer, Renal Cell (Kidney) Cancer, Retinoblastoma, Rhabdomyosarcoma, Salivary Gland Cancer, Sarcoma, Ewing Cancer, Kaposi Cancer, Osteosarcoma (Bone Cancer), Soft Tissue Cancer, Uterine Cancer, Sézary Syndrome, Skin Cancer, Childhood Melanoma, Merkel Cell Carcinoma, Nonmelanoma, Small Cell Lung Cancer, Small Intestine Cancer, Soft Tissue Sarcoma, Squamous Cell Carcinoma, Skin Cancer (Nonmelanoma), Childhood Squamous Neck Cancer with Occult Primary, Metastatic Cancer, Stomach (Gastric) Cancer, T-Cell Lymphoma, Cutaneous Cancer, Testicular Cancer, Throat Cancer, Thymoma and Thymic Carcinoma, Thyroid Cancer, Transitional Cell Cancer of the Renal Pelvis and Ureter, Unknown Primary, Carcinoma of Childhood, Unusual Cancers of Childhood, Urethral Cancer, Uterine Cancer, Endometrial Cancer, Uterine Sarcoma, Vaginal Cancer, Vulvar Cancer, Waldenström Macroglobulinemia, Wilms Tumor, or Women's Cancers.

61. The method of claim 59, wherein the cancer cells are one or more cells selected from sarcoma, lymphoma, colon cancer, lung cancer, or ovarian cancer.

62. The method of any one of claims 58-61, wherein the human cells harbors a mutated protein selected from one or more of the group consisting of CDK4, CDK6, E2F, Rb, pRb, cyclin D, cyclin D1, cyclin E, cyclin E1, Ki67, INK4, p53, p21, p27, p14, p15, and p16.

63. The method of any one of claims 58-62, wherein the mutated protein is selected from one or more of the group consisting of p21, p53, R_{b}, pRb, cyclin D1 and cyclin E.

64. The method of any one of claims 58-61, wherein the mutated protein is from a point mutation.

65. The method of any on one of claims 58-61, wherein the mutated protein is a cyclin D1 having at least one point mutation.

66. The method of claim 65, wherein the point mutation of cyclin D1 is on R260H.

67. The method of any one of claims 58-61, wherein the subject has cyclin D1 with a copy number variation (CNV) greater than 2.

68. The method of any one of claims 58-61, wherein the mutated protein is a p14, pi 5, p16, and/or p21 having at least one point mutation.

69. The method of claim 68, wherein the point mutation of p14, p15, p16, and/or p21 is on at least one of H83Y, D84Y, D84V, R19H, and/or R67.

70. The method of any one of claims 58-69, wherein the subject has a loss of p21.

71. The method of any one of claims 58-70, wherein the subject has a wildtype Rb.

72. The method of any on one of claims 58-61, wherein the mutated protein is a p53 having at least one point mutation.

73. The method of claim 72, wherein the point mutation of p53 is on at least one of R175H, R43H, M237I, R273H, C176W, R280K, L52R, L145R, R248W, and/or L130V.

74. The method of claim 64, wherein the point mutation on is on only one allele.

75. The method of claim 64, wherein the point mutation is on two alleles.

76. The method of any one of claims 58-61, wherein the mutation is on a protein, or wherein the mutation provides overexpression, amplification, or deletion of one or more protein coding genes of the Rb checkpoint pathway or p53.

77. The method of claim 76, wherein the one or more protein coding genes is selected from a protein coding gene of CDK4, CDK6, E2F, Rb, pRb, cyclin D, cyclin D1, cyclin E, cyclin E1, Ki67, INK4, p53, p21, p27, p14, p15, and/or p16.

78. The method of claim 76 or 77, wherein the mutation is on a protein selected from R_{b}, cyclin D1, p53, p16, p15 and/or p21.

79. The method of claim 76-78, wherein the gene is selected from CCND1, CDKN2A, CDKN2B, CDKN1A, RB, and/or TP53.

80. The method of any one of claims 76-78, wherein the mutation provides overexpression or amplification of cyclin D or cyclin E genes.

81. The method of claim 74, wherein the mutation is on a protein, wherein the mutation provides overexpression of cyclin D1 gene.

82. The method of any one of claims 76-81, wherein the mutant form of the protein is provided by chromosome translocation of one or more protein coding genes of the Rb checkpoint pathway or p53.

83. The method of claim 82, wherein the one or more protein coding genes is selected from a protein coding gene of CDK4, CDK6, E2F, Rb, pRb, cyclin D, cyclin D1, cyclin E, cyclin E1, Ki67, INK4, p53, p21, p27, p14, p15, and/or p16.

84. The method of any one of claims 58-61, wherein the mutant form of the protein has a copy number variation (CNV) greater than 2.

85. The method of claim 84, wherein the mutant form of the protein comprises CNV3, CNV4, CNV5, CNV6, CNV7, CNV8, CNV9, or CNV10.

86. The method of treating a subject having a dysregulated Rb checkpoint pathway that results in increased cell growth, comprising administering to the subject a Parkin ligase activator or a pharmaceutically acceptable salt thereof.

87. The method of claim 86, wherein the pathway is dysregulated by increased or decreased expression of a wild type protein in the pathway.

88. The method of claim 87, wherein the increased or decreased expression results from a mutation of regulatory elements that control protein expression.

89. The method of claim 88, wherein the mutation of regulatory elements may be through alteration of enhancers or promoters, or that control transcription and translation of a protein in the pathway, or microRNAs that control transcription and degradation of mRNAs.

90. The method of claim 86, wherein the pathway is dysregulated by a mutation of a protein in the Rb checkpoint pathway.

91. The method of any one of claims 86-89, wherein there is altered expression of any one of proteins CDK4, CDK6, E2F, Rb, pRb, cyclin D, cyclin D1, cyclin E, cyclin E1, Ki67, INK4, p53, p21, p27, p14, p15, and/or p16.

92. The method of claim 91, wherein the protein is selected from Rb, cyclin D1, p53, p16, p15 and/or p21.

93. The method of claim 86, wherein the pathway is dysregulated by a mutated protein.

94. The method of claim 93, wherein the mutated protein is selected from CDK4, CDK6, E2F, Rb, pRb, cyclin D, cyclin D1, cyclin E, cyclin E1, Ki67, INK4, p53, p21, p27, p14, p15, and/or p16.

95. The method of claim 94, wherein the mutation protein is selected from Rb, cyclin D1, p53, p16, p15 and/or p21.

96. The method of any one of claims 86-95, wherein the dysregulated Rb checkpoint pathway is associated with cancer.

97. The method of claim 96, wherein the cancer is selected from Acute Lymphoblastic Leukemia, Acute Myeloid Leukemia, Adrenocortical Carcinoma, AIDS-Related Cancers, Kaposi Sarcoma, liposarcoma, soft tissue sarcoma, Lymphoma, Anal Cancer, Appendix Cancer, Astrocytomas, Childhood Atypical Teratoid/Rhabdoid Tumor, Basal Cell Carcinoma, Skin Cancer (Nonmelanoma), Childhood Bile Duct Cancer, Extrahepatic Bladder Cancer, Bone Cancer, Ewing Sarcoma Family of Tumors, Osteosarcoma and Malignant Fibrous Histiocytoma, Brain Stem Glioma, Brain Tumors, Embryonal Tumors, Germ Cell Tumors, Craniopharyngioma, Ependymoma, Bronchial Tumors, Burkitt Lymphoma (Non-Hodgkin Lymphoma), Carcinoid Tumor, Gastrointestinal Carcinoma of Unknown Primary, Cardiac (Heart) Tumors, Lymphoma, Primary, Cervical Cancer, Childhood Cancers, Chordoma, Chronic Lymphocytic Leukemia, Chronic Myelogenous Leukemia, Chronic Myeloproliferative Neoplasms Colon Cancer, Colorectal Cancer, Cutaneous T-Cell Lymphoma, Ductal Carcinoma In Situ, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Intraocular Melanoma, Retinoblastoma, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors, Extragonadal Cancer, Ovarian Cancer, Testicular Cancer, Gestational Trophoblastic Disease, Glioma, Brain Stem Cancer, Hairy Cell Leukemia, Head and Neck Cancer, Heart Cancer, Hepatocellular (Liver) Cancer, Histiocytosis, Langerhans Cell Cancer, Hodgkin Lymphoma, Hypopharyngeal Cancer, Intraocular Melanoma, Islet Cell Tumors, Pancreatic Neuroendocrine Tumors, Kidney Cancer, Renal Cell Cancer, Wilms Tumor and Other Childhood Kidney Tumors, Langerhans Cell Histiocytosis, Laryngeal Cancer, Leukemia, Chronic Lymphocytic Cancer, Chronic Myelogenous Cancer, Hairy Cell Cancer, Lip and Oral Cavity Cancer, Liver Cancer (Primary), Lobular Carcinoma In Situ (LCIS), Lung Cancer, Non-Small Cell Cancer, Small Cell Cancer, Lymphoma, Mantle cell lymphoma, Cutaneous T-Cell (Mycosis Fungoides and Sézary Syndrome), Hodgkin Cancer, Non-Hodgkin Cancer, Macroglobulinemia, Waldenström, Male Breast Cancer, Malignant Fibrous Histiocytoma of Bone and Osteosarcoma, Melanoma, Intraocular (Eye) Cancer, Merkel Cell Carcinoma, Mesothelioma, Malignant, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic, Myeloid Leukemia, Acute, Myeloma Multiple, Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and Oropharyngeal Cancer, Osteosarcoma and Malignant Fibrous Histiocytoma of Bone, Epithelial Cancer, Low Malignant Potential Tumor, Pancreatic Cancer, Pancreatic Neuroendocrine Tumors (Islet Cell Tumors), Papillomatosis, Paraganglioma, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System Lymphoma, Rectal Cancer, Renal Cell (Kidney) Cancer, Retinoblastoma, Rhabdomyosarcoma, Salivary Gland Cancer, Sarcoma, Ewing Cancer, Kaposi Cancer, Osteosarcoma (Bone Cancer), Soft Tissue Cancer, Uterine Cancer, Sézary Syndrome, Skin Cancer, Childhood Melanoma, Merkel Cell Carcinoma, Nonmelanoma, Small Cell Lung Cancer, Small Intestine Cancer, Soft Tissue Sarcoma, Squamous Cell Carcinoma, Skin Cancer (Nonmelanoma), Childhood Squamous Neck Cancer with Occult Primary, Metastatic Cancer, Stomach (Gastric) Cancer, T-Cell Lymphoma, Cutaneous Cancer, Testicular Cancer, Throat Cancer, Thymoma and Thymic Carcinoma, Thyroid Cancer, Transitional Cell Cancer of the Renal Pelvis and Ureter, Unknown Primary, Carcinoma of Childhood, Unusual Cancers of Childhood, Urethral Cancer, Uterine Cancer, Endometrial Cancer, Uterine Sarcoma, Vaginal Cancer, Vulvar Cancer, Waldenström Macroglobulinemia, Wilms Tumor, and/or Women's Cancers.

98. The method of claim 97, wherein the cancer is selected from breast cancer, sarcoma, lymphoma, colon cancer, lung cancer, or ovarian cancer.

99. The method of any one of claims 1-98, wherein the Parkin ligase activator is a compound of formula (I): 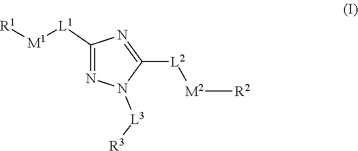
or a pharmaceutically acceptable salt or solvate thereof, wherein:
L¹, L² and L³ are each independently selected from a bond, alkylene, or alkenylene;
M¹ and M² are each independently selected from —NR⁴—, —NR⁴C(O)—, —N(C(O)R¹)—, —C(O)NR⁴—, —NR⁴C(O)NR⁴—, —C(O)—, —C(═NR⁴)—, —C(═NOR⁴)—, —OC(O)—, —C(O)O—, —OC(O)O—, —OC(O)NR⁴—, —NR⁴C(O)O—, —S(O)ₘ—, —S(O)ₘNR⁴—, or —NR⁴S(O)ₘ—, provided that M¹ and M² are not both —NR⁴—;
R¹ and R² are each independently selected from an alkyl, alkenyl, cycloalkyl, aryl, biphenyl, heterocyclyl heterocycloalkyl, heteroaryl, cycloalkylalkyl, arylalkyl, arylalkenyl, arylalkynyl, heterocyclylalkyl, heteroarylalkyl, heteroarylalkenyl, or heteroarylalkynyl, wherein each cycloalkyl, aryl, heteroaryl, and heterocyclyl portion is optionally substituted with one or more R⁵;
R³ is selected from an alkyl, alkenyl, cycloalkyl, aryl, biphenyl, heterocyclyl heterocycloalkyl, heteroaryl, cycloalkylalkyl, arylalkyl, arylalkenyl, arylalkynyl, heterocyclylalkyl, heteroarylalkyl, heteroarylalkenyl, or heteroarylalkynyl, wherein each cycloalkyl, aryl, heteroaryl, and heterocyclyl portion is optionally substituted with one or more R⁷;
R⁴ is each independently H, alkyl, wherein each alkyl is optionally substituted with one or more R⁵;
R⁵ is each independently I, Br, Cl, F, —CH₂F, —CHF₂, —CF₃, —OCF₃, —CN, -alkyl-CN, —CONH₂, —CONHR⁶, —CONR⁶R⁶, —COOH, —NH₂, —NHR⁶, —NO₂, —NR⁶R⁶, —N₃, —OH, OR⁶, —COOR⁶, —OSO₃R⁶, oxo, R⁶, —SH, —SO₂R⁶, —SO₃H, —SO₃R⁶, or —SR⁶;
R⁶ is each independently alkyl, alkenyl, alkynyl, haloalkyl, haloalkenyl, haloalkynyl; or alternatively two R⁶ on the same N atom can together form a 3-6 membered N-heterocyclyl;
R⁷ is each independently I, Br, Cl, F, —CH₂F, —CHF₂, —CF₃, —OCF₃, —CN, -alkyl-CN, —CONH₂, —CONHR⁶, —CONR⁶R⁶, —COOH, —NH₂, —NHR⁶, —NO₂, —NR⁶R⁶, —N₃, —OH, OR⁶, —COOR⁶, —OSO₃R⁶, oxo, R⁶, —SH, —SO₂R⁶, —SO₃H, —SO₃R⁶, —SR⁶, cycloalkyl, heterocyclyl, aryl, or heteroaryl, wherein each cycloalkyl, heterocyclyl, aryl, and heteroaryl is optionally substituted with one or more R⁵; and
m is 0, 1, or 2.

100. The method of claim 99, wherein L¹, L² and L³ are each independently a bond.

101. The method of claim 99 or 100, wherein M¹ and M² are each independently selected from —NR⁴—, —NR⁴C(O)—, —C(O)NR⁴—, —N(C(O)R¹)—, or —NR⁴S(O)ₘ—.

102. The method of any one of claims 99-101, wherein M¹ and M² are each independently selected from —NR⁴—, —NR⁴C(O)— or —C(O)NR⁴—.

103. The method of claim 101 or 102, wherein R⁴ at each occurrence is independently H or C₁-C₃ alkyl.

104. The method of any one of claims 99-103, wherein L³ is a bond and R³ is an aryl or a heteroaryl, optionally substituted with one or more R⁷.

105. The method of claim 104, wherein R³ is a phenyl or phenyl fused bicycle, optionally substituted with one or more R⁷.

106. The method of claim 104, wherein R³ is heteroaryl selected from imidazolyl or pyrazolyl, optionally substituted with one or more R⁷.

107. The method of any one of claims 104-106, wherein R⁷ is each independently I, Br, Cl, F, —CH₃, —CH₂F, —CHF₂, —CF₃, —OCF₃, —CN, —NH₂, —NMe₂, —NO₂, —N₃, —OH, OR⁶, R⁶, —SH, cycloalkyl, heterocyclyl, aryl, or heteroaryl, wherein each cycloalkyl, heterocyclyl, aryl, and heteroaryl is optionally substituted with one or more R⁵.

108. The method of claim 105, wherein R³ is a phenyl substituted with a 4-6 membered heterocyclyl, which is optionally substituted with one or more R⁷.

109. The method of claim 106, wherein R¹ and R² are each independently selected from phenyl, 6-10 membered aryl, 5-10 membered heteroaryl, 4-10 membered heterocyclyl, phenyl-(C₁-C₃ alkyl)-, phenyl-(C₂-C₃ alkenyl)-, 5-6 membered heteroaryl-(C₁-C₃ alkyl)-, or heteroaryl-(C₂-C₃ alkenyl)-, wherein each cycloalkyl, aryl, heteroaryl portion is optionally substituted with one or more R⁵.

110. The method of claim 109, wherein the 6-10 membered aryl or 5-10 membered heteroaryl is a bicyclic ring.

111. The method of any one of claims 99-110, wherein R⁵ is selected from I, Br, Cl, F, C₁-C₆ alkyl, alkynyl, —CN, —(C₁-C₃ alkylene)-CN, —NH₂, —NO₂, —N₃, —OH, —OCF₃, —OMe, —NMe₂, or —NEt₂.

112. The method of any one of claims 99-111, wherein at least one of R¹, R², and R³ is phenyl and substituted with at least one of methyl, ethyl, —C≡CH, I, Br, Cl, F, —CF₃, —CN, —CH₂CN, —CH₂CH₂CN, —NH₂, —NO₂, —N₃, —OH, —OCF₃, —OMe or —NMe₂.

113. The method of claim 99-105, wherein at least two of R¹, R², and R³ is phenyl and substituted with at least one of methyl, ethyl, —C≡CH, I, Br, Cl, F, —CF₃, —CN, —CH₂CN, —CH₂CH₂CN, —NH₂, —NO₂, —N₃, —OH, —OCF₃, —OMe or —NMe₂.

114. The method of claim 112 or 113, wherein at least one of R¹, R², and R³ is pyridyl, optionally substituted with one or more of methyl, ethyl, —C≡CH, I, Br, Cl, F, —CF₃, —CN, —CH₂CN, —CH₂CH₂CN, —NH₂, —NO₂, —N₃, —OH, —OCF₃, —OMe or —NMe₂.

115. The method of claim 99, wherein the Parkin ligase activator has the structure of formula (I′) 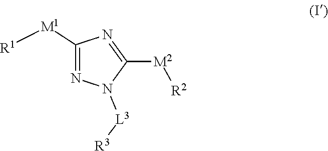
or a pharmaceutically acceptable salt or solvate thereof, wherein:
L³, M¹, M², R¹, R², and R³ are as defined in claim 1.

116. The method of claim 115, wherein M¹ and M² are each independently selected from —NR⁴—, —NR⁴C(O)—, —C(O)NR⁴—, —N(C(O)R¹)—, or —NR⁴S(O)ₘ—.

117. The method of claim 115, wherein R¹ and R² are each independently selected from phenyl, 6-10 membered aryl, 5-10 membered heteroaryl, 4-10 membered heterocyclyl, phenyl-(C₁-C₃ alkyl)-, phenyl-(C₂-C₃ alkenyl)-, 5-6 membered heteroaryl-(C₁-C₃ alkyl)-, or heteroaryl-(C₂-C₃ alkenyl)-, wherein each cycloalkyl, aryl, heteroaryl portion is optionally substituted with one or more R⁵; and
R³ is an aryl or a heteroaryl, optionally substituted with one or more R⁷.

118. The method of claim 99, wherein the Parkin ligase activator has the structure of formula (IA′) 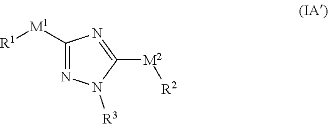
or a pharmaceutically acceptable salt or solvate thereof, wherein:
M¹ and M² are each independently selected from —NR⁴C(O)— or —C(O)NR⁴—;
R¹ and R² are each independently 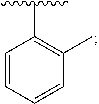 [SMILES: *c1ccccc1C]
R³ is selected from 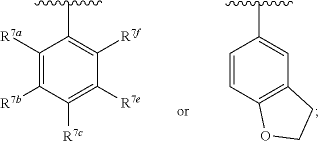
R⁴ is each independently H or C₁-C₃ alkyl; and
R^{7a}, R^{7b}, R^{7e}, and R^{7f} is each independently H, I, Br, Cl, F, —CH₂F, —CHF₂, —CF₃, —OCF₃, —N₃, —CN, —OH, methyl, ethyl, propyl, C₁-C₃ haloalkyl, C₁-C₃ alkoxy, or C₁-C₃ haloalkoxy;
R^{7c} is H, I, Br, Cl, F, —CH₂F, —CHF₂, —CF₃, —OCF₃, —N₃, —CN, —OH, C₁-C₃ alkyl, C₁-C₃ haloalkyl, C₁-C₃ alkoxy, C₁-C₃ haloalkoxy, 4-6 membered heterocyclyl, or 5-6 membered heteroaryl, wherein the heterocyclyl and heteroaryl is optionally substituted with one or more R⁵;
R⁵ is I, Br, Cl, F, —CH₂F, —CHF₂, —CF₃, —C₁-C₆ alkyl, alkynyl, —CN, —(C₁-C₃ alkylene)-CN, —NH₂, —NO₂, —N₃, —OH, —OCF₃, —OMe, —NMe₂, —NEt₂, or—C(O)O(C₁-C₆ alkyl);
wherein at least one of R^{7a}, R^{7b}, R^{7c}, R^{7e}, and R^{7f} is not H.

119. The method of claim 118, wherein R³ is selected from R³ is

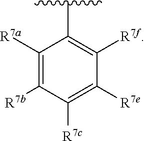

120. The method of claim 119, wherein four of R^{7a}, R^{7b}, R^{7c}, R^{7e}, and R^{7f} is H.

121. The method of claim 119, wherein three of R^{7a}, R^{7b}, R^{7c}, R^{7e}, and R^{7f} is H.

122. The method of any one of claims 118-121, wherein R^{7a}, R^{7b}, R^{7c}, R^{7e}, and R^{7f} is each independently H, I, Br, Cl, F, —CH₂F, —CHF₂, —CF₃, —OCF₃, —N₃, —CN, —OH, methyl, ethyl, propyl, —C≡CH; —CN, —NH₂, —NO₂, —N₃, —OH, —OCF₃, —OMe, —NMe₂, or —NEt₂.

123. The method of claim 118, wherein R³ is

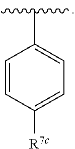

124. The method of claim 123, wherein R^{7c} is I, Br, —CH₂F, —CHF₂, —CF₃, methyl, ethyl, propyl, —C≡CH; —CN, —NH₂, —NO₂, —N₃, —OH, —OCF₃, —OMe, —NMe₂, or —NEt₂.

125. The method of claim 123, wherein R^{7c} is I, Br, —CH₂F, —CHF₂, —CF₃, —OCF₃, or —OMe.

126. The method of claim 123, wherein R^{7c} is azetidinyl, pyrrolidinyl, piperidinyl, morpholinyl, piperazinyl, or pyrazolyl, each optionally substituted with one or more R⁵.

127. The method of claim 99, wherein the Parkin ligase activator has the structure of formula (IA): 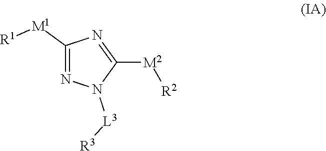
or a pharmaceutically acceptable salt or solvate thereof, wherein:
L³ is a bond;
M¹ and M² are each independently selected from —NR⁴C(O)— or —C(O)NR⁴—;
R¹ and R² are each phenyl, substituted with one or more R^{5a};
R³ is phenyl, optionally substituted with one or more R^{5b};
R⁴ is each independently H or C1-C3 alkyl;
R^{5a} is each independently I, Br, Cl, F, C1-C6 alkyl, C1-C3 haloalkyl, —(C1-C6)-O—(C1-C6), C1-C3 alkoxy, C1-C3 haloalkoxy, OH, or COOH;
R^{5b} is each independently I, Br, Cl, F, CN, CONH₂, CONHR⁶, CONR⁶R⁶, COOH, NH₂, NHR⁶, NO₂, NR⁶R⁶, OH, OR⁶, —COOR⁶, OSO₃R⁶, oxo, R⁶, SH, SO₂R⁶, SO₃H, SO₃R⁶, or SR⁶; and
R⁶ is each independently alkyl or haloalkyl.

128. The method of claim 127, wherein R1 is phenyl substituted with one or more R5a and R5a comprises at least one OH.

129. The method of claim 99, wherein the Parkin ligase activator has the structure of formula 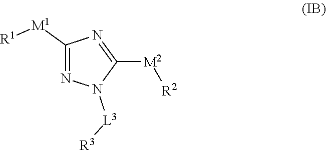
or a pharmaceutically acceptable salt or solvate thereof, wherein:
L³ is a bond;
M¹ and M² are each independently selected from —NR⁴C(O)— or —C(O)NR⁴—;
R¹ and R² are each phenyl, substituted with one or more R^{5a};
R³ is phenyl, optionally substituted with one or more R^{5b};
R⁴ is each independently H or C1-C3 alkyl;
R^{5a} is each independently C1-C6 alkyl;
R^{5b} is each independently I, Br, Cl, F, CN, CONH₂, CONHR⁶, CONR⁶R⁶, COOH, NH₂, NHR⁶, NO₂, NR⁶R⁶, OH, OR⁶, —COOR⁶, OSO₃R⁶, oxo, R⁶, SH, SO₂R⁶, SO₃H, SO₃R⁶, or SR⁶; and
R⁶ is each independently alkyl or haloalkyl.

130. The method of claim 99, wherein the Parkin ligase activator has the structure of formula (IC): 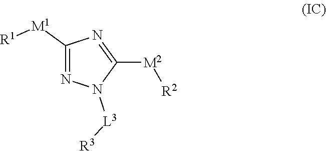
or a pharmaceutically acceptable salt or solvate thereof, wherein:
L³ is a bond;
M¹ and M² are each independently selected from —NR⁴C(O)— or —C(O)NR⁴—;
R¹ and R² are each phenyl, substituted with one or more R^{5a}, wherein at least one of R¹ and R² is 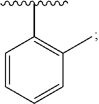 [SMILES: *c1ccccc1C]
R³ is phenyl, optionally substituted with one or more R^{5b};
R⁴ is each independently H or C1-C3 alkyl;
R^{5a} is each independently I, Br, Cl, F, C1-C6 alkyl, C1-C3 haloalkyl, C1-C3 alkoxy, C1-C3 haloalkoxy, OH, or COOH;
R^{5b} is each independently I, Br, Cl, F, CN, CONH₂, CONHR⁶, CONR⁶R⁶, COOH, NH₂, NHR⁶, NO₂, NR⁶R⁶, OH, OR⁶, —COOR⁶, OSO₃R⁶, oxo, R⁶, SH, SO₂R⁶, SO₃H, SO₃R⁶, or SR⁶; and
R⁶ is each independently alkyl or haloalkyl.

131. The method of claim 99, wherein the Parkin ligase activator has the structure of formula (ID): 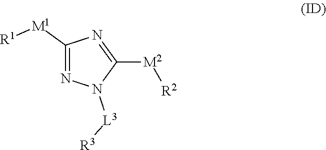
or a pharmaceutically acceptable salt or solvate thereof, wherein:
L³ is a bond;
M¹ and M² are each independently selected from —NR⁴C(O)— or —C(O)NR⁴—;
R¹ and R² are each 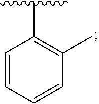 [SMILES: *c1ccccc1C]
R³ is phenyl, optionally substituted with one or more R^{5b};
R⁴ is each independently H or C1-C3 alkyl;
R^{5b} is each independently I, Br, Cl, F, CN, CONH₂, CONHR⁶, CONR⁶R⁶, COOH, NH₂, NHR⁶, NO₂, NR⁶R⁶, OH, OR⁶, —COOR⁶, OSO₃R⁶, oxo, R⁶, SH, SO₂R⁶, SO₃H, SO₃R⁶, or SR⁶; and
R⁶ is each independently alkyl or haloalkyl.

132. The method of claim 99, wherein the Parkin ligase activator has the structure of formula (IE): 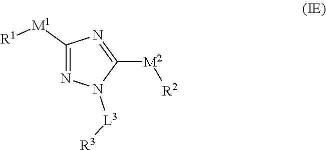
or a pharmaceutically acceptable salt or solvate thereof, wherein:
L³ is a bond;
M¹ and M² are each —NHC(O)—;
R¹ and R² are each 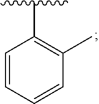 [SMILES: *c1ccccc1C]
R³ is phenyl, optionally substituted with one or more R^{5b}; and
R^{5b} is each independently I, Br, Cl, F, C1-C3 alkyl, C1-C3 haloalkyl, C1-C3 alkoxy, C1-C3 haloalkoxy, OH, or COOH.

133. The method of claim 99, wherein the Parkin ligase activator has the structure of formula (IF): 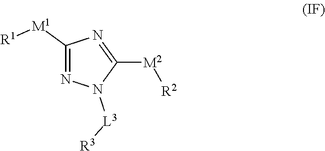
or a pharmaceutically acceptable salt or solvate thereof, wherein:
L³ is a bond;
M¹ and M² are each —NHC(O)—;
R¹ and R² are each 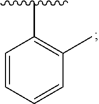 [SMILES: *c1ccccc1C]
R³ is phenyl, optionally substituted with one or more R^{5b}; and
R^{5b} is each independently C1-C3 alkyl, C1-C3 haloalkyl, C1-C3 alkoxy, C1-C3 haloalkoxy, OH, or COOH.

134. The method of claim 99, wherein the Parkin ligase activator has the structure of formula (IG): 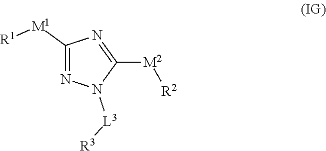
or a pharmaceutically acceptable salt or solvate thereof, wherein:
L³ is a bond;
M¹ and M² are each —NHC(O)—:
R¹ and R² are each 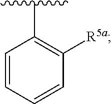
R³ is phenyl; and
R^{5a} is each independently C1-C6 alkyl, C1-C3 haloalkyl, C1-C3 alkoxy, C1-C3 haloalkoxy, OH, or COOH.

135. The method of any one of claims 1-98, wherein the Parkin ligase activator is a compound of formula (II): 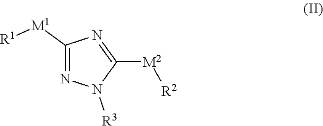
or a pharmaceutically acceptable salt or solvate thereof, wherein:
M¹ and M² are each independently selected from a bond, —NR⁴—, or —NR⁴C(O)—, —C(O)NR⁴—;
R¹ and R² are each independently selected from an alkyl, alkenyl, cycloalkyl, aryl, biphenyl, heterocyclyl heterocycloalkyl, heteroaryl, cycloalkylalkyl, arylalkyl, arylalkenyl, arylalkynyl, heterocyclylalkyl, heteroarylalkyl, heteroarylalkenyl, or heteroarylalkynyl, wherein each cycloalkyl, aryl, heteroaryl, and heterocyclyl portion is optionally substituted with one or more R⁵;
wherein at least one of M¹ and M² is a bond or —NR⁴—;
wherein when M¹ is —NR⁴—, then R¹ is cycloalkylalkyl, heterocyclylalkyl, arylalkyl, or heteroarylalkyl, wherein cycloalkyl, aryl, heteroaryl, and heterocyclyl portion is optionally substituted with one or more R⁵;
wherein when M² is —NR⁴—, then R² is cycloalkylalkyl, heterocyclylalkyl, arylalkyl, or heteroarylalkyl, wherein cycloalkyl, aryl, heteroaryl, and heterocyclyl portion is optionally substituted with one or more R⁵;
R³ is selected from an alkyl, alkenyl, cycloalkyl, aryl, biphenyl, heterocyclyl heterocycloalkyl, heteroaryl, cycloalkylalkyl, arylalkyl, arylalkenyl, arylalkynyl, heterocyclylalkyl, heteroarylalkyl, heteroarylalkenyl, or heteroarylalkynyl, wherein each cycloalkyl, aryl, heteroaryl, and heterocyclyl portion is optionally substituted with one or more R⁷;
R⁴ is each independently H, alkyl, wherein each alkyl is optionally substituted with one or more R⁵;
R⁵ is each independently I, Br, Cl, F, —CH₂F, —CHF₂, —CF₃, —OCF₃, —CN, -alkyl-CN, —CONH₂, —CONHR⁶, —CONR⁶R⁶, —COOH, —NH₂, —NHR⁶, —NO₂, —NR⁶R⁶, —N₃, —OH, OR⁶, —COOR⁶, —OSO₃R⁶, oxo, R⁶, —SH, —SO₂R⁶, —SO₃H, —SO₃R⁶, or —SR⁶;
R⁶ is each independently alkyl, alkenyl, alkynyl, haloalkyl, haloalkenyl, haloalkynyl; or alternatively two R⁶ on the same N atom can together form a 3-6 membered N-heterocyclyl; and
R⁷ is each independently I, Br, Cl, F, —CH₂F, —CHF₂, —CF₃, —OCF₃, —CN, -alkyl-CN, —CONH₂, —CONHR⁶, —CONR⁶R⁶, —COOH, —NH₂, —NHR⁶, —NO₂, —NR⁶R⁶, —N₃, —OH, OR⁶, —COOR⁶, —OSO₃R⁶, oxo, R⁶, —SH, —SO₂R⁶, —SO₃H, —SO₃R⁶, —SR⁶, cycloalkyl, heterocyclyl, aryl, or heteroaryl, wherein each cycloalkyl, heterocyclyl, aryl, and heteroaryl is optionally substituted with one or more R⁵.

136. The method of any one of claims 1-98, wherein the Parkin ligase activator is a compound of formula (II′): 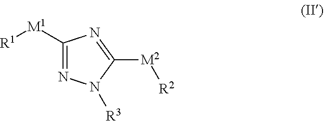
or a pharmaceutically acceptable salt or solvate thereof, wherein:
M¹ and M² are each independently selected from a bond, —NR⁴—, —NR⁴C(O)—, —C(O)NR⁴—, provided that M¹ and M² are not both —NR⁴— or both a bond;
R¹ and R² are each independently selected from a cycloalkyl, aryl, heterocyclyl, or heteroaryl, wherein each cycloalkyl, aryl, heteroaryl, and heterocyclyl is optionally substituted with one or more R^{5a}, provided that R¹ and R² are not 1,3-dioxoisoindolin-2-yl;
R³ is selected from an alkyl, alkenyl, cycloalkyl, aryl, heterocyclyl, or heteroaryl, wherein each cycloalkyl, aryl, heteroaryl, and heterocyclyl is optionally substituted with one or more R^{5a};
R⁴ is each independently H or alkyl;
R^{5a} is each independently I, Br, Cl, F, CN, NH₂, NHR^{6a}, NO₂, NR^{6a}R^{6a}, OH, OR^{6a}, or R^{6a}; and
R^{6a} is each independently alkyl or haloalkyl; or alternatively two R^{6a} on the same N atom can together form a 3-6 membered N-heterocyclyl.

137. The method of any one of claims 1-98, wherein the Parkin ligase activator is a compound of formula (IIB): 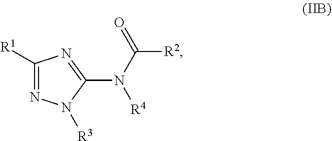
or a pharmaceutically acceptable salt or solvate thereof, wherein:
R¹ and R² are each independently selected from aryl or heteroaryl, each optionally substituted with one or more R⁵;
R³ is selected from aryl or heteroaryl, each optionally substituted with one or more R⁷;
R⁴ is each independently H or —C₁-C₃ alkyl;
R⁵ is I, Br, Cl, F, —CH₂F, —CHF₂, —CF₃, —C₁-C₆ alkyl, alkynyl, —CN, —(C₁-C₃ alkylene)-CN, —NH₂, —(C₁-C₃ alkylene)-NH₂, —(C₁-C₃ alkylene)-NHR⁶, —(C₁-C₃ alkylene)-NR⁶R⁶, —NO₂, —N₃, —OH, —OCF₃, —OMe, —NMe₂, —NEt₂, or —C(O)O(C₁-C₆ alkyl);
R⁶ is each independently alkyl, alkenyl, alkynyl, haloalkyl, haloalkenyl, haloalkynyl; or alternatively two R⁶ on the same N atom can together form a 3-6 membered N-heterocyclyl; and
R⁷ is each independently I, Br, Cl, F, —CH₂F, —CHF₂, —CF₃, —OCF₃, —CN, -alkylene-CN, —CONH₂, —CONHR⁶, —CONR⁶R⁶, —COOH, —NH₂, -alkylene-NH₂, —NHR⁶, -alkylene-NHR⁶, —NO₂, —NR⁶R⁶, -alkylene-NR⁶R⁶, —N₃, —OH, OR⁶, —COOR⁶, —OSO₃R⁶, oxo, R⁶, —SH, —SO₂R⁶, —SO₃H, —SO₃R⁶, —SR⁶, cycloalkyl, heterocyclyl, aryl, or heteroaryl, wherein each cycloalkyl, heterocyclyl, aryl, and heteroaryl is optionally substituted with one or more R⁵;
wherein the compound is not N-benzyl-N-(5-(benzylamino)-1-phenyl-1H-1,2,4-triazol-3-yl)acetamide, N-(5-((2-chlorobenzyl)amino)-1-phenyl-1H-1,2,4-triazol-3-yl)-2-fluorobenzamide and N³, N⁵-bis(4-methylbenzyl)-1-phenyl-1H-1,2,4-triazole-3,5-diamine.

138. The method of claim 137, wherein
R³ is 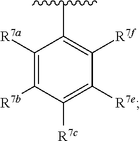
R^{7a}, R^{7b}, R^{7e}, and R^{7f} is each independently H, I, Br, Cl, F, —CH₂F, —CHF₂, —CF₃, —OCF₃, —N₃, —CN, —OH, methyl, ethyl, propyl, C₁-C₃ haloalkyl, C₁-C₃ alkoxy, or C₁-C₃ haloalkoxy;
R^{7c} is H, I, Br, Cl, F, —CH₂F, —CHF₂, —CF₃, —OCF₃, —N₃, —CN, —OH, —C₁-C₃ alkyl, —C₁-C₃ haloalkyl, —C₁-C₃ alkoxy, —C₁-C₃ haloalkoxy, 4-6 membered heterocyclyl, or 5-6 membered heteroaryl, wherein the heterocyclyl and heteroaryl is optionally substituted with one R^{5b};
R^{5b} is I, Br, Cl, F, —CH₂F, —CHF₂, —CF₃, —C₁-C₆ alkyl, alkynyl, —CN, —(C₁-C₃ alkylene)-CN, —NH₂, —(C₁-C₃ alkylene)-NH₂, —(C₁-C₃ alkylene)-NHR⁶, —(C₁-C₃ alkylene)-NR⁶R⁶, —NO₂, —N₃, —OH, —OCF₃, —OMe, —NMe₂, —NEt₂, or —C(O)O(C₁-C₆ alkyl); and
wherein at least one of R^{7a}, R^{7b}, R^{7c}, R^{7e}, and R^{7f} is not H.

139. The method of any one of claims 1-98, wherein the Parkin ligase activator is a compound of formula (III): 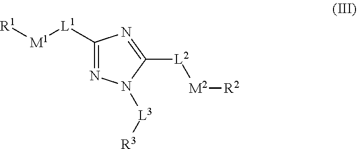
or a pharmaceutically acceptable salt or solvate thereof, wherein:
L¹, L² and L³ are each independently selected from a bond, alkylene, or alkenylene;
M¹ and M² are each independently selected from —NR⁴—, —NR⁴C(O)—, —N(C(O)R¹)—, —C(O)NR⁴—, —NR⁴C(O)NR⁴—, —C(O)—, —C(═NR⁴)—, —C(═NOR⁴)—, —OC(O)—, —C(O)O—, —OC(O)O—, —OC(O)NR⁴—, —NR⁴C(O)O—, —S(O)ₘ—, —S(O)ₘNR⁴—, or —NR⁴S(O)ₘ—, provided that M¹ and M² are not both —NR⁴—;
R¹ and R² are each independently selected from an alkyl, alkenyl, cycloalkyl, aryl, biphenyl, heterocyclyl heterocycloalkyl, heteroaryl, cycloalkylalkyl, arylalkyl, arylalkenyl, arylalkynyl, heterocyclylalkyl, heteroarylalkyl, heteroarylalkenyl, or heteroarylalkynyl, wherein each cycloalkyl, aryl, heteroaryl, and heterocyclyl portion is optionally substituted with one or more R⁵;
R³ is selected from an alkyl, alkenyl, cycloalkyl, aryl, biphenyl, heterocyclyl heterocycloalkyl, heteroaryl, cycloalkylalkyl, arylalkyl, arylalkenyl, arylalkynyl, heterocyclylalkyl, heteroarylalkyl, heteroarylalkenyl, or heteroarylalkynyl, wherein each cycloalkyl, aryl, heteroaryl, and heterocyclyl portion is optionally substituted with one or more R⁷;
R⁴ is each independently H, alkyl, wherein each alkyl is optionally substituted with one or more R⁵;
R⁵ is each independently I, Br, Cl, F, —CH₂F, —CHF₂, —CF₃, —OCF₃, —CN, -alkylene-CN, —CONH₂, —CONHR⁶, —CONR⁶R⁶, —COR⁶, —COOH, —NH₂, -alkylene-NH₂, —NHR⁶, -alkylene-NHR⁶, —NO₂, —NR⁶R⁶, -alkylene-NR⁶R⁶, —NR⁶COR⁶, -(alkylene)NR⁶COR⁶, —N₃, —OH, OR⁶, —COOR⁶, —OSO₃R⁶, oxo, R⁶, —SH, —SO₂R⁶, —SO₃H, —SO₃R⁶, —SR⁶, heterocyclyl, or -alkylene-heterocyclyl, wherein heterocyclyl is optionally substituted with one or more R⁸;
R⁶ is each independently alkyl, alkenyl, alkynyl, haloalkyl, haloalkenyl, haloalkynyl; or alternatively two R⁶ on the same N atom can together form a 3-6 membered N-heterocyclyl;
R⁷ is each independently I, Br, Cl, F, —CH₂F, —CHF₂, —CF₃, —OCF₃, —CN, -alkylene-CN, —CONH₂, —CONHR⁶, —CONR⁶R⁶, —COOH, —NH₂, -alkylene-NH₂, —NHR⁶, -alkylene-NHR⁶, —NO₂, —NR⁶R⁶, -alkylene-NR⁶R⁶, —N₃, —OH, OR⁶, —COOR⁶, —OSO₃R⁶, oxo, R⁶, —SH,—SO₂R⁶, —SO₃H, —SO₃R⁶, —SR⁶, cycloalkyl, heterocyclyl, aryl, or heteroaryl, wherein each cycloalkyl, heterocyclyl, aryl, and heteroaryl is optionally substituted with one or more R⁵;
R⁸ is each independently I, Br, Cl, F, —CH₂F, —CHF₂, —CF₃, —OCF₃, —CN, -alkyl-CN, —CONH₂, —CONHR⁶, —CONR⁶R⁶, —COOH, —NH₂, —NHR⁶, —NO₂, —NR⁶R⁶, —N₃, —OH, OR⁶, —COOR⁶, —OSO₃R⁶, oxo, R⁶, —SH, —SO₂R⁶, —SO₃H, —SO₃R⁶, or —SR⁶;
m is 0, 1, or 2; and
wherein the compound is not N,N′-(1-phenyl-1H-1,2,4-triazole-3,5-diyl)dibenzamide, N-(3-benzamido-1-phenyl-1H-1,2,4-triazol-5-yl)furan-2-carboxamide, N-(5-cinnamamido-1-phenyl-1H-1,2,4-triazol-3-yl)benzamide, N-(1-phcnyl-5-(phcnylamino)-1H-1,2,4-triazol-3-yl)benzamide, 4-fluoro-N-(5-(4-mcthoxybenzamido)-1-phenyl-1H-1,2,4-triazol-3-yl)benzamide, N,N′-(1-phenyl-1H-1,2,4-triazole-3,5-diyl)bis(4-methylbenzamide), N-(5-((2-chlorobenzyl)amino)-1-phenyl-1H-1.2,4-triazol-3-yl)-2-fluorobenzamide, N-(3-benzamido-1-phenyl-1H-1,2,4-triazol-5-yl)-4-fluorobenzamide, N-(3-benzamido-1-phenyl-1H-1,2,4-triazol-5-yl)-4-nitrobenzamide, N-(3-benzamido-1-phenyl-1H-1,2,4-triazol-5-yl)-3-nitrobenzamide, and 4-((3-benzamido-1-phenyl-1H-1,2,4-triazol-5-yl)carbamoyl)benzoic acid.

140. The method of claim 139, wherein the Parkin ligase activator is a compound of formula (IIIA): 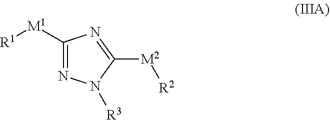
or a pharmaceutically acceptable salt or solvate thereof, wherein:
M¹ and M² are each independently selected from —NR⁴—, —NR⁴C(O)— or —C(O)NR⁴—, provided that M¹ and M² are not both —NR⁴—;
R¹ and R² are each independently phenyl, optionally substituted with one or more R⁵;
wherein at least one of R¹ or R² is substituted with —(C₁-C₆ alkylene)NHCO(C₁-C₁₀ alkyl) or —(C₁-C₆ alkylene)N(C₁-C₃ alkyl)CO(C₁-C₁₀ alkyl);
R³ is 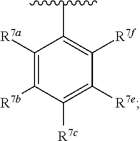
R⁴ is each independently H or C₁-C₃ alkyl;
R⁵ is each independently I, Br, Cl, F, —CH₂F, —CHF₂, —CF₃, —C₁-C₆ alkyl, alkynyl, —CN, —(C₁-C₃ alkylene)-CN, —NH₂, —NO₂, —N₃, —OH, —OCF₃, —OMe, —NMe₂, —NEt₂, —C(O)O(C₁-C₆ alkyl), —CO(C₁-C₁₀ alkyl), —NHCO(C₁-C₁₀ alkyl), —N(C₁-C₃ alkyl)CO(C₁-C₁₀ alkyl), —(C₁-C₆ alkylene)NHCO(C₁-C₁₀ alkyl), or —(C₁-C₆ alkylene)N(C₁-C₃ alkyl)CO(C₁-C₁₀ alkyl);
R^{7a}, R^{7b}, R^{7e}, and R^{7f} is each independently H, I, Br, Cl, F, —CH₂F, —CHF₂, —CF₃, —OCF₃, —N₃, —CN, —OH, methyl, ethyl, propyl, C₁-C₃ haloalkyl, C₁-C₃ alkoxy, or C₁-C₃ haloalkoxy; and
R^{7c} is H, I, Br, Cl, F, —CH₂F, —CHF₂, —CF₃, —OCF₃, —N₃, —CN, —OH, C₁-C₃ alkyl, C₁-C₃ haloalkyl, C₁-C₃ alkoxy, C₁-C₃ haloalkoxy, 4-6 membered heterocyclyl, or 5-6 membered heteroaryl, wherein the heterocyclyl and heteroaryl is optionally substituted with one or more R⁵.

141. The method of claim 139, wherein the Parkin ligase activator is a compound of formula (IIIB): 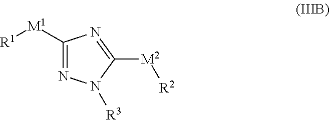
or a pharmaceutically acceptable salt or solvate thereof, wherein:
M¹ and M² are each independently selected from —NR⁴—, —NR⁴C(O)— or —C(O)NR⁴—, provided that M¹ and M² are not both —NR⁴—;
R¹ and R² are each independently phenyl, optionally substituted with one or more R⁵;
R³ is phenyl, substituted with one or more R⁷;
R⁴ is each independently H or C₁-C₃ alkyl;
R⁵ is each independently I, Br, Cl, F, —CH₂F, —CHF₂, —CF₃, —C₁-C₆ alkyl, alkynyl, —CN, —(C₁-C₃ alkylene)-CN, —NH₂, —NO₂, —N₃, —OH, —OCF₃, —OMe, —NMe₂, —NEt₂, —C(O)O(C₁-C₆ alkyl), —CO(C₁-C₁₀ alkyl), —NHCO(C₁-C₁₀ alkyl), —N(C₁-C₃ alkyl)CO(C₁-C₁₀ alkyl), —(C₁-C₆ alkylene)NHCO(C₁-C₁₀ alkyl), or —(C₁-C₆ alkylene)N(C₁-C₃ alkyl)CO(C₁-C₁₀ alkyl); and
wherein at least one R⁷ is heterocyclyl substituted with —CO(C₁-C₁₀ alkyl), which is optionally further substituted with one or more R⁵.

142. The method of claim 139, wherein the Parkin ligase activator is a compound of formula (IIIC): 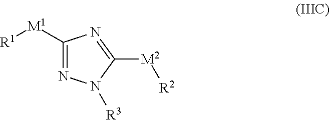
or a pharmaceutically acceptable salt or solvate thereof, wherein:
M¹ and M² are each independently selected from —NR⁴C(O)— or —C(O)NR⁴—;
R¹ and R² are each independently phenyl optionally substituted with one or more R⁵;
R³ is 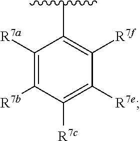
R⁴ is each independently H or C₁-C₃ alkyl; and
R^{7a}, R^{7b}, R^{7e}, and R^{7f} is each independently H, I, Br, Cl, F, —CH₂F, —CHF₂, —CF₃, —OCF₃, —N₃, —CN, —OH, methyl, ethyl, propyl, C₁-C₃ haloalkyl, C₁-C₃ alkoxy, or C₁-C₃ haloalkoxy;
R^{7c} is H, I, Br, Cl, F, —CH₂F, —CHF₂, —CF₃, —OCF₃, —N₃, —CN, —OH, —C₁-C₃ alkyl, —C₁-C₃ haloalkyl, —C₁-C₃ alkoxy, —C₁-C₃ haloalkoxy, 4-6 membered heterocyclyl, or 5-6 membered heteroaryl, wherein the heterocyclyl and heteroaryl is optionally substituted with one or more R⁵;
R⁵ is I, Br, Cl, F, —CH₂F, —CHF₂, —CF₃, —C₁-C₆ alkyl, alkynyl, —CN, —(C₁-C₃ alkylene)-CN, —NH₂, —(C₁-C₃ alkylene)-NH₂, —(C₁-C₃ alkylene)-NHR⁶, —(C₁-C₃ alkylene)-NR⁶R⁶, —NO₂, —N₃, —OH, —OCF₃, —OMe, —NMe₂, —NEt₂, —C(O)O(C₁-C₆ alkyl), 4-6 membered heterocyclyl, or —(C₁-C₃ alkylene)-(4-6 membered heterocyclyl), wherein heterocyclyl is optionally substituted with one or more R⁸;
R⁶ is —C₁-C₃ alkyl;
R⁸ is I, Br, Cl, F, —CH₂F, —CHF₂, —CF₃, or —C₁-C₆ alkyl; and
wherein at least one of R^{7a}, R^{7b}, R^{7c}, R^{7e}, and R^{7f} is not H.

143. The method of claim 139, wherein the Parkin ligase activator is a compound of formula (IIID): 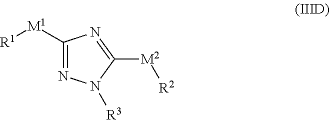
or a pharmaceutically acceptable salt or solvate thereof, wherein:
M¹ and M² are each independently selected from —NR⁴C(O)— or —C(O)NR⁴—;
R¹ is phenyl optionally substituted with one R^{5a};
R² is phenyl optionally substituted with one R^{5b};
R³ is 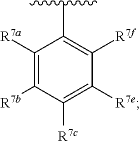
R⁴ is each independently H or C₁-C₃ alkyl; and
R^{7a}, R^{7b}, R^{7e}, and R^{7f} is each independently H, I, Br, Cl, F, —CH₂F, —CHF₂, —CF₃, —OCF₃, —N₃, —CN, —OH, methyl, ethyl, propyl, C₁-C₃ haloalkyl, C₁-C₃ alkoxy, or C₁-C₃ haloalkoxy;
R^{7c} is H, I, Br, Cl, F, —CH₂F, —CHF₂, —CF₃, —OCF₃, —N₃, —CN, —OH, —C₁-C₃ alkyl, —C₁-C₃ haloalkyl, —C₁-C₃ alkoxy, —C₁-C₃ haloalkoxy, 4-6 membered heterocyclyl, or 5-6 membered heteroaryl, wherein the heterocyclyl and heteroaryl is optionally substituted with one R^{5b};
R^{5a} is —(C₁-C₃ alkylene)-(4-6 membered heterocyclyl), wherein heterocyclyl is optionally substituted with one R⁸;
R^{5b} is I, Br, Cl, F, —CH₂F, —CHF₂, —CF₃, —C₁-C₆ alkyl, alkynyl, —CN, —(C₁-C₃ alkylene)-CN, —NH₂, —(C₁-C₃ alkylene)-NH₂, —(C₁-C₃ alkylene)-NHR⁶, —(C₁-C₃ alkylene)-NR⁶R⁶, —NO₂, —N₃, —OH, —OCF₃, —OMe, —NMe₂,—NEt₂, or —C(O)O(C₁-C₆ alkyl);
R⁶ is —C₁-C₃ alkyl;
R⁸ is —C₁-C₃ alkyl; and
wherein at least one of R^{7a}, R^{7b}, R^{7c}, R^{7e}, and R^{7f} is not H.

144. The method of claim 139, wherein the Parkin ligase activator is a compound of formula (IIIE): 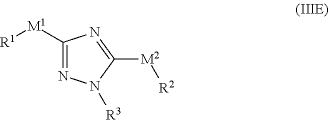
or a pharmaceutically acceptable salt or solvate thereof, wherein:
M¹ and M² are each independently selected from —NR⁴C(O)— or —C(O)NR⁴—;
R¹ is phenyl optionally substituted with —(C₁-C₃ alkylene)-(5-6 membered heterocyclyl), wherein heterocyclyl is optionally substituted with one R⁸;
R² is phenyl optionally substituted with one R^{5b};
R³ is 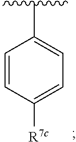
R⁴ is each independently H or C₁-C₃ alkyl;
R^{7a}, R^{7b}, R^{7e}, and R^{7f} is each independently H, I, Br, Cl, F, —CH₂F, —CHF₂, —CF₃, —OCF₃, —N₃, —CN, —OH, methyl, ethyl, propyl, C₁-C₃ haloalkyl, C₁-C₃ alkoxy, or C₁-C₃ haloalkoxy;
R^{7c} is H, I, Br, Cl, F, —CH₂F, —CHF₂, —CF₃, —OCF₃, —N₃, —CN, —OH, —C₁-C₃ alkyl, —C₁-C₃ haloalkyl, —C₁-C₃ alkoxy, —C₁-C₃ haloalkoxy, 4-6 membered heterocyclyl, or 5-6 membered heteroaryl, wherein the heterocyclyl and heteroaryl is optionally substituted with one R^{5b};
R^{5b} is I, Br, Cl, F, —CH₂F, —CHF₂, —CF₃, —C₁-C₆ alkyl, alkynyl, —CN, —(C₁-C₃ alkylene)-CN, —NH₂, —(C₁-C₃ alkylene)-NH₂, —(C₁-C₃ alkylene)-NHR⁶, —(C₁-C₃ alkylene)-NR⁶R⁶, —NO₂, —N₃, —OH, —OCF₃, —OMe, —NMe₂,—NEt₂, or —C(O)O(C₁-C₆ alkyl);
R⁶ is —C₁-C₃ alkyl; and
R⁸ is —C₁-C₃ alkyl.

145. The method of any one of claims 1-98, wherein the Parkin ligase activator is selected from Tables 1, 1A, 2, 3, 3A, 3B, and/or 3C.

146. The method of any one of claims 1-98, wherein the Parkin ligase activator is selected from 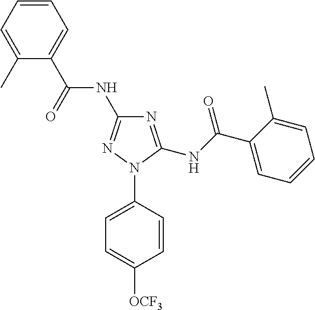 [SMILES: Cc1ccccc1C(=O)Nc1nc(NC(=O)c2ccccc2C)n(-c2ccc(OC(F)(F)F)cc2)n1]
or a pharmaceutically acceptable salt thereof.

147. The method of any one of claims 1-98, wherein the Parkin ligase activator is selected from 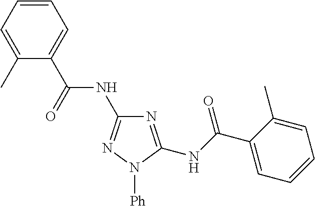 [SMILES: Cc1ccccc1C(=O)Nc1nc(NC(=O)c2ccccc2C)n(-c2ccccc2)n1]
or a pharmaceutically acceptable salt thereof.
